(19) United States
(12) Patent Application Publication
(10) Pub. No.: US 2026/0036590 A1
(43) Pub. Date: Feb. 5, 2026

(54) MULTIVALENT PROTEINS AND SCREENING METHODS

(71) Applicant: Valink Therapeutics Ltd, London (GB)

(72) Inventors: Irsyad KHAIRIL, London (GB); Arne Hagen August SCHEU, London (GB); Benjamin DORGAN, London (GB); Georgi NIKOV, London (GB); Ricarda Melina HOFFMANN, London (GB); Reto BREM, London (GB); Jack WILSON, London (GB); Oswaldo Javier Aguay CHAVEZ, London (GB)

(73) Assignee: Valink Therapeutics Ltd, London (GB)

(21) Appl. No.: 19/115,922

(22) PCT Filed: Sep. 28, 2023
(86) PCT No.: PCT/GB2023/052517

§ 371 Date: Mar. 27, 2025

(30) Foreign Application Priority Data
Sep. 28, 2022 (GB) 2214235.0
Jun. 16, 2023 (GB) 2309124.2

(51) Int. Cl. G01N 33/68; C07K 14/435

(52) U.S. Cl. CPC G01N 33/6845; C07K 14/435

(57) ABSTRACT

Provided herein are multivalent protein scaffolds useful as therapeutics, and useful in identifying new therapeutic compounds. The invention also relates to multi-domain polypeptide constructs having multiple binding domains and a structural domain. Also provided herein are methods of using the provided multivalent protein scaffolds to identify new candidate therapeutics, and new therapeutics thereby identified.

### FIELD

The present invention relates to multivalent protein scaffolds and their use as a modular system for phenotypic screening of combinations of target molecules and as therapeutics. The invention also relates to multi-domain polypeptide constructs comprising multiple binding domains and a structural domain. The invention also relates to methods of identifying new therapeutics using the described protein scaffolds and to therapeutics that can be identified in this manner.

### BACKGROUND

There is an ongoing need to identify new therapeutics for many pathological conditions.

Protein-based therapies have offered an attractive approach to address many common diseases. Such therapeutics have proven to have high clinical success and many protein therapeutics have been approved by regulators for clinical use around the world.

Protein-based therapeutics can operate in various ways: for example, by replacing a protein that is deficient or abnormal; by augmenting existing pathways; by providing novel functions or activities with therapeutic utility; by interfering with a molecule or organism; and by delivering other compounds or proteins, such as a radionuclide, cytotoxic drug, or effector proteins. Therapeutic proteins can be grouped based on their physical and structural properties, and can for example be divided into antibody-based drugs, Fc fusion proteins, anticoagulants, blood factors, bone morphogenetic proteins, engineered protein scaffolds, enzymes, growth factors, hormones, interferons, interleukins, thrombolytics, and the like. Therapeutic proteins can also be classified based on their molecular mechanism of activity. For example, monoclonal antibodies typically operate by binding non-covalently to targets. Enzymes may affect covalent bonds in targets. Other proteins may exert activity without any specific interaction, e.g. serum albumin.

One class of protein-based therapeutics that has enjoyed clinical success are therapeutic antibodies. Antibodies, also known as immunoglobulins (Ig), have been assessed as potential treatments for many disease conditions. For example, monoclonal antibody therapy has been employed to treat diseases including rheumatoid arthritis, multiple sclerosis, psoriasis, and various forms of cancer. Marketed antibody therapeutics include muromomab, abciximab, rituximab, daclizumab, basiliximab, palivizumab, infliximab, trastuzumab, etanercept, gemtuzumab, alemtuzumab, ibritomomab, adalimumab, alefacept, omalizumab, tositumomab, efalizumab, cetuximab, bevacizumab, natalizumab, ranibizumab, panitumumab, eculizumab, and certolizumab.

Antibodies typically comprise four polypeptide chains, forming an Fc region and two antigen-binding (Fab) regions. Each Fab region contains variable regions (Fv) that form the paratope and contact the antigen. Naturally occurring antibodies typically display symmetrical binding at the variable regions. However, this limitation means that typically only a single receptor type (or other target) can be targeted by a given antibody at the variable regions.

To seek to address this, much interest recently has turned to bispecific antibodies. Bispecific antibodies differ from conventional monospecific antibodies in that each of the two Fab sites binds to two different antigens. Bispecific antibodies are often classed as being either Ig-like or non-Ig-like, the latter of which may consist of chemically linked Fab regions.

Bispecific antibodies are being actively researched for clinical use. Two examples of marketed bispecific antibodies include Blinatumomab, sold under the brand name Blincyto and which comprises both a CD3 site for targeting T cells and a CD19 site for targeting B cells, with utility in treating Philadelphia chromosome-negative relapsed or refractory acute lymphoblastic leukemia; and Emicizumab (sold under brand name Hemlibra), which targets both clotting factors IXa and X, and which is used in the treatment of hemophilia A. Bispecific antibodies are commonly used to bind to multiple cell types at the same time, for example, by simultaneously binding tumor cell receptors and recruiting cytotoxic immune cells.

Despite the promise offered by some bispecific antibodies, problems remain. Antibody therapeutics are associated with high production costs, not least due to their size and complex post-translational modification chemistry, including complex glycosylation patterns. Antibody production necessitates the use of very large cultures of mammalian cells followed by extensive purification steps, leading to extremely high production costs and limiting the wide use of these drugs. Antibodies have also been associated with poor tumor targeting, limiting their use in treating cancer (for example, studies have shown that in murine xenograft models less than 20% of administered antibody typically interacts with the tumour). The Fc portion of antibodies, for example IgG antibodies, can interact with various receptors expressed at the surface of several cell types, which increase their retention in the circulation. Their large size can also lead to slow diffusion in vivo. IgG-like antibodies can be immunogenic, leading to detrimental downstream immune reactions via Fc-receptor activation. Regarding bispecific antibodies specifically, the Ig-like approach of “knobs into holes” that has been described previously is not readily adaptable for screening large numbers of antigen-binding domains, due to a lack of modularity. Furthermore, the bispecific antibody approach is practically limited to screening of Fv/Fab regions, and thus is not used to investigate the therapeutic potential of other, non-immunoglobulin protein domains. The non-Ig-like approach of tandem fusions is more adaptable, but not readily scalable.

Blanco-Toribio et al (MAbs. 2013 Jan. 1; 5 (1): 70-79) describes the generation and characterization of monospecific and bispecific hexavalent trimerbodies. The molecules, termed “trimerbodies,” use a modified version of the N-terminal trimerization region of human collagen XVIII noncollagenous 1 (NC1) domain flanked by two flexible linkers as trimerizing scaffold. By fusing single-chain variable fragments (scFv) with the same or different specificity to both N- and C-terminus of the trimerizing scaffold domain, the authors produced monospecific or bispecific hexavalent molecules that were efficiently secreted as soluble proteins by transfected mammalian cells. A bispecific anti-laminin×anti-CD3 N-/C-trimerbody was found to be trimeric in solution. One drawback of this method is that the required use of transfection is not always a convenient production method.

WO-A-2020/0188346 describes bispecific antigen-binding proteins wherein two antigen-binding domains (“ABD”) are covalently bound to a fusion protein formed of two or more domains that form an isopeptide linkage with the antigen-binding proteins. The isopeptide-linkage forming domains are typically catcher domains such as Spycatcher (“SC”), and the resulting bispecific proteins are in the format ABD-SC-SC-ABD.

Brune et al 2017 (Bioconjugate Chem. 2017, 28, 5, 1544-1551) describes a plug-and-display synthetic assembly using orthogonal reactive proteins for twin antigen immunization. The authors prepared a dually addressable synthetic nanoparticle by engineering the multimerizing coiled-coil IMX313 and two orthogonally reactive split proteins. The construct is in the format SpyCatcher-IMX-SnoopCatcher and provides a modular platform, whereby SpyTag-antigen and SnoopTag-antigen can be multimerized on opposite faces of the particle simply upon mixing.

Accordingly, there is a need for a new paradigm to allow rapid, scalable and adaptable identification and design of new protein therapeutics which overcome some or all of these problems. In particular, there is a need for new platform technologies to rival traditional antibody platforms.

### SUMMARY OF THE INVENTION

The present inventors have recognized the issues described above. It has now been recognized that non-antibody assembly platforms can be used to provide a customizable, reproducible, scalable and adaptable scaffold for screening, identifying and developing novel therapeutics. The approach described herein allows for multiple different protein geometries, valencies and/or functionalities to be assessed for potential therapeutic benefit.

Part of the inventors' approach was to develop protein constructs with favourable properties. These constructs can be prepared recombinantly, by expression as a fusion protein, or the component domains can be joined by other means known in the art such as chemical conjugation. In particular, the inventors identified that a polypeptide can advantageously be modified at both N and C termini to provide a polypeptide with two modified termini. The modifications are typically the addition of polypeptide domains that are each able to bind to a target molecule, for example an antigen-binding region or an isopeptide bond-forming region. A different target molecule may be bound by the N and C terminus, to provide a so-called bispecific binding construct. The resulting protein construct is able to bind to the target molecule at the modified N terminus and at the modified C terminus. The inventors have, in particular, engineered protein constructs wherein the N and C termini have the same general orientation, and the resulting construct is able to bind to the binding partner of each terminus when the binding partners are in the same general space and orientation, for example when bound to a solid surface such as a plate or bead, or on the surface of a cell. This may be referred to as providing modified N and C termini of a single polypeptide chain in a “cis” orientation. Typically, a cis-oriented bispecific construct is provided. Two of more of these protein constructs can combine to form an oligomeric protein.

These protein constructs allow for the creation of a combinatorial system that can be used to screen for useful combinations of effector moieties such as binding regions (for example antigen binding regions). Furthermore, once a useful combination has been identified, the construct can be modified to remove (or replace e.g. with a linker) the features necessary for the combinatorial screen, thereby providing a simpler protein construct with the identified favourable combination of binding regions. These constructs may find particular utility as therapeutic, diagnostic or analytical agents, as monomers or as oligomers of more than one construct.

Accordingly, provided herein is a multivalent protein scaffold comprising:
an oligomeric core comprising a plurality of subunit monomers; and
at least two first binding sites orthogonal to at least two second binding sites;
   wherein said first binding sites and said second binding sites are positioned on the same face of the scaffold.

Also provided is a multivalent protein scaffold comprising:
an oligomeric core comprising a plurality of subunit monomers;
at least one first binding site orthogonal to at least one second binding site;
   wherein said first binding site(s) and said second binding site(s) are positioned on the same face of the scaffold; and
wherein said first binding site comprises a first protein domain capable of forming a covalent bond to a first polypeptide target; and said second binding site comprises a second protein domain capable of forming a covalent bond to a second polypeptide target.

Further provided is a multivalent protein scaffold comprising:
an oligomeric core comprising a plurality of subunit monomers;
at least one first binding site orthogonal to at least one second binding site;
   wherein said first binding site(s) and said second binding site(s) are positioned on the same face of the scaffold; and
wherein said oligomeric core does not comprise an Fc region of an antibody.

Preferably, the oligomeric core comprises at least three subunit monomers. More preferably, the oligomeric core comprises from 3 to 6 subunit monomers.

Preferably, in one embodiment the subunit monomers are non-covalently attached together. Preferably, in another embodiment the subunit monomers are covalently attached together. Preferably, when the subunit monomers are covalently attached together, the subunit monomers are genetically fused together. In some embodiments, the subunit monomers are expressed as a single polypeptide chain from a recombinant nucleic acid.

In one embodiment the oligomeric core is preferably a homooligomeric core. In such embodiments, preferably each monomer in the oligomeric core comprises at least one first binding site and at least one second binding site, and wherein the at least one first binding site is orthogonal to the at least one second binding site. Preferably, in one aspect each monomer comprises a first binding site attached at a first terminus of said monomer and a second binding site at a second terminus of said monomer. Preferably the first terminus and the second terminus of each monomer are positioned on the same face of said monomer. Preferably, in another aspect, each monomer comprises a first binding site attached at a first terminus of said monomer and a second binding site attached to said first binding site.

In another embodiment the oligomeric core is preferably a hetero-oligomeric core. Preferably, in such embodiments said core comprises at least one first subunit monomer comprising a first binding site, and at least one second subunit monomer comprising a second binding site, and wherein the first binding site is orthogonal to the second binding site.

Preferably, in the invention, the protein scaffold provided herein typically is such that each subunit monomer comprises less than 300 amino acids; preferably less than 200 amino acids; more preferably less than 150 amino acids. Preferably, the oligomeric core has a molecular weight of less than about 150 kDa, preferably less than about 100 kDa; more preferably less than about 70 kDa.

In some embodiments, the oligomeric core does not comprise an Fc region of an antibody. In some embodiments, the oligomeric core does not comprise a CH2 domain. In some embodiments, the oligomeric core does not comprise a CH3 domain. In some embodiments, the oligomeric core does not comprise a CH2 domain and does not comprise a CH3 domain.

Preferably, the oligomeric core and/or the scaffold does not generate an immune response when administered to a human subject. This is described in more detail herein.

In some embodiments, the oligomer core and/or scaffold, or the structural domain, does not generate a deleterious immune response when administered to a human subject. For example, an active B cell or T cell response is not raised against the structural domain, and/or the structural domain does not specifically bind to immunoglobulin receptors or activate antibody-dependent cell medicated toxicity (ADCC).

Preferably, the oligomeric core comprises a soluble multimerising structural element of a multimeric protein. Preferably, the multimeric protein comprises a collagen NC (noncollagenous) domain (e.g. an NC1 domain), a CutA1, a C1q head domain, a TNF, a p53, a fibrinogen, a C4, Bacillus subtillus AbrB or a homolog or paralog thereof.

Preferably, the multimeric protein comprises a Collagen VIII NC1 (noncollagenous) domain, a Collagen X NC1 (noncollagenous) domain, a C1q head domain, a CutA1 protein, a Macrophage Migration Inhibitory Factor (MIF) or Macrophage Migration Inhibitory Factor 2 (MIF-2), a Tumor Necrosis Factor (TNF), a TNF family member including TL1A, CD40L or OX40L, or a homolog or paralog thereof.

Preferably, the multimerising structural element comprises a polypeptide have at least 30% or at least 50% amino acid identity to SEQ ID NO: 1, SEQ ID NO: 2, SEQ ID NO: 3, SEQ ID NO: 29, SEQ ID NO: 60, SEQ ID NO: 25, SEQ ID NO: 26, SEQ ID NO: 27, SEQ ID NO: 42, SEQ ID NO: 31, SEQ ID NO: 58, SEQ ID NO: 78, SEQ ID NO: 80 or SEQ ID NO: 19.

The inventors have, in particular, identified CutA1, typically human CutA1, as a favourable structural element. In some embodiments, a CutA1 protein is engineered to comprise one or more substitutions or deletions relative to wild-type CutA1. The CutA1 domain, optionally as engineered, finds particular utility as a scaffold. In some embodiments, therefore, the CutA1 domain is connected by a peptide linkage as a fusion protein to a different polypeptide domain at the N-terminus and/or the C terminus of the CutA1.

In certain embodiments, the multimerising structural element is human CutA1 (SEQ ID NO: 19). In certain embodiments, the multimerising structural element is a truncated form of SEQ ID NO: 19 (human CutA1), as described elsewhere herein. In certain embodiments, the multimerising structural element is a modified form of SEQ ID NO: 19 (human CutA1) wherein at least one cysteine residue in SEQ ID NO: 19 is substituted for a different amino acid residue, as described elsewhere herein. In certain embodiments, the multimerising structural element is a modified and truncated form of SEQ ID NO: 19 (human CutA1) wherein at least one N and/or C terminus amino acid residue is removed and wherein at least one cysteine residue in SEQ ID NO: 19 is substituted for a different amino acid residue, as described elsewhere herein.

In certain embodiments, the multimerising structural element is a cytokine. In certain embodiments, the multimerising structural element belongs to the TNF superfamily. In certain embodiments, the multimerising structural element is TNF (SEQ ID NO: 80) or OX40L (SEQ ID NO: 78) or CD40L (SEQ ID NO: 58) or TL1A (SEQ ID NO: 31) or is derived from (e.g. is a truncated and/or modified form of) TNF or OX40L or CD40L or TL1A, as described elsewhere herein. Accordingly, the multimerising structural element may comprise or consist of a polypeptide having at least 30%, such as at least 50%, at least 60%, at least 70%, at least 80%, at least 90%, at least 95%, at least 97%, at least 98%, at least 99% or 100%, amino acid identity to the amino acid sequence of human CutA1 (SEQ ID NO: 19), OX40L (SEQ ID NO: 78), CD40L (SEQ ID NO: 58), TL1A (SED ID NO: 31), or TNF (SEQ ID NO: 80).

In certain embodiments, the multimerising structural element is a modified and truncated form of SEQ ID NO: 78, SEQ ID NO: 58, SEQ ID NO: 80 or SEQ ID NO: 31, wherein at least one N and/or C terminus amino acid residue is removed and wherein at least one cysteine residue is substituted for a different amino acid residue, as described elsewhere herein.

CutA1 is structurally similar to the non-human family of PII proteins (Bagautdinov et al., Acta Crystallogr Sect F Struct Biol Cryst Commun. 2008 May 1; 64 (Pt 5): 351-7). In some embodiments, the structural element comprises or consists of a protein from the PII protein family, or a truncated or modified version thereof. PII proteins include, for example signal transduction protein PII GlnB (SEQ ID NO: 81) from Synechococcus elongatus PCC 7942 (Xu et al., Acta Crystallogr D Biol Crystallogr. 2003; 59:2183-2190), GlnK (SEQ ID NO: 82) from Escherichia coli (Xu et al., J Mol Biol. 1998; 282:149-165), the PII-like domains of YqfO (SEQ ID NO: 83) from Bacillus cereus (Godsey et al., Protein Sci. 2007 July; 16:1285-1293) or hypothetical protein SA13888 (SEQ ID NO: 84) from Staphylococcus aureus (Saikatendu et al., BMC Struct Biol. 2006; 6:27), or a truncated or modified version thereof, as described by Lüddecke et al., Sci Rep.

Preferably, the first binding site and/or said second binding site comprises a protein domain. Typically, said first binding site comprises a first protein domain and said second binding site comprises a second protein domain. Preferably, the first binding site and/or second binding site is genetically fused to the subunit monomer(s) to which they are attached to form a single polypeptide chain.

Preferably, the first binding site comprises a first protein domain capable of forming a covalent bond to a first polypeptide target. Preferably, the said second binding site comprises a second protein domain capable of forming a covalent bond to a second polypeptide target. More preferably, the first binding site comprises a first protein domain capable of forming a covalent bond to a first polypeptide target and the said second binding site comprises a second protein domain capable of forming a covalent bond to a second polypeptide target. Preferably said first protein domain is capable of forming an isopeptide bond with said first polypeptide target and said second protein domain is capable of forming an isopeptide bond with said second binding target.

Preferably, said first binding site and said second binding site each comprise a different split ligand-binding protein domain. More preferably, one of said first binding site and said second binding site comprises a split Streptococcus pyogenes fibronectin-binding protein domain and the other of said first binding site and said second binding site comprises a split Streptococcus pneumoniae adhesin domain.

Preferably, said first and said second binding site each independently have at least 50% amino acid identity to any one of SEQ ID NOs: 4-9, 11-13, 23 or 15-18. In some embodiments, said first and said second binding site each independently have at least 60%, at least 70%, at least 80% or at least 90% amino acid identity to any one of SEQ ID NOs: 4-9, 11-13, 23 or 15-18.

Also provided herein is a protein complex comprising a protein scaffold as described herein, wherein the first binding site is bound to a first polypeptide target attached to a first effector moiety, and the second binding site is bound to a second polypeptide target attached to a second effector moiety.

Preferably, in said complex the first binding site/polypeptide target pair and the second binding site/polypeptide target pair are each independently selected from the following combinations: (i) any one of SEQ ID NO: 4, 6 or 8 with any one of SEQ ID NOS: 5, 7 or 9; (ii) SEQ ID NO: 12 with SEQ ID NO: 13 or 15; (iii) SEQ ID NO: 5 with SEQ ID NO: 11; (iv) SEQ ID NO: 15 with SEQ ID NO: 16); (v) SEQ ID NO: 17 with SEQ ID NO: 18; or (vi) SEQ ID NO: 23 with SEQ ID NO: 16).

Also provided is a screening platform comprising a library, wherein said library comprises a plurality of populations of protein complexes as described herein, wherein the populations of protein complexes each comprise a different combination of first effector moieties, second effector moieties and/or oligomeric core.

Also provided is a method for identifying a therapeutic drug or drug analog, the method comprising:
providing a protein complex as described herein;
contacting the protein complex with a biological system; and
measuring whether the protein complex induces a desired change in a property of the biological system;
and optionally further comprising selecting a protein complex that induces a desired change in a property of the biological system.

The method may further comprise synthesizing a therapeutic drug or drug candidate comprising the oligomeric core of the scaffold of the protein complex of the identified therapeutic drug analog attached to the first and second effector moieties of said protein complex.

Also provided is a therapeutic drug candidate obtainable according to the methods described herein.

Also provided is a therapeutic drug obtainable according to the methods described herein.

Also provided is a therapeutic drug or drug candidate comprising or consisting of one or more constructs or polypeptides as described herein.

Also provided herein is a therapeutic drug or drug candidate, comprising an oligomeric core comprising a plurality of subunit monomers attached to one or more first effector moieties and one or more second effector moieties; wherein said one or more first effector moieties and said one or more second effector moieties are positioned on the same face of the oligomeric core; and wherein (i) said one or more first effector moieties comprise two or more first effector moieties and said one or more second effector moieties comprise two or more second effector moieties; and/or (ii) said oligomeric core does not comprise an antibody or antibody fragment. Preferably, the oligomeric core of the therapeutic drug counterpart is as described in more detail herein.

Preferably, in the provided therapeutic drug candidate, the oligomeric core comprises a plurality of subunit monomers and: (i) each subunit monomer comprises a collagen NC1 domain, a CutA1, a C1q domain, a TNF, a p53, a fibrinogen, a C4, Bacillus subtillus AbrB or a homolog or paralog thereof; and/or (ii) each subunit monomer comprises a multimerising structural element comprising a polypeptide having at least 50% amino acid identity to SEQ ID NO: 1, SEQ ID NO: 2, SEQ ID NO: 3 or SEQ ID NO: 19.

Preferably, in the provided therapeutic drug or drug candidate, the oligomeric core comprises a plurality of subunit monomers and: (i) each subunit monomer comprises a Collagen VIII NC1 (noncollagenous) domain, a Collagen X NC1 (noncollagenous) domain, a C1q head domain, a CutA1 protein, a Macrophage Migration Inhibitory Factor (MIF) or Macrophage Migration Inhibitory Factor 2 (MIF-2), a Tumor Necrosis Factor (TNF), a TNF family member including TL1A or CD40L or OX40L or a homolog or paralog thereof; and/or (ii) each subunit monomer comprises a multimerising structural element comprising a polypeptide having at least 50% amino acid identity to SEQ ID NO: 1, SEQ ID NO: 2, SEQ ID NO: 3, SEQ ID NO: 29, SEQ ID NO: 60, SEQ ID NO: 25, SEQ ID NO: 26, SEQ ID NO: 27, SEQ ID NO: 42, SEQ ID NO: 31, SEQ ID NO: 58, SEQ ID NO: 78, SEQ ID NO: 80 or SEQ ID NO: 19.

In certain aspects, the invention provides a polypeptide comprising a first binding domain at the N terminus and/or a second binding domain at the C terminus, wherein the first and second binding domains are separated by a structural domain. The polypeptide is typically a single engineered polypeptide chain, expressed as a fusion protein from a recombinant nucleic acid. Typically, the first binding domain and second binding domain are able to bind to their targets when the target molecules are expressed on a single cell or immobilised onto a plate or single bead. This is sometimes described herein as providing the first and second binding domains in “cis” orientation.

In some aspects, therefore, the first binding domain is at the N terminus of the polypeptide and the second binding domain is at the C terminus, wherein the first and second binding domains are separated by a structural domain. In some aspects, however, the first binding domain is connected to the second binding domain, and the second binding domain is connected to the structural domain. Typically, the second binding domain is connected to the N-terminus or C-terminus of the structural domain. As noted elsewhere herein, the first and second binding domains are typically different but in some embodiments may be the same. A linker may be included between any two domains, or between all domains in a construct, or between any selection of domains in a construct, for example a linker peptide of between 2 and 30 amino acid residues, typically between 5 and 25 amino acid residues.

In some embodiments, the first binding domain and second binding domain are different antigen-binding domains. The construct is then a bispecific construct.

In some embodiments, the first binding domain and/or the second binding domain is a protein or peptide capable of specific binding with a biological molecule. This may be a signalling molecule capable of specific interaction with a binding partner, such as a protein or peptide ligand or a receptor, for example a cytokine or a cell surface receptor.

In other embodiments, the first binding domain and the second binding domain are catcher domains (i.e. split ligand-binding protein domains) that are each able to form an isopeptide linkage with a cognate peptide. These cognate peptides are often referred to as tag peptides, for example a SpyTag forms an isopeptide bond with a SpyCatcher domain as is known in the art and as discussed below. Typically, the cognate peptide for the first binding domain is different from the cognate peptide for the second binding domain. However, in some embodiments the cognate peptide may be the same for both first and second binding domains. The polypeptide with the catcher at each terminus may be provided separately from the molecule (e.g protein) comprising the tag, for example as part of a kit. In some embodiments, each tag peptide is covalently attached to its cognate catcher domain, optionally wherein one or both cognate peptides are linked to the first and/or second catcher domain by an isopeptide bond. In some embodiments, one or both cognate peptide tags are present as a fusion polypeptide with an effector moiety, typically an antigen binding domain. The linkage of the catcher to its cognate peptide tag therefore links the effector moiety (e.g. antigen binding domain) to its binding domain.

In the embodiments where the cognate peptide is the same for both first and second binding domains, then preferential, selective or sequential binding or conjugation can be achieved by temporal or sequential control, spatial control, or spatiotemporal control. This may be, for example, by activation or inactivation of one binding domain and/or by means of competitive binding. Without limitation, activation may for example be achieved by enzymatic cleavage of an inhibiting peptide (as described for example by Driscoll et al, September 2023, bioRxiv 2023.08.31.555700; doi https://doi.org/10.1101/2023.08.31.555700, which is incorporated herein by reference in its entirety) or by light activation, for example using light triggering of covalent bond formation in a photocaged binding domain created by site-specific incorporation of an unnatural residue such as coumarin-lysine at the reactive site (as described for example, with reference to SpyCatcher003, by Rahikainen et al, 2023 “Visible light-induced specific protein reaction delineates early stages of cell adhesion”, bioRxiv 2023.07.21.549850; doi: https://doi.org/10.1101/2023.07.21.549850, which is incorporated herein by reference in its entirety). A further photoactivation approach, using photocaged glutamic acid analogues, is described by Yang et al, Angewendte Chemie Volume 62, Issue 40 Oct. 2, 2023 (published online 16 Aug. 2023) “Photoactivatable Protein with Genetically Encoded Photocaged Glutamic Acid”. Control over the availability of the reactive sites may alternatively be achieved by removal of any inhibiting domain, for example by means of steric hindrance, competition for binding, or blocking of the reactive or catalytic residue.

In some embodiments, two antigen-binding domains each containing the same isopeptide bond-forming tag can both be precisely and selectively conjugated onto a construct of the present invention comprising two of the same isopeptide-bond-forming catcher domains (to which the tag binds), wherein one of the Catcher domains is unreactive until unveiling of reactivity using light activation. Typically, one of the Catcher domains is unreactive due to it being caged by the presence of an unnatural light-reactive residue, for example a coumarin-lysine (7-hydroxycoumarin lysine, “HCK”) amino acid or a photocaged glutamic acid analogue, in the reactive isopeptide-bond-forming site. Once the first tagged-peptide has been attached to the first catcher by formation of the isopeptide bond between first catcher and tag, application of the appropriate light (e.g. 405 nm light) releases the photocaging residue from the second catcher. The second catcher is then available for binding, and the second Tagged peptide can be added.

In some embodiments, two antigen-binding domains each containing the same isopeptide bond-forming tag can both be precisely and selectively conjugated onto a construct of the present invention comprising two of the same isopeptide-bond-forming catcher domains (to which the tag binds), wherein one of the Catcher domains is unreactive until unveiling of reactivity using a site-specific protease. Typically, one of the Catcher domains is unreactive due to the presence of a non-reactive Tag mutant sequence that is fused to the one of the catcher domains via a flexible linker containing a protease cleavage site. Once the first tagged-peptide has been attached to the first catcher by formation of the isopeptide bond between first catcher and tag, addition of the protease releases the non-reactive Tag mutant from the second catcher. The second catcher is then available for binding, and the second Tagged peptide can be added.

In certain embodiments, the non-reactive tag mutant is SpyTag003 D117A (SpyTag003DA) (Keeble et al., 2019 Proc. Natl. Acad. Sci. U.S.A 116, 26523-26533, incorporated herein by reference in its entirely) that is fused to the C-terminus of a second SpyCatcher003 via a flexible linker containing a tobacco etch virus (TEV) protease cleavage site. Following cleavage at the TEV site, the SpyTag003DA peptide will be free to dissociate, unmasking the reactive Lys of SpyCatcher003 to enable reaction with a second supplied SpyTag-linked binder. Cleavage can be carried out with any suitable protease, for example superTEV protease.

In some embodiments, the polypeptide comprises a first binding domain at the N terminus and a second binding domain at the C terminus, wherein the first and second binding domains are separated by a structural domain, the first binding domain and the second binding domain are catcher domains that are each able to form an isopeptide linkage with a cognate peptide, wherein the first catcher domain is linked to its cognate peptide tag by an isopeptide bond and wherein the second catcher domain is linked to its cognate peptide tag by an isopeptide bond. In some embodiments, each peptide tag is attached to an antigen binding domain.

The polypeptide of the previous four paragraphs is useful as a monomer, or combined to form an oligomer.

In some aspects, an oligomer is provided comprising two or more polypeptides as defined above and elsewhere herein.

In some aspects, a polypeptide or oligomer as defined in the preceding paragraphs comprises the features described elsewhere herein. For example, the structural domain of the polypeptide construct is typically the “subunit monomer” as described extensively herein, so definitions and description of the subunit monomer apply to the structural domain. Similarly, the first binding domain and second binding domain are typically the first binding site and second binding site as described elsewhere herein, so definitions and description of the first and second binding sites apply to the first binding domain and second binding domain of the polypeptide construct.

In some particular aspects, the structural domain of the polypeptide construct (or the subunit monomer of the oligomeric core) is a CutA1 protein, typically a human CutA1 protein. The data presented below, in particular FIG. 23, demonstrates various fusions of HsCutA1 (“Homo sapiens CutA1”) with effector proteins, as well as the ability of these direct fusions to exert biological effects. Accordingly, in certain aspects the invention provides a polypeptide comprising a first binding domain at the N terminus and a second binding domain at the C terminus, wherein the first and second binding domains are separated by a human CutA1 structural domain. In certain aspects, the CutA1, typically human CutA1, is engineered to remove one or more cysteine residues from the native sequence (presented herein as SEQ ID NO: 19). Typically, this is a substitution of one or more cysteine residues with one or more non-cysteine residues. The removal of one or more cysteine residues beneficially allows for targeted cysteine conjugation at non-native sites or in fusion proteins. Without being bound by theory, unpaired cysteines routinely interfere with stability and downstream application, so it can be beneficial to remove unpaired cysteines. In some embodiments, it is beneficial to remove one or more unpaired cysteines and reintroduce one or more cysteine residues only at optimised targeted positions.

In some embodiments, one or more cysteine residues in CutA1 are substituted with one or more Alanine residues. In some embodiments, one or more cysteine residues in CutA1 are substituted with one or more Valine residues. In some embodiments, one or more cysteine residues in CutA1 are substituted with one or more Serine residues. In some embodiments, the substitution comprises or consists of two cysteines substituted with two alanines, which is referred to herein as a “CACA” substitution. In some embodiments, the substitution comprises or consists of one cysteine substituted with a valine and one cysteine substituted with a serine, which is referred to herein as a “CVCS” substitution. In some embodiments, the cysteine residues at positions 75 and 96 of wild-type human CutA1 (e.g. SEQ ID NO: 19) are substituted for different residues. In some embodiments, human CutA1 is engineered to substitute two cysteine residues, wherein the cysteine substitutions comprise or consist of (i) C75A, C96A or (ii) C75V, C96S. Accordingly, in some embodiments, the structural domain of the polypeptide construct (or the subunit monomer of the oligomeric core) is a human CutA1 protein engineered to substitute two cysteine residues, wherein the cysteine substitutions comprise or consist of (i) C75A, C96A or (ii) C75V, C96S. The generation and biological effects of polypeptide constructs comprising such cysteine-substituted human CutA1 domains are depicted herein (FIG. 25).

In some embodiments, it is beneficial to remove one or more unpaired cysteines and reintroduce one or more cysteine residues only at optimised targeted positions. The reintroduced cysteines can be useful, for example, as conjugation sites for a drug or dye, such as in the production of a labelled antibody (or antibody-type molecule) or antibody-drug conjugate (“ADC”)-type molecule. Example 13 exemplifies the substitution of non-cysteine residues to cysteine residues, and observes that CutA1 with newly-introduced Cys residues have a higher conjugation efficiency when conjugating a dye or a drug to the CutA1, compared to the wild-type and the negative control (CC041, i.e. CutA1 CACA-engineered to remove native cysteines). The newly-introduced cysteine or cysteines can be substituted into the sequence at a favourable position. As exemplified in Example 13, exemplary positions in human CutA1 include one or more of V64, E78, K79, K82, E83, K91, Q102, K110, E114, F136, S139, F158 and Q166. In some embodiments, human CutA1 (e.g. SEQ ID NO: 19) has cysteine residues substituted-in at 2, 3, 4, 5 or 6 of residues E78, K82, Q102, E114, F136 and Q166.

In some embodiments, the CutA1 is conjugated to an effector molecule, which is any molecule that provides a desired effect. An effector molecule is sometimes referred to as a “payload” in the art, in particular in the context of conjugating an effector molecule to an antibody to form an antibody-drug conjugate or ADC. Accordingly, such payloads, or effector molecules, are well-known in the art. Typically, the effector molecule will comprise or consist of a label, a dye molecule, or a drug i.e a substance that has a therapeutic or prophylactic physiological effect on a human or animal body when consumed by the body. A drug is typically a pharmaceutical drug.

In some embodiments, the effector molecule is a nucleic acid, polynucleotide or oligonucleotide. The nucleic acid, polynucleotide or oligonucleotide may be DNA, RNA, XNA, LNA, or a mixture thereof. Typically it is DNA or RNA. The nucleic acid, polynucleotide or oligonucleotide may be single stranded or double-stranded. In some embodiments, the oligonucleotide comprises or consists of 3 to 50 nucleotides (or pairs of nucleotides when double-stranded), for example 5 to 30 nucleotides (or pairs). Antibody-oligonucleotide conjugates are often known in the art as a subset of ADCs.

In some embodiments, the CutA1 is conjugated to a drug to form a CutA1-drug conjugate. The drug may be of any type, as will be apparent to the skilled person. Drug types that may be conjugated to CutA1 include but are not limited to analgesic, antibiotic, anticancer, anticoagulant, antidepressant, antidiabetic, antiepileptic, antipsychotic, antispasmodic, antiviral, cardiovascular, depressant, sedative, and stimulant drugs.

In some embodiments, two or more different drugs are conjugated to a single CutA1 molecule. Antibody-drug conjugates with dual payloads are described by Yamazaki et al, Nature Communications volume 12, Article number: 3528 (2021), wherein MMAE and MMAF are both conjugated and described for combatting breast tumor heterogeneity and drug resistance.

In some embodiments, the drug is an anticancer drug, for example a cytotoxic drug. In some embodiments, the anticancer drug is a tubulin inhibitor, for example a maytansinoid such as mertansine (also known as DM1), or auristatin, or a taxol derivative. Examples of drugs for inhibiting tubulin polymerization are auristatins such as the tissue factor directed monomethyl auristatin E (MMAE) and monomethyl auristatin F (MMAF), compounds derived from dolastatin 10 (e.g. TZT-1027 as described by Kobayashi et al Jpn J Cancer Res. 1997 March; 88 (3): 316-27.), and tubulysins such as Tubulysin A. In some embodiments, the anticancer drug is a DNA-damaging agent, which causes cell death by cleaving the DNA and/or causing DNA alkylation. Examples of DNA-damaging molecules are duocarmycins, calicheamicins, and pyrrolobenzodiazepines. In some embodiments, the drug is a topoisomerase I inhibitor, which binds to a complex between topoisomerase I and DNA, stabilises it and thereby prevents DNA re-ligation, leading to DNA damage. Examples of drugs inhibiting topoisomerase I are DXd and SN-38. In some embodiments, the drug is an RNA polymerase II inhibitor such as α-amanitin. In some embodiments, the drug is an immunomodulator such as a TLR agonist or a STING agonist (as desired, for example, in Table A of Fu et al, Signal Transduction and Targeted Therapy volume 7, Article number: 93 (2022)).

In some embodiments, the drug is a small molecule drug. Typically, a small molecule drug has a molecular weight of 1000 Da or lower, more typically 750 Da or lower, or 500 Da or less. This molecular weight is of the drug molecule itself and excludes any linker.

In some embodiments the drug is a cytotoxic drug, typically a small molecule cytotoxic drug, for example DXd, mertansine, Monomethyl auristatin E (MMAE), or Monomethyl auristatin F (MMAF). A cytotoxic small molecule drug is often called a chemotherapy or a chemotherapy agent, in particular in the context of cancer treatment.

Conjugation of the drug to CutA1 can be via any known method. Typically, the drug is conjugated to the CutA1 at a cysteine residue, more typically a cysteine residue that is not present in the native sequence. When conjugating to cysteine, the molecule to be conjugated may comprise a maleimide. Cysteine residues react readily with maleimides to form succinimidyl thioether conjugates.

In some embodiments, the drug has a molecular weight of greater than 1000 Da. In some embodiments, the drug is or comprises an oligopeptide or a polypeptide, comprising 2 or more, for example 3, 4, 5, 6, 7, 8 9, 10 or more, or 20 or more, or 50 or more, or 100 or more, amino acid residues covalently linked by peptide bonds. In some embodiments, the drug is an immunotoxin, for example Pseudomonas exotoxin A (PE) which is described as an anticancer agent by Wolf & Beile (Int J Med Microbiol. 2009 March; 299 (3): 161-76. doi: 10.1016/j.ijmm.2008.08.003).

In some embodiments, the CutA1 is conjugated to a dye molecule. In some embodiments, the dye molecule is a small molecule dye. Typically, a small molecule dye has a molecular weight of 1000 Da or lower, more typically 750 Da or lower, or 500 Da or less. In some embodiments the dye is fluorescent, for example fluorescein. Conjugation can be via any known method. Typically, the dye is conjugated to the CutA1 at a cysteine residue, more typically a cysteine residue that is not present in the native sequence. When conjugating to cysteine, the molecule to be conjugated may comprise a maleimide. Cysteine residues react readily with maleimides to form succinimidyl thioether conjugates.

In some embodiments, the effector molecule is a nanoparticle, for example a gold nanoparticle, a silica nanoparticle, a lipid nanoparticle, or a lipid polydopamine hybrid nanoparticle (“LPN”) as described by Yang et al, Acta Pharmaceutica Sinica B Volume 10, Issue 11, November 2020, pages 2212-2226. Typically, the nanoparticle is conjugated to one or more cysteine residues on the CutA1, by site-specific conjugation onto a maleimide group of a lipid nanoparticle or a polydopamine (PDA) hybrid nanoparticle.

In certain aspects, the CutA1, typically human CutA1 (e.g. SEQ ID NO: 19), is engineered to delete one or more residues from either or both termini of the native sequence, to form a truncated CutA1 domain that is incorporated into the construct of the invention. In some embodiments, one or more residues are deleted from the N terminus of CutA1, typically human CutA1. Typically, 5 to 70 residues are deleted from the N terminus of CutA1, typically human CutA1, for example 10 to 59 residues. In some embodiments, 25, 26, 27, 28, 29, 30, 31, 32, 33, 34, 35, 36, 37, 38, 39, 40, 41, 42, 43, 44, 45, 50, 55, 59, 60, 61, 62, 63, 64, 65 or 66 residues are deleted from the N terminus of CutA1. In some embodiments, the truncated CutA1 begins at residue 30 (i.e 29 N-terminal residues are deleted). In some embodiments, the truncated CutA1 begins at residue 33 (i.e 32 N-terminal residues are deleted). In some embodiments, the truncated CutA1 begins at residue 44 (i.e 43 N-terminal residues are deleted). In some embodiments, the truncated CutA1 begins at residue 60 (i.e 59 N-terminal residues are deleted). In some embodiments, the truncated CutA1 begins at residue 67 (i.e 66 N-terminal residues are deleted), for example the “HsCutA1₆₇₋₁₇₁ CACA” direct fusion construct exemplified in FIG. 23b.

In some embodiments, one or more residues are deleted from the C terminus of CutA1, typically human CutA1. The C-terminal deletion may be instead of, or in addition to, the N-terminal deletion. Typically, 5 to 20 residues are deleted from the C terminus of CutA1, typically human CutA1, for example 6 to 12 residues are deleted. In some embodiments, approximately 8 residues are deleted. In human CutA1 an 8 residue deletion leaves a truncated protein with a C-terminus at what is residue 171 (valine) in the wild-type sequence shown in FIG. 24 and SEQ ID NO: 19. In some embodiments, the truncated protein has a C-terminus at what is residue 168 (threonine) in the wild-type sequence shown in FIG. 24 and SEQ ID NO: 19. In some embodiments, the truncated protein has a C-terminus at what is residue 169, 170, 172, 173, 174, 175 or 176 in the wild-type sequence shown in FIG. 24 and SEQ ID NO: 19.

In some embodiments, the truncated CutA1 starts at any of residues 30 to 67 of SEQ ID NO: 19. In some embodiments, the truncated CutA1 starts at any of residues 44 to 67 of SEQ ID NO: 19. In certain embodiments, the truncated CutA1 consists of residues 44-179 (SEQ ID NO: 29), residues 61-168 of SEQ ID NO: 19 or residues 60-171 of SEQ ID NO: 19, as shown in FIG. 24. In some embodiments, the truncated CutA1 starts at any of residues 44 to 67 of SEQ ID NO: 19 and ends at any of residues 168 to 179. In other embodiments, the truncated CutA1 starts at any of residues 30 to 65 of SEQ ID NO: 19 and ends at any of residues 165 to 179. In some embodiments, the truncated CutA1 starts at any of residues 44 to 67 of SEQ ID NO: 19 and ends at any of residues 171 to 179.

The truncation of CutA1 can improve the precision by which fusion constructs can be constructed. FIG. 24 demonstrates the generation, yield and biological effects of polypeptide constructs comprising such truncated human CutA1 domains.

In certain aspects, the CutA1 is a human CutA1 that is engineered to remove one or more cysteine residues from the native sequence and that is truncated at the N and/or C termini. In some embodiments, the truncated CutA1 consists of residues 44-179 or residues 60-171 of SEQ ID NO: 19, as shown in FIG. 24, and has cysteine substitutions that comprise or consist of (i) C75A, C96A or (ii) C75V, C96S. In other embodiments, the truncated CutA1 starts at any of residues 30 to 65 of SEQ ID NO: 19 and end at any of residues 165 to 179, and also has cysteine substitutions that comprise or consist of (i) C75A, C96A or (ii) C75V, C96S.

In certain aspects, the CutA1 is a human CutA1 that is engineered to remove one or more cysteine residues from the native sequence, is truncated at the N and/or C termini, and has at least one non-cysteine residue substituted to a cysteine residue. In some embodiments, the truncated CutA1 consists of residues 44-179 of human CutA1 (SEQ ID NO:19), has cysteine residues substituted out of the native CutA1 sequence that comprise or consist of (i) C75A, C96A or (ii) C75V, C96S, and has at least one cysteine present at a residue position that is not a cysteine residue in the native sequence.

In some embodiments, the truncated CutA1 consists of residues 44-179 of human CutA1 (SEQ ID NO:19), has cysteine substitutions-out that comprise or consist of C75A, C96A, and has a cysteine residue substituted-in at one, two or three of residues K82, E114 and F136.

In some embodiments, the truncated CutA1 consists of residues 44-179 of human CutA1 (SEQ ID NO:19), has cysteine substitutions-out that comprise or consist of C75A, C96A, and has a cysteine residue substituted-in at one or more of residues V64, E78, K79, E83, K91, Q102, K110, S139, F158 and Q166. In some embodiments, this variant CutA1 has cysteine residues substituted-in at 2, 3, 4, 5, 6, 7, 8, 9 or 10 of residues V64, E78, K79, E83, K91, Q102, K110, S139, F158 and Q166.

In some embodiments, the truncated CutA1 consists of residues 44-179 of human CutA1 (SEQ ID NO:19), has cysteine substitutions-out that comprise or consist of C75A, C96A, and has a cysteine residue substituted-in at one or more of residues E78, Q102 and Q166. In some embodiments, this variant CutA1 has cysteine residues substituted-in at two or three of residues E78, Q102 and Q166.

In some embodiments, the truncated CutA1 consists of residues 44-179 of human CutA1 (SEQ ID NO:19), has cysteine substitutions-out that comprise or consist of C75A, C96A, and has a cysteine residue substituted-in at one or more of residues E78, K82, Q102, E114, F136 and Q166. In some embodiments, this variant CutA1 has cysteine residues substituted-in at 2, 3, 4, 5 or 6 of residues E78, K82, Q102, E114, F136 and Q166.

In some embodiments, the variant CutA1 comprises or consists of a sequence as defined above, or any of the specific sequences described in the Examples, and comprises between 1 and 10 amino acid substitutions at residues that are not specified as being a cysteine residue substitution-in or substitution-out. For example, in some embodiments variant human CutA1 sequences can be provided that contain 1, 2, 3, 4, 5, 6, 7, 8, 9 or 10 amino acid substitutions at positions other than 95, 64, 76, 78, 79, 82, 83, 91, 95, 102, 110, 114, 136, 139, 158 and 166. These 1 to 10 substitutions can be to any of the standard 20 amino acids, although typically they will not replace a non-cysteine residue with cysteine. Typically, these 1 to 10 substitutions will be conservative substitutions. In some embodiments, one, two, three or more of these 1-10 substitutions may introduce an unnatural amino acid (UAA), also known as a non-proteinogenic amino acid. Unnatural amino acids can be useful to enable conjugation of a molecule of interest via biorthogonal chemistry, for instance via click-chemistry. Unnatural amino acids are known in the art and include D-amino acids, homo amino acids, N-methyl amino acids, hydroxyproline (Hyp), beta-alanine, citrulline (Cit), ornithine (Orn), norleucine (Nle), 3-nitrotyrosine, nitroarginine, and pyroglutamic acid (Pyr). For example, propargyllysine is an unnatural amino acid which, when incorporated into a protein, can be exploited to attach commercially available fluorescent azide dyes through copper-catalyzed alkyne-azide cycloaddition click reaction (also known as click reaction). Other UAAs suitable for site-specific modification of a polypeptide sequence include 1:3-(6-acetylnaphthalen-2-ylamino)-2-aminopropanoic acid (Anap), 2: (S)-1-carboxy-3-(7-hydroxy-2-oxo-2H-chromen-4-yl) propan-1-aminium (CouAA), 3:3-(5-(dimethylamino) naphthalene-1-sulfonamide) propanoic acid (Dansylalanine), 4: Nε-p-azidobenzyloxycarbonyl lysine (PABK), 5: Propargyl-L-lysine (PrK), 6: Nε-(1-methylcycloprop-2-enecarboxamido) lysine (CpK), 7: Nε-acryllysine (AcrK), 8: Nε-(cyclooct-2-yn-1-yloxy) carbonyl) L-lysine (CoK), 9: bicyclo[6.1.0]non-4-yn-9-ylmethanol lysine (BCNK), 10: trans-cyclooct-2-ene lysine (2′-TCOK), 11: trans-cyclooct-4-ene lysine (4′-TCOK), 12: dioxo-TCO lysine (DOTCOK), 13:3-(2-cyclobutene-1-yl) propanoic acid (CbK), 14: NE-5-norbornene-2-yloxycarbonyl-L-lysine (NBOK), 15: cyclooctyne lysine (SCOK), 16:5-norbornen-2-ol tyrosine (NOR), 17: cyclooct-2-ynol tyrosine (COY), 18: (E)-2-(cyclooct-4-en-1-yloxyl) ethanol tyrosine (DS1/2), 19: azidohomoalanine (AHA), 20: homopropargylglycine (HPG), 21: azidonorleucine (ANL), 22: NE-2-azideoethyloxycarbonyl-L-lysine (NEAK).

Without being bound by theory, trimerisation or multimerisation of binder proteins can be useful properties to improve or enable biological effects and novel components for trimerization or multimerisation have been pursued extensively (Cuesta, A M et al, Trends in Biotechnology, 2010). In addition to its suitability for fusion of effector proteins or binding domains to both the N terminus and the C terminus of CutA1, CutA1 presents a novel component for stable multimerisation of binder proteins. The data presented below, in particular FIG. 22 and FIG. 23, demonstrate that CutA1 can be a suitable domain for conjugation or fusion of binder proteins or effector proteins in general, for instance facilitating trimerisation to exert biological effects after conjugation to binder proteins via Catcher domains or as direct fusions to binder proteins. Accordingly, the invention also provides a polypeptide comprising a binding domain or effector protein fused N-terminally or C-terminally of CutA1. In some embodiments, the CutA1, typically human CutA1, is modified as described elsewhere herein.

In some aspects, the structural domain of the polypeptide construct (or the subunit monomer of the oligomeric core) is a TNF family protein including a TNF protein, a TL1A protein, an OX40L protein, or a CD40L protein, typically a human protein. In certain aspects, the TNF, TL1A, OX40L or CD40L protein, typically human protein, is engineered to remove one or more cysteine residues from the native sequence. Typically, this is a substitution of one or more cysteine residues with one or more non-cysteine residues. The removal of one or more cysteine residues beneficially allows for targeted cysteine conjugation at non-native sites or in fusion proteins. Without being bound by theory, unpaired cysteines routinely interfere with stability and downstream application, so it can be beneficial to remove unpaired cysteines. In certain aspects, the TNF, TL1A, OX40L or CD40L protein, typically human protein, is engineered to delete one or more residues from either or both termini of the native sequence, to form a truncated TNF, TL1A, OX40L or CD40L domain that is incorporated into the construct of the invention. In some embodiments, one or more residues, for example 2, 3, 4, 5, 6, 7, 8, 9, 10, 15, 20, or more residues are deleted from the N terminus of the native sequence. In some embodiments, one or more residues, for example 2, 3, 4, 5, 6, 7, 8, 9, 10, 15, 20, or more residues are deleted from the C terminus of the native sequence. In some embodiments, In some embodiments, one or more residues, for example 2, 3, 4, 5, 6, 7, 8, 9, 10, 15, 20, or more residues are deleted from the N terminus of the native sequence, and one or more residues, for example 2, 3, 4, 5, 6, 7, 8, 9, 10, 15, 20, or more residues are deleted from the C terminus of the native sequence.

In some aspects, as shown in the Examples, the modular assembly provided by the invention can be combined with simple post-assembly clean-up. This is particularly useful for the manufacture of drug candidates for downstream analysis. In some embodiments, these methods can be automated. In some embodiments the clean-up uses beads, for example paramagnetic beads, that can be bound with appropriate catchers to quench unconjugated tagged proteins from an assembly of tagged binders to a Catcher-core (see e.g. FIG. 37). This allows for cost-effective, rapid purification of assemblies (by removing unbound tagged binders) that is easily scalable.

The clean-up method typically comprises contacting a post-conjugation reaction mixture with clean-up binding domains that can bind to unbound binders. The clean-up binding domains, bound to the previously unbound binders, can then be removed from the reaction mixture.

These clean-up methods are particularly suitable for plate-based formats that can be automated through the use of a liquid handling robot. For example, GST-Catchers can be loaded onto glutathione-conjugated beads (e.g. FIG. 35) and the unbound excess removed via washing with PBS (e.g. FIG. 36). The GST-Catcher bound beads can then added to each conjugation reaction, typically in excess (for example a 2× excess of GST-Catcher) to the corresponding unconjugated tagged binders. The beads can then be removed by an appropriate means, for example when the beads are magnetic they can be removed via magnetic capture of the particles, leaving a supernatant that contains only the fully assembled molecule (e.g. FIG. 38). This method is advantageous, relative to covalent-conjugation of catchers to beads, as it can be performed on-demand at a variety of scales. There are also no complex chemical processes required or reducing conditions. Furthermore, the design of the catcher constructs, the ratio of catcher proteins, or resin can easily be changed as demand requires.

This clean-up technology is a general improvement to the art and can also be applicable to other modular assembly approaches, in particular the method described by Driscoll et al, September 2023, bioRxiv 2023.08.31.555700; doi: https://doi.org/10.1101/2023.08.31.555700.

The clean-up technology may be adapted for approaches such as Sortase-based assembly, for example as described by Andres et al, Mol Cancer Ther (2020) 19 (4): 1080-1088 “High-Throughput Generation of Bispecific Binding Proteins by Sortase A-Mediated Coupling for Direct Functional Screening in Cell Culture”.

The clean-up step typically uses fusion proteins that are able to bind excess binders in the reaction mix after the assembly of catchers and tagged binders. These clean-up fusion proteins typically comprise a protein that is useful in the clean-up method described immediately above, fused to at least one binding domain as described herein, typically a Catcher domain that is able to bind to excess binder in the reaction mix. The protein that is useful in the clean-up step is typically able to form a protein-domain based association useful in a purification process. This association may be covalent (e.g. halo-tag, which is a modified haloalkane dehalogenase designed to bind covalently to synthetic ligands that comprise a chloroalkane linker attached to a variety of useful molecules, such as fluorescent dyes, affinity handles, or solid surfaces) or non-covalent (e.g. maltose binding protein [MBP]). As noted above, this construct is typically a glutathione-S-transferase “GST” sequence that is able to bind to GSH on a bead. In some embodiments, the clean-up fusion protein is attached to a solid support such as a bead. In some embodiments, the bead may be magnetic or para-magnetic.

GST/GSH is a particularly suitable system due to commercial availability of paramagnetic beads with high protein capacity at comparably low cost.

The clean-up fusion protein can comprise a single type of binding domain, which will be able to clean up one excess binder. This single binding domain may be provided in a single copy (e.g. GST-SpyCatcher), or optionally be provided in multiple copies (for example in the format GST-SpyCatcher-SpyCatcher). When there are two or more post-assembly excess binders to clean-up, then two or more clean-up fusion proteins can be used in combination, each with a different single type of binding domain. GST-SpyCatcher and GST-DogCatcher are examples of clean-up fusion proteins. In some exemplary embodiments, for example, a first clean-up fusion protein is GST-SpyCatcher and a second clean-up fusion protein is GST-DogCatcher. Specific sequences are provided in the Examples for GST-SpyCatcher003 and GST-DogCatcher. In other exemplary embodiments, clean-up fusion proteins comprise GST-SpyCatcher002 or GST-SpyCatcher-003. In other exemplary embodiments, clean-up fusion proteins comprise GST-SpyCatcher002 and GST-SpyCatcher-003, used in combination. Other clean-up fusion proteins may include inactive variants of SpyCatchers such as SpyCatcher002 KA, SpyDock or SpySwitch (e.g. as described in Khairil Anuar et al, Nature Communications volume 10, Article number: 1734 (2019), in particular FIG. 7).

MBP-SpyCatcher and MBP-DogCatcher are further examples of clean-up fusion proteins. In some exemplary embodiments, for example, a first clean-up fusion protein is MBP-SpyCatcher and a second clean-up fusion protein is MBP-DogCatcher. In other exemplary embodiments, clean-up fusion proteins comprise MBP-SpyCatcher002 or MBP-SpyCatcher-003. In other exemplary embodiments, clean-up fusion proteins comprise MBP-SpyCatcher002 and MBP-SpyCatcher-003, used in combination.

Halotag-SpyCatcher and Halotag-DogCatcher are further examples of clean-up fusion proteins. In some exemplary embodiments, for example, a first clean-up fusion protein is Halotag-SpyCatcher and a second clean-up fusion protein is Halotag-DogCatcher. In other exemplary embodiments, clean-up fusion proteins comprise Halotag-SpyCatcher002 or Halotag-SpyCatcher-003. In other exemplary embodiments, clean-up fusion proteins comprise Halotag-SpyCatcher002 and Halotag-SpyCatcher-003, used in combination.

In some embodiments, two or more binding domains are attached to a single protein that is useful in the clean-up method described above. The multiple binding domains, typically multiple Catcher domains, may be connected at any suitable location on the protein useful in clean-up (e.g. GST, MBP or halo-tag). The multiple binding (e.g. catcher) domains may for example be arranged in series at one terminus of the clean-up protein, or be arranged at different termini. As with other embodiments described herein, a linker peptide (e.g. 2 to 30 amino acid residues) may be included between separate components of the fusion as appropriate. Some exemplary formats, wherein each Catcher is different, include Catcher1-GST-Catcher2, Catcher1-Catcher2-GST, GST-Catcher1-Catcher2, Catcher1-MBP-Catcher2, Catcher1-Catcher2-MBP, MBP-Catcher1-Catcher2, Catcher1-halotag-Catcher2, Catcher1-Catcher2-halotag, or halotag-Catcher1-Catcher2.

In some embodiments, the invention provides a polypeptide comprising a first binding domain at the N terminus and a second binding domain at the C terminus, wherein the first and second binding domains are separated by a structural domain that is useful in the clean-up method described immediately above, and wherein the first and second binding domains are the same or are different. In these embodiments, the structural domain is typically able to form a protein-domain based association useful in a purification process. This association may be covalent (e.g. halo-tag) or non-covalent (e.g. maltose binding protein [MBP]). As noted above, this construct is typically a glutathione-S-transferase “GST” sequence that is able to bind to GSH on a bead.

In some aspects, the disclosure provides a TNF Receptor Superfamily (TNFRSF) agonist, such as a TRAIL Receipt agonist, for example a DR5 agonist, comprising CutA1 or a variant thereof as described herein. TNFRSF agonists are known in the art. Typically, the agonism is provided by one or more binding regions (e.g. Fab, ScFv, nanobody) that are connected to the CutA1, for example at the N-terminus and/or C-terminus. Agonist antibodies for TRAIL receptors such as DR5 are known in the art, for example IGM8444 and TAS266 (See e.g. (i) Wang et al, ASCO 2020 Annual Meeting Abstract: “IGM-8444 as a potent agonistic Death Receptor 5 (DR5) IgM antibody: Induction of tumor cytotoxicity, combination with chemotherapy and in vitro safety profile.”; (ii) Huet et al Cancer Res (2012) 72 (8_Supplement): 3853. “Abstract 3853: TAS266, a novel tetrameric nanobody agonist targeting death receptor 5 (DR5), elicits superior antitumor efficacy than conventional DR5-targeted approaches”; and (iii) WO-A-2017/011837).

DR5 binders by themselves may not be agonising. Typically, and without wishing to be bound by theory, anti-DR5 antibodies (including antigen-binding fragments and engineered formats thereof, including those according to the present invention) need to have a particular valency state, e.g. bivalent, trivalent or tetravalent, to effectuate an agonising effect on DR5. The valency of a binder can be increased by association with a multimerising scaffold (e.g. CutA1). The data in at least Examples 11 and 14 demonstrate that at least trivalency provides effective agonism with the constructs of the invention. This demonstrates that CutA1 is particularly beneficial for targeting TNFRSF, which may be due to CutA1's geometry (including its trimeric nature) and small size similar to the natural ligands of TNFRSF receptors. FIG. 11 exemplifies ligands of the TNFSF family, for reference. Accordingly, in one embodiment, trivalent CutA1-DR5 agonist is provided. In some embodiments, a hexavalent CutA1-DR5 agonist is provided.

The C-terminal and N-terminal position may be highly different for two trimers, and one may be preferred over the other. For example, for DR5, CutA1 C-terminal fusions may sometimes be preferred (see FIG. 22, FIG. 23c, FIG. 32).

In some embodiments, a multimeric CutA1-DR5 agonist is provided. In some embodiments, a trimer of CutA1-DR5 agonist constructs is provided. In some embodiments, the multimer may be dimeric, or may be a higher order, for example tetrameric, pentameric, hexameric, heptameric or an even higher order.

Typically, the CutA1-DR5 agonist construct includes a second binding domain to a different target, i.e. is a bispecific construct. The second binding domain is typically provided at a different location on the CutA1, most typically at the opposite terminus. However, as described elsewhere herein it can also be provided in series with the DR5 agonist, at the same location CutA1. Suitable construct formats therefore include (depicted in the usual N to C orientation): Binding Domain 2—CutA1—DR5 binder; DR5 binder—CutA1-BindingDomain2; DR5 binder—BindingDomain2-CutA1; CutA1—DR5 binder—BindingDomain2; BindingDomain2-DR5binder—CutA1; or CutA1—BindingDomain2 DR5 binder. Each of these constructs are provided as above, for example in multivalent (e.g. trivalent, hexavalent) and/or multimeric (e.g. trimeric or hexameric) format.

TAS266 is a tetrameric nanobody agonist targeting Death Receptor 5 (DR5). As discussed in the examples, the inventors hypothesised that the severe hepatotoxicity which lead to clinical failure of TAS266 was a result of antidrug antibody-dependent hyperclustering of DR5 and consequent overinduction of apoptosis in liver cells (Inhibrx, WO2017-A-011837). Such antibodies are present in commercially available intravenous immunoglobulin (IVIG) solutions (Inhibrx, WO2017-A-011837). In contrast to TAS266, a CutA1-based format (exemplified as L11-HsCutA1-L7) did not increase IVIG-dependent toxicity in the HepG2 hepatocyte line (see e.g. FIG. 41). The inventors observe that, without wishing to be bound by theory, the decreased valency of the DR5 targeting binder from four to three and simultaneous introduction of bi-specificity may allow the CutA1-based construct exemplified by L11-HsCutA1-L7 to elicit less severe adverse effects and display increased tumour-specificity.

In some embodiments, a construct is provided comprising CutA1 or a variant thereof, and a DR5 binding domain. Typically this construct will also comprise a second binding domain for another target. In some embodiments, the DR5 binding domain competes for binding to DR5 with the TAS266 tetrameric construct. In some embodiments, the DR5 binding domain comprises the “VHH” binding domain from TAS266 (e.g. SEQ ID NO: 106). In some embodiments, the construct comprises CutA1 or a variant thereof as described herein and the “VHH” binding domain from TAS266 (as exemplified by SEQ ID NO: 102, for example) and optionally a second binding domain (for example an scFv, VHH or Fab) that binds to a different target protein. A linker sequence may be included between the anti-DR5 VHH and the CutA1, and/or between the CutA1 and the second binding domain. Typically, the two binding domains are at opposite termini of the CutA1, although they may be provided in sequence at one end of the CutA1.

SEQ ID NO:106 provides one example of a DR5 binder useful in constructing a multivalent DR5 agonist, such as tetravalent TAS266 or trimeric CutA1-DR5. In some embodiments, a DR5 binder competes with SEQ ID NO:106 for binding to DR5. This competition may be determined, for example, in a surface plasmon resonance assay such as a Biacore assay. In some embodiments, a DR5 agonist has at least 70% identity, at least 80% identity, at least 90% identity, for example at least 95% identity or at least 99% identity to SEQ ID NO: 106. Typically, any variation is only present in the framework residues and no variation is permitted in the CDR residues.

SEQ ID NO:102 provides one example of an agonistic hsCutA1-DR5 binder. Sequences with at least 70% identity, at least 80% identity, at least 90% identity, for example at least 95% identity or at least 99% identity to SEQ ID NO: 102 may be used. Typically, any variation is only present in the framework residues and no variation is permitted in the CDR residues.

In a further aspect, as exemplified in Example 14, the invention provides a single chain CutA1, which is a further engineered variant of CutA1. This is an engineered variant of CutA1 comprising multiple CutA1 molecules (whether full-length or truncated, and optionally substituted or otherwise engineered, as described extensively herein) in which every “monomer” of CutA1 is covalently connected, optionally by a linker.

In one embodiment of this aspect, a single polypeptide chain comprises multiple CutA1 sequences, optionally wherein a linker polypeptide between 3 and 30 amino acids in length lies between some or all of the CutA1 sequences, optionally at least some and optionally all of the CutA1 sequences are is as defined elsewhere herein. In a further embodiment, a single polypeptide chain comprises three human CutA1 sequences each separated by a linker polypeptide, optionally comprising an isopeptide bond-forming domain at the N and/or C terminus.

Typically, the single-chain CutA1 is expressed as a fusion protein comprising two or more CutA1 molecules, for example three or more CutA1 molecules, for example 4, 5, 6, 7 8 or more CutA1 molecules. Three CutA1 molecules is typical. Some or all of the CutA1 molecules in the chain are typically separated by a polypeptide linker, typically wherein the polypeptide linker is 3 to 30 amino acid residues in length, more typically 10 to 20 amino acid residues in length, for example 12, 13, 14, 15, 16, or 17 amino acids in length. A typical non-limiting linker is 15 amino acids in length, for example (GGGGS)₃.

For example, in a trimeric single chain CutA1 construct each of the three CutA1 components can be connected by means of a linker to generate a construct. The construct may also include the binding moieties of interest, typically (although not necessarily) at the termini. An exemplary single chain CutA1 construct is: SpyCatcher-Linker-CutA1-Linker-CutA1-Linker-CutA1-DogCatcher. This single-chain construct can particularly useful to provide a 1+1 bivalent control with the same geometry as a 3+3 molecule. This single-chain CutA1 has a number of potential uses, including but not limited to: as an in vitro or in vivo control scaffold compared to 3+3 CutA1; as a scaffold to assess 1+1 in a fixed geometry; and in in vivo applications when combined with other CutA1 technologies, such as ADC (e.g. to make high Drug Antibody ratio “DAR” ADC).

The present invention allows for the highly adaptable screening of large numbers of effector moieties. The effector moieties may be any protein domain and are not limited to Fab/Fv regions or other antigen-binding domains. The present invention also may also be used to investigate effects of molecules achieved by higher valency interactions that would not be observed using conventional bispecific antibody or other approaches. Accordingly, the invention provides a system for high throughput screening of bispecific combinations of molecules, that can pick up effects that are only observed through higher-valency interactions. The invention also provides new therapeutic candidates which may be identified according to the methods provided herein. The therapeutic candidates provide the benefits of multiple functionalities and increased valency.

Limited use of multivalent protein scaffolds has been described in the art. One attempt described by Brune et al., Bioconjugate Chemistry, 28 (5), pp. 1544-1551, relates to the use of a heptameric assembly in which antigens were assembled on opposing faces of an IMX313 heptameric core as a potential malaria vaccine. However, such constructs are not suitable for same-cell multi-receptor engagement due to opposing display of the attached antigens, limiting their applicability in same-cell binding for treating disorders such as cancer and autoimmune disorders.

Accordingly, there is a need for new and/or improved methods for phenotypic screening of combinations of effector moieties (also referred to herein as ligands) and for methods of developing new therapeutics. Prior methods do not allow for increased valency of the combinations of functionalities investigated, and/or do not recognize the synergistic benefits of presenting antigens on the same face of a protein scaffold. There is also a need for new and/or improved therapeutics, including the therapeutics provided herein which can be designed and identified according to the present disclosure.

### BRIEF DESCRIPTION OF THE FIGURES

FIG. 1 is a schematic showing a multivalent protein scaffold as described herein in which a first binding site and a second binding site are positioned on the same face of the oligomeric core and thus on the same face of the multivalent protein scaffold.

FIG. 2 is a schematic showing a multivalent protein scaffold as described herein in which a first binding site and a second binding site are positioned on opposite faces of the oligomeric core and thus on opposite faces of the multivalent protein scaffold.

FIG. 3 is a schematic showing a multivalent protein scaffold as described herein in which a plurality of first binding sites and a plurality of second binding sites are positioned on the same face of the oligomeric core and thus on the same face of the multivalent protein scaffold for engagement with a surface.

FIG. 4 is a schematic showing a multivalent protein scaffold as described herein in which a plurality of first binding sites and a plurality of second binding sites are positioned on opposite faces of the oligomeric core and thus on opposite faces of the multivalent protein scaffold and therefore cannot simultaneously engage with a surface.

FIG. 5 is a schematic showing a multivalent protein scaffold as described herein in which a first binding site and a second binding site are positioned on the same face of the oligomeric core and thus on the same face of the multivalent protein scaffold in a front-front orientation.

FIG. 6 is a schematic showing a multivalent protein scaffold as described herein in which a first binding site and a second binding site are positioned on the same face of the oligomeric core and thus on the same face of the multivalent protein scaffold in a front-side orientation.

FIG. 7 is a schematic showing a multivalent protein scaffold as described herein in which a first binding site and a second binding site are positioned on the same face of the oligomeric core and thus on the same face of the multivalent protein scaffold in an orientation intermediate between a front-front orientation and a front-side orientation.

FIG. 8 is a schematic showing the angle (X) that is formed between a first binding site and a second binding site attached to a subunit monomer of the oligomeric core of a multivalent protein scaffold as described herein.

FIG. 9 is a schematic showing an embodiment of the invention in which a tandem fusion of first and second binding sites is attached to an oligomeric core as described herein allowing production of a multivalent protein scaffold. Various different geometries and stoichiometries can be produced using the methods disclosed herein.

FIG. 10 illustrates a 2D exposition of fusion sites in “cis”. a) For a given target plane, the longest cross-section of the core protein via an orthogonal line drawn from that target plane is determined, featuring a distance dc. A parallel plane crossing the midpoint of this cross-section is shown. Here, sites for protein conjugation (stars, circles) are considered to preferentially be in “cis” if, for all conjugation sites, the distance of the shortest path from a conjugation site to the target plane that does not intersect with the protein surface is less than a certain % of the distance d_{c}, such as less than 50% of d_{c}. b) An example protein in which all binding sites are in cis. c) An example protein in which all binding sites are in cis to each other according to a), as all second binding sites (circles) feature minimal path lengths that are just below the threshold of 50% d_{c}. d) Even though the projected distances (crossing the protein) from all binding sites to the target plane are the same as in c), this protein features a geometry that obstructs the shortest paths from all second binding sites (circles), therefore these binding sites are not accessible to and/or do not fall onto the same side of the scaffold. e,f) The binding sites are too far apart from each other to be considered cis towards any target plane.

FIG. 11 provides an overview of protein structures referred to herein. Protein structures for given PDB IDs are visualized as cartoon, with chains in differing hues. N-terminus and C-terminus are each annotated for a single monomer, and approximately oriented towards a binding surface. The symmetry of the protein structure is shown in parenthesis, e.g. cyclic C3 symmetry, cyclic C4 symmetry, and dihedral D2 symmetry. *: protein symmetry is estimated from the NMR structure. †1PK6 is a heteromer featuring C1 symmetry, however the domains are homomeric and their arrangement resembles C3 symmetry. By selection of a multimeric protein core with suitable monomer configuration, multiple binding sites can be projected per monomer to form a single binding surface. In addition to recombinant fusion, such a protein could then be utilised for modular assembly, for instance by recombinant fusion at N-terminus and C-terminus with SpyCatcher and SnoopCatcher or DogCatcher, to quickly confer multivalency or other properties onto suitably modified peptides or proteins, for instance, via a recombinant fusion of SpyCatcher at the N-terminus and SnoopCatcher at the C-terminus of the monomer of an oligomeric core protein. Examples include: (1) HsCutA1, a human copper-binding protein with high thermostability, featuring C3 geometry with both N- and C-termini of each monomer in close proximity to each other, projecting onto a single plane in the assembled trimer (PDB ID 2ZFH). (2) The hyper-thermostable homologue PhCutA1 from Pyrococcus horikoshii (PDB ID 4NYO) which has high structural similarity to HsCutA1. (3) NC1-domain of Collagen X (PDB ID: 1GR3), (4) NC1-domain of Collagen VIII (PDB ID: 1091), (5) Macrophage Migration Inhibitory Factor 2 (PDB ID: 7MSE), (6) Tumor Necrosis Factor (PDB ID: 1TNF), (7) TNF-like protein TL1A (PDB ID: 2RE9).

FIG. 12 shows that cis-oriented multimeric protein complexes can be readily expressed and prepared by standard protein purification methods. a) Ni-NTA purification of H6-SpC-PhCutA1-SnC [SEQ ID NO: 21]. H6-SpC-PhCutA1-SnC is readily expressed in F. coli BL21 (DE3) and retains an intact trimeric structure characteristic to hyperstable PhCutA1, even after boiling with SDS-PAGE loading buffer. Washes 1 and 2 were performed with 10 column volumes equilibration buffer (50 mM Tris, pH 7.8; 300 mM NaCl; 10 mM imidazole). Washes 3 and 4 were performed with 10 column volumes of wash buffer (50 mM Tris, pH 7.8; 300 mM NaCl; 30 mM imidazole). All elution steps were performed with 2 column volumes of elution buffer (50 mM Tris, pH 7.8; 300 mM NaCl; 200 mM imidazole). Samples were analysed using 12% SDS-PAGE gels with Coomassie staining. P—Lysate pellet; CL—Clarified lysate; FT—Flow-through; W—Wash; E—Elution. b,c) Result of size exclusion chromatography using HiLoad 16/600 Superdex 200 pg for H6-SpC-PhCutA1-SnC. b) UV A280 absorbance chromatogram with the H6-SpC-PhCutA1-SnC peak highlighted. c) SDS-PAGE of the H6-SpC-PhCutA1-SnC 2 mL fractions from the highlighted region in the above chromatogram.

FIG. 13 shows transition from a dihedral hexamer to a circularly symmetric trimeric core protein for cis-oriented display. a) A homo-hexameric antiparallel coiled-coil features N-termini and C-termini on two opposing sides of the protein assembly (PDB ID: 5W0J). b) A heteromeric assembly can be derived from a homomeric assembly by point mutagenesis, e.g. by introduction or modification of salt-bridge formation, “locking” the assembly in one orientation (PDB ID: 5VTE). c) By linking the termini of a heteromeric assembly, a homomeric assembly suitable for cis-oriented display would be derived (compare to HIV GP41, PDB ID: 115Y). Black to white (structure, schematic) and arrow direction (schematic): N-terminus to C-terminus. Structures visualised in PyMOL.

FIG. 14 shows that SpC-PhCutA1-SnC is a highly stable trimeric protein. a) Samples of SpC-PhCutA1-SnC were heated at 97° C. for 2 h in either 0%, 0.5% or 1% SDS in addition to PBS, before adding SDS-loading dye. Samples were resolved on 12% SDS-PAGE and stained with Coomassie. A control sample that was not heated and had no SDS present is shown in the first lane. At increased SDS concentrations, partial monomerisation of the trimer was observed, confirming that the trimer is not covalently crosslinked. b) SpC-PhCutA1-SnC was retained after prolonged storage. Protein aliquots were stored at 4° C., ambient temperature (21° C.), or 37° C. for 7 days before preparing samples with SDS-loading buffer and resolving on SDS-PAGE. Compared to storage at 4° C., the protein showed little sign of degradation at 21° C. to 37° C. Optionally, the protease inhibitor PMSF was added, with similar effect.

FIG. 15 shows the preparation of SpyTagged and SnoopTagged ligand components for modular assembly to platform proteins. a) Purification of SnT-L1 from 200 mL BL21 (DE3) culture using Ni-NTA chromatography. Each washing step was performed with 5 mL Ni-NTA wash buffer. Each elution step was performed with 2 mL of Ni-NTA elution buffer. Samples were analyzed using 12% SDS-PAGE gels with Coomassie staining. Pel.—Lysate pellet; FT—Flow-through; W—Wash; E—Elution; L—Ladder. b) Purification of L2-SpT as in a). c) Size exclusion chromatography of SnT-L1 after Ni-NTA purification. UV A280 absorbance chromatogram for SnT-L1 from AKTA Pure 25 with a HiLoad Superdex 16/600 75 pg column. Inset: SDS-PAGE of the SnT-L1 2 mL fractions from the highlighted region in the chromatogram. d) Size exclusion chromatography of L2-SpT as in c).

FIG. 16 shows that SpC-PhCutA1-SnC facilitates stable trimerization of SpyTagged and SnoopTagged proteins. a) H6-SpC-PhCutA1-SnC was conjugated with a 1:2:2 molar excess of SnT-L1 or L2-SpT for the indicated time before samples were supplemented with SDS-loading buffer and all samples were denatured by boiling at 95° C. for 5 min. Samples were resolved on 8% and 16% SDS-PAGE gels followed by Coomassie staining. We observed time-dependent conjugation of SpC-PhCutA1-SnC to SnT-L1 or L2-SpT, with consumption of ligand components. b) Conjugation of SpC-PC-SnC with SnT-L1 and L2-SpT as in a). c) Conjugation of SpC-PhCutA1-SnC with SnT-L1 and L2-SpT. SpC-PhCutA1-SnC was incubated with SnT-L1 and/or L2-SpT at 1:2:2 molar excess and samples were incubated at 25° C. for 64 h. Samples were analysed using 16% SDS-PAGE gels with Coomassie staining. Notably, SpC-PhCutA1-SnC and SnT-L1/L2-SpT conjugate to completion while retaining characteristic hyper-thermostability of PhCutA1. d) Conjugation of SpC-PC-SnC with SnT-L1 and L2-SpT as in c). SpC-PC-SnC and SnT-L1/L2-SpT conjugation was performed to completion.

FIG. 17 shows that a high-molecular weight scaffold enables post-assembly purification via dialysis. a) Ni-NTA purified SpC-PhCutA1-SnC and b) SpC-PhCutA1-SnC: SnT-L1:L2-SpT assembly were dialysed utilizing a 96-well format. a) SpC-P2-SnC was purified using Ni-NTA chromatography and the elution fractions were combined and concentrated. b) Protein conjugation was performed for SpC-PhCutA1-SnC, SnT-L1 and L2-SpT, at 25° C. for 2 h. Dialysis was performed using a 100 kDa MWCO membrane with a 1:1 ratio of sample to dialysis buffer in a HTDialysis 96-well block. The dialysis was performed with orbital shaking at ambient temperature. PBS dialysate was changed every 30 min up to 90 min. 24 h of dialysis show removal of protein impurities (a-b) and unconjugated ligands (b) to equilibrium (at 1:1 ratio of sample and dialysis buffer). Samples were boiled with reducing SDS-loading dye, resolved on 12% SDS-PAGE and visualized by Coomassie staining. c) Alternatively, SpC-PhCutA1-SnC: SnT-L1:L2-SpT conjugate was purified via dialysis in 12-well plate format, featuring a 1:30 ratio of sample to dialysis buffer. A 1:1 conjugation was set up for SpC-PhCutA1-SnC, SnT-L1 and L2-SpT at 25° C. for 24 h. Dialysis was performed using a HTDialysis 12-well block and a 100 kDa MWCO cellulose membrane over 16 h at ambient temperature with no agitation. A sample volume of 100 μL and a PBS dialysate volume of 3 mL was used, both of which contained 1×PMSF. Sample and dialysate were taken at the following timepoints: 2 h, 4 h, 8 h and 16 h. Samples were analysed using a 14% SDS-PAGE gel and Coomassie staining. S=dialysed sample, D=dialysate (PBS).

FIG. 18 shows changes in species core components (PhCutA1 to MIF2m or HsCutA1), protein components for conjugation (SpC/SnC to SpC3/DgC), and variable linker lengths (GGGGSGGGGSGGGGS for MIF2m and GGGGS for HsCutA), highlighting the potential for rapid prototyping a,b) Samples from Ni-NTA purification of H6-SpC3-HsCutA1-DgC or H6-SpC3-MIF2m-DgC. TL—Total lysate; P—lysate pellet; CL—cleared lysate; FT—flow-through; W—Wash; E—Elution. Samples were analyzed using SDS-PAGE gels with Coomassie staining. c) Both SpC3-MIF2m-DgC and SpC3-HsCutA1-DgC are capable of rapid conjugation to DogTagged or SpyTagged proteins. Platform proteins were incubated at 25° C. for 16 h in 1:1.5:1.5 molar ratio. d) Incubation of H6-SpC3-HsCutA1-DgC with 0.1% glutaraldehyde demonstrates crosslinking of trimeric protein in solution. 10 μM H6-SpC3-HsCutA1-DgC was crosslinked for 0-20 min using 0.1% glutaraldehyde. The reaction was incubated at 37° C. and stopped by the addition of 100 mM Tris, pH 8.8. Trimeric crosslinked species are rapidly formed; upon prolonged incubation, monomeric H6-SpC3-HsCutA1-DgC is consumed, with some formation of crosslinked species at the molecular weight expected for a crosslink between two trimers. e) SpC3-HsCutA1-DgC was reacted with L1-SpT and L3-DgT at a 1:2:2 molar ratio for 16 h at 25° C. Each sample of SpC3-HsCutA1-DgC only, L1-SpT: SpC3-HsCutA1-DgC, SpC3-HsCutA1-DgC:L3-DgT, and L1-SpT: SpC3-HsCutA1-DgC:L3-DgT were then subjected to size exclusion chromatography with Superose 6 Increase 5/150 GL column, showing an increase in hydrodynamic radius of each protein scaffold or protein assembly. Peak fractions from each chromatogram peak from the shaded area were loaded onto the SDS page gel to show only the scaffold and assembly proteins, with the excess ligands removed. These samples were used as the input for FIG. 20c.

FIG. 19 shows how Alphafold v2.0 was utilized to predict cis-orientation of fusion proteins. The highest ranked model of each SpC3-Scaffold-DgC assembly as visualized in PyMOL. A single chain is highlighted showing SpyCatcher3 (medium grey), Scaffold (dark grey) and DogCatcher (light grey). Additional chains are presented as a transparent surface with a cartoon in white. A GSGS linker was used between the catchers and scaffolds for all simulations. In the case of Collagen XV NC1 the structural prediction collapsed using the GSGS linker. Therefore, the prediction was repeated using a (GGGGS)2 linker and this structure is presented here. Monomer sequences used for Alphafold predictions: TL1A-SEQ ID NO: 32; Col XV NC1-SEQ ID NO: 33; MIF2m-SEQ ID NO: 34; HsCutA1-SEQ ID NO: 54; PhCutA1-SEQ ID NO: 55; Col X NC1-SEQ ID NO: 56; TNF-SEQ ID NO: 57.

FIG. 20 shows suitability of the scaffold for in vitro testing. a) H6-SpC-PhCutA1-SnC was conjugated with two different ligands, SnT-L1 and L2-SpT. Serum-starved NCI-N87 cells were grown for 7 days in the presence of a relevant growth factor and dual-conjugated assembly (H6-SpC-PhCutA1-SnC: SnT-L1:L2-SpT), single-conjugated assemblies (H6-SpC-PhCutA1-SnC: SnT-L1, H6-SpC-PhCutA1-SnC:L2-SpT) as well as ligand only controls (SnT-L1, L2-SpT, SnT-L1+L2-SpT), followed by MTT cell viability measurements. Control antibodies against L1, L2 and L1+L2 at 10 nM were used as controls (data not shown) and resulted in similar results compared to single- and dual-conjugated assembly samples; error bars show technical replicates of n=3. b) Fully assembled scaffold H6-SpC-PhCutA1-SnC: SnT-L1:L2-SpT represses activation of Akt and Erk1/2. NCI-N87 cells were treated with scaffold only (S; H6-SpC-PhCutA1-SnC), ligands only (L1; SnT-L1 and L2; L2-SpT) and single-(SxL1, SxL2) and dual-conjugated (SxL1xL2) assemblies for 1 h and showed repression of downstream activation of Akt/ERK signalling with the full assembly H6-SpC-PhCutA1-SnC: SnT-L1:L2-SpT. c) H6-SpC-HsCutA1-SnC was conjugated with two different ligands, SnT-L1 and L3-SpT. Serum-starved NCI-N87 cells were treated for 2 days with dual-conjugated assembly (H6-SpC-HsCutA1-SnC: SnT-L1:L3-SpT), single-conjugated assemblies (H6-SpC-HsCutA1-SnC: SnT-L1, H6-SpC-HsCutA1-SnC:L3-SpT) as well as ligand only controls (SnT-L1, L3-SpT, SnT-L1+L3-SpT), followed by MTT cell viability measurements; error bars show technical replicates of n=3.

FIG. 21 shows the purification of L1-PhCutA1-L2 as a direct fusion multi-domain polypeptide both by Ni-NTA and size exclusion chromatography. a) L1-PhCutA1-L2 is readily expression in E. coli BL21 (DE3) and is purified by Ni-NTA chromatography using HisPur resin (ThermoFisher). Wash 1 and 2 were performed with 10 column volumes equilibration buffer (50 mM Tris, pH 7.8, 300 mM NaCl, 10 mM imidazole). Wash 3 and 4 were performed with 2 column volumes of wash buffer (50 mM Tris, pH 7.8, 300 mM NaCl, 30 mM imidazole). All elution steps were performed with 2 column volumes of elution buffer (50 mM Tris, pH 7.8, 300 mM NaCl, 200 mM imidazole). Samples were analysed on a 12% SDS-PAGE gel and stained with Coomassie. P—Lysate Pellet, CL—Cleared Lysate, FT—Flow-through, W—Wash, E—Elution. b) Result of size exclusion chromatography using HiLoad 16/600 Superdex 200 pg for L1-PhCutA1-L2 b) UV A280 absorbance chromatogram with the L1-PhCutA1-L2 peak highlighted. Inset: SDS-PAGE of the L1-PhCutA1-L2 2 mL fractions from the highlighted region in the above chromatogram.

FIG. 22 shows cell survival upon treatment of two cancer cell lines with assemblies consisting of various ligands (L1, L2, L4, L5, L6, L7) and SpC3-HsCutA1₄₄₋₁₇₉-DgC (SEQ ID NO: 24). Cell viability of Colo 205 and NCI-N87 cells is presented in a heat map. Cells were treated with single assemblies (SpyTag-ligand or DogTag-ligand only on a scaffold) and fully conjugated assemblies. Colo 205 and NCI-N87 were treated with doses ranging from 0.16 to 100 nM and 4 to 100 nM, respectively. Data shown represents cell survival at highest doses used. The lower panel shows the scale of relative cell survival.

FIG. 23 shows HsCutA1 direct fusions (without modular protein binding (Catcher). a) Structural models contrasting the lengths of SpyCatcher003 (SpC3) with SpyTag003 (SpT3) and DogCatcher (DgC) with DogTag (DgT) compared to alternative linkers such as (G4S) n where n=1, 2, 3, (EAAAK) n where n=1, 2, 3, and ribosomal L9 linker to mediate the linkage between polypeptides (protein binders) and the multivalent scaffold in place of the modular binding site (SpyCatcher003 and DogCatcher). Length between respective Tags and Catchers are shown for when the protein binders are fused to the Tags either N- or C-terminally. b) Cytotoxicity of SpC3-(G4S)-HsCutA1-(G4S)-DgC (SEQ ID NO: 24) assembled with the shown protein pairs (e.g. L5/L4 where L5 is Spy Tagged and L4 is DogTagged), compared with direct fusions for which the modular display platform was replaced as follows:L5/L4 to L5-GGGGS-[C75A, C96A]HsCutA 1₆₇₋₁₇₁-GGGGS-L4, L1/L2 to L1-(GGGGS)3-[C75A, C96A]HsCutA1₆₇₋₁₇₁-(GGGGS)3-L2, L2/L4 to L2-(GGGGS)4-HsCutA 1₄₄₋₁₇₉-(GGGGS)4-L4. Cell survival of NCI-N87 cells was determined after 7 days of treatment with 20 nM of assemblies and direct fusions. c) Cytotoxicity induced by SpC3-[C75V, C96S]HsCutA1₄₄₋₁₇₉-DgC (SEQ ID NO: 73) assembled with L7 at either SpC3 or DgC, forming trivalent monospecific molecules at N- or C-terminus end, and the comparison with their direct fusion equivalent. The direct fusion equivalents were L7-(GGGGS)5-[C75V, C96S]HsCutA 1₄₄₋₁₇₉ and [C75V, C96S]HsCutA 1₄₄₋₁₇₉-(GGGGS)8-L7. Fractions of surviving Colo 205 cells were determined 48 h after the start of the treatment at indicated doses. The same assay was conducted for d) and e). d) Levels of apoptosis induced by SpC3-[C75V, C96S]HsCutA1₄₄₋₁₇₉-DgC (SEQ ID NO: 73) and SpC3-[C75V, C96S]HsCutA1₆₀₋₁₇₁-DgC (SEQ ID NO: 76) assembled with L7 at both SpC3 and DgC, forming hexavalent monospecific molecules, and the comparison with their direct fusion equivalent. The direct fusion equivalents were L7-(GGGGS)5-[C75V, C96S]HsCutA 1₄₄₋₁₇₉-(GGGGS)8-L7 and L7-(GGGGS)5-[C75V, C96S]HsCutA1₆₀₋₁₇₁-(GGGGS)8-L7. e) Levels of apoptosis induced by several direct fusion molecules of different cores fused N- and C-terminally to L7 in the format of L7-(GGGGS)5-[core]-(GGGGS)8-L7, where [core] were [C75V, C96S]HsCutA1₄₄₋₁₇₉ (SEQ ID NO: 68), [C75V, C96S]HsCutA1₆₀₋₁₇₁ (SEQ ID NO: 70), OX40L (SEQ ID NO: 78), CD40L (SEQ ID NO: 79), and full-length soluble TNF (SEQ ID NO: 80).

FIG. 24 shows truncation of the HsCutA1 termini. a) Coding sequence schematic of differentially truncated HsCutA1 fused to SpyCatcher003 (SpC3) and DogCatcher (DgC) via example linkers L1 and L2. The termini truncations are noted as 44-179 (SEQ ID NO: 29) and 60-171 (SEQ ID NO: 66) respectively. The sequences are compared in the multiple sequence alignment of WT HsCutA1 (33-179) with HsCutA1 truncated to 44-179 and 60-171. b) Cartoon of sites of truncation on HsCutA1. At the N-terminus, residues 44-59 are shown and at the C-terminus, residues 172-179 are shown. Cartoon modelled from PDB ID: 2ZFH. c) Comparison of expression of HsCutA1₄₄₋₁₇₉ to HsCutA1₆₀₋₁₇₁. Cell lysates from E. coli were compared to show any changes imparted by the truncation on the expression of the proteins. d) Cytotoxicity of SpC3-(GGGGS)3-[C75V, C96S]HsCutA1₄₄₋₁₇₉-(GGGGS)3-DgC (SEQ ID NO: 73) and SpC3-(GGGGS)3-[C75V, C96S]HsCutA1₆₀₋₁₇₁-(GGGGS)3-DgC (SEQ ID NO: 76) variably assembled with L7 at either SpC3, DgC or both, forming trivalent or hexavalent monospecific molecules. Fractions of surviving Colo 205 cells were determined 48 h after the start of the treatment at indicated doses.

FIG. 25 shows the removal of cysteines from HsCutA1. a) Cartoon representation of sites of cysteines natively present in HsCutA1 for removal. Cartoon modelled from PDB ID: 2ZFH. b) Table of amino acid frequency at sites similar to HsCutA1's C75 and C96, following a multiple sequence alignment (MSA) of 406 homologs. Amino acids with frequency of zero are not shown. c) Comparison of expression of HsCutA1₄₄₋₁₇₉ to HsCutA1₆₀₋₁₇₁ with native WT cysteines (C75, C96), alanine mutated cysteines (C75A, C96A) and MSA-informed mutations (C75V, C96S). Cell lysates from E. coli were compared to show any changes imparted by the mutagenesis on protein expression. d) Cytotoxicity induced by SpC3-GGGGS-HsCutA1₄₄₋₁₇₉-GGGGS-DgC (SEQ ID NO: 24), SpC3-(GGGGS)3—[C75V, C96S]HsCutA1₄₄₋₁₇₉-(GGGGS)3-DgC (SEQ ID NO: 73), SpC3-(GGGGS)3-HsCutA1₆₀₋₁₇₁-(GGGGS)3-DgC (SEQ ID NO: 74), SpC3-(GGGGS)3—[C75A, C96A]HsCutA1₆₀₋₁₇₁-(GGGGS)3-DgC (SEQ ID NO: 75), and SpC3-(GGGGS)3—[C75V, C96S]HsCutA1₆₀₋₁₇₁-(GGGGS)3-DgC (SEQ ID NO: 76) assembled with L7 at both SpC3 and DgC, forming hexavalent monospecific molecules. Fractions of surviving Colo 205 cells were determined 48 h after the start of the treatment at indicated doses.

FIG. 26 illustrates a general thiol-maleimide reaction with a monomeric protein, as well as various linker-payload structures. A) A thiol group of the monomeric protein reacts with the maleimide moiety of a payload-linker compound to produce a thiosuccinimide payload-linker protein adduct. B) Examples of linker-payload compounds conjugated to Catcher core proteins: 1—fluorescein-5-maleimide; 2—deruxtecan; 3—maleimidocaproyl-Val-Cit-PAB-DM1; 4—maleimidocaproyl-Val-Cit-PAB-MMAF.

FIG. 27 shows the positions of Cys mutations on a cartoon representation of the CutA1 structure (modelled from PDB ID: 2ZFH) and a model of a fluorescein-5-thiosuccinimide-CutA1 conjugate. A) Spatial locations of Cys mutations on one monomer of the CutA1 structure. Cys thiols are shown as a sphere. B) Model of a fluorescein-5-thiosuccinimide conjugate onto the K82C mutant of CutA1 (SEQ ID NO: 86). All images were produced in PyMOL.

FIG. 28 shows fluorescein-5-maleimide conjugation of all Cys mutant CutA1 Catcher core variants (SEQ ID NO: 95, 93, 96, 92, 94, 97) as well as wild-type (SEQ ID NO: 24) and cysteine-free (SEQ ID NO: 91) controls, in addition to deconvoluted ESI mass spectra for unconjugated CC042 and a fluorescein-5-thiosuccinimide-CC042 conjugate. A) Dye/protein ratio for all mutants was calculated as described in methods for conjugated samples after PD-10 purification. B) 12% SDS-PAGE gel of unconjugated and conjugated CutA1 Catcher core mutants. Little to no conjugation is observed for the CC041 negative control but all other mutants conjugate to fluorescein-5-maleimide. CC denotes Catcher core protein gel bands. C) ESI deconvoluted mass spectrum for unconjugated Catcher core: the main peak is within one mass unit of the expected molecular weight for the CC042 monomer. D) Mass spectrum for fluorescein-5-thiosuccinimide-CC042 conjugate: the main peak is approximately 18 Da larger than the expected molecular weight for a monomer, possibly due to thiosuccinimide ring hydrolysis after conjugation.

FIG. 29 shows SDS-PAGE (4-20% gradient) of fluorescein-5-maleimide conjugation and Catcher/Tag assembly of CC042 (SEQ ID NO: 93) and the CC041 cysteine-free negative control (SEQ ID NO: 91). Only CC042 efficiently conjugates to fluorescein-5-maleimide. A low level of background signal indicative of conjugation is observed for CC041, suggesting that maleimide conjugation is mostly specific to Cys residues. The conjugated CC042 shows a clear change in migration in the gel on reaction with a DogTagged-binder (L7), SpyTagged-binder (L8), or both binders simultaneously. A similar profile of bands is observed for the CC041 control, suggesting that maleimide conjugation does not impair assembly efficiency.

FIG. 30 shows specific membrane binding and internalisation by RTK binders, single assemblies and full assemblies. HsCutA1 refers to CC7, while HsCutA1* refers to CC042 conjugated to fluorescein-5-maleimide. A) His-tagged binders L1 and L2, along with full assembly with His-tagged CC7 (SEQ ID NO: 76) demonstrate membrane binding and internalisation of all compounds when staining with an anti-His antibody. (Note differential ratio of His-tags.) B) CutA1 staining of full assembly of CC7 with binders L1 and L2 confirms membrane binding and internalisation. C) Fluorescein-5-maleimide (F-5-M) conjugated CC042 (SEQ ID NO: 93) alone as single assemblies and full assembly shows RTK specific internalisation of all assemblies without off-target binding of HsCutA1*.

FIG. 31 shows the structures of cytotoxic drug-linker compounds and corresponding deconvoluted ESI mass spectra of CC042 (SEQ ID: 93) conjugated to these compounds. For reference, the mass spectrum of unconjugated CC042 can be found in FIG. 28C. A) Deruxtecan; the main peak for the conjugate is within one mass unit of the expected molecular weight. B) Maleimidocaproyl-Val-Cit-PAB-DM1: the main peak for the conjugate is within one mass unit of the expected molecular weight. C) Maleimidocaproyl-Val-Cit-PAB-MMAF: the main peak for the conjugate is within one mass unit of the expected molecular weight.

FIG. 32 shows cell survival of two cancer cell lines upon treatment with assemblies consisting of SpyTagged-ligand L7 and/or DogTagged-ligand L7 and SpC3-(GGGGS)3—[C75A, C96A]-HsCutA1₆₀₋₁₇₁-(GGGGS)3-DgC (SEQ ID NO: 75), SpC3-(GGGGS)—scHsCutA1-(GGGGS)-DgC (SEQ ID NO: 99) or SpC3-(GGGGS)3-L9-(GGGGS)3-DgC (SEQ ID NO: 98). Colo205 and HCT116 cells were treated with doses ranging from 0.0008 to 2 nM. Data shown is normalised to mock-treated controls.

FIG. 33 shows cell survival of the cancer cell line Colo205 upon treatment with assemblies consisting of DogTag-ligand L7 and SpyTag-ligand L8 or -L10, both in scFv and Fab formats, and SpC3-GGGGS—[C75A, C96A, K82C]-HsCutA₄₄₋₁₇₉-GGGGS-DgC (SEQ ID NO: 93). The hexavalent nanobody-based L7 assembly served as a control. Colo205 cells were treated with doses ranging from 0.0008 to 2 nM. Data shown is normalised to mock-treated controls.

FIG. 34 shows cell survival of the cancer cell line HCT116 upon treatment with the [C75A, C96A]HsCutA1₆₀₋₁₇₁ based multivalent bispecific fusion containing the L7 and L11 binders, or the failed clinical candidate, a tetrameric L7 nanobody fusion. The monovalent nanobody-based L7 binder and [C75A, C96A]HsCutA1₆₀₋₁₇₁ were used as controls. HCT116 cells were treated with doses ranging from 0.000256 to 0.8 nM. Data shown is normalised to mock-treated controls.

FIG. 35 shows a schematic of the coupling of SpyCatcher003 and DogCatcher to magnetic beads to be used during the assembly purification pipeline. Glutathione (GSH)-conjugated magnetic beads are mixed with GST-SpC3 and GST-DgC both at equimolar concentrations. The binding of the GST-Catchers to the beads is incubated for 1 hour at 25° C. The beads are then sedimented using a magnetic block and washed 3× using PBS, or until a baseline absorbance reading at 280 nm is achieved. The GST bound beads containing both Catchers are retained for use during the assembly purification.

FIG. 36 shows SDS-PAGE (4-20% gradient) of the preparation of glutathione-conjugated beads with GST-SpC3 and GST-DgC. MagneGST resin was prepared with SDS-loading buffer to check for the presence of contaminating proteins in the stock. W1-3: PBS washes.

FIG. 37 shows a schematic of another variation of Catcher-based protein assembly and clean-up. SpC3-Core-DgC is combined with SpT-protein and DgT-protein with a 1.4× excess of tagged-protein to Catcher-core. The conjugation reaction is incubated for 1 hour at 25° C. to allow full capture of the tagged-proteins. Pre-prepared paramagnetic glutathione-conjugated beads bound to GST-SpC3 and GST-DgC as described in FIG. 35 are added to give a 2× excess of each GST-Catcher to their corresponding tagged-protein. The sample is transferred to a magnetic block to capture the bead-GST-Catcher-Tag conjugates and supernatant containing the Core-Catcher-Tag conjugates is retained for further analysis and downstream assays.

FIG. 38 shows SDS-PAGE (4-20% gradient) of protein assembly and MagneGST purification. L7^{SpT}:CC7:L2^{DgT} protein assembly prior to purification (L7^{SpT}:CC7:L2^{DgT} (pre)) shows excess of both binders, whereas L7^{SpT}:CC7:L2^{DgT} protein assembly after purification (L7^{SpT}:CC7:L2^{DgT} (post)) shows fully assembled molecule only. GST-Catchers+Resin demonstrate the binder excess has been captured during MagneGST purification. (CC7 corresponds to SEQ ID NO: 76).

FIG. 39 shows the design and production of single-chain SpC3-HsCutA1-DgC. (A) shows a schematic of the SpC3-HsCutA1-DgC as a linear construct and a tertiary arrangement. A predicted structural of the single-chain HsCutA1 core generated by AlphaFold2 is also shown. (B) Shows the UV A280 absorbance from size exclusion chromatography using HiLoad 16/600 Superdex 200 pg for single-chain SpC3-HsCutA1-DgC. The 2 mL fractions form the highlighted peaks were separated by SDS-PAGE and fractions B6-B11 retained as purified single-chain SpC3-HsCutA1-DgC.

FIG. 40 shows induction of apoptosis by trivalent and hexavalent L7 assemblies, measured as activation of caspase 3/7. L7 was conjugated to SpC3-HsCutA₄₄₋₁₇₉-DgC (SEQ ID NO: 24) to produce trivalent and hexavalent assembled molecules. Assemblies were used to treat Colo205 cells and caspase 3/7 activity was measured 1 h (A) and 24 h (B) after the start of the treatment. After 1 h, only the hexavalent, and not the trivalent or combination of both trivalent molecules, induced apoptosis. After 24 h, caspase 3/7 activity was observed upon treatment with trivalent assemblies.

FIG. 41 shows cell survival of the liver cancer cell line HepG2 upon treatment with the L11-HsCutA1-L7 direct fusion, the failed clinical asset TAS266 and HsCutA1 in the presence or absence of intravenous immunoglobulin (IGIV 0.2-10 mg/ml). Untreated cells growing in the absence of IVIG were considered 100% survival. L11-HsCutA1-L7 did not trigger an increase in ADA-associated cytotoxicity, in contrast to TAS266.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

In addition as used in this specification and the appended claims, the singular forms “a”, “an”, and “the” include plural referents unless the content clearly dictates otherwise. Thus, for example, reference to “a scaffold” includes two or more scaffolds, reference to “an oligomer” includes two or more such oligomers and the like.

All publications, patents and patent applications cited herein, whether supra or infra, are hereby incorporated by reference in their entirety.

### Definitions

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypeptides that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” typically has at least 50%, 60%, 70%, 80%, 90%, 95%, 96%, 97%, 98% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy Scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide may be chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

### Polypeptide Constructs

The invention relates in part to multi-domain polypeptide constructs that are useful when two or more are combined to form an oligomeric protein and may, in some embodiments, also be useful as monomers. The multi-domain polypeptide is typically engineered to combine domains that do not exist together in nature. In some embodiments, 3, 4, 5 or 6 polypeptide constructs are combined to form an oligomer. In some embodiments, 3 constructs are combined to form a trimer, for example a homotrimer.

The description provided herein describes an oligomeric core of subunit monomers. The subunit monomer is typically the structural domain of the multi-domain polypeptide construct. The oligomerisation of these structural domains in turn may form the core of a multivalent protein scaffold.

Accordingly, discussion of the features of the subunit monomer also applies to the disclosure and definition of the structural domain of the individual polypeptide constructs.

Similarly, the first binding domain and second binding domain of the polypeptide construct may form the first binding site and second binding site as described elsewhere herein, or may form the first effector moiety and the second effector moiety as described elsewhere herein, depending on the context. For example, when the binding domain is an isopeptide bond-forming “catcher” domain (or other binding site as described herein) then it is a binding site as described elsewhere, below. When the binding domain is e.g. an antibody, an antigen-binding fragment, an antibody mimic, a protein or peptide ligand, a protein or peptide signalling molecule (e.g. cytokine) a biological receptor or other molecule described as an effector moiety herein, then the binding domain is an effector moiety as described elsewhere herein.

Accordingly, definitions and description of the first and second binding sites, or of the first and second effector moieties, apply as appropriate to the first binding domain and second binding domain of the polypeptide construct.

In some embodiments, a polypeptide construct comprises a first binding domain at the N terminus and a second binding domain at the C terminus, wherein the first and second binding domains are separated by a structural domain. The N terminus refers to the terminal amino acid residue at the amino terminus of a polypeptide. The C terminus refers to the terminal amino acid residue at the carboxy terminus of a polypeptide. Typically, the first binding domain and second binding domain are able to bind to their targets when the target molecules are expressed on a single cell or immobilised onto a plate or single bead. This is sometimes described herein as providing the first and second binding domains in “cis” orientation. Typically, the first binding domain and the second binding domain are able to bind to their cell targets on the surface of a single cell, and cluster both targets in the cell membrane. A cis orientation can therefore be preferential for some cis acting agents (i.e. that can act on a single cell). Cis orientation of bispecific antibodies is discussed in Dickopf et al (Computational and Structural Biotechnology Journal, Volume 18, 2020, Pages 1221-1227), along with the converse “trans” orientation.

In the context of geometry, “cis-orientation” is used herein to refer to a spatial arrangement in which two components are (from Latin) “on the same side” of a plane, as opposed to “trans” or “trans-orientation” in the context of geometry in which two components are “across” (from Latin), i.e. on different sides of a plane, similar to cis-trans-isomerism and previously used to describe bispecific antibody architecture. This geometric definition is distinct from “cis-acting” and “trans-acting” in the context of biological effect, in which a single bispecific molecule acts on a single or adjacent cell (cis) or on distinct cell populations (trans), e.g. recruiting an effector cell to a target cell. There is active interest and effort towards the exploration of different format geometries (e.g. see Dengl et al Nat Commun. 2020; 11:4974). “Cis-orientation” can also be beneficial in certain “trans-acting” bispecifics, e.g. by reducing intermolecular distance (Dickopf et al, Computational and Structural Biotechnology Journal, Volume 18, 2020, pp. 1221-1227). Cis-orientation is of particular interest in multivalent cis-orientation towards cis-acting bispecifics via multivalent binding or clustering of targets on a single cell (compare for example to bispecific tandem-fusion described by Veggiani et al Biochemistry Jan. 19, 2016, 113 (5) 1202-1207 or higher-order monospecific clustering Khairil Anuar et al, Nature Communications volume 10, Article number: 1734 (2019)).

The function of the structural domain is to provide a defined structural support for the binding domains. Advantageously, the structural domain can ensure that the binding domains have the desired orientation so that they can bind their targets, typically with both binding domains in the cis orientation. The constructs can therefore present a single binding surface.

In certain embodiments, the attachment site for the binding domains on the structural domain (oligomeric core) allows binding even for short linkers.

The structural domain may be any polypeptide domain comprising a defined secondary structure, typically an alpha helix or a beta sheet. In some particularly advantageous embodiments, the structural domain has its N and C termini in the same spatial region, for example substantially adjacent or adjacent to each other. Attaching the binding domains to the termini of the structural domain then provides the two binding domains substantially adjacent in the three-dimensional conformation. In some embodiments, the N and C termini are oriented to face in substantially the same direction. As described elsewhere herein, the provision of N and C termini that are adjacent in space can result in the binding domains being situated on the same face of the construct, or on the same face of the oligomer comprising multiple constructs. Accordingly, the constructs typically present a single binding surface. The constructs typically present the binding regions in cis orientation.

In some embodiments, the structural domain presents the binding domains in a suitable orientation via the tertiary structure of a monomer (e.g. suitable relative positioning of N and C termini of a single monomer). In some embodiments, the structural domain presents the binding domains in a suitable orientation via association of monomers to a quaternary structure (e.g. suitable relative positioning of N and/or C termini between monomers). In some preferred embodiments, the structural domain presents the binding domains in such a suitable orientation via the tertiary structure of a monomer combined with association of monomers to a quaternary structure (e.g. suitable relative positioning of N and C termini within and between monomers).

The structural domains may comprise a single polypeptide chain, or may be formed of two or more separate polypeptide chains that associate to form a single structural domain, for example two anti-parallel (N—C C—N) alpha helices or two or more beta strands that associate to form a beta sheet. In some embodiments, two or more polypeptide chains with appropriate characteristics are identified and then fused, typically by recombinant means to form a single polypeptide chain (i.e. a fusion protein), but also by chemical conjugation or bonding to form a single covalent molecule.

The structural domain is different from the two binding domains. Therefore, when the binding domains are catcher polypeptides such as SpyCatcher, DogCatcher or SnoopCatcher, the structural domain is not a catcher polypeptide.

Typically, the structural domain does not comprise a CH2 domain. Typically, the structural domain does not comprise a CH3 domain. In some embodiments, the structural domain does not comprise a CH2 domain and does not comprise a CH3 domain.

A number of exemplary structural domains have been identified by the inventors, as discussed below and in the Examples. In some embodiments, the structural domain comprises or consists of the Collagen X NC1 domain (SEQ ID NO:2), or a polypeptide with at least 50%, at least 60%, at least 70% or at least 80%, for example at least 90% or at least 95% identity thereto. In some embodiments, the structural domain comprises or consists of the Collagen VIII NC1 domain (SEQ ID NO:3), or a polypeptide with at least 50%, at least 60%, at least 70% or at least 80%, for example at least 90% or at least 95% identity thereto. In some embodiments, the structural domain comprises or consists of a CutA1 polypeptide (e.g. SEQ ID NO:1 or SEQ ID NO: 19), or a polypeptide with at least 50%, at least 60%, at least 70% or at least 80%, for example at least 90% or at least 95% identity thereto.

In certain embodiments, the structural element comprises or consists of a polypeptide with at least 50% amino acid identity, for example at least 90% identity, to SEQ ID NO: 1, SEQ ID NO: 2, SEQ ID NO: 3, SEQ ID NO: 19, SEQ ID NO: 29, SEQ ID NO: 60, SEQ ID NO: 25, SEQ ID NO: 26, SEQ ID NO: 27, SEQ ID NO: 42, SEQ ID NO: 31, SEQ ID NO: 78, SEQ ID NO: 80 or SEQ ID NO: 58.

The Collagen IV C4 domain (also known as the collagen IV NC1 domain) (PDB ID: 1m3d, SEQ ID NO: 49) is a suitable structural domain, or a polypeptide with at least 50%, at least 60%, at least 70% or at least 80%, for example at least 90% or at least 95% identity thereto.

Collagen NC1 domains can generally be used as structural domains according to the invention, including the NC1 domains from Collagen IV, Collagen VIII and Collagen X.

However, not all collagen NC1 domains are appropriate as structural domains. In particular NCI domains from Collagen XV and from Collagen XVIII do not have the required orientation.

In certain embodiments, the structural domain comprises human macrophage migration inhibitory factor (MIF) (PDB ID: 1CA7, or SEQ ID NO: 25, or with Y99G mutation PDB ID: 60Y8) or human macrophage migration inhibitory factor 2 (MIF2) (PDB ID: 7MSE, or SEQ ID NO: 26, or with S62A and F99A mutation SED ID NO: 27) or a homolog or paralog thereof.

In certain embodiments, the structural domain comprises TNF family proteins including TNF (PDB ID: 1TNF, SEQ ID NO: 42; full-length soluble domain SEQ ID NO: 80), TL1A (PDB ID: 2RE9, SEQ ID NO: 31), OX40L (SEQ ID NO: 78), or CD40L (PDB ID: 3LKJ, SEQ ID NO: 58).

Further examples of structural domains include suitably modified antiparallel coiled-coil hexamer (PDB ID: 5W0J, see example 4, SEQ ID NO: 43), HIV-1 GP41 core (PDB ID: 115Y or SEQ ID NO: 44), cytochrome c555 (PDB ID: 5Z25 or SEQ ID NO: 45), MHC Class II associated chaperonin and targeting protein invariant chain (Ii) (PDB ID: 1iie or SEQ ID NO: 46), p53 (PDB ID: 1C26 or SEQ ID NO: 47); a fibrinogen-like domain (PDB ID: 4M7F or SEQ ID NO: 48), a Bacillus subtilis AbrB (PDB ID: 1YFB or SEQ ID NO: 50), bacteriophage lambda head protein D (e.g. PDB ID: 1C5E or PDB ID: 1C5E or SEQ ID NO: 51); the domain-swapped trimer variant of HCRBPII (PDB ID: 6VIS or SEQ ID NO: 52); the T1L reovirus attachment protein sigma1 (chain A,B,C of PDB ID: 40 DB or SEQ ID NO: 53).

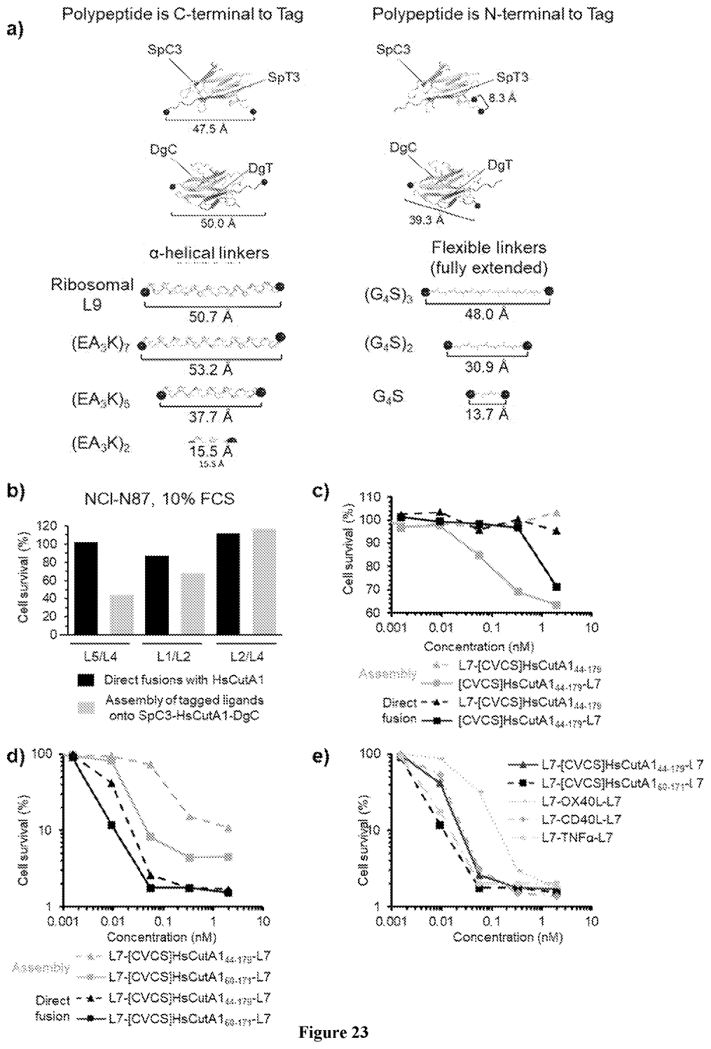

In some particular aspects, the structural domain of the polypeptide construct (or the subunit monomer of the oligomeric core) is a CutA1 protein, typically a human CutA1 protein. The data presented below, in particular FIG. 23, demonstrates various fusions of HsCutA1 (“Homo sapiens CutA1”) with effector proteins, as well as the ability of these direct fusions to exert biological effects. Accordingly, in certain aspects the invention provides a polypeptide comprising a first binding domain at the N terminus and a second binding domain at the C terminus, wherein the first and second binding domains are separated by a human CutA1 structural domain.

In certain aspects, the CutA1, typically human CutA1, is engineered to remove one or more cysteine residues from the native sequence (presented herein as SEQ ID NO: 19). Typically, this is a substitution of one or more cysteine residues with one or more non-cysteine residues. The removal of one or more cysteine residues beneficially allows for targeted cysteine conjugation at non-native sites or in fusion proteins. Without being bound by theory, unpaired cysteines routinely interfere with stability and downstream application, so it can be beneficial to remove unpaired cysteines.

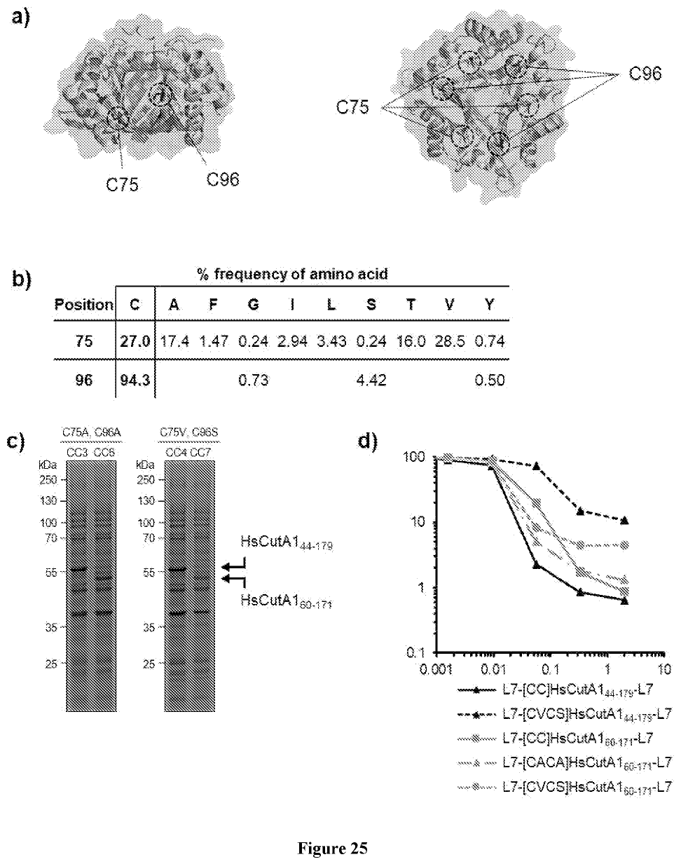

In some embodiments, one or more cysteine residues in CutA1 are substituted with one or more Alanine residues. In some embodiments, one or more cysteine residues in CutA1 are substituted with one or more Valine residues. In some embodiments, one or more cysteine residues in CutA1 are substituted with one or more Serine residues. In some embodiments, the substitution comprises or consists of two cysteines substituted with two alanines, which is referred to herein as a “CACA” substitution. In some embodiments, the substitution comprises or consists of one cysteine substituted with a valine and one cysteine substituted with a serine, which is referred to herein as a “CVCS” substitution. In some embodiments, the cysteine residues at positions 75 and 96 of wild-type human CutA1 (e.g. SEQ ID NO: 19) are substituted for different residues. In some embodiments, human CutA1 is engineered to substitute two cysteine residues, wherein the cysteine substitutions comprise or consist of (i) C75A, C96A or (ii) C75V, C96S. Accordingly, in some embodiments, the structural domain of the polypeptide construct (or the subunit monomer of the oligomeric core) is a human CutA1 protein engineered to substitute two cysteine residues, wherein the cysteine substitutions comprise or consist of (i) C75A, C96A or (ii) C75V, C96S. The generation, yield and biological effects of polypeptide constructs comprising such cysteine-substituted human CutA1 domains are described below (FIG. 25).

In certain aspects, the CutA1, typically human CutA1 (e.g. SEQ ID NO: 19), is engineered to delete one or more residues from either or both termini of the native sequence, to form a truncated CutA1 domain that is incorporated into the construct of the invention. In some embodiments, one or more residues are deleted from the N terminus of CutA1, typically human CutA1. Typically, 5 to 70 residues are deleted from the N terminus of CutA1, typically human CutA1, for example 10 to 59 residues. In some embodiments, 25, 26, 27, 28, 29, 30, 31, 32, 33, 34, 35, 36, 37, 38, 39, 40, 41, 42, 43, 44, 45, 50, 55, 59, 60, 61, 62, 63, 64, 65 or 66 residues are deleted from the N terminus of CutA1. In some embodiments, the truncated CutA1 begins at residue 33 (i.e 32 N-terminal residues are deleted). In some embodiments, the truncated CutA1 begins at residue 44 (i.e 43 N-terminal residues are deleted). In some embodiments, the truncated CutA1 begins at residue 60 (i.e 59 N-terminal residues are deleted). In some embodiments, the truncated CutA1 begins at residue 67 (i.e 66 N-terminal residues are deleted), for example the “HsCutA1 67-171 CACA” direct fusion construct exemplified in FIG. 23b.

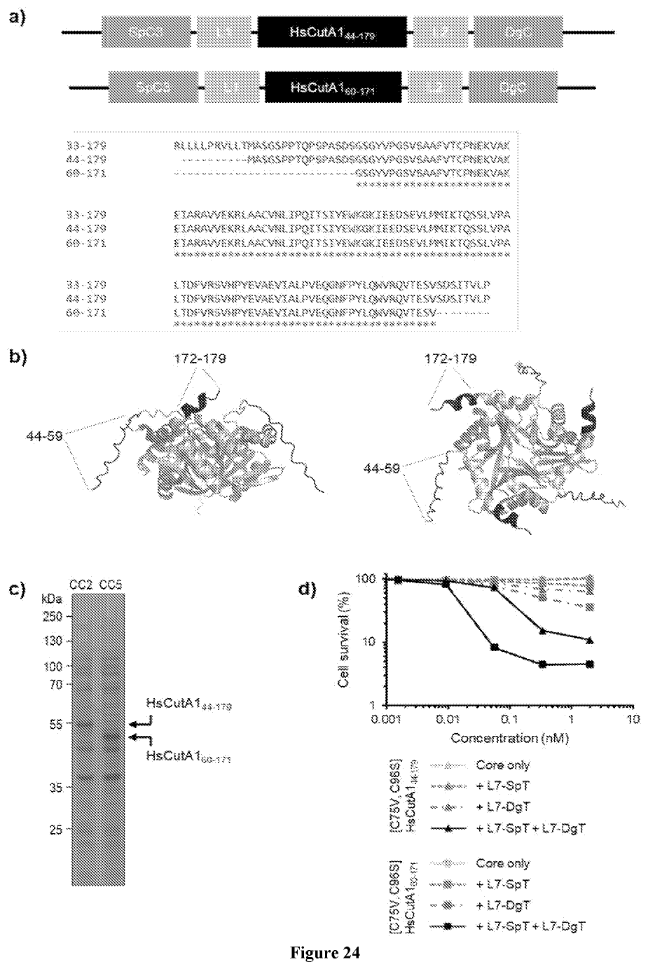

In some embodiments, one or more residues are deleted from the C terminus of CutA1, typically human CutA1. The C-terminal deletion may be instead of, or in addition to, the N-terminal deletion. Typically, 5 to 20 residues are deleted from the C terminus of CutA1, typically human CutA1, for example 6 to 12 residues are deleted. In some embodiments, approximately 8 residues are deleted, which in human CutA1 leaves a truncated protein with a C-terminus at what is residue 171 (valine) in the wild-type sequence shown in FIG. 24 and SEQ ID NO: 19. In some embodiments, the truncated protein has a C-terminus at what is residue 168 (threonine) in the wild-type sequence shown in FIG. 24 and SEQ ID NO: 19. In some embodiments, the truncated protein has a C-terminus at what is residue 169, 170, 172, 173, 174, 175 or 176 in the wild-type sequence shown in FIG. 24 and SEQ ID NO: 19.

In certain embodiments, the truncated CutA1 consists of residues 44-179 or residues 60-171 of SEQ ID NO: 19, as shown in FIG. 24. In some embodiments, the truncated CutA1 starts at any of residues 44 to 67 of SEQ ID NO: 19 and ends at any of residues 168 to 179. In other embodiments, the truncated CutA1 starts at any of residues 30 to 65 of SEQ ID NO: 19 and ends at any of residues 165 to 179. In some embodiments, the truncated CutA1 starts at any of residues 44 to 60 of SEQ ID NO: 19 and ends at any of residues 171 to 179.

The truncation of CutA1 can improve the precision by which fusion constructs can be constructed. FIG. 24 demonstrates the generation, yield and biological effects of polypeptide constructs comprising such truncated human CutA1 domains.

In certain aspects, the CutA1 is a human CutA1 that is engineered to remove one or more cysteine residues from the native sequence and that is truncated at the N and/or C termini. In some embodiments, the truncated CutA1 consists of residues 44-179 or residues 60-171 of SEQ ID NO: 19, as shown in FIG. 24, and has cysteine substitutions that comprise or consist of (i) C75A, C96A or (ii) C75V, C96S. In other embodiments, the truncated CutA1 starts at any of residues 30 to 65 of SEQ ID NO: 19 and end at any of residues 165 to 179, and also has cysteine substitutions that comprise or consist of (i) C75A, C96A or (ii) C75V, C96S.

A polypeptide construct according to the invention comprises a first binding domain and a second binding domain, in addition to the structural domain.

In certain embodiments, the binding domains are able to form an isopeptide linkage with a cognate peptide, for example the various catcher domains that are well-known in the art. Constructs comprising these isopeptide bond-forming domains are particularly well-suited to screening of different pairs of effector molecules such as antigen binding proteins. As discussed elsewhere herein, many combinations of effector molecules can be linked to the constructs comprising isopeptide bond forming binding domains, via isopeptide-forming peptide tags. Accordingly, the constructs comprising binding domains able to form an isopeptide linkage with a cognate peptide are particularly useful as a drug discovery platform.

The aspects of the invention relating to isopeptide bond-formation are generally exemplified with reference to a larger molecule (domain), typically referred to as a catcher, attached to the structural domain and a smaller polypeptide or peptide, typically referred to as the tag, forming part of the binding region (e.g. antigen-biding domain) of interest. However, all aspects and embodiments can be performed in the reverse orientation wherein the larger (e.g. catcher) molecule forms part of the binding region (e.g. antigen-binding domain) of interest and the smaller tag peptide forms the binding domain attached to the structural domain.

In some embodiments, the first binding domain and the second binding domain in the polypeptide construct are effector molecules such as antigen-binding domains. In these embodiments, the constructs are particularly suited for use as diagnostic, analytical or therapeutic agents.

In some embodiments, an interesting or effective pair of antigen-binding regions is identified using the drug discovery platform of the invention (e.g. wherein the construct comprises isopeptide bond-forming binding domains) and the construct is then expressed without the isopeptide bond-forming binding regions, and with the identified combination of antigen-binding domains (or other effector moiety) connected directly to a structural domain without the intermediary catcher domains on the structural domain and without the peptide tags on the antigen-binding domains. For the avoidance of doubt, these direct fusion constructs may still comprise a linker region between the terminal residue of the structural domain and the terminal residue of the or each effector moiety (e.g. antigen-binding region), as described in detail elsewhere herein.

Accordingly, one aspect of the invention provides a system for large-scale high-throughput screening of many possible combinations of effector molecules using combinatorial pairs of tagged effector proteins, and the combinations identified as useful can be used in the form that they were provided in the screening construct or converted into a simpler format (e.g. for therapeutic candidates) by creating a direct-fusion of the effector molecules (e.g. antigen-binding regions) onto the same structural domain as was used in the drug discovery platform. This provides a simple, fast and reliable technology to identify and develop bispecific and multispecific agents.

Antigen-binding domains are a typical domain that can be used and applied according to the invention. In certain aspects of the invention, antigen-binding domains comprise a peptide tag that can form an isopeptide bond, such as a SpyTag or a SnoopTag, and can be bound by an isopeptide bond to a construct comprising a cognate catcher domain, for example to create a platform for a combinatorial or modular screen. In other aspects of the invention, the construct of the invention comprises a first antigen-binding domain at the N terminus and a second antigen-binding domain at the C terminus, wherein the first and second binding domains are separated by a structural domain. There may optionally be a linker sequence between one or each of the antigen-binding domains and the structural domain. As discussed below, suitable peptide linkers for use in connecting a binding domain (binding site) to a structural domain (monomer subunit) are typically between 1 and 150, 1 and 100, 1 and 50, 1 and 25, 1 and 20, 1 and 15, or 1 to 10 amino acids in length. An example of a linker sequence is GSGS, GGGGS, GGGGSGGGGS, or GGGGSGGGGSGGGGS. Other suitable linkers include linkers that form an alpha-helix secondary structure, for example EAAAK and multiples thereof, for example (EAAAK)₂, (EAAAK)₅ or (EAAAK)₇, or an alpha-helical linker derived from Ribosomal L9 protein with sequence PANLKALEAQKQKEQRQAAEELANAKKLKEQLEK (Kuhlman et al 1997 J Mol Biol 270, 5, 640-647) or a derivative (e.g. the derivative comprising 1, 2, 3, 4, 5 or up to 10 substitutions, insertions or deletions) or multiples thereof, for example a multiple of 2, 3, 4, 5 or up to 10 repeats of the L9 linker. Other suitable linkers include linkers that impart variable function to the fused protein such as those discussed in Chen et al 2013 (Adv Drug Deliv Rev. 2013, 65, 10, 1357-1369), including but not limited to PAPAP, CC, AP, VSQTSKLTRAETVFPDV, PLGLWA, RVLAEA, EDVVCCSMSY, GGIEGRGS, TRHRQPRGWE, AGNRVRRSVG, RRRRR, RRRRRRRRR, GFLG, or LE, or a derivative (e.g. the derivative comprising 1, 2, 3, 4, 5 or up to 10 substitutions, insertions or deletions) or multiples thereof, for example a multiple of 2, 3, 4, 5 or up to 10 repeats of the PAPAP linker. Catcher domains share structural similarities with antibody domains, having immunoglobulin folds (Kang et al 2007, Science. 318, 5856, 1625-1628). Substitution of Catcher domains with human antibody domains enables an alternate route for generation of humanised direct fusion constructs. Therefore, further suitable linkers include the domains of antibody fragments, for example CH1, CH2, or CH3 from various antibody isotypes or from various species and their modifications thereof, preferably of human origin. Examples include CH1 domain from human IgG1 isotype with sequence ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKV, CH2 domain from human IgG1 isotype with sequence PCPAPELLGGPSVFLFPPKPKDTLMISRTPEVTCVVVDVSHEDPEVKFNWYVDGVE VHNAKTKPREEQYNSTYRVVSVLTVLHQDWLNGKEYKCKVSNKALPAPIEKTISK AK, or CH3 domain from human IgG1 isotype with sequence GQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK.

The antigen-binding domain is typically an antigen-binding fragment of an antibody. An antigen-binding antibody fragment is not a full-length intact antibody, and typically lacks at least the CH2 and/or CH3 domains. Such antigen-binding fragments are well-known, and include a Fab, F(ab′)2, Fv, or a single chain Fv fragment (scFv). Antigen binding fragments typically comprise the CDRs (typically six CDRs) required for antigen binding, and the framework residues necessary for correct CDR structure. In some embodiments, the antigen-binding domain comprises a heavy (H) chain variable domain sequence (VH), and a light (L) chain variable domain sequence (VL).

In some embodiments, the antigen-binding region can be a single domain antibody. Single domain antibodies (sdAb) can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from species including, but not limited to mouse, human, camel, llama, fish, shark, goat, rabbit, and bovine. A single domain antibody may be a naturally occurring single domain antibody known as heavy chain antibody devoid of light chains. Such single domain antibodies are disclosed in WO-A-94/04678, for example. For clarity reasons, this variable domain derived from a heavy chain antibody naturally devoid of light chain is sometimes referred to as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The antigen-binding domain may also comprise or consist of an antibody mimetic, such as an affibody or a DARPin. An affibody is, as known in the art, is a small polypeptide containing three alpha helices typically with around 58 amino acids and having a molecular mass of about 6 kDa. As is known in the art, a DARPIN (designed ankyrin repeat protein) is a genetically engineered antibody mimetic protein typically exhibiting highly specific and high-affinity target protein binding.

The binding domain may comprise naturally occurring ligands, such as cytokines, as an alternative to an antigen-binding domain.

When two different antigen binding domains are present on a bispecific molecule, they typically bind to different epitopes. This may be different epitopes on the same target molecule, or may be different epitopes on different target molecules. In some embodiments, the epitopes are both on therapeutic targets, wherein binding of an antigen-binding domain to the therapeutic target modifies a biological mechanism, typically a pathological mechanism, for therapeutic benefit.

In some embodiments, the antigen binding domains may each agonise a biological target. In some embodiments, the antigen binding domains may each antagonise a biological target. In some embodiments, one antigen-binding domain may agonise a first target and the other antigen-binding domain may antagonise a second target.

In some embodiments, a construct may comprise two binding regions that bind to the same epitope but have different affinities for that epitope. In some embodiments, a construct may comprise two binding regions that bind to the same epitope, optionally with different affinities, and the two binding regions have a different format, for example one antigen-binding region is an scFv and the second is a Fab.

Multi-domain polypeptide constructs of the invention typically have the following format, depicted in the N to C orientation according to the usual convention:
(Binding domain 1)-Linker1-Structural Domain-Linker2-(Binding domain 2)
   wherein Linker1 and Linker2 are optional linker sequences, optionally between 1 and 20 amino acids, for example GSGS. An optional purification tag, for example a His tag, for example 6×His, can be incorporated at either end of the construct.

Binding domains may be the same or typically are different. Binding domains may typically be catcher polypeptides, or antigen-binding domains. A number of illustrative constructs are provided below.
SpC-Linker1-CutA1-Linker2-SpC
SnC-Linker1-CutA1-Linker2-SnC
SnC-Linker1-CutA1-Linker2-SpC
SpC-Linker1-CutA1-Linker2-SnC
SpC3-Linker1-CutA1-Linker2-DgC
scFv-Linker1-CutA1-Linker2-ScFv
Fab-Linker1-CutA1-Linker2-Fab
ScFv-Linker1-CutA1-Linker2-Fab
Fab-Linker1-CutA1-Linker2-ScFv
nanobody 1-Linker1-CutA1-Linker2-nanobody2
nanobody-Linker1-CutA1-Linker2-DgC
SpC3-Linker1-CutA1-Linker2-nanobody
   wherein SpC is SpyCatcher, SpC3 is SpyCatcher003, SnC is SnoopCatcher, Fab is an antibody “fragment antigen binding” and scFv is a single chain Fv. In each case, the Linker1 and Linker2 linkers are optional. The CutA1 sequences may be human or from Pyrococcus horikoshii, or a homologue from another species, or have at least 30%, at least 50%, at least 70% or at least 90% identity to the human or Pyrococcus horikoshii sequence.

Further illustrative constructs are:
SpC-Linker1-NC1-Linker2-SpC
SnC-Linker1-NC1-Linker2-SnC
SnC-Linker1-NC1-Linker2-SpC
SpC-Linker1-NC1-Linker2-SnC
scFv-Linker1-NC1-Linker2-ScFv
Fab-Linker1-NC1-Linker2-Fab
ScFv-Linker1-NC1-Linker2-Fab
Fab-Linker1-NC1-Linker2-ScFv
nanobody 1-Linker1-NC1-Linker2-nanobody2
nanobody-Linker1-NC1-Linker2-DgC
SpC3-Linker1-NC1-Linker2-nanobody
   wherein NC1 is a collagen NC1 domain from Collagen VIII or Collagen X. In each case, the Linker1 and Linker2 linkers are optional.

Further illustrative constructs comprise the macrophage migration inhibitory factor (MIF) (SEQ ID NO: 25) or macrophage migration inhibitory factor 2 (MIF2) (SEQ ID NO: 26) or S62A F99A mutant of MIF2 (MIF2m, SEQ ID NO: 27) as the structural domain, including:
SpC-Linker1-MIF2-Linker2-SpC
SnC-Linker1-MIF2-Linker2-SnC
SnC-Linker1-MIF2-Linker2-SpC
SpC-Linker1-MIF2-Linker2-SnC
scFv-Linker1-MIF2-Linker2-ScFv
Fab-Linker1-MIF2-Linker2-Fab
ScFv-Linker1-MIF2-Linker2-Fab
Fab-Linker1-MIF2-Linker2-ScFv
nanobody 1-Linker1-MIF2-Linker2-nanobody2
nanobody-Linker1-MIF2-Linker2-DgC
SpC3-Linker1-MIF2-Linker2-nanobody

Any suitable structural domain, in particular any of the structural domains described herein, may be used in these constructs instead of the exemplary structural domains provided above. Accordingly, these exemplary formats are described for use with structural domains in general and with the structural domains described herein.

The orientation of the binding domains in the multi-domain construct, in monomeric or oligmeric form, can be assessed functionally using a variety of assays. A selection of exemplary assays are described below.

FRET: To demonstrate cis-orientation of the selected scaffolds compared to non-cis-oriented proteins, a FRET assay can be performed. The scaffold polypeptide with Catcher components, for example SpC3-HsCutA1-DgC, can be conjugated to fluorescent protein FRET pairs fused to the respective Tag pairs, for example mCherry (6+)-SpT3-H6 and H6-DgT-mCitrine (4-). Upon conjugation of the tagged FRET pairs to the scaffold protein, the emission of the acceptor FRET protein can be measured via standard fluorescence reading and compared to the sensitised emission of the donor FRET protein. Protein scaffolds that show preferential cis-orientation will show higher acceptor emission, whereas protein scaffolds that show preferential trans-orientation will show higher donor sensitised emission.

SPR: To demonstrate that cis-oriented scaffolds conjugated to suitable ligands may preferentially bind to targets on the same plane compared to non-cis-oriented proteins, an SPR experiment can be performed. Target proteins against the scaffold-conjugated ligands, for example the targets against L1 and L2 in SpC-PhCutA1-SnC: SnT-L1:L2-SpT, can be immobilized on the surface of the SPR sensor chip, either together, or separately as L1-target only or L2-target only for controls. Cis-oriented or non-cis-oriented scaffolds conjugated to L1 and L2 are then loaded to the SPR sensor chip with the L1- and/or L2-targets and the conjugated assemblies' binding to the immobilised targets on the chip is determined. Assemblies with cis-orientation is expected to show highly measurable binding to both L1- and L2-targets when both targets are immobilised on the same chip, whereas assemblies with non-cis-orientation is expected to not have highly measurable binding to such chip. Both assembly types are expected to have highly measurable binding to immobilised L1- or L2-targets only.

SEC-MALS: To demonstrate the native oligomeric state of the scaffold and assembly proteins in solution, a SEC-MALS experiment can be performed. Scaffold and assembly proteins can be prepared as described in the methods section. The samples are then injected into an FPLC machine coupled to a MALS machine and detector to separate the samples by size and the native protein mass is approximated by calculations of light scattering. The oligomeric state of the proteins can then be derived by dividing the native protein mass by predicted monomeric mass calculated through softwares such as ProtParam. Both scaffold and assembly proteins, for example SpC-PhCutA1-SnC and SpC-PhCutA1-SnC: SnT-L1:L2-SpT, are expected to show oligomeric state of 3.

Crosslinking and LC-MS/MS: To demonstrate the simultaneous binding of two targets by a cis-oriented conjugated assembly, for example the targets against L1 and L2, targets-expressing cells can be incubated with a biotinylated version of the conjugated assembly, biotin-SpC-PhCutA1-SnC: SnT-L1:L2-SpT, and subsequently the binding between targets and biotinylated-assembly can be crosslinked with BS3 (bis(sulfosuccinimidyl) suberate), followed by cell lysis and the extraction of the crosslinked target-assembly complex via streptavidin. This complex is then tryptic-digested to be feed into LC-MS/MS to confirm the binding of L1 and L2 to their respective targets. The same methodology can be run for non-cis-oriented assemblies, whereby the output of the LC-MS/MS data can be compared between the two protein assemblies. It is expected that cis-oriented assemblies may preferentially bind to both L1- and L2-targets, whereas non-cis-oriented assemblies may preferentially bind to either L1- or L2-targets only.

### Multivalent Protein Scaffold

One aspect of the invention relates to a modular system for screening target molecules. The system allows for the multivalent presentation of the target molecules. In one aspect, a multivalent protein scaffold is provided. The multivalent protein scaffold comprises an oligomeric core comprising a plurality of subunit monomers. The multivalent protein scaffold also comprises at least one first binding site orthogonal to at least one second binding site. Suitable binding sites are described in more detail herein.

The scaffold acts as a platform to which other molecules may be bound. Different combinations of molecules may be bound to the scaffold in a modular fashion. The scaffold allows multivalent binding of the molecules. Generally, the molecules bound to the scaffold have a potential therapeutic benefit, and the scaffold bound to the molecules can be used to investigate whether multivalent assemblies of different molecules may have a desired effect. For example, different combinations of potential anti-cancer polypeptides may be attached to the scaffold and the resulting assembly can then be used in a screening assay to see if the combination has an effect on, such as binding to and causing the death of, a cancer cell. Once a combination of molecules has been identified, a therapeutic drug candidate may be produced by modifying the multivalent protein scaffold so that it is directly attached to the identified molecules, rather than by using a modular system. The multivalent protein scaffold ‘presents’ the molecules on the same face of the scaffold, thereby allowing all of the molecules to potentially interact with a target cell.

The provided scaffold typically comprises at least two first binding sites and at least two second binding sites. The presence of at least two first and at least two second binding sites allows the scaffold to be used to screen for multivalent interactions, that may not necessarily be seen when screening with, for example, a bispecific antibody format. It also allows to test different scaffolds as a “toolbox” with regards to the same combination. Accordingly, provided herein is a multivalent protein scaffold comprising:
an oligomeric core comprising a plurality of subunit monomers; and
at least two first binding sites orthogonal to at least two second binding sites;
wherein said first binding sites and said second binding sites are positioned on the same face of the scaffold.

The provided scaffold typically comprises binding sites capable of forming covalent bonds to their respective targets. Covalent bonds lead to strong irreversible association. Complexes generated by covalently attaching the scaffold of the invention to targets for the binding sites are physically robust and can be readily produced. Such complexes can be produced in high yields and with high homogeneity. Accordingly the biological response produced when such complexes are administered to a biological system such as a subject as described herein are reproducible and controllable.

Accordingly, also provided herein is a multivalent protein scaffold comprising:
an oligomeric core comprising a plurality of subunit monomers;
at least one first binding site orthogonal to at least one second binding site;
wherein said first binding site(s) and said second binding site(s) are positioned on the same face of the scaffold; and wherein said first binding site comprises a first protein domain capable of forming a covalent bond to a first polypeptide target; and said second binding site comprises a second protein domain capable of forming a covalent bond to a second polypeptide target.

As explained herein, the scaffold provided herein has significant advantages over conventional antibodies including bispecific antibodies. The oligomeric core of the provided scaffold typically does not comprise an Fc region of an antibody. In some embodiments, the oligomeric core does not comprise a CH2 region. In some embodiments, the oligomeric core does not comprise CH3 region. In some embodiments, the oligomeric core does not comprise a CH2 region and does not comprise a CH3 region. The use of immunoglobulin domains, typically constant domains such as in an Fc region, of an antibody, typically may not display the advantages of the provided scaffold described herein. For example, a bispecific antibody lacks the modularity of the present invention and may not be useful for investigating multivalent interactions.

Accordingly, also provided herein is a multivalent protein scaffold comprising:
an oligomeric core comprising a plurality of subunit monomers;
at least one first binding site orthogonal to at least one second binding site;
wherein said first binding site(s) and said second binding site(s) are positioned on the same face of the scaffold; and wherein said oligomeric core does not comprise an Fc region of an antibody.

Also provided herein is a multivalent protein scaffold comprising:
an oligomeric core comprising a plurality of subunit monomers;
at least one first binding site orthogonal to at least one second binding site;
wherein said first binding site(s) and said second binding site(s) are positioned on the same face of the scaffold; and wherein said oligomeric core does not comprise a CH2 domain of an antibody, or does not comprise a CH3 domain of an antibody, or does not comprise a CH2 domain and does not comprise a CH3 domain.

The multivalent protein scaffold comprises an oligomeric core and at least one first binding site and at least one second binding site. The multivalent protein scaffold may also comprise other features, such as linkers, domain insertions and/or functional groups as described in more detail herein.

Preferably, the diameter of the multivalent protein scaffold is less than less than about about 100 nm, e.g. less than about 50 nm, e.g. less than about 25 nm, e.g. less than about 10 nm. Preferably, the height of the multivalent protein scaffold is less than less than about 100 nm, e.g. less than about 50 nm, such as less than about 30 nm, e.g. less than about 20 nm for example less than about 10 nm. The multivalent protein scaffold is preferably from 1 to 500 Å in size, such as from 2 to 250 Å, e.g. from about 10 to about 100 Å, such as from about 20 to about 80 Å.

Preferably, the multivalent protein scaffold itself preferably essentially does not induce an immune response in a biological system, cell culture or subject, such as in a human subject. In other words, typically in the absence of binding sites on the oligomeric core and/or effector moieties attached to the binding sites of the protein scaffold, no immune response (or essentially no immune response; e.g an immune response no greater than when a non-immunogenic protein is administered) is induced when the protein scaffold is administered to a biological system such as a human subject. For example, administration of the protein scaffold (in the absence of binding sites on the multivalent protein scaffold and/or effector moieties attached to the binding sites of the multivalent protein scaffold) to a biological system (e.g. a subject as defined herein) typically does not induce a biological system's innate or adaptive immunity. For example, the protein scaffold typically does not induce activation of the complement system, B cells, T cells, natural killer cells, mast cells, basophils, eosinophils, neutrophils, dendritic cells or macrophages.

The multivalent protein scaffold preferably does not comprise an antibody or antibody fragment; although as described further herein antibodies and/or antibody fragments may be attached as effector moieties to the scaffold. The multivalent protein scaffold (e.g. absent any effector moieties) more preferably does not comprise an Fc region of an antibody. An Fc region is the tail region of an antibody that interacts with cell surface receptors called Fc receptors and some proteins of the complement system. In some cases, the multivalent protein scaffold does not comprise an immunoglobulin constant region. In some embodiments, the multivalent protein scaffold does not comprises a CH2 domain. In some embodiments, the multivalent protein scaffold does not comprises a CH3 domain. In some embodiments, the multivalent protein scaffold does not comprises a CH2 domain and does not comprise a CH3 domain.

Preferably the multivalent protein scaffold is thermodynamically stable. Preferably the multivalent protein scaffold is stable at a temperature of from about 0 to about 100° C., e.g. from about 4° C. to about 90° C., such as from about 10° C. to about 50° C. e.g. from about 20 to about 38° C. e.g., from about 25 to about 37° C. In other words, preferably the multivalent protein scaffold does not dissociate into its substituent subunit monomers, and/or the binding sites do not disassociate from the oligomeric core, when in aqueous solution at a temperature of from about 0 to about 100° C. (e.g. from about 4° C. to about 90° C., such as from about 10° C. to about 50° C. e.g. from about 20 to about 38° C. e.g., from about 25 to about 37° C.); for example, at least 90%, such as at least 95%, e.g. at least 99%, such as at least 99.9%, e.g. at least 99.99% or 99.999% of the multivalent protein scaffold does not dissociate into its substituent subunit monomers, and/or the binding sites do not disassociate from the oligomeric core, when in aqueous solution at such a temperature. More preferably the multivalent protein scaffold is stable at a temperature of from about 0 to about 100° C., e.g. from about 4° C. to about 90° C., such as from about 10° C. to about 50° C., e.g. from about 20 to about 38° C. e.g., from about 25 to about 37° C. The multivalent protein scaffold preferably has a lifetime of at least 10 minutes, more preferably at least one hour, e.g. at least one day, such as at least one week, e.g. at least one month or at least one year, when determined at a temperature of from about 0° C. to about 100° C., e.g. from about 4° C. to about 90° C., such as from about 10° C. to about 50° C. e.g. from about 20 to about 38° C. e.g., from about 25 to about 37° C.

Preferably, the interactions between the constituents of the multivalent protein scaffold is not a weak transient interaction. Weak transient complexes show a dynamic mixture of different oligomeric states in vivo, whereas strong transient complexes change their quaternary state only when triggered by, for example, ligand binding. Weak transient interactions are characterized by a dissociation constant (K_{D}) in the micromolar range and lifetimes of seconds. Strong transient interactions, stabilized by binding of an effector molecule, may have a longer lifetime and have a lower K_{D} in the nanomolar range. The constituents of the multivalent protein scaffold preferably interact with at least strong transient reactions, more preferably the constituents of the multivalent protein scaffold form a permanent interaction. A permanent interaction means that the multivalent protein scaffold does not disassociate into its constituents under normal conditions, for example between 20° C. and 40° C. and between pH 6 and pH 8. A multivalent protein scaffold in which the constituent parts form a permanent interaction typically disassociates only under denaturing conditions that denature the tertiary structure of the subunit monomers themselves.

Preferably, therefore, the constituents of the multivalent protein scaffold interact with a K_{D} of less than 1 μM, e.g. less than 100 nM, more preferably less than 10 nM, at a temperature of from about 0° C. to about 100° C., e.g. from about 4° C. to about 90° C., such as from about 10° C. to about 50° C. e.g. from about 20 to about 38° C. e.g., from about 25 to about 37° C.

Preferably, the multivalent protein scaffold and the constituent parts thereof is stable to proteases. For example, the multivalent protein scaffold and the constituents of the multivalent protein scaffold may be exposed to proteases, such as trypsin, without loss of the tertiary or quaternary structure of the scaffold. The multivalent protein scaffold and the constituent parts of the multivalent protein scaffold are stable to proteases for at least 1 hour, e.g. at least 2 hours, e.g. at least 4 hours, e.g. at least 8 hours, such as at least 24 hours, or more, at a temperature of from about 10 to about 40° C., e.g. from about 20 to about 38° C. e.g., from about 25 to about 37° C.

### Oligomeric Core

As explained above, the multivalent protein scaffold provided herein comprises an oligomeric core comprising a plurality of subunit monomers. A subunit monomer of the oligomeric core is typically the structural domain of the polypeptide construct as described elsewhere herein.

Any suitable number of subunit monomers may be used. For example, the oligomeric core may comprise from about 2 to about 20 subunit monomers, e.g. from about 2 to about 10 subunit monomers, more preferably 3 to 7 subunit monomers, more preferably 3 to 6 subunit monomers. For example, the oligomeric core may comprise two, three, four, five, six, seven, eight, nine or 10 subunit monomers. Preferably, the oligomeric core comprises at least 3 subunit monomers. Most preferably the oligomeric core comprises three subunit monomers. Preferably, the oligomeric core does not comprise or consist of 7 subunit monomers.

Preferably, the subunit monomers have rotational symmetry when multimerised, such as three-fold rotational symmetry, four-fold rotational symmetry, five-fold rotational symmetry, six-fold rotational symmetry, or seven-fold rotational symmetry. The oligomeric core may have C2, C3, C4, D2, C5, C6, D3, C7, C8, D4, C9, C10, D5, C11, C12, D6 or T symmetry.

Some or all of the subunit monomers in the oligomeric core may be non-covalently attached together. Some or all of the subunit monomers in the oligomeric core may be covalently attached together. The oligomeric core may comprise a mix of subunit monomers attached covalently and non-covalently. For example, the oligomeric core may comprise a first monomer covalently bound to a second monomer to form a heterodimer. The oligomeric core may comprise at least two such heterodimers non-covalently bound together. For example, the oligomeric core may comprise three non-heterodimers non-covalently attached together, wherein each heterodimer comprises two monomers covalently bound together.

Some or all of the subunit monomers in the oligomeric core may be attached by non-covalent interactions. Suitable non-covalent interactions include, but are not limited to, electrostatic interactions, such as ionic bonds, hydrogen bonds and halogen bonds, Van der Waals forces, such as dipole-dipole interactions, π-π stacking, cation-π interactions, anion-π interactions or polar-π interactions.

Some or all of the subunit monomers in the oligomeric core may be covalently attached together. When subunit monomers are covalently attached together, the monomer is typically the amino acid sequence corresponding to the original or naturally occurring monomeric domain.

Two or more subunit monomers may be covalently linked by disulphide bonds. Disulphide bonds typically form between cysteine residues in polypeptides. Artificial amino acids having free thiol groups may also participate in disulphide bond formation.

Two or more subunit monomers may be covalently attached via chemical cross Cross linking reagents include homobifunctional crosslinking reagents, linking. heterobifunctional crosslinking reagents, and photoreactive crosslinking reagents. Homobifunctional crosslinking reagents have identical reactive groups at either end. Examples of homobifunctional crosslinking reagents include disuccinimidyl suberate (DSS), disuccinimidyl tartrate (DST) and dithiobis succinimidyl propionate (DSP). Some common examples of sulfhydryl-to-sulfhydryl crosslinkers include BMOE and DTME. Heterobifunctional crosslinking reagents possess two different reactive groups and can be used to link dissimilar functional groups. Examples of heterobifunctional crosslinking reagents include MDS (m-Maleimidobenzoyl-N-hydroxysuccinimide ester), GMBS (N-γ-Maleimidobutyryloxysuccinimide ester), EMCS (N-(ε-Maleimidocaproyloxy) succinimide ester) and sulfo-EMCS (N-(ε-Maleimidocaproyloxy) sulfo succinimide ester). Photoreactive crosslinking reagents are heterobifunctional crosslinkers that become reactive only upon exposure to ultraviolet or visible light. Two classes of common photoreactive chemical groups are aryl-azides and diazirines. Aryl azides (N-((2-pyridyldithio)ethyl)-4-azidosalicylamide) are widely used. Upon exposure to 250-350 nm UV light, these reagents can facilitate the formation of a nitrene group that may set off an addition reaction with the double bonds. Additionally, these crosslinkers may initiate the production of C—H insertion products or react with a nucleophile. Some common crosslinking reagents that belong to this group include ANB-NOS (N-5-Azido-2-nitrobenzyloxysuccinimide) and Sulfo-SANPAH. NHS-ester diazirines or azipentanoates contain a photoactivatable diazirine ring and an N-hydroxysuccinimide (NHS) ester which efficiently reacts with primary amino groups in neutral to basic buffers to form stable amide bonds. They exhibit better photostability compared to the phenyl azide group and can be easily activated with long-wave ultraviolet light (330 to 370 nm) to produce carbene intermediates that form covalent bonds with any peptide backbones or amino acid side chains within the spacer arm distance.

More preferably, two or more subunit monomers in the oligomeric core may be genetically fused together. Subunit monomers are genetically fused together if they are expressed are encoded in a single polynucleotide sequence such that they are expressed in a single polypeptide chain. Accordingly, when the subunit monomers are genetically fused together, the oligomeric core may comprise a single polypeptide chain.

Genetically fused subunit monomers may be genetically fused together via peptide linkers. Suitable peptide linkers for use in linking subunit monomers are amino acid sequences include those that can act as a hinge region between subunit monomers, thus allowing them to fold independently from one another and providing sufficient flexibility to allow the subunit monomers to retain their ability to multimerise. The length, flexibility and hydrophilicity of the peptide linker are typically designed such that subunit monomers can readily assemble to form the oligomeric core. Preferably, subunit monomers linked by peptide linkers can assemble to form an oligomeric core wherein the interaction between adjacent subunit monomers is substantially identical to the interaction between the same subunit monomers when not.

Suitable peptide linkers for use in connecting monomer subunits of the oligomeric core are typically between 1 and 100, 1 and 50, 1 and 25, 1 and 20, 1 and 15, or 1 to 10 amino acids in length. The linkers may, for example, be composed of one or more of the following amino acids: lysine, serine, arginine, proline, glycine and alanine. Examples of suitable flexible peptide linkers are stretches of 2 to 20, such as 4, 6, 8, 10 or 16, serine and/or glycine amino acids. Examples of rigid linkers are stretches of 2 to 30, such as 4, 6, 8, 16 or 24, proline amino acids. Examples of suitable linkers include, but are not limited to, the following: GGGS, PGGS, PGGG, RPPPPP, RPPPP, VGG, RPPG, PPPP, RPPG, PPPPPPPPP, PPPPPPPPPPPPPPPPG, GGG, SG, SGSG, SGSGSG, SGSGSGSG, SGSGSGSGSG and SGSGSGSGSGSGSGSG wherein G is glycine, P is proline, R is arginine, S is serine and Vis valine. Additional exemplary linkers include GSGS, GGGGS, GGGGSGGGGS and GGGGSGGGGSGGGGS. Appropriate linking groups may be designed using conventional modelling techniques. The linker is typically sufficiently flexible to allow the monomers, or subunits thereof, to assemble into their respective protein oligomers.

Preferably, the total molecular weight of the oligomeric core is less than about 1000 kDa, such as less than about 500 kDa e.g. less than about about 250 kDa. The total molecular weight of the oligomeric core is preferably from about 10 kDa to about 1000 kDa, such as from about 10 kDa to about 500 kDa, e.g from about 10 kDa to about 250 kDa, such as from about 10 kDa to about 150 kDa. The total molecular weight of the oligomeric core is more preferably from about 20 kDa to about 150 kDa.

Preferably, the diameter of the oligomeric core is less than less than about 100 nm, e.g. less than about 50 nm, such as less than about 30 nm, e.g. less than about 20 nm for example less than about 10 nm. Preferably, the height of the oligomeric core is less than less than about 100 nm, e.g. less than about 50 nm, such as less than about 30 nm, e.g. less than about 20 nm for example less than about 10 nm. The oligomeric core is preferably from 1 to 50 nm in size, such as from 2 to 40 nm e.g. from about 2 to about 20 nm, such as from about 5 to about 10 nm.

Preferably the oligomeric core is thermodynamically stable. Preferably the oligomeric core is stable at a temperature of from about 0 to about 50° C. In other words, preferably the oligomeric core does not spontaneously dissociate into its substituent monomers when in solution at a temperature of from about 0 to about 50° C. More preferably the oligomeric core is stable at a temperature of from about 10 to about 40° C., e.g. from about 20 to about 38° C. e.g., from about 25 to about 37° C.

Preferably, the subunit monomers stably interact to form the oligomeric core. The interactions between subunit monomers is preferably not a weak transient interaction. Weak transient complexes typically show a dynamic mixture of different oligomeric states in vivo, whereas strong transient complexes change their quaternary state only when triggered by, for example, ligand binding and exist in a single predominant oligomeric state (e.g. at least 90%, such as at least 95%, e.g. at least 99%, such as at least 99.9%, e.g. at least 99.99% or 99.999% of the complex may exist in a stable oligomeric state under standard conditions). Weak transient interactions are characterized by a dissociation constant (K_{D}) in the micromolar range and lifetimes of seconds. Strong transient interactions, stabilized by binding of an effector molecule, typically have a longer lifetime and have a lower K_{D} in the nanomolar range. The subunit monomers more preferably interact with at least strong transient reactions, more preferably form a permanent interaction. A permanent interaction means that the oligomer does not or substantially does not disassociate into its constituent subunit monomers under normal conditions (for example when in aqueous solution at a temperature of from about 0 to about 100° C.; under these conditions typically at least 90%, such as at least 95%, e.g. at least 99%, such as at least 99.9%, e.g. at least 99.99% or 99.999% of the oligomeric core does not dissociate), usually the oligomeric core only disassociates when denaturing conditions are used that denature the tertiary structure of the subunit monomers themselves. Accordingly, preferably, the subunit monomers multimerise with a K_{D} of less than 1 μM, e.g. less than 100 nM, more preferably less than 10 nM. The oligomeric core typically has a lifetime of at least 10 minutes, more preferably at least one hour, e.g. at least one day, such as at least one week, e.g. at least one month or at least one year. Lifetime may be determined at any suitable temperature, such as from about 0° C. to about 100° C., e.g. from about 4° C. to about 90° C., such as from about 10° C. to about 50° C. e.g. from about 20 to about 38° C. e.g., from about 25 to about 37° C.

Preferably, the oligomeric core is stable to proteases. For example, the oligomeric core may be exposed to dilute concentrations of a protease, such as trypsin, for a limited period of time, such as 4 hours, without loss of the tertiary of quaternary structure of the oligomeric core.

Preferably the oligomeric core is human, or humanised. A human oligomeric core is a multimeric region of a human protein. A humanised oligomeric core is an oligomeric core which is a multimeric region of a non-human protein, which has been modified to more closely resemble the corresponding multimeric region of a human protein. A humanised oligomeric core may comprise at least 50% amino acid identity to the amino acid sequence of the multimeric region of the corresponding human protein, such as at least 60%, at least 70%, at least 80%, at least 90%, at least 95%, at least 98% or at least 99% amino acid identity. The corresponding multimeric region of a human protein is the multimeric region of a human protein with the greatest amino acid sequence identity to the humanised oligomeric core. This information can be identified using a Blast search (blast.ncbi.nlm.nih.gov) and limiting the search results to Homo sapiens.

Preferably, the oligomeric core of the multivalent protein scaffold does not itself induce an immune response in a biological system, cell culture or subject (such as a non-human or human subject). In other words, typically in the absence of binding sites on the oligomeric core and/or effector moieties attached to the binding sites of the oligomeric core, no immune response is induced when the oligomeric core is administered to a biological system. For example, administration of the oligomeric core (in the absence of binding sites on the oligomeric core and/or effector moieties attached to the binding sites of the oligomeric core) to a biological system (e.g. a subject as defined herein) typically does not induce a biological system's innate or adaptive immunity. For example, the oligomeric core typically does not induce activation of the complement system, B cells, T cells, natural killer cells, mast cells, basophils, eosinophils, neutrophils, dendritic cells or macrophages. However, molecules attached to the oligomeric core may be designed specifically to generate an immune response in a human subject.

Preferably, the oligomeric core of the protein scaffold does not comprise an antibody or antibody fragment; although as described further herein antibodies and/or antibody fragments may be attached as effector moieties to the oligomeric core. The oligomeric core (e.g. absent any effector moieties) more preferably does not comprise an Fc region of an antibody. In some cases, the oligomeric core does not comprise an immunoglobulin constant region.

In some embodiments the oligomeric core of the multivalent protein scaffold may be a homooligomeric core, i.e. the oligomeric core may comprise only one type of monomer. The homooligomeric core may comprise two or more, such as three or more, four or more, five or more, six or more, or seven or more, identical subunit monomers.

In some other embodiments the oligomeric core of the multivalent protein scaffold may be a heterooligomeric core, i.e. the oligomeric core may comprise more than one type of monomer. The different types of subunit monomer are capable of forming an oligomeric core. In other words, the subunit monomers are capable of attaching together. The heterooligomeric core may comprise two or more, such as three or more, four or more, five or more, six or more, or seven or more, different subunit monomers. For example, if a heterooligomeric core comprises three monomers, it may comprise two first monomers and one second monomer; or it may comprise one first monomer, one second monomer, and one third monomer. If a heterooligomeric core comprises four monomers it may comprise two first monomers and two second monomers; two first monomer, one second monomer and one third monomer; or one first monomer, one second monomer, one third monomer and one fourth monomer. Preferably, when the oligomeric core is a heterooligomeric core, the oligomeric core comprises two types of subunit monomer; i.e. for a heterooligomeric core comprising monomers of type A and type B, and having n subunit monomers in total, said heterooligomer core may have a stoichiometry of (AₐB_{b}), wherein a+b=n. When the oligomeric core is a heterooligomeric core, the subunit monomers comprised therein may be modified such that a first type of subunit monomer binds preferentially to a second type of subunit monomer rather than another monomer of the first type (in other words, for a heterooligomeric core comprising monomers of type A and monomers of type B, the heterooligomeric core is of the form ABABAB . . . rather than AAABBB . . . ).

In still further embodiments the oligomeric core may comprise a plurality of multimeric subunits. For example, two monomers may be fused (e.g. as a tandem fusion) and the monomer fusions may assemble to form the oligomeric core. In this regard, the fused monomers may be the same or different.

For example, two or more identical monomers may be fused, with the fusion product assembling with further identical fusion products to form a homooligomeric core. The oligomeric core may comprise a plurality of homodimers, for example wherein the homodimer is ‘AA’, the oligomeric core may comprise ‘AA’, ‘AAAA’, or ‘AAAAAA’ etc.

Alternatively, two or more different monomers may be fused together, with the fusion product assembling with further identical fusion products to form an oligomeric core. As used herein, such a core is typically considered as a homooligomeric core wherein each fusion product is considered as the monomer subunit. The oligomeric core may comprise a plurality of heterodimers, for example wherein the heterodimer is ‘AB’, the oligomeric core may comprise ‘AB’, ‘ABAB’, or ‘ABABAB’ etc.

Alternatively, two or more identical monomers may be fused, with the fusion product assembling with further non-identical fusion products to form a heterooligomeric core. The oligomeric core may comprise a plurality of homodimers, for example wherein a first homodimer is ‘AA’ and a second homodimer is ‘BB’, the oligomeric core may comprise ‘AABB’, etc.

Alternatively, two or more different monomers may be fused together, with the fusion product assembling with further non-identical fusion products to form a heterooligomeric core. The oligomeric core may comprise a plurality of heterodimers, for example wherein a first heterodimer is ‘AB’ and a second heterodimer is ‘CD’, the oligomeric core may comprise ‘ABCD’, etc.

A homooligomeric core comprises a plurality of subunit monomers, wherein each monomer comprises at least one first binding site and at least one second binding site. For example, if a homooligomeric core comprises three subunit monomers, the oligomeric core will comprise at least three first binding sites and at least three second binding sites. If the homooligomeric core comprises four, five, six or seven subunit monomers, the oligomeric core will comprise at least four first binding sites and at least four second binding sites; at least five first binding sites and at least five second binding sites; at least six first binding sites and at least six second binding sites; or at least seven first binding sites and at least seven second binding sites; respectively. Thus, the multivalent protein scaffold preferably comprises at least two first binding sites and at least two second binding sites, i.e, wherein all of first binding sites are the same as the other first binding sites, and all of the second binding sites are the same as the other second binding sites. The multivalent protein scaffold more preferably comprises at least three first binding sites and at least three second binding sites. In some embodiments the multivalent protein scaffold comprises at least four, at least five, at least six, at least seven, or at least eight of each first and second binding sites.

A heterooligomeric core comprises a plurality of subunit monomers comprising at least two types of subunit monomer, wherein one type of subunit monomer comprises at least one first binding site and a second type of subunit monomer comprises at least one second binding site. For example, if a heterooligomeric core comprises three subunit monomers, it may comprise three different binding sites, or it may comprise two first binding sites and one second binding site. If a heterooligomeric core comprises four subunit monomers, it may comprise four different binding sites, or it may comprise two first binding sites and one second binding site and one third binding site, or two first binding sites and two second binding site.

Binding sites are described in more detail herein.

### Monomers

As explained herein, the multivalent protein scaffold provided herein comprises an oligomeric core comprising a plurality of subunit monomers. A subunit monomer is typically the structural domain of the multi-domain polypeptide construct as described elsewhere herein.

Each subunit monomer (excluding any binding site(s) attached thereto, as described in more detail herein) preferably comprises less than 300 amino acids, preferably less than 200 amino acids, more preferably less than 150 amino acids. For example, each subunit monomer (excluding any binding site(s) attached thereto) preferably has a molecular weight of less than 40 kDa, such as less than 30 kDa, such as less than 20 kDa. Protein scaffolds as described herein which comprise such monomers may be of relatively low mass allowing efficient diffusion in vivo. They are typically capable of being expressed and correctly folded in bacterial cell expression or yeast cell expression systems. Such expression systems can often yield far higher yields than mammalian cell cultures typically required to produce antibodies.

The subunit monomers preferably do not comprise or consist of an antibody or antibody fragment. The oligomeric core or subunit monomer preferably does not comprise or consist of an Fc region of an antibody. In some embodiments, the subunit monomer does not comprise or consist of a CH2 domain. In some embodiments, the subunit monomer does not comprise or consist of a CH3 domain. In some embodiments, the subunit monomer does not comprise a CH2 domain and does not comprise a CH3 domain.

Preferably each monomer subunit of the oligomeric core is human, or humanised. A human monomer is a monomer of a human oligomeric protein. A humanised monomer is a monomer of a non-human oligomeric protein, which has been modified to more closely resemble a monomer of the corresponding human protein. A humanised monomer may thus comprise at least 50% amino acid identity to the amino acid sequence of the corresponding human protein, such as at least 60%, at least 70%, at least 80%, at least 90%, at least 95%, at least 98% or at least 99% amino acid identity. Typically, a human or humanised protein will not cause a deleterious immune response in a patient to which it is administered.

The subunit monomers comprised in the oligomeric core preferably each comprise a multimerising structural element, which is the structural and/or functional features of the subunit monomer that allow the subunit monomers to multimerise.

The multimerising structural element may be a protein domain. The multimerising structural element of the oligomeric core may be a multimerisation domain of a naturally-occurring multimeric protein, or a de novo multimeric domain. A protein domain is an autonomously folding unit of a protein. A multimerisation domain is typically a protein domain that is involved in protein-protein interactions with another protein domain. A multimerising structural element is preferably soluble, such that the monomer and oligomeric core is soluble.

In view of the disclosure herein, those skilled in the art will be able to identify suitable multimerising structural elements for use in the present invention. For example, the skilled person can identify multimeric proteins. For example, numerous multimeric proteins are listed in databases such as the NCBI databases (www.ncbi.nlm.nih.gov) and the Protein Data Bank (PDB; www.rscb.org) which can be searched for multimeric proteins having rotational symmetry axes.

Preferably, the multimeric proteins identified are homooligomers, such as homodimers, homotrimers, homotetramers, homopentamers, homohexamers, homoheptamers and so on. The multimeric proteins may be heterooligomers, such as heterodimers, heterotrimers, heterotetramers, heteropentamers, heterohexamers, heteroheptamers and so on. Functional and or structural information can be used to identify which domains of the multimeric protein are responsible for multimerisation, i.e. the multimerisation domains

The multimerising structural element preferably comprises the multimerisation interface of the multimerisation domains (i.e. the structural or functional element of the multimerisation domain that allows the domains to multimerise). Other aspects of the multimerisation domains may be modified without affecting the multimerisation of the subunit monomers. Thus, the subunit monomers of the oligomeric core preferably comprise a multimerising structural element. The subunit monomers preferably comprise at least 50%, at least 60%, at least 70%, at least 80%, at least 90%, at least 95%, at least 98%, at least 99% or 100% amino acid identity to the multimerisation domain, from which they are derived. The subunit monomers retain the ability to form a multimer (i.e. an oligomeric core).

Preferably, the subunit monomers of the oligomeric core comprise a soluble multimerising structural element of a multimeric protein. Soluble domains are preferred, over, for example, multimerisation domains found within membranes. Preferably, each of the subunit monomers of the oligomeric core comprise a soluble multimerising structural element of a soluble multimeric protein.

A multimerising structural element may be derived from any multimeric protein of suitable symmetry (e.g. rotational or dihedral symmetry, as described in more detail herein), such as a collagen (e.g. a collagen NCI domain), a CutA, a TNF, a p53, a fibrinogen, a C4, Bacillus subtillus AbrB or a homolog or paralog thereof.

Preferably, a subunit monomer may comprise a monomer of, or the multimerisation domain of, a protein selected from: collagen X (PDB ID: 1GR3) (e.g. the NCI domain thereof), collagen VIII (PDB ID: 1091) (e.g. the NCI domain thereof), a C1q head domain (for example, PDB ID: 1PK6 is the globular head of human C1q), or a CutA (copper tolerance A) protein such as the CutA1 proteins from Pyrococcus horikoshii, Homo sapiens (PDB ID: 2ZFH), Thermus thermophiles (PDB ID: 1V6H); Oryza sativa (PDB ID: 2ZOM); or Shewanella sp. SIBI (PDB ID: 3AHP); or a polypeptide having at least 30% or at least 50% amino acid sequence identity to any one of the preceding polypeptides; more preferably at least 60% amino acid sequence identity, at least 70%, at least 80%, at least 90%, at least 95% or at least 97%, at least 98% or at least 99% amino acid sequence identity to any one of the preceding polypeptides.

In some embodiments, a subunit monomer may comprise or consist of collagen X (PDB ID: 1GR3, or SEQ ID NO: 2) (e.g. the NCI domain thereof), collagen VIII (PDB ID: 1091, or SEQ ID NO: 3) (e.g. the NCI domain thereof), a heteromeric C1q head domain (for example, PDB ID: 1PK6 is the globular head of human C1q, see SEQ ID NO: 36-38), or a CutA (copper tolerance A) protein such as the CutA1 proteins from Pyrococcus horikoshii (PDB ID: 4YNO, or SEQ ID NO: 1), Homo sapiens (PDB ID: 2ZFH, or SEQ ID NO: 19), Thermus thermophilus (PDB ID: 1V6H, or SEQ ID NO: 39), Oryza sativa (PDB ID: 2ZOM, or SEQ ID NO: 40); or Shewanella sp. SIB1 (PDB ID: 3AHP, or SEQ ID NO: 41); TNF-like protein TL1A (PDB ID: 2RE9, or SEQ ID NO: 31); TNF (PDB ID: 1TNF, or SEQ ID NO: 42; full-length soluble domain SEQ ID NO: 80); TNF family protein CD40L (PDB ID: 3LKJ, or SEQ ID NO: 58); human macrophage migration inhibitory factor (MIF) (PDB ID: 1CA7, or SEQ ID NO: 25, or with Y99G mutation PDB ID: 60Y8); human macrophage migration inhibitory factor 2 (MIF2) (PDB ID: 7MSE, or SEQ ID NO: 26, or with S62A and F99A mutation SED ID NO: 27) or a homolog or paralog thereof.

Other multimerising domains include the multimerising domains of: antiparallel coiled-coil hexamer (PDB ID: 5W0J, see example 4, SEQ ID NO: 43), HIV-1 GP41 core (PDB ID: 115Y or SEQ ID NO: 44), cytochrome c555 (PDB ID: 5Z25 or SEQ ID NO: 45), MHC Class II associated chaperonin and targeting protein invariant chain (Ii) (PDB ID: 1iie or SEQ ID NO: 46); p53 (PDB ID: 1C26 or SEQ ID NO: 47); a fibrinogen-like domain (PDB ID: 4M7F or SEQ ID NO: 48); a Collagen IV C4 (PDB ID: 1LI1 or SEQ ID NO: 49); a Bacillus subtilis AbrB (PDB ID: 1YFB or SEQ ID NO: 50); or a polypeptide having at least 50% amino acid sequence identity to any one of the preceding polypeptides; more preferably at least 60% amino acid sequence identity, such as at least 70%, at least 80%, at least 90%, at least 95% or at least 97%, at least 98% or at least 99% amino acid sequence identity to any one of the preceding polypeptides.

Other multimerising domains include the multimerising domains of: bacteriophage lambda head protein D (e.g. PDB ID: 1C5E or PDB ID: 1C5E or SEQ ID NO: 51); the domain-swapped trimer variant of HCRBPII (PDB ID: 6VIS or SEQ ID NO: 52); the T1L reovirus attachment protein sigma1 (chain A,B,C of PDB ID: 40 DB or SEQ ID NO: 53); or a polypeptide having at least 50% amino acid sequence identity to any one of the preceding proteins; more preferably at least 60% amino acid sequence identity, such as at least 70%, at least 80%, at least 90%, at least 95% or at least 97%, at least 98% or at least 99% amino acid sequence identity to any one of the preceding proteins.

Preferably, in one embodiment, the oligomeric core may comprise monomers derived from the multimerising structural element of Pyrococcus horikoshii CutA1. Accordingly, the or each subunit monomer may comprise or consist of a polypeptide having at least 30%, such as at least 50%, at least 60%, at least 70%, at least 80%, at least 90%, at least 95%, at least 97%, at least 98%, at least 99% or 100%, amino acid identity to the amino acid sequence of SEQ ID NO: 1.

As discussed above and elsewhere herein, CutA1 (e.g. Pyrococcus horikoshii) is a typical structural domain of the multi-domain polypeptide construct.

In certain embodiments, the oligomeric core may comprise monomers derived from the multimerising structural element of human CutA1 (SEQ ID NO: 19). Accordingly, the or each subunit monomer may comprise or consist of a polypeptide having at least 30%, such as at least 50%, at least 60%, at least 70%, at least 80%, at least 90%, at least 95%, at least 97%, at least 98%, at least 99% or 100%, amino acid identity to the amino acid sequence of SEQ ID NO: 19.

In certain embodiments, the multimerising structural element is a truncated form of SEQ ID NO: 19 (human CutA1), as described elsewhere herein. In certain embodiments, the multimerising structural element is a modified form of SEQ ID NO: 19 (human CutA1) wherein at least one cysteine residue in SEQ ID NO: 19 is substituted for a different amino acid residue, as described elsewhere herein.

Without wishing to be bound by theory, one advantage of CutA1, TNF, OX40L, CDL40L or TL1A is a reduced multimeric state by utilising single surface display, even when used as monospecific assemblies (e.g. as “direct fusion” constructs such as a bispecific therapeutic agent). For example, the inventors have observed that Catcher-based display via CutA1 can reach effective inhibition as a hexavalent and trivalent assembly with a trimeric core protein. The reduced multimeric state is expected to lower molecular weight and/or increase stability.

In certain embodiments, the oligomeric core may comprise monomers derived from the multimerising structural element of human TNF (SEQ ID NO: 80) or OX40L (SEQ ID NO: 78) or CD40L (SEQ ID NO: 58) or TL1A (SEQ ID NO: 31). Accordingly, the or each subunit monomer may comprise or consist of a polypeptide having at least 30%, such as at least 50%, at least 60%, at least 70%, at least 80%, at least 90%, at least 95%, at least 97%, at least 98%, at least 99% or 100%, amino acid identity to the amino acid sequence of SEQ ID NO: 80, 78, 58 or 31.

In certain embodiments, the multimerising structural element is a truncated form of SEQ ID NO: 80, 78, 58 or 31, as described elsewhere herein. An exemplary truncated sequence is SEQ ID NO: 79. In certain embodiments, the multimerising structural element is a modified form of SEQ ID NO: SEQ ID NO: 80, 78, 58 or 31 wherein at least one cysteine residue in the sequence is substituted for a different amino acid residue, as described elsewhere herein. In certain embodiments, the multimerising structural element is truncated and at least one cysteine residue is substituted relative to SEQ ID NO: 80, 78, 58 or 31.

Preferably, in another embodiment, the oligomeric core may comprise monomers derived from the multimerising structural element of collagen X NC1. Accordingly, the or each subunit monomer may comprise or consist of a polypeptide having at least 30%, such as at least 50%, at least 60%, at least 70%, at least 80%, at least 90%, at least 95%, at least 97%, at least 98%, at least 99% or 100%, amino acid identity to the amino acid sequence of SEQ ID NO: 2.

As discussed above and elsewhere herein, Collagen X NC1 is a typical structural domain of the multi-domain polypeptide construct.

Preferably, in another embodiment, the oligomeric core may comprise monomers derived from the multimerising structural element of collagen VIII NC1. Accordingly, the or each subunit monomer may comprise or consist of a polypeptide having at least 30%, such as at least 50%, at least 60%, at least 70%, at least 80%, at least 90%, at least 95%, at least 97%, at least 98%, at least 99% or 100%, amino acid identity to the amino acid sequence of SEQ ID NO: 3.

As discussed above and elsewhere herein, Collagen VIII NC1 is a typical structural domain of the multi-domain polypeptide construct.

Preferably, in another embodiment, the oligomeric core may comprise monomers derived from the multimerising structural element of CutA1 from Homo sapiens. Accordingly, the or each subunit monomer may comprise or consist of a polypeptide having at least 30%, such as at least 50%, at least 60%, at least 70%, at least 80%, at least 90%, at least 95%, at least 97%, at least 98%, at least 99% or 100%, amino acid identity to the amino acid sequence of SEQ ID NO: 19.

As discussed above and elsewhere herein, CutA1 (e.g. human) is a typical structural domain of the multi-domain polypeptide construct. The inventors have, furthermore, engineered human CutA1 to optimise and refine its characteristics and suitability as a structural domain, as described in the Examples.

Preferably, in another embodiment, the oligomeric core may comprise monomers derived from the multimerising structural element of MIF or MIF-2. Accordingly, the or each subunit monomer may comprise or consist of a polypeptide having at least 30%, such as at least 50%, at least 60%, at least 70%, at least 80%, at least 90%, at least 95%, at least 97%, at least 98%, at least 99% or 100%, amino acid identity to the amino acid sequence of or SEQ ID NO: 25 or SEQ ID NO: 26 or SEQ ID NO: 27.

As discussed above and elsewhere herein, MIF is a typical structural domain of the multi-domain polypeptide construct. As discussed above and elsewhere herein, MIF-2 is a typical structural domain of the multi-domain polypeptide construct.

Preferably, in another embodiment, the oligomeric core may comprise monomers derived from the multimerising structural element TNF family proteins including TNF and TNF-like TL1A, OX40L or CD40L. Accordingly, the or each subunit monomer may comprise or consist of a polypeptide having at least 30%, such as at least 50%, at least 60%, at least 70%, at least 80%, at least 90%, at least 95%, at least 97%, at least 98%, at least 99% or 100%, amino acid identity to the amino acid sequence of SEQ ID NO: 42 or SEQ ID NO: 31 or SEQ ID NO: 58

As discussed above and elsewhere herein, TNF is a typical structural domain of the multi-domain polypeptide construct. As discussed above and elsewhere herein, proteins from the TNF superfamily are typical structural domains of the multi-domain polypeptide construct. The TNF superfamily is well known as a protein superfamily of type II transmembrane proteins containing TNF homology domain and forming trimers. The superfamily contains 19 members that bind to 29 members of TNF receptor superfamily, including CD40L, OX40L and TNF-α (herein also referred to as “TNF”). Other members of the superfamily include TNF-b, TNF-g, Fas ligand, CD27 ligand, CD30 ligand, CD137 ligand, CD253, CD354, APO-3L, CD256, CD257, CD258, TL1, TITR ligand and ED1-A1.

When the oligomeric core is a heterooligomeric core, each monomer in the oligomeric core may be derived from the same protein. For example, each monomer in the oligomeric core may be derived from one of the proteins described above. The oligomeric core may be heteromeric due to differences in the binding sites attached to the monomer subunits, even if the multimerising domains of each monomer subunit are identical. The oligomeric core may be heteromeric due to differences in the multimerising domains of the monomer subunits, even if all monomer subunits are derived from the same protein.

Those skilled in the art will appreciate that the monomer subunits of the oligomeric core of the multivalent protein scaffold provided herein may be further altered to provide additional functionality or beneficial properties.

For example, a monomer may be a fragment, derivative or variant of a monomer or multimerising structural element described herein. As those skilled in the art will appreciate, fragments of amino acid sequences include deletion variants of such sequences wherein one or more, such as at least 1, 2, 5, 10, 20, 50 or 100 amino acids are deleted. Deletion may occur at the C-terminus or N-terminus of the native sequence or within the native sequence. Typically, deletion of one or more amino acids does not influence the residues immediately surrounding the multimerising structural element of a subunit monomer.

Derivatives of amino acid sequences include post-translationally modified sequences including sequences which are modified in vivo or ex vivo. Many different protein modifications are known to those skilled in the art and include modifications to introduce new functionalities to amino acid residues, modifications to protect reactive amino acid residues or modifications to couple amino acid residues to chemical moieties such as reactive functional groups on linkers or substrates (surfaces) for attachment to such amino acid residues.

Derivatives of amino acid sequences also include addition variants of such sequences wherein one or more, such as at least 1, 2, 5, 10, 20, 50 or 100 amino acids are added or introduced into the native sequence. Addition may occur at the C-terminus or N-terminus of the native sequence or within the native sequence. Typically, addition of one or more amino acids does not influence the residues immediately surrounding the multimerising structural element of a subunit monomer.

Variants of amino acid sequences include sequences wherein one or more amino acid such as at least 1, 2, 5, 10, 20, 50 or 100 amino acid residues in the native sequence are exchanged for one or more non-native residues. Such variants can thus comprise point mutations or can be more profound e.g. native chemical ligation can be used to splice non-native amino acid sequences into partial native sequences to produce variants of native enzymes. Variants of amino acid sequences include sequences carrying naturally occurring amino acids and/or unnatural amino acids.

Variants, derivatives and functional fragments of the aforementioned amino acid sequences typically retain the ability of the wild-type sequence to oligomerise. Preferably, variants, derivatives and functional fragments of the aforementioned sequences have improved properties, such as increased stability, reduced toxicity, additional functionalities including binding sites, etc, compared to the wild-type or native sequence.

### Binding Sites

The multivalent protein scaffold comprises at least one first binding site and at least one second binding site. In some embodiments, typically in the modular system useful to identify useful combinations of effector molecules in drug discovery. the at least one first binding site is orthogonal to the at least one second binding site. In other words, the chemistry by which the first binding site binds to its target (the first target) it orthogonal to the chemistry by which the second binding site binds to its target (the second target). Thus, the first target will bind to the first binding site but will not bind to the second binding site; and the second target will bind to the second binding site but will not bind to the first binding site. Accordingly, the term orthogonal is given its usual meaning in the field of protein-protein interactions, and the first binding interaction (i.e. the first binding site and the first ligand) is independent of the second binding interaction (i.e. the second binding site and the second ligand).

The binding sites of the multivalent protein scaffold allows the scaffold to be used as a modular system to bind effector moieties. The first and second binding sites bind to their cognate target on the effector moieties.

The first and second binding sites may be incorporated into the multivalent protein scaffold provided herein in any suitable manner. In one embodiment the first and second binding sites are provided as a tandem fusion which is attached as described herein to a or each monomer of the oligomeric core to form the multivalent protein scaffold. For comparison, SEQ ID NO: 22 is an example of two binding sites (described herein) provided as a fusion linked by an αH linker.

Binding sites are described in more detail below.

The interaction between a binding site and its target may be a non-covalent interaction. Preferably, the or each binding site can form a covalent bond to its respective target. A reactive functional group may be present naturally in the subunit monomer or effector moiety, or may be introduced, e.g. by genetic manipulation or by chemical modification of the monomer. The reactive group may originate from a non-natural amino acid incorporated into the monomer during its synthesis or expression, e.g. during cell-free expression, e.g. via in vitro transcription/translation.

A binding site on the multivalent protein scaffold may bind to its target via a reactive group. Any suitable reactive group can be used. For example, a reactive group may be an amine-reactive group; a carboxyl-reactive group; a sulfhydryl-reactive group or a carbonyl-reactive group. A reactive group may comprise a cysteine-reactive group. A reactive group may comprise a maleimide, an azide, a thiol, an alkyne, an NHS ester or a haloacetamide.

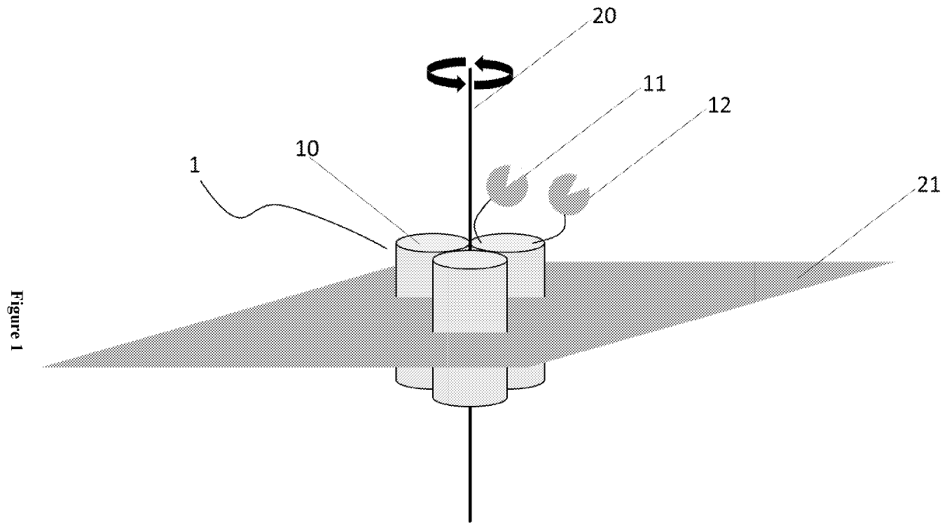

A reactive group may be a group capable of reacting with a non-natural amino acid such as 4-azido-L-phenylalanine (Faz) and any one of the amino acids numbered 1-71 in FIG. 1 of Liu C. C. and Schultz P. G., Annu. Rev. Biochem., 2010, 79, 413-444. Such groups are particularly useful when corresponding non-natural amino acids are comprised in the binding site and the cognate target.

A reactive group may be a click chemistry group. Click chemistry is a term first introduced by Kolb et al. in 2001 to describe an expanding set of powerful, selective, and modular building blocks that work reliably in both small- and large-scale applications (Kolb HC, Finn, MG, Sharpless KB, Click chemistry: diverse chemical function from a few good reactions, Angew. Chem. Int. Ed. 40 (2001) 2004-2021). They have defined the set of stringent criteria for click chemistry as follows: “The reaction must be modular, wide in scope, give very high yields, generate only inoffensive by-products that can be removed by non-chromatographic methods, and be stereospecific (but not necessarily enantioselective). The required process characteristics include simple reaction conditions (ideally, the process should be insensitive to oxygen and water), readily available starting materials and reagents, the use of no solvent or a solvent that is benign (such as water) or easily removed, and simple product isolation. Purification if required must be by non-chromatographic methods, such as crystallization or distillation, and the product must be stable under physiological conditions”.

For example, the first and second binding sites may comprise orthogonal click chemistry reagents. Suitable examples of click chemistry include, but are not limited to, the following:
(a) copper-free variant of the 1,3 dipolar cycloaddition reaction, where an azide reacts with an alkyne under strain, for example in a cyclooctane ring;
(b) the reaction of an oxygen nucleophile on one linker with an epoxide or aziridine reactive moiety on the other;
(c) the Staudinger ligation, where the alkyne moiety can be replaced by an aryl phosphine, resulting in a specific reaction with the azide to give an amide bond;
(d) nitrone dipole cycloaddition;
(e) norbornene cycloaddition;
(f) oxanorbornadiene cycloaddition;
(g) tetrazine ligation;
(h) [4+1] Cycloaddition;
(i) tetrazole photoclick chemistry; and
(j) quadricyclane ligation.

A reactive group may be a haloacetamide, for example, iodoacetamide, bromoacetemide or chloroacetamide.

A reactive group may be selected from a vinyl group, TCO, tetrazine and a strained alkyne; DBCO; an activated acid e.g. an acid chloride; and piperazine and reactive amines.

Host-guest chemistry can also be used to provide the reaction between a binding site and its target. For example, a binding site may comprise a ligand for binding to a metal complex, and the target comprises a metal complex, or vice-versa. Thus, a binding site may comprise a metal complex which can interact non-covalently via chelation or supramolecular association with its target containing a site that can act as a ligand to complex with the modifier molecule by forming a stable association; or vice-versa.

A reactive group may be any of those disclosed in Sakamoto and Hamachi, “Recent progress in chemical modification of proteins”, Anal. Sci 2019 (35) 5-27; or Mckay and Finn, “Click chemistry in complex mixtures: bioorthogonal bioconjugation”, Chem. Biol. 2014, 21 (9) 1075-1101, both of which are hereby incorporated by reference in their entirety.

A binding site of the multivalent protein scaffold preferably comprises a polypeptide, such as a protein domain. More preferably, the first binding site comprises a first protein domain and said second binding site comprises a second protein domain.

When the first binding site comprises a first protein domain and the second binding site comprises a second protein domain, the first binding site and/or the second binding site is preferably genetically fused to the subunit monomer(s) to which they are attached to form a single polypeptide chain. Typically, the first binding site and/or the second binding site is expressed as a single polypeptide chain with the subunit monomer(s) to which they are attached, for example as a fusion protein from a recombinant nucleic acid molecule. This can be beneficial in that the multivalent protein scaffold can be expressed ready for binding effector moieties without further chemical modification needed to, for example, attach a click chemistry reagent. The attachment between a protein binding site and the protein to which it is attached, e.g. the monomer subunit of the oligomeric core of the multivalent protein scaffold, is described below.

The first binding site may comprise a first protein domain capable of forming a non-covalent bond to a first polypeptide target; and said second binding site may comprise a second protein domain capable of forming a non-covalent bond to a second polypeptide target. More preferably, the first binding site comprises a first protein domain capable of forming a covalent bond to a first polypeptide target; and said second binding site comprises a second protein domain capable of forming a covalent bond to a second polypeptide target. Any suitable covalent bond can be formed, with examples above. Preferably, the first protein domain is capable of forming an isopeptide bond with the first polypeptide target and the second protein domain is capable of forming an isopeptide bond with the second binding target. An isopeptide bond is an amide bond that can form for example between the carboxyl group of one amino acid and the amino group of another. At least one of these joining groups is typically part of the side chain of one of these amino acids.

Preferably, the first binding site and the second binding site each comprise a different split protein domain, such as a split ligand-binding protein domain. As used herein a ligand-binding protein domain is a domain of a protein-binding ligand. Any suitable protein can be used, however proteins which natively are stabilised by an intra-strand covalent bond such as an isopeptide bond are particularly beneficial. In such cases, a portion of the protein containing the isopeptide bond donor residue is split from the portion of the peptide containing the isopeptide bond receiver residue. The two protein fragments can be attached, e.g. by genetic fusion, to further polypeptides such as a monomer of an oligomeric core and/or a polypeptide target as described herein. Contacting the two separate fragments leads to the creation of the isopeptide bond which attaches, typically irreversibly, the two fragments together. Accordingly, the split protein approach for producing binding sites and complementary tags is particularly useful. Pairs of such binding site/tags are typically orthogonal as the fragment of one protein will bind preferentially or solely to its native partner (i.e. the complementary portion of the protein from which it was derived) over any other potential partner. These principles are discussed in e.g. Reddington & Howarth, Curr. Op. Chem. Biol. 29, 94-99 (2015) and Keeble et al, PNAS 2019 116 (52) 26523.

Preferably, one of said first binding site and said second binding site comprises a split Streptococcus pyogenes fibronectin-binding protein domain and the other of said first binding site and said second binding site comprises a split Streptococcus pneumoniae adhesin domain.

Preferably, the first protein domain and the first polypeptide target, and the second protein domain and the second polypeptide target, may each comprise a peptide linker pair, such as those disclosed in WO 2016/193746 A1, WO 2018/197854 A1, WO 2018/189517 A1, Keeble et al. (PNAS 116 (52), 2019:26523-26533), Fierer et al. (PNAS 111 (13), 2014: E1176-E1181).

Preferably, said first and said second binding site each independently have at least 50% amino acid identity to any one of SEQ ID NOs: 4-9, 11-13, 23 or 15-18.

Preferably, said first binding site/polypeptide target pair and the second binding site/polypeptide target pair are each independently selected from the following combinations: (i) any one of SEQ ID NO: 4, 6 or 8 with any one of SEQ ID NOs: 5, 7 or 9; (ii) SEQ ID NO: 12 with SEQ ID NO: 13 or 15; (iii) SEQ ID NO: 5 with SEQ ID NO: 11; (iv) SEQ ID NO: 15 with SEQ ID NO: 16); (v) SEQ ID NO: 17 with SEQ ID NO: 18; or (vi) SEQ ID NO: 23 with SEQ ID NO: 16).

More preferably, the first protein domain and the first polypeptide target, and the second protein domain and the second polypeptide target are each independently selected from the following pairs:

| Binding group | Target | |
|---|---|---|
| SpyCatcher (SEQ ID NO: 4) | SpyTag (SEQ ID NO: 5) | |
| SpyCatcher (SEQ ID NO: 4) | SpyTag002 (SEQ ID NO: 7) | |
| SpyCatcher (SEQ ID NO: 4) | SpyTag003 (SEQ ID NO: 9) | |
| SpyTag (SEQ ID NO: 5) | SpyCatcher (SEQ ID NO: 4) | |
| SpyTag002 (SEQ ID NO: 7) | SpyCatcher (SEQ ID NO: 4) | |
| SpyTag003 (SEQ ID NO: 9) | SpyCatcher (SEQ ID NO: 4) | |
| SpyCatcher002 (SEQ ID NO: | SpyTag002 (SEQ ID NO: 7) | |
| 6) | | |
| SpyCatcher002 (SEQ ID NO: | SpyTag (SEQ ID NO: 5) | |
| 6) | | |
| SpyCatcher002 (SEQ ID NO: | SpyTag003 (SEQ ID NO: 9) | |
| 6) | | |
| SpyTag002 (SEQ ID NO: 7) | SpyCatcher002 (SEQ ID NO: | |
| | 6) | |
| SpyTag (SEQ ID NO: 5) | SpyCatcher002 (SEQ ID NO: | |
| | 6) | |
| SpyTag003 (SEQ ID NO: 9) | SpyCatcher002 (SEQ ID NO: | |
| | 6) | |
| SpyCatcher003 (SEQ ID NO: | SpyTag003 (SEQ ID NO: 9) | |
| 8) | | |
| SpyCatcher003 (SEQ ID NO: | SpyTag (SEQ ID NO: 5) | |
| 8) | | |
| SpyCatcher003 (SEQ ID NO: | SpyTag002 (SEQ ID NO: 7) | |
| 8) | | |
| SpyTag003 (SEQ ID NO: 9) | SpyCatcher003 (SEQ ID NO: | |
| | 8) | |
| SpyTag (SEQ ID NO: 5) | SpyCatcher003 (SEQ ID NO: | |
| | 8) | |
| SpyTag002 (SEQ ID NO: 7) | SpyCatcher003 (SEQ ID NO: | |
| | 8) | |
| SpyTag (SEQ ID NO: 5) | K-tag (SEQ ID NO: 11) | Binding mediated by |
| K-tag (SEQ ID NO: 11) | SpyTag (SEQ ID NO: 5) | SpyLigase: (SEQ ID |
| | | NO: 10) |
| SnoopCatcher (SEQ ID NO: | SnoopTag (SEQ ID NO: 13) | |
| 12) | | |
| SnoopTag (SEQ ID NO: 13) | SnoopCatcher (SEQ ID NO: | |
| | 12) | |
| SnoopCatcher (SEQ ID NO: | SnoopTagJr (SEQ ID NO: 15) | |
| 12) | | |
| SnoopTagJr (SEQ ID NO: 15) | SnoopCatcher (SEQ ID NO: | |
| | 12) | |
| SnoopTagJr (SEQ ID NO: 15) | DogTag (SEQ ID NO: 16) | Binding mediated by |
| DogTag (SEQ ID NO: 16) | SnoopTagJr (SEQ ID NO: 15) | SnoopLigase (SEQ ID |
| | | NO: 14) |
| Pilin-C (SEQ ID NO: 17) | IsopepTag (SEQ ID NO: 18) | |
| IsopepTag (SEQ ID NO: 18) | Pilin-C (SEQ ID NO: 17) | |
| DogCatcher (SEQ ID NO: 23) | DogTag (SEQ ID NO: 16) | |

The protein domain and the targeting domain may have at least 50% amino acid identity, such as at least 60%, 70%, 80%, 90%, 95%, 97%, 98%, 99% or 100% amino acid identity with the sequences set forth above, whilst retaining the ability of the protein domain to specifically bind to the targeting domain.

In some embodiments, the first binding site is a protein domain and the first target is a tag which binds to the first protein domain; and the second binding site is a protein domain and the second target is a tag which binds to the second protein domain. In some embodiments, the first binding site is a tag and the first target is a protein domain which binds to the first tag; and the second binding site is a tag and the second target is a protein domain which binds to the second tag. In some embodiments, the first binding site is a protein domain and the first target is a tag which binds to the first protein domain; and the second binding site is a tag and the second target is a protein domain which binds to the second tag. Preferably both the first and second binding sites are protein domains and the first and second targets are tags which specifically bind to the first and second protein domains, respectively.

More preferably the first protein domain and the first polypeptide target, and the second protein domain and the second polypeptide target are each independently selected from the following pairs:

| Binding group | Target | |
|---|---|---|
| SpyCatcher (SEQ ID NO: 4) | SpyTag (SEQ ID NO: 5) | |
| SpyCatcher (SEQ ID NO: 4) | SpyTag002 (SEQ ID NO: 7) | |
| SpyCatcher (SEQ ID NO: 4) | SpyTag003 (SEQ ID NO: 9) | |
| SpyCatcher002 (SEQ ID NO: | SpyTag002 (SEQ ID NO: 7) | |
| 6) | | |
| SpyCatcher002 (SEQ ID NO: | SpyTag (SEQ ID NO: 5) | |
| 6) | | |
| SpyCatcher002 (SEQ ID NO: | SpyTag003 (SEQ ID NO: 9) | |
| 6) | | |
| SpyCatcher003 (SEQ ID NO: | SpyTag003 (SEQ ID NO: 9) | |
| 8) | | |
| SpyCatcher003 (SEQ ID NO: | SpyTag (SEQ ID NO: 5) | |
| 8) | | |
| SpyCatcher003 (SEQ ID NO: | SpyTag002 (SEQ ID NO: 7) | |
| 8) | | |
| SpyTag (SEQ ID NO: 5) | K-tag (SEQ ID NO: 11) | Binding mediated by |
| K-tag (SEQ ID NO: 11) | SpyTag (SEQ ID NO: 5) | SpyLigase: (SEQ ID |
| | | NO: 10) |
| SnoopCatcher (SEQ ID NO: | SnoopTag (SEQ ID NO: 13) | |
| 12) | | |
| SnoopCatcher (SEQ ID NO: | SnoopTagJr (SEQ ID NO: 15) | |
| 12) | | |
| SnoopTagJr (SEQ ID NO: 15) | DogTag (SEQ ID NO: 16) | Binding mediated by |
| DogTag (SEQ ID NO: 16) | SnoopTagJr (SEQ ID NO: 15) | SnoopLigase (SEQ ID |
| | | NO: 14) |
| Pilin-C (SEQ ID NO: 17) | IsopepTag (SEQ ID NO: 18) | |
| DogCatcher (SEQ ID NO: 23) | DogTag (SEQ ID NO: 16) | |

The protein domain and the targeting domain may have at least 50% amino acid identity, such as at least 60%, 70%, 80%, 90%, 95%, 97%, 98%, 99% or 100% amino acid identity with the sequences set forth above, whilst retaining the ability of the protein domain to specifically bind to the targeting domain.

The binding groups and targets above may be divided into the following subgroups: Subgroup A:
SpyCatcher (SEQ ID NO: 4)/SpyTag (SEQ ID NO:5);
SpyCatcher (SEQ ID NO: 4)/SpyTag002 (SEQ ID NO:7);
SpyCatcher (SEQ ID NO: 4)/SpyTag003 (SEQ ID NO:9);
SpyCatcher002 (SEQ ID NO: 6)/SpyTag (SEQ ID NO:5);
SpyCatcher002 (SEQ ID NO: 6)/SpyTag002 (SEQ ID NO:7);
SpyCatcher002 (SEQ ID NO: 6)/SpyTag003 (SEQ ID NO:9);
SpyCatcher003 (SEQ ID NO: 8)/SpyTag (SEQ ID NO:5);
SpyCatcher003 (SEQ ID NO: 8)/SpyTag002 (SEQ ID NO:7);
SpyCatcher003 (SEQ ID NO: 8)/SpyTag003 (SEQ ID NO:9);
SpyTag (SEQ ID NO:5)/K-tag (SEQ ID NO: 11) (mediated by SpyLigase: (SEQ ID NO: 10))

### Subgroup B:

SnoopCatcher (SEQ ID NO: 12)/SnoopTag (SEQ ID NO: 13);
SnoopCatcher (SEQ ID NO: 12)/SnoopTagJr (SEQ ID NO: 15);
SnoopTagJr (SEQ ID NO: 15)/DogTag (SEQ ID NO: 16) (mediated by SnoopLigase (SEQ ID NO: 14)/
DogCatcher (SEQ ID NO: 23)/DogTag (SEQ ID NO: 16)

### Subgroup C:

Pilin-C(SEQ ID NO: 17)/Isopep Tag (SEQ ID NO: 18)

Preferably, the first binding site/target pair is selected from subgroup A and the second binding site/target pair is selected from subgroup B and subgroup C; or the first binding site/target pair is selected from subgroup B and the second binding site/target pair is selected from subgroup A and subgroup C; or the first binding site/target pair is selected from subgroup C and the second binding site/target pair is selected from subgroup A and subgroup B.

Still more preferably, the first protein domain-polypeptide target pair, and the second protein domain-polypeptide target may be selected from the group consisting of (i) SpyCatcher/SpyTag and SnoopCatcher/SnoopTag; (ii) SpyCatcher002/SpyTag002 and SnoopCatcher/SnoopTag; (iii) SpyCatcher003/SpyTag003 and SnoopCatcher/SnoopTag; (iv) SpyCatcher/SpyTag and Pilin-C/Isopeptag; (v) SpyCatcher002/SpyTag002 and Pilin-C/Isopeptag; (vi) SpyCatcher003/SpyTag003 and Pilin-C/Isopeptag; (vii) Pilin-C/Isopeptag and SnoopCatcher/SnoopTag; (viii) SpyCatcher/Spy Tag and SnoopTagJr/DogTag; (ix) SpyCatcher002/SpyTag002 and SnoopTagJr/DogTag; (x) Spy Tag/K-tag and SnoopCatcher/SnoopTag; (xi) SpyTag/K-tag and SnoopTagJr/DogTag; (xii) SpyTag/K-tag and Pilin-C/Isopeptag; and (xiii) SnoopTagJr/DogTag and Pilin-C/Isopeptag; and (xiv) SpyCatcher003/SpyTag003 and DogCatcher/DogTag; and (xv) SpyCatcher003/SpyTag002 and DogCatcher/DogTag; and (xvi) SpyCatcher003/SpyTag and DogCatcher/DogTag; (xvii) SpyCatcher003/SpyTag003 and SnoopCatcher/SnoopTagJr; and (xviii) SpyCatcher003/SpyTag002 and SnoopCatcher/SnoopTagJr; and (xix) SpyCatcher003/SpyTag and SnoopCatcher/SnoopTagJr.

Those skilled in the art will appreciate that when SpyLigase/SpyTag/K-tag, or SnoopLigase/SnoopTagJr/DogTag are used the ‘ligase’ catalyses the attachment of the two tags. Accordingly, the first binding site and the first polypeptide target, and the second binding site and the second polypeptide target are selected from the two ‘tags’. The ligase may be added exogenously to catalyse the attachment of the two ‘tags’, or may be associated with the multivalent protein scaffold, non-covalently or covalently, such as genetically fused with the multivalent protein scaffold. It is interchangeable which of the tags is/are comprised within the multivalent protein scaffold.

Other binding site/tag pairs include SdyTag/SdyCatcher (Tan et al, PLOS One 11(1) e0165074) and the Cpe0147₄₃₉₋₅₆₃/Cpe0147₅₆₅₋₅₈₇ pair derived from Clostridium perfringens cell-surface adhesin protein Cpe0147 (Young et al, Chem Comm. 53(9) 1502).

“Specifically binds” as used herein in the context of binding between a binding site and its target, refers to the ability of a binding site to bind to its complementary binding site with greater affinity than it binds to an unrelated control. The unrelated control may be an unrelated control protein. For example, SnoopCatcher specifically binds to SnoopTag with greater affinity than it binds to an unrelated control protein. The binding is preferably covalent, such as the formation of an isopeptide bond. Preferably, the control protein is bovine serum albumin, and the binding site binds to the complementary binding site with an affinity that is at least 10, at least 50, at least 100, at least 500, or at least 1000 times greater than the control protein. Affinity may be determined by methods known in the art. For example, affinity may be determined by ELISA assay, biolayer interferometry, surface plasmon resonance, kinetic methods or equilibrium/solution methods. The skilled person will recognize which pairs of binding sites specifically bind to produce a protein complex that can be used in the methods of the invention.

The at least one first binding site(s) and the at least one second binding site(s) preferably do not comprise an antibody or antibody fragment. The at least one first binding site(s) and the at least one second binding site(s) more preferably do not comprise an antigen binding fragment of an antibody, such as a Fab, or a Fc region.

In all discussions concerning a ‘binding site’ which binds to a ‘target’ herein, the skilled person will readily understand that the chemical binding groups are reversible, In other words, whereas binding above has been described in terms of reaction of a reactive group A at the binding site with a corresponding reactive group B of the target for that binding site, equivalent chemistry is possible wherein reactive group B at the binding site reacts with reactive group A of the target.

When the multivalent protein scaffold comprises one or more binding sites which are protein domains e.g. protein domains attached to monomer subunits of the oligomeric core of the multivalent protein scaffold, the protein domains may be attached to the multivalent protein scaffold (e.g. attached to monomer subunits of the oligomeric core of the multivalent protein scaffold) by any suitable means.

A binding site may be attached to the multivalent protein scaffold (e.g. attached to monomer subunits of the oligomeric core of the multivalent protein scaffold) by a linker. In one embodiment the same linker may be used at each terminus of a subunit monomer of the oligomeric core. In another embodiments a different linker may be used at each terminus of a subunit monomer of the oligomeric core.

The binding site is preferably covalently attached to the oligomeric core (or subunit monomer). The covalent linkage may for example be a peptide bond, a disulphide bond or a click chemistry linkage. More preferably, the covalent linkage comprises at least one amino acid, i.e. a peptide linker, and forms part of the same polypeptide chain as the subunit monomer at the binding site.

A peptide linker used to attach a binding site to the monomer subunit of the oligomeric core of the multivalent protein scaffold may be genetically fused to the subunit monomer and/or the binding site. A linker is genetically fused if the linker is expressed as a single construct with the subunit monomer and/or the binding site from a single polynucleotide coding sequence. The length, flexibility and hydrophilicity of the peptide linker are typically designed such that the binding sites may be positioned on the same face of the oligomeric core or multivalent protein scaffold. The peptide linker typically allows for directional tethering of the binding sites.

Suitable peptide linkers for use in connecting a binding site to a monomer subunit are typically between 1 and 100, 1 and 50, 1 and 25, 1 and 20, 1 and 15, or 1 to 10 amino acids in length. The linkers may, for example, be composed of one or more of the following amino acids: lysine, serine, arginine, proline, glycine and alanine. Examples of suitable flexible peptide linkers are stretches of 2 to 20, such as 4, 6, 8, 10 or 16, serine and/or glycine amino acids. Examples of rigid linkers are stretches of 2 to 30, such as 4, 6, 8, 16 or 24, proline amino acids. Examples of suitable linkers include, but are not limited to, the following: GGGS, PGGS, PGGG, RPPPPP, RPPPP, VGG, RPPG, PPPP, RPPG, PPPPPPPPP, PPPPPPPPPPPPPPPPG, GGG, SG, SGSG, SGSGSG, SGSGSGSG, SGSGSGSGSG and SGSGSGSGSGSGSGSG wherein G is glycine, P is proline, R is arginine, S is serine and V is valine. Other exemplary linkers include GSGS, GGGGS, GGGGSGGGGS and GGGGSGGGGSGGGGS. Appropriate linking groups may be designed using conventional modelling techniques. The linker is typically sufficiently flexible to allow the binding site and the monomer subunit to assemble into their respective protein oligomers.

The oligomeric core of the multivalent protein scaffold preferably comprises at least one first binding site at a terminus of a subunit monomer, and at least one second binding site at a terminus of a subunit monomer. For example, a first binding site may be positioned at the first terminus of a subunit monomer, and a second binding site may be positioned at the second terminus of a subunit monomer.

The termini are preferably determined by reference to termini of the subunit monomers of the oligomeric core, not including any linker or binding site. More preferably, the terminus of a subunit monomer is selected from the N-terminus and/or the C-terminus of the subunit monomer. If a binding site (e.g. a protein domain) forms part of the same polypeptide as a subunit monomer, the N-terminus and C-terminus preferably relate to the amino acids corresponding to the respective termini of the monomer in the absence of the binding site. Similarly, if a linker forms part of the same polypeptide as a subunit monomer, the N-terminus and C-terminus preferably relate to the amino acids corresponding to the respective termini of the monomer in the absence of the linker.

Preferably, the termini of the subunit monomers to which the binding sites are attached are on the same face of the oligomeric core or the multimeric protein scaffold, as defined in more detail above.

In some cases, the oligomeric core only comprises a single terminus of each subunit monomer on a given face. In this case, the at least one first binding site and the at least one second binding site are typically both attached to the same terminus thereby being on the same face of the multivalent protein scaffold. For example, the oligomeric core may comprise a plurality of subunit monomers, wherein each subunit monomer comprises a first binding site attached at a first terminus of said monomer and a second binding site attached to said first binding site. The oligomeric core may comprise a plurality of subunit monomers, wherein the at least one first binding site(s) are attached at a first terminus of a first subunit monomer and the at least one second binding sites(s) are attached at a second terminus of a second subunit monomer (e.g. a heterooligomeric core). The oligomeric core may comprise a combination of the methods of attachment described above.

The subunit monomers in the oligomeric core of the multivalent protein scaffold preferably each comprise two termini on a single face of the monomer (and thereby on a single face of the oligomeric core and multivalent protein scaffold). The two termini are preferably the N-terminus and C-terminus of the monomer polypeptide. Each monomer more preferably comprises a first binding site attached at a first terminus of said monomer and a second binding site at a second terminus of said monomer. The first and second binding sites may be the N-terminus and the C-terminus, respectively, or the C-terminus and N-terminus respectively.

Sometimes, a monomer may comprise more than one binding site at each terminus. For example, a subunit monomer may comprise at each terminus: (i) a first binding site attached at a terminus of said monomer and a second binding site attached to said first binding site, or vice-versa, (ii) a first binding site attached at a terminus of said monomer and at least one further first binding site attached to said first binding site, (iii) a second binding site attached at a terminus of said monomer and at least one further second binding site attached to said second binding site, (iv) a single first or second binding site attached at a terminus of said monomer.

### Positioning of Binding Sites on the Multivalent Protein Scaffold

As described above, in the multivalent protein scaffold provided herein the at least one first binding site(s) and the at least one second binding site(s) are positioned on the same face of the scaffold. Likewise, in typical embodiments of the multi-domain polypeptide construct, the first binding domain and the second binding domain are positioned on the same face of the polypeptide construct.

By being positioned on the same face of the multivalent protein scaffold (or multi-domain polypeptide construct), the at least one first binding site(s) and the at least one second binding site(s) are arranged such that the effector moieties ultimately bound to the multivalent protein scaffold via the binding sites can interact with their respective biological targets (e.g. receptors on cell surfaces) on a single surface or plane.

The at least one first binding site(s) and the at least one second binding site(s) are positioned on the same face of the multivalent protein scaffold (or multi-domain polypeptide construct). Preferably, the at least one first binding site(s) and the at least one second binding site(s) are positioned on the same face of the oligomeric core. Preferably, the at least one first binding site(s) and the at least one second binding site(s) are positioned on the same face of the subunit monomer(s) to which they are attached. As described herein, a subunit monomer is typically the structural domain of the multi-domain polypeptide construct.

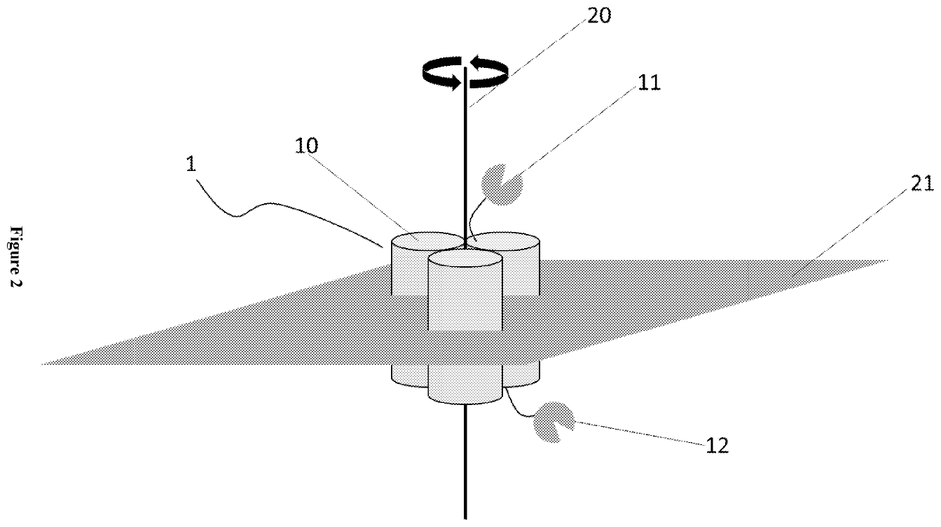

The term “the at least one first binding site(s) and the at least one second binding site(s) are positioned on the same face of the scaffold” can be understood as follows, with reference to FIGS. 1 and 2.

The multivalent protein scaffold (1) or oligomeric core (10) comprises a conceptual rotational symmetry axis (20) corresponding to the number of monomers in the core. For example, a homotrimeric core comprises a C3 symmetry axis. For example, a homooligomeric pentameric core comprises a C5 symmetry axis. Heterooligomeric cores similarly comprise a conceptual rotational axis that runs through the centre of the oligomeric core and parallel to the interfaces between each subunit, for example, the rotational axis for a heterodimer runs through the oligomeric core and parallel to the length of the interface between the monomers; the rotational axis for a heterotrimer runs through the oligomeric core and as parallel as possible to the length of the at least two interfaces between the monomers. A plane (21) can be defined as being perpendicular or approximately perpendicular (e.g. between about 80° and about 100°, such as between about 85° and about 95° e.g. between about 88° and about 92°, e.g. about) 90° to the rotational axis and running through the centre of the oligomeric core. The at least one first binding site(s) (11) and the at least one second binding site(s) (12) are positioned on the same side of that plane and thus on the same face of the multivalent protein scaffold (1). This is illustrated schematically in FIG. 1, which depicts a trimeric oligomeric core and in which only one first binding site and one second binding site is depicted for clarity. By contrast, FIG. 2 depicts the contrasting situation where the at least one first binding site(s) (11) and the at least one second binding site(s) (12) are positioned on opposite side of the plane (21) and thus on opposite faces of the multivalent protein scaffold (1).

Those skilled in the art will appreciate that the conceptual symmetry axis remains even when monomers of the oligomeric core are linked e.g. by being covalently fused as described herein.

Thus, the “same face of the protein scaffold” may be the solvent accessible surface of the multivalent protein scaffold on one side of the plane perpendicular to the highest-order rotational symmetry axis of the oligomeric core of the multivalent protein scaffold and running though the centre of the multivalent protein scaffold. Similarly, a face of the oligomeric core may be the solvent accessible surface of the oligomeric core (preferably defined in the absence of the binding sites attached thereto) on one side of the plane perpendicular to the highest-order rotational symmetry axis of the oligomeric core and running though the centre of the oligomeric core.

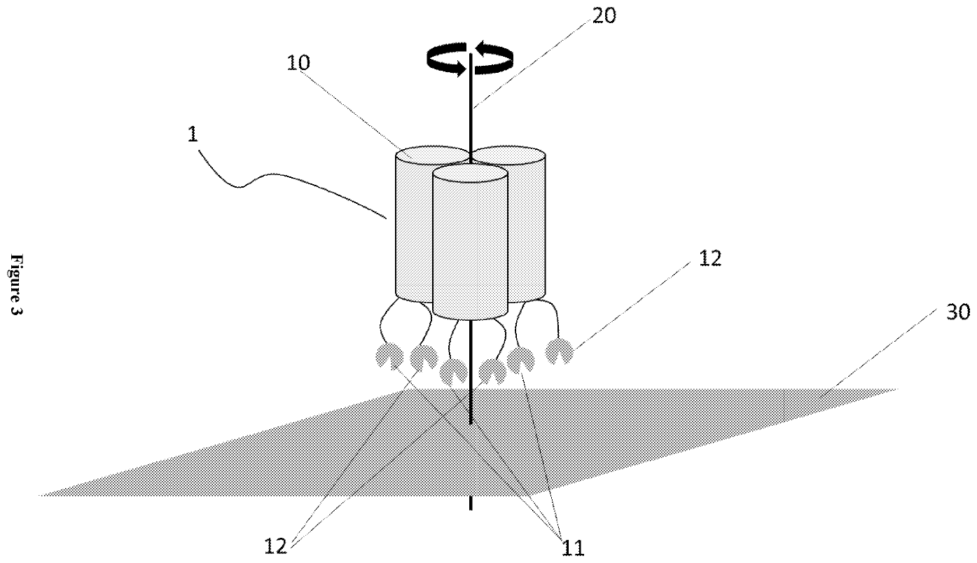

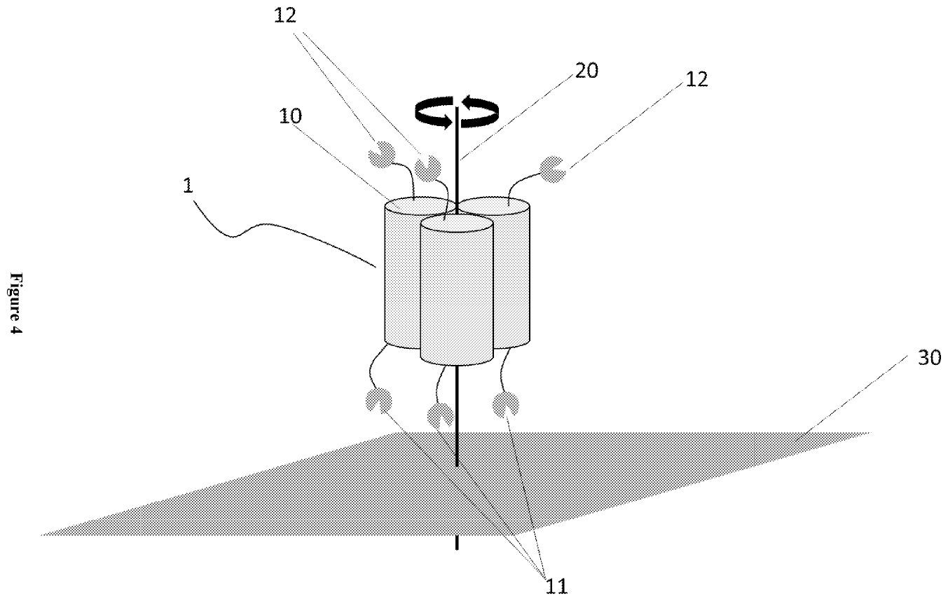

Preferably, the face of the multivalent protein scaffold is the solvent-accessible portion of the multivalent protein scaffold which makes contact with a single surface, e.g. the surface of a cell such as a cell wall, cell membrane or protein complex. Thus, with reference to the schematic depiction in FIG. 3 (which for clarity depicts a plurality of first and second binding sites (11, 12) attached to the oligomeric core (10) of the multivalent protein scaffold (1)), the at least one first binding site(s) (11) and the at least one second binding site(s) (12) are preferably positioned on the multivalent protein scaffold (1) so that they can both contact the said surface (30). This is not possible if the at least one first binding site(s) (11) and the at least one second binding site(s) (12) are positioned on opposite faces of the multivalent protein scaffold (1), as illustrated schematically in FIG. 4. For example, the first binding site(s) may be capable of contacting the surface (30) but the second binding sites may not be capable of contacting the surface (30).

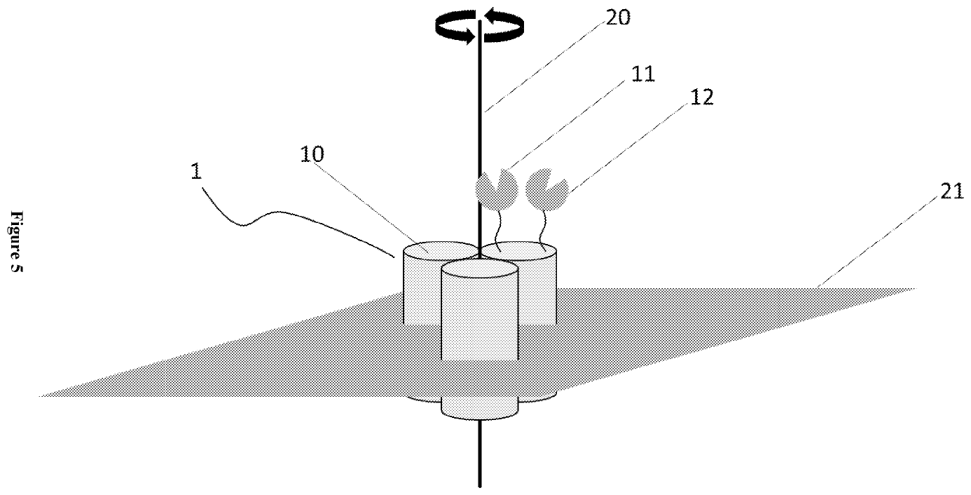

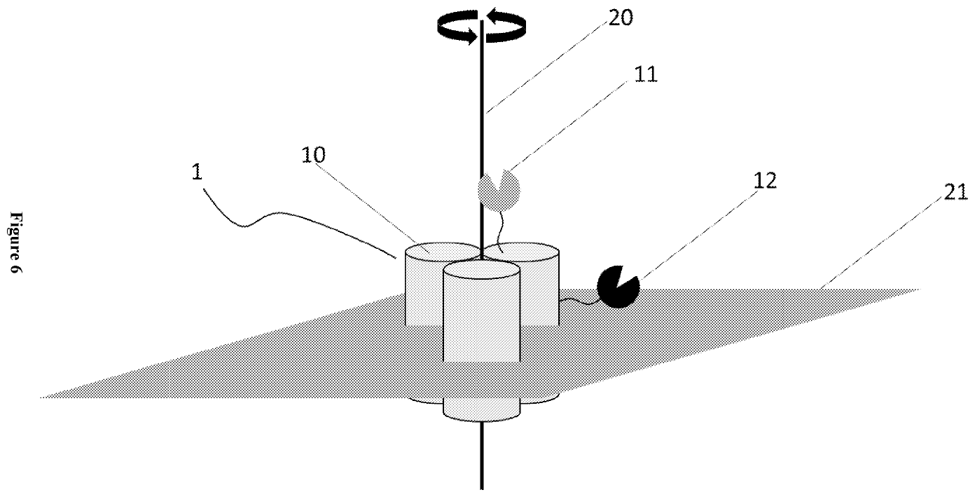

The at least one first binding site(s) and the at least one second binding site(s) are preferably arranged in a front-front orientation or a side-front orientation, or any position in between. As used herein, a front-front orientation refers to both the first binding site(s) and the second binding site(s) both being positioned on the same face of the multivalent protein scaffold with attachment substantially parallel to the rotational symmetry axis through the multivalent protein scaffold. This is illustrated schematically in FIG. 5. A side-front orientation refers to one of the first binding site(s) and the second binding site(s) being positioned on a face of the multivalent protein scaffold with attachment substantially parallel to the rotational symmetry axis through the multivalent protein scaffold; and the other of the first binding site(s) and the second binding site(s) both being positioned on the same face of the multivalent protein scaffold with attachment substantially perpendicular to the rotational symmetry axis through the multivalent protein scaffold (i.e. substantially parallel to plane (21)). This is illustrated schematically in FIG. 6. Of course, any position between these extremes can be used, for example where the first binding site(s) and/or the second binding site(s) are positioned on the same face of the multivalent protein scaffold with attachment at approximately 45° to the rotational symmetry axis through the multivalent protein scaffold. This is depicted schematically in FIG. 7.

Those skilled in the art will appreciate that the angles between the one or more first binding site(s) (11) and axis (20) and the angles between the one or more second binding site(s) (12) and axis (20) need not be the same. For example, the one or more first binding site(s) (11) may be on the “front” of the multivalent protein scaffold and the one or more second binding site(s) (12) may be on the “side” of the multivalent protein scaffold; i.e. in front-side orientation as described above. Alternatively, the one or more first binding site(s) (11) may be on the “side” of the multivalent protein scaffold and the one or more second binding site(s) (12) may be on the “front” of the multivalent protein scaffold; i.e. in side-front orientation as described above. Both the one or more first binding site(s) (11) and the one or more second binding site(s) (12) may be on the “front” of the multivalent protein scaffold; i.e. in front-front orientation as described above.

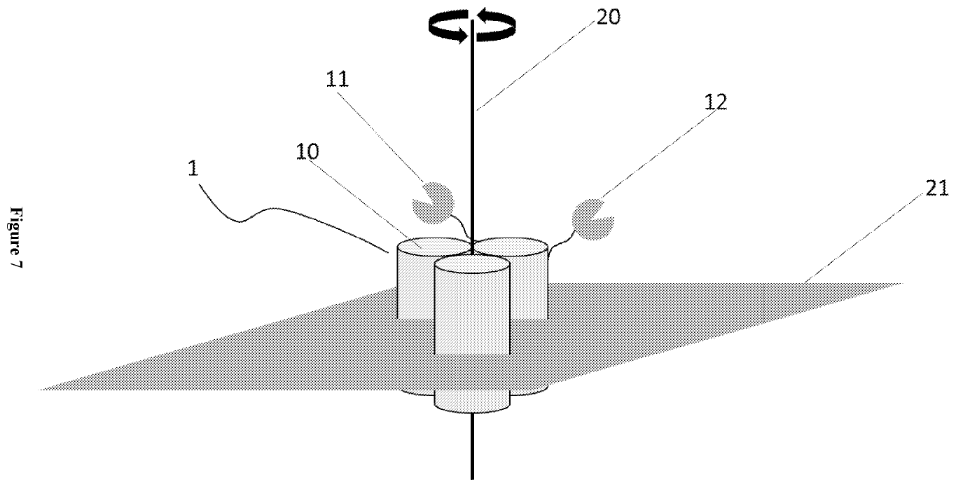

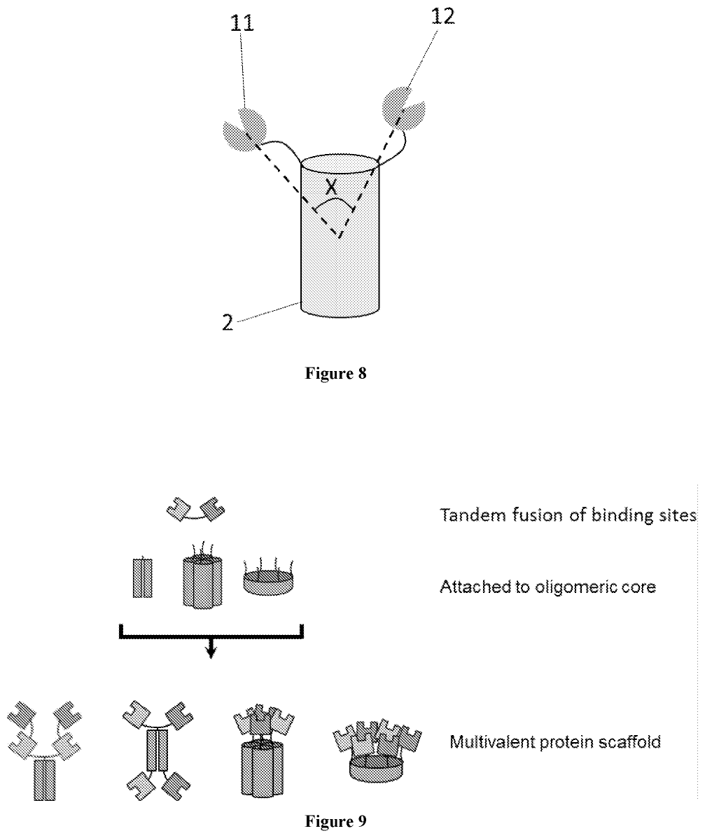

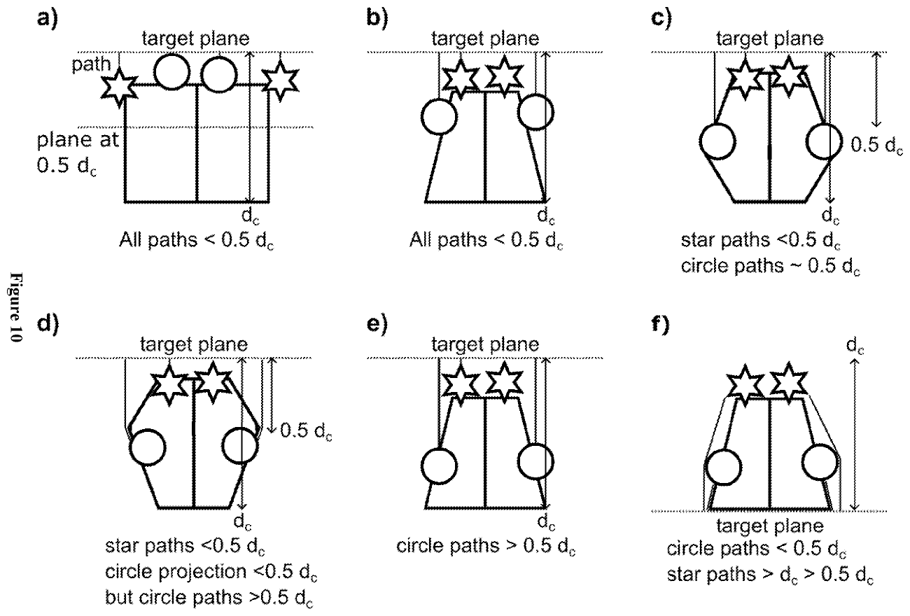

Subject to both the first and second binding site(s) being on the same face of the multivalent protein scaffold, when both first and second binding sites are attached to a given subunit monomer (2), typically the angle formed between the first and second binding site(s) and the centre of that monomer (depicted X in FIG. 8) is at most at an 160° angle, e.g. at most a 140° angle, e.g. at most a 120° angle, such as at most a 100° angle or at most a 90° angle. Typically, the angle formed between the first and second binding site(s) and the centre of that monomer is at least a 10° angle, e.g. at least a 20° angle, e.g. at least a 30° angle, such as at least a 45° angle or at least a 60° angle.

Structures may also be visualized by placing a flat target plane in a 3D coordinate system in an arbitrary position such that it does not intersect with the surface of an oligomeric core as determined from protein structural data (NMR, X-Ray) or structure prediction. For each fusion site, the distance of the shortest path to the target plane that does not intersect with the surface of an oligomeric core (other than at the originating fusion site) may be determined. Preferably, for a given structure a position of a target plane can be found such that all such shortest paths are less than 50%, 45%, or 40% of the longest protein's cross-section orthogonal to the target plane.

In some embodiments, the maximum shortest path length to contact the same plane is less than 100 nm, e.g. less than 50 nm, e.g. less than 20 nm, e.g. less than 10 nm, e.g. less than 5 nm, e.g. less than 2 nm. In a preferred situation, all shortest path lengths to the target plane end within a circular area on the target plane with a radius less than 50 nm, such as less than 25 nm, such as less than 10 nm, such as less than 5 nm.

In some embodiments, cis-orientation of proteins fused to a scaffold core can be determined by way of structural prediction. In addition to distance alone, Alphafold can take linker geometry and interactions between fused binding domains and scaffold core protein into account. Preferably, the scaffold core is predicted to retain its oligomerization properties even after fusion to preferred binding sites via linkers, preferably via short linkers, such as via GSGS, such as via GGGGS, such as via GGGGSGGGGS, such as via GGGGSGGGGSGGGGS, while the binding sites are predicted to be presented approximately in a cis geometry.

### Domain Insertions

The multivalent protein scaffold comprises an oligomeric core comprising a plurality of subunit monomers, and at least one first binding site orthogonal to at least one second binding site, preferably wherein the at least one first binding site and the at least one second binding site are positioned on the same face of the multivalent protein scaffold. The multivalent protein scaffold may further comprise a domain insertion. A domain insertion is a protein domain. A domain insertion may be located on the same face of the multivalent protein scaffold as the binding sites, or on a different face.

In some cases, wherein the oligomeric core and/or the subunit monomers comprise at least one free terminus that is not attached a binding site, the multivalent protein scaffold comprises a domain insertion at the free terminus. A domain insertion may also be located within loop regions of the oligomeric core, thus many be located within a loop region of a subunit monomer. Preferably, the multimeric protein scaffold comprises at least one domain insertion at an opposite face of the oligomeric core and/or multimeric protein scaffold to the face at which the binding sites are positioned, for example, within 90° of the opposite end of the axis.

As used herein, a domain insertion is a polypeptide sequence encoding a protein domain, i.e. an autonomously folding functional unit of a protein. The domain insertion does not interfere with the structure and folding of the oligomeric core or the binding sites.

A domain insertion preferably has an effector function. The domain insertion may comprise an antibody, an antibody fragment or an antigen-binding fragment, such as an antigen-binding fragment capable of binding to CD3 or CD16. For example, a domain insertion may bind be an immune modulatory protein, such as a cytokine, or a chemotherapeutic agent, or a cancer immunotherapy agent (i.e. a treatment that makes use of a subject's immune system to treat cancer). The domain insertion may form a protein that induces cell death when contacted with a biological system. The domain insertion may induce apoptosis, increase an anti-tumor response, or have other beneficial activity. The domain insertion may have complement-inhibiting or complement-stimulating activity.

For the avoidance of doubt, the domain insertion is typically made into the structural domain of the multi-domain polypeptide construct as described herein.

### Protein Complexes

Also provided herein is a protein complex comprising a multivalent protein scaffold as described in more detail herein, attached to at least one first effector moiety and at least one second effector moiety. Each first effector moiety is attached to a first target bound to a first binding site on the multivalent protein scaffold. Each second effector moiety is attached to a second target bound to a second binding site on the multivalent protein scaffold.

The targets are preferably polypeptide targets, more preferably a partner of the peptide linker pairs described above.

Each effector moiety is attached to the target by which it is bound to the multivalent protein scaffold. The first and second effector moieties may be the same or different, preferably different. Any of the attachment routes described above in the context of binding sites may be used. Conventional organic chemistry routes accessible to those skilled in the art may be used to directly attach each effector moiety to the target by which it is bound to the multivalent protein scaffold. Suitable chemical approaches are described in textbooks such as March's Advanced Organic Chemistry (Wiley 2020).

Preferably, each effector moiety is covalently linked to the target. More preferably, the target is a polypeptide target and may be genetically fused to the effector moiety. In other words, preferably an or each effector moiety is genetically fused to a polypeptide target by being encoded in the same polynucleotide such that they are expressed as a single polypeptide chain. The effector may be genetically fused to a first polypeptide target, a cleavage site, and a second polypeptide target, wherein the first polypeptide target is orthogonal to the second polypeptide target. The cleavage site may be a TEV cleavage site. When both the first and second polypeptide targets are present on the effector moiety, only the terminal polypeptide target is functional (i.e. able to bind its cognate binding site on the multivalent protein scaffold). The cleavage site may be employed to separate the terminal polypeptide target, such that only a single target is present. After complete conjugation of the effector moiety, the terminal polypeptide target can then be deployed specifically.

An effector moiety is preferably a protein domain. The protein domain is preferably a soluble protein domain. The protein domain preferably comprises a domain of a secreted protein or an extracellular domain of a transmembrane protein. The protein domain more preferably comprises an extracellular domain of a cell-surface receptor or a ligand of a cell-surface receptor, such as a human cell-surface receptor.

An effector moiety is preferably a moiety which exert a therapeutic effect when contacted with a biological system. For example, an effector moiety may be an immune modulatory protein, such as a cytokine, or a chemotherapeutic agent, or a cancer immunotherapy agent (i.e. a treatment that makes use of a subject's immune system to treat cancer). The effector moiety may induce cell death when contacted with a biological system. The effector moiety may induce apoptosis, increase an anti-tumor response, or have other beneficial activity. The effector moiety may have complement-inhibiting or complement-stimulating activity. The effector moiety may result in altered gene expression, receptor internalization, cytokine release, cell death, or susceptibility to therapeutic molecules.

In one embodiment an effector moiety may be a synthetic organic or inorganic molecule. A suitable molecule may be a chemotherapeutic agent. A suitable molecule may be a toxic agent, e.g. an agent having an EC50 of less than about 100 μM, e.g. less than about 10 μM, such as less than about 1 μM or less than about 100 nM; wherein EC50 is the concentration of the agent required to result in 50% cell toxicity when assessed in a suitable cell assay. A suitable cell assay may be for example a sulforhodamine B (SRB) assay.

A suitable synthetic molecule may be an enzyme activator or inhibitor. A suitable molecule may be an inhibitor of one or more of serine/threonine/tyrosine kinases, matrix metalloproteinases (MMPs), heat shock proteins (HSPs), and proteasomes. A suitable molecule may act as an alkylating agent (e.g. nitrogen mustards, nitrosoureas, tetrazines, aziridines, cisplatins and derivatives thereof); an antimetabolite (e.g. anti-folates, fluoropyrimidines, deoxynucleoside analogues and thiopurines); an anti-microtubule agent (e.g. a vinca alkyloid or taxanes); a topoisomerase inhibitor (e.g an inhibitor of topoisomerase I, e.g. irinotecan and topotecan; a topoisomerase II poison such as etoposide, doxorubicin, mitoxantrone and teniposidel or a topoisomerase II inhibitor such as novobiocin, merbarone, and aclarubicin) or a cytotoxic antibiotic (e.g. anthracyclines and bleomycins). A suitable molecule may have a molecular mass of from about 50 to about 5000 g/mol, e.g. from about 100 to about 1000 g/mol such as from about 250 to about 500 g/mol.

In another embodiment the effector moiety preferably comprises an antibody or an antigen-binding fragment thereof. The term ‘antibody or an antigen-binding fragment thereof’ as used herein in relation to effector moieties may relate to whole antibodies (i.e. comprising the elements of two heavy chains and two light chains inter-connected by disulphide bonds) as well as antigen-binding fragments thereof. Antibodies typically comprise immunologically active portions of immunoglobulin (Ig) molecules, i.e., molecules that contain an antigen binding site that specifically binds (immunoreacts with) an antigen. As used herein, the term “specifically binds” or “immunoreacts with” in the context of the interaction of an antibody or fragment thereof with an antigen, means that the antibody reacts with one or more antigenic determinants of the desired antigen preferentially compared with other polypeptides. Each heavy chain is comprised of a heavy chain variable region (abbreviated herein as HCVR or VH) and at least one heavy chain constant region. Each light chain is comprised of a light chain variable region (abbreviated herein as LCVR or VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). Antibodies may include, but are not limited to, polyclonal, monoclonal, chimeric, dAb (domain antibody), single chain, Fab, Fab′ and F(ab′)2 fragments, scFvs, and Fab expression libraries. An antibody may for example be selected from the group consisting of single chain antibodies, single chain variable fragments (scFvs), variable fragments (Fvs), fragment antigen-binding regions (Fabs), recombinant antibodies, monoclonal antibodies, fusion proteins comprising the antigen-binding domain of a native antibody or an aptamer, single-domain antibodies (sdAbs), also known as VHH antibodies, nanobodies (Camelid-derived single-domain antibodies), shark IgNAR-derived single-domain antibody fragments called VNAR, diabodies, triabodies, Anticalins, aptamers (DNA or RNA) and active components or fragments thereof.

A “Fab fragment” (also referred to as fragment antigen-binding, or Fab region) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CHI domain including one or more cysteines from the antibody hinge region.

A “Single-chain Fv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). As an example of scFv fragments, antibody scFv fragments are described in WO93/16185; U.S. Pat. Nos. 5,571,894; and 5,587,458.

The effector moiety may be a Fab region of a therapeutic antibody. For example, the effector moiety may be the Fab region of a monoclonal antibody such as muromomab, abciximab, rituximab, daclizumab, basiliximab, palivizumab, infliximab, trastuzumab, etanercept, gemtuzumab, alemtuzumab, ibritomomab, adalimumab, alefacept, omalizumab, tositumomab, efalizumab, cetuximab, bevacizumab, natalizumab, ranibizumab, panitumumab, eculizumab, or certolizumab.

The effector moiety may target any receptor associated with a pathological condition, e.g. a pathological condition described herein. For example, the effector moiety may target any receptor the binding of which is associated with a clinical benefit. For example, hormone receptors.

In some embodiments the effector moiety may have a target (e.g. a receptor) not previously known to be associated with a pathological condition. For instance, targeting of such receptors has been found to have therapeutic benefit in some cases.

Those skilled in the art will appreciate that the protein complexes provided herein can therefore be used to simultaneously engage two targets within the biological system thereby contacted simultaneously. The targets may for example be from the same cell. For example, the protein complexes provided herein can be used to bind to two different types of receptor on the same cell surface.

The protein complexes provided herein typically comprise a plurality of first binding sites and a plurality of second binding sites on the multivalent protein scaffold; and thus can bind a plurality of first effector moieties and a plurality of second effector moieties. This is particularly beneficial as such “high valency” compounds may allow for improved or previously unseen effector functions. It has previously been shown that multiple copies of a single effector moiety may lead to an improved therapeutic response when contacted with a biological system (e.g. Brune et al. (above); and Khairil Anuar et al., Nature communications 10.1 (2019): 1-13). However the contacting of multiple copies of a plurality of different effector moieties is a complex technical challenge that is solved by the protein complexes provided herein.

In some embodiments an effector function may arise only as a result of the interaction of a combination of effector moieties with the biological system contacted therewith. For example, a first effector moiety (e.g. an effector moiety attached to the first binding site) may be shown to have therapeutic effects only in combination with a second effector moiety (e.g. an effector moiety attached to the second binding site) in circumstances where neither the first nor second effector moiety have therapeutic efficacy alone.

Those skilled in the art will appreciate that the platforms and methods identified herein allow new combinations of therapeutically useful effector moieties to be screened and useful candidates identified.

### Screening Platform

Also provided herein is a screening platform. The screening platform comprises a library, wherein said library comprises a plurality of populations of protein complexes of the invention, wherein the populations of protein complexes each comprise a different combination of first effector moieties, second effector moieties and/or oligomeric core. Such a library is also provided herein.

For example, the library may be used to screen new combinations of effector moieties. Thus, the library may comprise a plurality of samples of different protein complexes. Each sample may be homogeneous, i.e. each sample may contain just one type of protein complex. Each sample may be different to each other sample. Thus, each sample may contain protein complexes comprising a different combination of first and second effector moieties compared to the combination of first and second effector moieties in the protein complexes of each other sample. For example, the library may comprise from about 1 or about 2 to about 1,000,000 samples, e.g. from about 10 to about 100,000 samples, e.g. from about 50 to about 50,000 samples, such as from about 100 to about 10,000 samples, e.g. from about 500 to about 1,000 samples. Each sample may comprise a different type of protein complex, wherein the protein complex in each sample has a different combination of first effector moieties, second effector moieties and oligomeric cores compared to the protein complexes in all other samples.

In some embodiments the library may be a “ID” library. Thus, in some embodiments all samples in the library may have the same or substantially the same oligomeric core and first effector moiety and may differ in terms of the second effector moiety. In other embodiments all samples in the library may have the same or substantially the same oligomeric core and second effector moiety and may differ in terms of the first effector moiety. In some other embodiments all samples in the library may have the same or substantially the same first and second effector moieties and may differ in terms of the oligomeric core. A polypeptide (e.g. the oligomeric core, or the first or second polypeptide binding site) which is substantially the same as a given polypeptide may for example have at least 90% sequence identity to the given polypeptide, e.g. at least 95% sequence identity such as at least 97%, 98%, 99%, 99.9% or 99.99% sequence identity to the given polypeptide. A polypeptide (e.g. the oligomeric core, or the first or second polypeptide binding site) which is substantially the same as a given polypeptide may for example differ from the given polypeptide by comprising one or more sequence additions, deletions or insertions or variations as described herein. A polypeptide (e.g. the oligomeric core, or the first or second polypeptide binding site) which is substantially the same as a given polypeptide may for example differ from the given polypeptide in terms of post-translational modifications made to the polypeptide, e.g. its glycosylation or phosphorylation pattern.

In some embodiments the library may be a “2D” library. Thus, in some embodiments all samples in the library may have the same or substantially the same oligomeric core and differ in terms of the combination of the first effector moiety and the second effector moiety. In other embodiments all samples in the library may have the same or substantially the same first effector moiety and differ in terms of the combination of the oligomeric core and the second effector moiety. In some other embodiments all samples in the library may have the same or substantially the same second effector moiety and may differ in terms of the combination of the oligomeric core and the first effector moiety.

In some embodiments the library may be a “3D” library. Thus in some embodiments all samples in the library may differ in terms of the combination of oligomeric core, the first effector moiety and the second effector moiety.

The screening platform may also comprise other constituent parts in addition to the library. For example, the screening platform may comprise any or all of:
a biological system for contacting with the samples in the library;
a detector system for detecting changes in the biological system resulting from contacting the biological system with samples in the library;
reagents and/or buffer solutions; and
optical, electrical or spectroscopic means for detecting changes reported by the detector system

The biological system may be a cell culture, such as a mammalian cell culture, preferably a human cell culture, more preferably an immune cell culture and/or a cancer-cell line culture. The biological system may be a biological sample, such as blood sample, serum sample, plasma sample, or a sample of tissue or organ. The biological sample include tumor samples, cells, cell lysates, urine, amniotic fluid and other biological fluids. The biological sample is preferably mammalian. The sample may be human or non-human.

The detector system may be any suitable detector system. The detector system may be a dye or stain, e.g. a cell viability stain. Suitable stains may include for example trypan blue, (fluorescein diacetate)-green, propidium iodide, Hoechst 33258, and the like.

Reagents include components required for cell viability, including cell growth media components; and may include therapeutic molecules.

Buffers include aqueous compositions which may comprise e.g. buffer salts. Preferred buffer salts which can be used include Tris; phosphate; citric acid/Na₂HPO₄; citric acid/sodium citrate; sodium acetate/acetic acid; Na₂HPO₄/NaH₂PO₄; imidazole (glyoxaline)/HCl; sodium carbonate/sodium bicarbonate; ammonium carbonate/ammonium bicarbonate; MES; Bis-Tris; ADA; aces; PIPES; MOPSO; Bis-Tris Propane; BES; MOPS; TES; HEPES; DIPSO; MOBS; TAPSO; Trizma; HEPPSO; POPSO; TEA; EPPS; Tricine; Gly-Gly; Bicine; HEPBS; TAPS; AMPD; TABS; AMPSO; CHES; CAPSO; AMP; CAPS and CABS. Selection of appropriate buffers for a desired pH is routine to those skilled in the art, and guidance is available at e.g. http://www.sigmaaldrich.com/life-science/core-bioreagents/biological-buffers/learning-center/buffer-reference-center.html. Buffer salts are preferably used at concentrations of from 1 mM to 1 M, preferably from 10 mM to 100 mM such as about 50 mM in solution.

Means for detecting changes reported by the detector system include microscopes (optical or electronic), electrical means such as electrophysiology (e.g. patch clamp) apparatus; and spectroscopic means such as equipment for UV/VIS spectroscopy, NMR spectroscopy, mass spectroscopy, IR spectroscopy, Raman spectroscopy, circular dichroism spectroscopy, etc.

### Methods

Also provided is a method for identifying a therapeutic drug analog, the method comprising:
providing a protein complex as described herein;
contacting the protein complex with a biological system; and
measuring whether the protein complex induces a desired change in a property of the biological system.

The method may optionally further comprise selecting a protein complex that induces a desired change in a property of the biological system.

Also provided is a method for identifying a therapeutic combination of effector molecules (e.g. antigen-binding domains), the method comprising:
providing a protein complex as described herein;
contacting the protein complex with a biological system; and
measuring whether the protein complex induces a desired change in a property of the biological system.

The biological system may be a cell culture, such as a mammalian cell culture, preferably a human cell culture, more preferably an immune cell culture and/or a cancer-cell line culture.

The biological system may be a biological sample, such as blood sample, serum sample, plasma sample, or a sample of tissue or organ. The biological sample include tumor samples, cells, cell lysates, urine, amniotic fluid and other biological fluids. The biological sample is preferably mammalian. The sample may be human or non-human.

The change in the property of the biological system can be any change associated with the desired activity of the intended therapeutic. In some embodiments the desired change is cell death. This can be particularly used when developing cancer therapeutics.

Other changes include changes in effector functions. Thus, the method may comprise a step of measuring whether the protein complex induces an effector function in the biological system.

Changes in effector functions may include altered gene expression, altered protein modifications for example phosphorylation, receptor internalization, cytokine release, cell death, susceptibility to therapeutic molecules, etc. The effector function may be high affinity binding to the biological sample, which can be measured by a range of techniques, such as ELISA. High affinity binding to a target biological system may allow effector domains, discussed above, to specifically effect the targeted biological system, which may be a particular cell type, such as a cancer cell.

The effector function can be assessed with reference to a control. The control may be the protein complex without effector moieties.

The control may be a protein complex with only a single type of effector moiety attached (i.e. only one type of effector moiety is attached to the multivalent protein scaffold). In this case, the method can be used to identify effector moieties with a “synergistic function” or “synergistic biological function”, which refers to an effector function or level of effector function that: is not observed with individual fusion protein components until a bispecific multivalent protein complex is used; or higher or lower activity in comparison to the activity observed when the first and second effector moieties of the protein complex are employed individually, i.e. activity which is only observed when both effector moieties are used together in the complex.

The method may further comprise a step of identifying the molecules of the biological system that are bound by the effector moieties of the protein complex. The method may preferably comprise selecting a combination of effector moieties that specifically bind to the same molecules of the biological system as the selected protein complex, such as the effector moieties themselves.

The method may further comprise synthesising a therapeutic drug candidate or drug comprising the selected combination of effector moieties, or analogs thereof. The therapeutic drug candidate or drug may comprise the oligomeric core and effector moieties of the therapeutic drug analog, but wherein the binding site and target functionalities are replaced with a covalent linkage such as a genetic fusion as described in more detail herein. The therapeutic drug or drug candidate may comprise the same oligomeric core as the therapeutic drug analog identified in the disclosed methods. Alternatively the therapeutic drug candidate may comprise a different oligomeric core as the therapeutic drug analog identified in the disclosed methods. The therapeutic drug candidate may have an oligomeric core chosen or designed in order to impart an additional therapeutic benefit, for example a further effector function.

Also provided herein is a therapeutic drug candidate obtainable according to the disclosed methods.

### Therapeutic Drug Candidate, Therapeutic Drug

Further provided is a therapeutic drug candidate, comprising an oligomeric core comprising a plurality of subunit monomers attached to one or more first effector moieties and one or more second effector moieties; wherein said one or more first effector moieties and said one or more second effector moieties are positioned on the same face of the oligomeric core; and wherein (i) said one or more first effector moieties comprise two or more first effector moieties and said one or more second effector moieties comprise two or more second effector moieties; and/or (ii) said oligomeric core does not comprise an antibody or antibody fragment.

A therapeutic drug is also provided with the same features.

Typically, the oligomeric core is an oligomeric core as described in more detail herein. Typically, the subunit monomers are as described in more detail herein. Typically, the first effector moieties and second effector moieties are as described in more detail here. The first and second effector moieties may be attached to the subunit monomers of the oligomeric core in any appropriate manner, including by any of the attachment means described herein. In some embodiments the attachment of the first and second effector moieties comprises first and second binding sites and first and second polypeptide targets as described herein. However in other embodiments the attachment of the first and second effector moieties does not comprise first and second binding sites and first and second polypeptide targets as described herein and may instead comprise a simple covalent attachment, such as a genetic fusion and/or a click chemistry linkage as described herein.

### SPECIFIC EMBODIMENTS

In a first preferred aspect, provided herein is:

A multivalent protein scaffold comprising an oligomeric core comprising a plurality (e.g. from 3 to 6, preferably 3) of monomers each comprising an amino acid sequence having at least 30% or at least 50% amino acid identity (e.g. at least 60%, at least 70%, at least 80%, at least 90%, at least 95%, at least 98%, at least 99% or 100% % amino acid identity) to the amino acid sequence of SEQ ID NO: 1; wherein each monomer comprises a first binding site and a second binding site, wherein the first binding site is orthogonal to the second binding site, and wherein the first binding site and the second binding site each independently have at least 50% amino acid identity (e.g. at least 60%, at least 70%, at least 80%, at least 90%, at least 95%, at least 98%, at least 99% or 100% % amino acid identity) to any one of SEQ ID NOs: 4-9, 11-13, 23 or 15-18; and wherein each first binding site and each second binding site are independently genetically fused to the monomer to which they are attached. Preferably, one of the first and second binding sites has at least 50% amino acid identity to SEQ ID NO: 4, 6 or 8 and the other has at least 50% amino acid identity to SEQ ID N: 12. A preferred multivalent protein scaffold of this aspect comprises monomers of SEQ ID NO: 21, or a fragment thereof (e.g. comprising residues 14-348 thereof).

A protein complex comprising the multivalent protein scaffold of the first aspect, wherein the first binding site is bound to a first polypeptide target attached to a first effector moiety; and the second binding site is bound to a first polypeptide target attached to a second effector moiety; wherein the first and second effector moieties may be the same or different, preferably different; and wherein the first binding site/polypeptide target pair and the second binding site/polypeptide target pair are each independently selected from the following combinations: (i) any one of SEQ ID NO: 4, 6 or 8 with any one of SEQ ID NOs: 5, 7 or 9; (ii) SEQ ID NO: 12 with SEQ ID NO: 13 or 15; (iii) SEQ ID NO: 5 with SEQ ID NO: 11; (iv) SEQ ID NO: 15 with SEQ ID NO: 16); (v) SEQ ID NO: 17 with SEQ ID NO: 18; or (vi) SEQ ID NO: 23 with SEQ ID NO: 16).

A screening platform comprising a library comprising a plurality of populations of protein complexes of the first aspect, wherein each population comprises a different combination of first and second effector moieties.

A therapeutic drug candidate comprising an oligomeric core comprising a plurality (e.g. from 3 to 6, preferably 3) of monomers each comprising an amino acid sequence having at least 50% amino acid identity (e.g. at least 60%, at least 70%, at least 80%, at least 90%, at least 95%, at least 98%, at least 99% or 100% % amino acid identity) to the amino acid sequence of SEQ ID NO: 1; wherein each monomer is directly attached (e.g. as a genetic fusion) to a first effector moiety and a second effector moiety; wherein the first and second effector moieties may be the same or different, preferably different; and preferably wherein each monomer is directly attached via a polypeptide linker to the first effector moiety and the second effector moiety attached thereto.

In a second preferred aspect, specifically provided herein is:

A multivalent protein scaffold comprising an oligomeric core comprising a plurality (e.g. from 3 to 6, preferably 3) of monomers each comprising an amino acid sequence having at least 50% amino acid identity (e.g. at least 60%, at least 70%, at least 80%, at least 90%, at least 95%, at least 98%, at least 99% or 100% % amino acid identity) to the amino acid sequence of SEQ ID NO: 2; wherein each monomer comprises a first binding site and a second binding site, wherein the first binding site is orthogonal to the second binding site, and wherein the first binding site and the second binding site each independently have at least 50% amino acid identity (e.g. at least 60%, at least 70%, at least 80%, at least 90%, at least 95%, at least 98%, at least 99% or 100% % amino acid identity) to any one of SEQ ID NOs: 4-9, 11-13, 23 or 15-18; and wherein each first binding site and each second binding site are independently genetically fused to the monomer to which they are attached. Preferably, one of the first and second binding sites has at least 50% amino acid identity to SEQ ID NO: 4, 6 or 8 and the other has at least 50% amino acid identity to SEQ ID N: 12. A preferred multivalent protein scaffold of this aspect comprises monomers of SEQ ID NO: 20, or a fragment thereof (e.g. comprising residues 14-380 thereof).

A protein complex comprising the multivalent protein scaffold of the second aspect, wherein the first binding site is bound to a first polypeptide target attached to a first effector moiety; and the second binding site is bound to a first polypeptide target attached to a second effector moiety; wherein the first and second effector moieties may be the same or different, preferably different; and wherein the first binding site/polypeptide target pair and the second binding site/polypeptide target pair are each independently selected from the following combinations: (i) any one of SEQ ID NO: 4, 6 or 8 with any one of SEQ ID Nos: 5, 7 or 9; (ii) SEQ ID NO: 12 with SEQ ID NO: 13 or 15; (iii) SEQ ID NO: 5 with SEQ ID NO: 11; (iv) SEQ ID NO: 15 with SEQ ID NO: 16); (v) SEQ ID NO: 17 with SEQ ID NO: 18; or (vi) SEQ ID NO: 23 with SEQ ID NO: 16).

A screening platform comprising a library comprising a plurality of populations of protein complexes of the second aspect, wherein each population comprises a different combination of first and second effector moieties.

A therapeutic drug candidate comprising an oligomeric core comprising a plurality (e.g. from 3 to 6, preferably 3) of monomers each comprising an amino acid sequence having at least 50% amino acid identity (e.g. at least 60%, at least 70%, at least 80%, at least 90%, at least 95%, at least 98%, at least 99% or 100% % amino acid identity) to the amino acid sequence of SEQ ID NO: 2; wherein each monomer is directly attached (e.g. as a genetic fusion) to a first effector moiety and a second effector moiety; wherein the first and second effector moieties may be the same or different, preferably different; and preferably wherein each monomer is directly attached via a polypeptide linker to the first effector moiety and the second effector moiety attached thereto.

In a third preferred aspect, specifically provided herein is:

A multivalent protein scaffold comprising an oligomeric core comprising a plurality (e.g. from 3 to 6, preferably 3) of monomers each comprising an amino acid sequence having at least 50% amino acid identity (e.g. at least 60%, at least 70%, at least 80%, at least 90%, at least 95%, at least 98%, at least 99% or 100% % amino acid identity) to the amino acid sequence of SEQ ID NO: 3; wherein each monomer comprises a first binding site and a second binding site, wherein the first binding site is orthogonal to the second binding site, and wherein the first binding site and the second binding site each independently have at least 50% amino acid identity (e.g. at least 60%, at least 70%, at least 80%, at least 90%, at least 95%, at least 98%, at least 99% or 100% % amino acid identity) to any one of SEQ ID Nos: 4-9, 11-13, 23 or 15-18; and wherein each first binding site and each second binding site are independently genetically fused to the monomer to which they are attached. Preferably, one of the first and second binding sites has at least 50% amino acid identity to SEQ ID NO: 4, 6 or 8 and the other has at least 50% amino acid identity to SEQ ID N: 12.

A protein complex comprising the multivalent protein scaffold of the third aspect, wherein the first binding site is bound to a first polypeptide target attached to a first effector moiety; and the second binding site is bound to a first polypeptide target attached to a second effector moiety; wherein the first and second effector moieties may be the same or different, preferably different; and wherein the first binding site/polypeptide target pair and the second binding site/polypeptide target pair are each independently selected from the following combinations: (i) any one of SEQ ID NO: 4, 6 or 8 with any one of SEQ ID NOs: 5, 7 or 9; (ii) SEQ ID NO: 12 with SEQ ID NO: 13 or 15; (iii) SEQ ID NO: 5 with SEQ ID NO: 11; (iv) SEQ ID NO: 15 with SEQ ID NO: 16); (v) SEQ ID NO: 17 with SEQ ID NO: 18; or (vi) SEQ ID NO: 23 with SEQ ID NO: 16).

A screening platform comprising a library comprising a plurality of populations of protein complexes of the third aspect, wherein each population comprises a different combination of first and second effector moieties.

A therapeutic drug candidate comprising an oligomeric core comprising a plurality (e.g. from 3 to 6, preferably 3) of monomers each comprising an amino acid sequence having at least 50% amino acid identity (e.g. at least 60%, at least 70%, at least 80%, at least 90%, at least 95%, at least 98%, at least 99% or 100% % amino acid identity) to the amino acid sequence of SEQ ID NO: 3; wherein each monomer is directly attached (e.g. as a genetic fusion) to a first effector moiety and a second effector moiety; wherein the first and second effector moieties may be the same or different, preferably different; and preferably wherein each monomer is directly attached via a polypeptide linker to the first effector moiety and the second effector moiety attached thereto.

In a fourth preferred aspect, specifically provided herein is:

A multivalent protein scaffold comprising an oligomeric core comprising a plurality (e.g. from 3 to 6, preferably 3) of monomers each comprising an amino acid sequence having at least 50% amino acid identity (e.g. at least 60%, at least 70%, at least 80%, at least 90%, at least 95%, at least 98%, at least 99% or 100% % amino acid identity) to the amino acid sequence of SEQ ID NO: 19; wherein each monomer comprises a first binding site and a second binding site, wherein the first binding site is orthogonal to the second binding site, and wherein the first binding site and the second binding site each independently have at least 50% amino acid identity (e.g. at least 60%, at least 70%, at least 80%, at least 90%, at least 95%, at least 98%, at least 99% or 100% % amino acid identity) to any one of SEQ ID NOs: 4-9, 11-13, 23 or 15-18; and wherein each first binding site and each second binding site are independently genetically fused to the monomer to which they are attached. Preferably, one of the first and second binding sites has at least 50% amino acid identity to SEQ ID NO: 4, 6 or 8 and the other has at least 50% amino acid identity to SEQ ID N: 12.

A protein complex comprising the multivalent protein scaffold of the fourth aspect, wherein the first binding site is bound to a first polypeptide target attached to a first effector moiety; and the second binding site is bound to a first polypeptide target attached to a second effector moiety; wherein the first and second effector moieties may be the same or different, preferably different; and wherein the first binding site/polypeptide target pair and the second binding site/polypeptide target pair are each independently selected from the following combinations: (i) any one of SEQ ID NO: 4, 6 or 8 with any one of SEQ ID Nos: 5, 7 or 9; (ii) SEQ ID NO: 12 with SEQ ID NO: 13 or 15; (iii) SEQ ID NO: 5 with SEQ ID NO: 11; (iv) SEQ ID NO: 15 with SEQ ID NO: 16); (v) SEQ ID NO: 17 with SEQ ID NO: 18; or (vi) SEQ ID NO: 23 with SEQ ID NO: 16).

A screening platform comprising a library comprising a plurality of populations of protein complexes of the fourth aspect, wherein each population comprises a different combination of first and second effector moieties.

A therapeutic drug candidate comprising an oligomeric core comprising a plurality (e.g. from 3 to 6, preferably 3) of monomers each comprising an amino acid sequence having at least 50% amino acid identity (e.g. at least 60%, at least 70%, at least 80%, at least 90%, at least 95%, at least 98%, at least 99% or 100% % amino acid identity) to the amino acid sequence of SEQ ID NO: 4; wherein each monomer is directly attached (e.g. as a genetic fusion) to a first effector moiety and a second effector moiety; wherein the first and second effector moieties may be the same or different, preferably different; and preferably wherein each monomer is directly attached via a polypeptide linker to the first effector moiety and the second effector moiety attached thereto.

In a fifth preferred aspect, specifically provided herein is:

A polypeptide comprising a first binding domain at the N terminus and a second binding domain at the C terminus, wherein the first and second binding domains are separated by a structural domain and wherein the first antigen binding domain and a second antigen binding domain are able to bind to their targets when the target molecules are expressed on a single cell or immobilised onto a plate or single bead.

An oligomer of polypeptides, wherein each polypeptide in the oligomer comprises or consists of a polypeptide comprising a first binding domain at the N terminus and a second binding domain at the C terminus, wherein the first and second binding domains are separated by a structural domain and wherein the first antigen binding domain and a second antigen binding domain are able to bind to their targets when the target molecules are expressed on a single cell or immobilised onto a plate or single bead.

A polypeptide comprising a first binding domain at the N terminus and a second binding domain at the C terminus, wherein the first and second binding domains are separated by a structural domain, wherein the first binding domain and second binding domain are able to bind to their targets when the targets molecules are expressed on a single cell or immobilised onto a plate or single bead. The first binding domain and the second binding domain are catcher domains that are each able to form an isopeptide linkage with a cognate peptide. These cognate peptides are often referred to as tag peptides, for example a SpyTag forms an isopeptide bond with a SpyCatcher domain as is known in the art and as discussed above. The cognate peptide for the first binding domain is different from the cognate peptide for the second binding domain.

An oligomer of polypeptides, wherein each polypeptide in the oligomer comprises or consists of a polypeptide comprising a first binding domain at the N terminus and a second binding domain at the C terminus, wherein the first and second binding domains are separated by a structural domain, wherein the first binding domain and second binding domain are able to bind to their targets when the targets molecules are expressed on a single cell or immobilised onto a plate or single bead. The first binding domain and the second binding domain are catcher domains that are each able to form an isopeptide linkage with a cognate peptide. These cognate peptides are often referred to as tag peptides, for example a Spy Tag forms an isopeptide bond with a SpyCatcher domain as is known in the art and as discussed above. The cognate peptide for the first binding domain is different from the cognate peptide for the second binding domain.

### Additional Aspects of the Disclosure

Also provided herein is a polynucleotide encoding at least one monomer of the oligomeric core of the multivalent protein scaffold as described in more detail herein. Also provided herein is a polynucleotide encoding the multi-domain polypeptide construct as described in more detail herein, comprising a first binding domain, a second binding domain and a structural domain

Further provided is a vector comprising the polynucleotide; a cell comprising the vector; and a method of producing the monomer, oligomeric core and/or multivalent protein scaffold, comprising culturing the cell in a medium to produce the protein scaffold.

Further provided is a vector comprising the polynucleotide; a cell comprising the vector; and a method of producing the multi-domain polypeptide construct, comprising culturing the cell in a medium to produce the multi-domain polypeptide.

Selection of appropriate polynucleotide sequences to encode the at least one monomer; of appropriate expression vectors; and of appropriate cells in which to express the monomer, oligomeric core and/or multivalent protein scaffold is routine to those of skill in the art.

### Therapeutic Efficacy

The protein complexes, therapeutic drug analogs and therapeutic drug candidates provided herein are therapeutically useful. The multi-domain polypeptide construct is typically therapeutically useful. These provided substances are also referred to herein as “therapeutic protein complexes”.

The present invention therefore provides therapeutic protein complexes and constructs as described herein, for use in medicine. The present invention provides therapeutic protein complexes as described herein, for use in treating the human or animal body. The present invention provides therapeutic protein constructs as described herein, for use in treating the human or animal body.

The present invention provides a method of treating a human or animal in need of such treatment, comprising administering to the human or animal in need of treatment a protein complex, multi-domain polypeptide construct (in monomeric or oligomeric form), therapeutic drug analog, therapeutic drug candidate, or drug as described herein.

Also provided is a pharmaceutical composition comprising one or more therapeutic protein complexes as described herein together with a pharmaceutically acceptable carrier or diluent. Typically, the composition contains up to 85 wt % of a therapeutic protein complexes of the invention. More typically, it contains up to 50 wt % of a therapeutic protein complex of the invention. Preferred pharmaceutical compositions are sterile and pyrogen free.

Also provided is a pharmaceutical composition comprising one or more multi-domain polypeptide constructs as described herein together with a pharmaceutically acceptable carrier or diluent. Typically, the composition contains up to 85 wt % of a therapeutic multi-domain polypeptide construct of the invention. More typically, it contains up to 50 wt % of a therapeutic multi-domain polypeptide construct of the invention. Preferred pharmaceutical compositions are sterile and pyrogen free.

The composition of the invention may be provided as a kit comprising instructions to enable the kit to be used in the methods described herein or details regarding which subjects the method may be used for.

As explained above, the therapeutic protein complexes and constructs provided herein are useful in treating or preventing various disorders. Disorders for treatment using the provided therapeutic protein complexes may include cancer, autoimmune disorders (e.g. ankolysing spondylitis), psoriasis, eye disorders such as age-related macular degeneration, multiple sclerosis, cardiovascular disorders, infections including viral and bacterial infections, Crohn's disease, Rheumatoid arthritis, osteoarthritis, Alzheimer's disease, transplant and allograft rejection, etc. hematopoietic stem cell disorders, and the like. More broadly, therapeutic protein complexes as provided herein find utility in treating any and all conditions also treated using antibodies, particularly bispecific antibodies.

Cancer, e.g. acute lymphoblastic leukemia, acute myeloid leukemia, adrenocortical carcinoma, aids-related lymphoma, primary CNS lymphoma, anal cancer, astrocytomas, brain cancer, basal cell carcinoma, bile duct cancer, bladder cancer, bone cancer (e.g. ewing sarcoma, osteosarcoma and malignant fibrous histiocytoma), breast cancer, bronchial tumors, medulloblastoma and other CNS embryonal tumors, cervical cancer, chronic lymphocytic leukemia, chronic myelogenous leukemia, chronic myeloproliferative neoplasms, colorectal cancer, craniopharyngioma, endometrial cancer, ependymoma, esophageal cancer, esthesioneuroblastoma, ewing sarcoma, extragonadal germ cell tumor, intraocular melanoma, retinoblastoma, fallopian tube cancer, gallbladder cancer, gastric cancer, gastrointestinal carcinoid tumor, gastrointestinal stromal tumors (gist), germ cell tumors, extragonadal germ cell tumors, ovarian germ cell tumors, testicular cancer, gestational trophoblastic disease, hairy cell leukemia, hepatocellular cancer, histiocytosis, langerhans cell, hodgkin lymphoma, hypopharyngeal cancer, intraocular melanoma, islet cell tumors, pancreatic neuroendocrine tumors, kaposi sarcoma, kidney (renal cell) cancer, langerhans cell histiocytosis, laryngeal cancer, leukemia, liver cancer, lung cancer (non-small cell, small cell, pleuropulmonary blastoma, and tracheobronchial tumor), lymphoma, malignant fibrous histiocytoma of bone and osteosarcoma, merkel cell carcinoma, mesothelioma, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, neuroblastoma, non-hodgkin lymphoma, oropharyngeal cancer, osteosarcoma and undifferentiated pleomorphic sarcoma, pancreatic cancer, pancreatic neuroendocrine tumors (islet cell tumors), papillomatosis, paraganglioma, parathyroid cancer, penile cancer, pharyngeal cancer, pheochromocytoma, pituitary tumor, prostate cancer, rectal cancer, retinoblastoma, rhabdomyosarcoma, t-cell lymphoma, testicular cancer, throat cancer, thymoma and thymic carcinoma, thyroid cancer, tracheobronchial tumors and the like is particularly suitable to being treated with the therapeutic protein complexes provided herein. Autoimmune disorders amenable to being treated with the therapeutic protein complexes provided herein include rheumatoid arthritis, systemic lupus erythematosus (lupus), inflammatory bowel disease (IBD), multiple sclerosis (MS), Type 1 diabetes mellitus, Guillain-Barre syndrome, chronic inflammatory demyelinating polyneuropathy, psoriasis, Graves' disease, Hashimoto's thyroiditis, Myasthenia gravis and vasculitis.

The therapeutic protein complexes provided herein may be used as standalone therapeutic agents. Alternatively, they may be used in combination with other active agents such as chemotherapeutic agents. For example, they may be used in combination with an EGFR inhibitor (for instance erlotinib, gefitinib, lapatinib or cetuximab), an immunotherapy (for instance pembrolizumab or nivolumab), a tumour-agnostic therapy (for instance larotrectinib) or a chemotherapy (for instance 5-fluorouracil, cisplatin or docetaxel).

When used to treat a cancer, the therapeutic protein complexes provided herein may be used in alleviating, ameliorating or preventing aggravation of the symptoms of the cancer. Typically, treating a cancer may comprise reducing progression of the cancer, e.g. increasing progression free survival. Treating a cancer may comprise preventing or inhibiting growth of a tumour associated with the cancer. Treating a cancer may comprise preventing metastasis of the cancer. Preferably, treating a cancer may comprise reducing the size of a tumour associated with the cancer. As such, the treatment may cause tumour regression in the cancer. Treating a cancer may comprise reducing the number of tumours or lesions present in the patient. When the treatment reduces the size of a tumour associated with the cancer, the size of the tumour is typically reduced from base line by at least 10%. Base line is the size of the tumour at the date treatment with the compound is first started. The size of the tumour is typically as measured in accordance with version 1.1 of the RECIST criteria (for instance as described in Eisenhauer et al, European Journal of Cancer 45 (2009) 228-247).

The response to the treatment with the compound may be complete response, partial Response or stable disease, in accordance with version 1.1 of the RECIST criteria. Preferably, the response is partial response or complete response. The treatment may achieve progression free survival for at least 60 days, at least 120 days or at least 180 days.

The reduction in tumour size may be greater 20%, greater than 30% or greater than 50% reduction relative to base line. The reduction in tumour size may be observed after 30 days of treatment or after 60 days of treatment.

The therapeutic protein complexes provided herein may also be useful in treating infection, such as infection caused by Gram-positive and/or Gram-negative bacteria; and viral infections. The therapeutic protein complexes provided herein may be designed to interact with pathogens such as bacteria, fungi and viruses.

As explained here, the therapeutic protein complexes provided herein are useful in treating or preventing various disorders. The present invention therefore provides a therapeutic protein complex as provided herein for use in medicine. The invention also provides the use of a therapeutic protein complex as provided herein in the manufacture of a medicament. The invention also provides compositions and products comprising the therapeutic protein complexes provided herein. Such compositions and products are also useful in treating or preventing disorders. The present invention therefore provides a composition or product as defined herein for use in medicine. The invention also provides the use of a composition or product of the invention in the manufacture of a medicament. Also provided is a method of treating a subject in need of such treatment, said method comprising administering to the subject a therapeutic protein complex provided herein. In some embodiments the subject suffers from or is at risk of suffering from one of the disorders disclosed herein.

In one aspect, the subject is a mammal, in particular a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters. The subject can be any animal that is capable of being infected by a bacterium.

A subject is typically a human patient. The patient may be male or female. The age of the patient is typically at least 18 years, for instance from 30 to 70 years or from 40 to 60 years. The subject may also be paediatric or adolescent, for example between 6 months and 11 years or between 12 years and 17 years.

A therapeutic protein complex, polypeptide construct or composition of the invention can be administered to the subject in order to prevent the onset or reoccurrence of one or more symptoms of the disorder. This is prophylaxis. In this embodiment, the subject can be asymptomatic. A prophylactically effective amount of the agent or formulation is administered to such a subject. A prophylactically effective amount is an amount which prevents the onset of one or more symptoms of the disorder.

A therapeutic protein complex, polypeptide construct or composition of the invention can be administered to the subject in order to treat one or more symptoms of the disorder. In this embodiment, the subject is typically symptomatic. A therapeutically effective amount of the agent or formulation is administered to such a subject. A therapeutically effective amount is an amount effective to ameliorate one or more symptoms of the disorder.

The therapeutic protein complex, polypeptide construct or composition of the invention may be administered in a variety of dosage forms. Thus, it can be administered orally, for example as tablets, troches, lozenges, aqueous or oily suspensions, dispersible powders or granules. The therapeutic protein complex or composition of the invention may also be administered parenterally, whether subcutaneously, intravenously, intramuscularly, intrasternally, transdermally or by infusion techniques. The therapeutic protein complex, polypeptide construct or composition may also be administered as a suppository. Preferably, the compound, composition or combination may be administered via inhaled (aerosolised) or intravenous administration, most preferably by inhaled (aerosolised) administration.

The therapeutic protein complex or composition of the invention is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

The therapeutic protein complex, polypeptide construct or composition of the invention may be formulated for inhaled (aerosolised) administration as a solution or suspension. The therapeutic protein complex or composition of the invention may be administered by a metered dose inhaler (MDI) or a nebulizer such as an electronic or jet nebulizer. Alternatively, the therapeutic protein complex or composition of the invention may be formulated for inhaled administration as a powdered drug, such formulations may be administered from a dry powder inhaler (DPI). When formulated for inhaled administration, the therapeutic protein complex or composition of the invention may be delivered in the form of particles which have a mass median aerodynamic diameter (MMAD) of from 1 to 100 μm, preferably from 1 to 50 μm, more preferably from 1 to 20 μm such as from 3 to 10 μm, e.g. from 4 to 6 μm. When the therapeutic protein complex or composition of the invention is delivered as a nebulized aerosol, the reference to particle diameters defines the MMAD of the droplets of the aerosol. The MMAD can be measured by any suitable technique such as laser diffraction.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections or inhalation may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used.

A therapeutically or prophylactically effective amount of the therapeutic protein complex or composition of the invention is administered to a subject. The dose may be determined according to various parameters, especially according to the compound used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific inhibitor, the age, weight and conditions of the subject to be treated, the type and severity of the disease and the frequency and route of administration. Preferably, daily dosage levels are from 5 mg to 2 g.

When the therapeutic protein complex or composition of the invention is administered to a subject in combination with another active agent, the dose of the other active agent can be determined as described above. The dose may be determined according to various parameters, especially according to the agent used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific agent, the age, weight and conditions of the subject to be treated, the type and severity of the disease and the frequency and route of administration. Preferably, daily dosage levels are from 5 mg to 2 g.

The protein complexes, therapeutic drug analogs, and therapeutic drug candidates provided herein are also useful in diagnostic methods. The polypeptide constructs, and drugs provided herein are also useful in diagnostic methods. Accordingly, provided herein are protein complexes, therapeutic drug analogs or therapeutic drug candidates, or polypeptide constructs or drugs, as described herein for use in a method of diagnosing a pathology in a subject. The subject may be a subject as described in more detail herein. The pathology may be a pathology as described herein. The method may comprise contacting the protein complex, therapeutic drug analog or therapeutic drug candidate with a sample obtained from the subject (e.g. a biological fluid such as blood, serum, urine, or cerebrospinal fluid; and virology swab samples, biopsy and necropsy tissues) and detecting a change characteristic of the pathology occurs in the sample in the presence of the protein complex, therapeutic drug analog or therapeutic drug candidate compared to in the absence thereof.

The invention includes at least the following numbered embodiments:

1. A multivalent protein scaffold comprising:
an oligomeric core comprising a plurality of subunit monomers; and
at least two first binding sites orthogonal to at least two second binding sites;
   wherein said first binding sites and said second binding sites are positioned on the same face of the scaffold.

2. A multivalent protein scaffold comprising:
an oligomeric core comprising a plurality of subunit monomers;
at least one first binding site orthogonal to at least one second binding site;
   wherein said first binding site(s) and said second binding site(s) are positioned on the same face of the scaffold; and
wherein said first binding site comprises a first protein domain capable of forming a covalent bond to a first polypeptide target; and said second binding site comprises a second protein domain capable of forming a covalent bond to a second polypeptide target

3. A multivalent protein scaffold comprising:
an oligomeric core comprising a plurality of subunit monomers;
at least one first binding site orthogonal to at least one second binding site;
   wherein said first binding site(s) and said second binding site(s) are positioned on the same face of the scaffold; and
wherein said oligomeric core does not comprise an Fc region of an antibody.

4. A protein scaffold according to any one of the preceding embodiments, wherein the oligomeric core comprises at least three subunit monomers,
wherein preferably the oligomeric core comprises from 3 to 6 subunit monomers.

5. A protein scaffold according to any one of the preceding embodiments, wherein said subunit monomers are non-covalently attached together.

6. A protein scaffold according to embodiments 1 to 4, wherein said subunit monomers are covalently attached together;
wherein preferably said subunit monomers are genetically fused together.

7. A protein scaffold according to any one of the preceding embodiments, wherein said oligomeric core is a homooligomeric core.

8. A protein scaffold according to any one of the preceding embodiments, wherein each monomer in the oligomeric core comprises at least one first binding site and at least one second binding site, and wherein the at least one first binding site is orthogonal to the at least one second binding site.

9. A protein scaffold according to any one of the preceding embodiments, wherein each monomer comprises a first binding site attached at a first terminus of said monomer and a second binding site at a second terminus of said monomer.

10. A protein scaffold according to any one of the preceding embodiments, wherein the first terminus and the second terminus of each monomer are positioned on the same face of said monomer.

11. A protein scaffold according to any one of embodiments 1 to 8, wherein each monomer comprises a first binding site attached at a first terminus of said monomer and a second binding site attached to said first binding site.

12. A protein scaffold according to any one of embodiments 1 to 6, wherein said oligomeric core is a hetero-oligomeric core.

13. A protein scaffold according to embodiment 12, wherein said core comprises at least one first subunit monomer comprising a first binding site, and at least one second subunit monomer comprising a second binding site, and wherein the first binding site is orthogonal to the second binding site.

14. A protein scaffold according to any one of the preceding embodiments, wherein:
i) each subunit monomer comprises less than 300 amino acids;
   preferably wherein each subunit monomer comprises less than 200 amino acids;
   more preferably wherein each subunit monomer comprises less than 150 amino acids; and/or
ii) the oligomeric core has a molecular weight of less than about 150 kDa, preferably less than about 100 kDa; more preferably less than about 70 kDa.

15. A protein scaffold according to any one of the preceding embodiments, wherein the oligomeric core does not comprise an Fc region of an antibody.

16. A protein scaffold according to any one of the preceding embodiments, wherein the oligomeric core comprises a soluble multimerising structural element of a multimeric protein.

17. A protein scaffold according to embodiment 16, wherein the multimeric protein comprises a collagen NC1 domain, a CutA1, a C1q domain, a TNF, a p53, a fibrinogen, a C4, Bacillus subtillus AbrB or a homolog or paralog thereof.

18. A protein scaffold according to embodiment 16 or embodiment 17, wherein the multimerising structural element comprises a polypeptide have at least 50% amino acid identity to SEQ ID NO: 1, SEQ ID NO: 2, SEQ ID NO: 3 or SEQ ID NO: 19.

19. A protein scaffold according to any one of the preceding embodiments, wherein said first binding site and/or said second binding site comprises a protein domain; wherein preferably said first binding site comprises a first protein domain and said second binding site comprises a second protein domain; and the first binding site and/or second binding site is genetically fused to the subunit monomer(s) to which they are attached to form a single polypeptide chain.

20. A protein scaffold according to any one of the preceding embodiments, wherein said first binding site comprises a first protein domain capable of forming a covalent bond to a first polypeptide target; and said second binding site comprises a second protein domain capable of forming a covalent bond to a second polypeptide target;
wherein preferably said first protein domain is capable of forming an isopeptide bond with said first polypeptide target and said second protein domain is capable of forming an isopeptide bond with said second binding target.

21. A protein scaffold according to embodiment 20, wherein said first binding site and said second binding site each comprise a different split ligand-binding protein domain;
wherein preferably one of said first binding site and said second binding site comprises a split Streptococcus pyogenes fibronectin-binding protein domain and the other of said first binding site and said second binding site comprises a split Streptococcus pneumoniae adhesin domain.

22. A protein scaffold according to embodiment 21, wherein said first and said second binding site each independently have at least 50% amino acid identity to any one of SEQ ID Nos: 4-9, 11-13 or 15-18.

23. A protein complex comprising a protein scaffold according to any one the preceding embodiments, wherein the first binding site is bound to a first polypeptide target attached to a first effector moiety, and the second binding site is bound to a second polypeptide target attached to a second effector moiety.

24. The protein complex according to embodiment 23, wherein the first binding site/polypeptide target pair and the second binding site/polypeptide target pair are each independently selected from the following combinations: (i) any one of SEQ ID NO: 4, 6 or 8 with any one of SEQ ID NOs: 5, 7 or 9; (ii) SEQ ID NO: 12 with SEQ ID NO: 13 or 15; (iii) SEQ ID NO: 5 with SEQ ID NO: 11; (iv) SEQ ID NO: 15 with SEQ ID NO: 16); (v) SEQ ID NO: 17 with SEQ ID NO: 18.

25. A screening platform comprising a library, wherein said library comprises a plurality of populations of protein complexes according to embodiment 23 or embodiment 24, wherein the populations of protein complexes each comprise a different combination of first effector moieties, second effector moieties and/or oligomeric core.

26. A method for identifying a therapeutic drug analog, the method comprising: providing a protein complex according to embodiment 23 or embodiment 24;
contacting the protein complex with a biological system; and
measuring whether the protein complex induces a desired change in a property function of the biological system;
and optionally further comprising selecting a protein complex that induces a desired change in a property of the biological system.

27. A method according to embodiment 26, further comprising:
synthesizing a therapeutic drug candidate comprising the oligomeric core of the scaffold of the protein complex of the identified therapeutic drug analog attached to the first and second effector moieties of said protein complex.

28. A therapeutic drug candidate obtainable according to the method of embodiment 26 or embodiment 27.

29. A therapeutic drug candidate, comprising an oligomeric core comprising a plurality of subunit monomers attached to one or more first effector moieties and one or more second effector moieties; wherein said one or more first effector moieties and said one or more second effector moieties are positioned on the same face of the oligomeric core; and
wherein (i) said one or more first effector moieties comprise two or more first effector moieties and said one or more second effector moieties comprise two or more second effector moieties; and/or (ii) said oligomeric core does not comprise an antibody or antibody fragment.

30. The therapeutic drug candidate of embodiment 29, wherein the oligomeric core is as defined in any one of embodiments 1 to 22.

31. The therapeutic drug candidate of embodiment 29 or embodiment 30, wherein the oligomeric core comprises a plurality of subunit monomers and wherein: (i) each subunit monomer comprises a collagen NC1 domain, a CutA1, a C1q domain, a TNF, a p53, a fibrinogen, a C4, Bacillus subtillus AbrB or a homolog or paralog thereof; and/or (ii) each subunit monomer comprises a multimerising structural element comprising a polypeptide having at least 50% amino acid identity to SEQ ID NO: 1, SEQ ID NO: 2, SEQ ID NO: 3 or SEQ ID NO: 19.

The invention also includes at least the following second set of numbered embodiments:

A1. A multivalent protein scaffold comprising:
an oligomeric core comprising a plurality of subunit monomers;
at least one first binding site orthogonal to at least one second binding site;
   wherein said first binding site(s) and said second binding site(s) are positioned on the same face of the scaffold; and
wherein said first binding site comprises a first protein domain capable of forming a covalent bond to a first polypeptide target; and said second binding site comprises a second protein domain capable of forming a covalent bond to a second polypeptide target.

A2. A protein scaffold according to any one of the preceding numbered embodiments, wherein each monomer in the oligomeric core comprises at least one first binding site and at least one second binding site, and wherein the at least one first binding site is orthogonal to the at least one second binding site.

A3. A protein scaffold according to embodiment A1 or embodiment A2, wherein each monomer comprises a first binding site attached at a first terminus of said monomer and a second binding site at a second terminus of said monomer.

A4. A protein scaffold according to any of embodiments A1-A3, wherein the first terminus and the second terminus of each monomer are positioned on the same face of said monomer and/or on the same face of an oligomer of said monomer.

A5. A protein scaffold according to any one of the preceding embodiments, wherein said first binding site comprises a first protein domain capable of forming a covalent bond to a first polypeptide target; and said second binding site comprises a second protein domain capable of forming a covalent bond to a second polypeptide target;
wherein said first protein domain is capable of forming an isopeptide bond with said first polypeptide target and said second protein domain is capable of forming an isopeptide bond with said second binding target.

A6. A protein scaffold according to embodiment A5, wherein said first binding site and said second binding site each comprise a different split ligand-binding protein domain; wherein preferably one of said first binding site and said second binding site comprises a split Streptococcus pyogenes fibronectin-binding protein domain and the other of said first binding site and said second binding site comprises a split Streptococcus pneumoniae adhesin domain.

A7. A protein scaffold according to embodiment A6, wherein said first and said second binding site each independently have at least 50% amino acid identity to any one of SEQ ID NOs: 4-9, 11-13, 23 or 15-18.

A8. A protein scaffold according to any preceding embodiments, wherein the oligomeric core comprises at least three subunit monomers,
wherein preferably the oligomeric core comprises from 3 to 6 subunit monomers.

A9. A protein scaffold according to any preceding embodiments, wherein said subunit monomers are non-covalently attached together.

A10. A protein scaffold according to embodiment A1 to A8, wherein said subunit monomers are covalently attached together;
wherein preferably said subunit monomers form a recombinant fusion protein.

A11. A protein scaffold according to any one of the preceding embodiments, wherein said oligomeric core is a homooligomeric core.

A12. A protein scaffold according to any one of embodiment A1 to A6, wherein said oligomeric core is a hetero-oligomeric core.

A13. A protein scaffold according to any one of the preceding embodiments, wherein:
i) each subunit monomer comprises less than 300 amino acids;
   preferably wherein each subunit monomer comprises less than 200 amino acids;
   more preferably wherein each subunit monomer comprises less than 150 amino acids; and/or
ii) the oligomeric core has a molecular weight of less than about 150 kDa, preferably less than about 100 kDa; more preferably less than about 70 kDa.

A14. A protein scaffold according to any one of the preceding embodiments, wherein the oligomeric core (i) does not comprise an Fc region of an antibody, or (ii) does not comprise a CH2 domain, or (iii) does not comprise a CH3 domain, or (iv) does not comprise a CH2 domain or a CH3 domain.

A15. A protein scaffold according to any one of the preceding embodiments, wherein the oligomeric core comprises a soluble multimerising structural element of a multimeric protein.

A16. A protein scaffold according to embodiment A15, wherein the multimeric protein comprises a Collagen VIII NC1 (noncollagenous) domain, a Collagen X NC1 (noncollagenous) domain, a C1q head domain, a CutA1 protein, a Macrophage Migration Inhibitory Factor (MIF or MIF-2), a Tumor Necrosis Factor (TNF) or a homolog or paralog thereof.

A17. A protein scaffold according to embodiment A15 or embodiment A16, wherein the multimerising structural element comprises a polypeptide have at least 30% or at least 50% amino acid identity to SEQ ID NO: 1, SEQ ID NO: 2, SEQ ID NO: 3, SEQ ID NO: 19, SEQ ID NO: 29, SEQ ID NO: 60, SEQ ID NO: 25, SEQ ID NO: 26, SEQ ID NO: 27, SEQ ID NO: 42, SEQ ID NO: 31, or SEQ ID NO: 58.

A18. A protein complex comprising a protein scaffold according to any one the preceding embodiments, wherein the first binding site is bound to a first polypeptide target attached to a first effector moiety, and the second binding site is bound to a second polypeptide target attached to a second effector moiety.

A19. The protein complex according to embodiment A18, wherein the first binding site/polypeptide target pair and the second binding site/polypeptide target pair are each independently selected from the following combinations: (i) any one of SEQ ID NO: 4, 6 or 8 with any one of SEQ ID NOs: 5, 7 or 9; (ii) SEQ ID NO: 12 with SEQ ID NO: 13 or 15; (iii) SEQ ID NO: 5 with SEQ ID NO: 11; (iv) SEQ ID NO: 15 with SEQ ID NO: 16); (v) SEQ ID NO: 17 with SEQ ID NO: 18; or (vi) SEQ ID NO: 23 with SEQ ID NO: 16).

A20. A screening platform comprising a library, wherein said library comprises:
a plurality of populations of protein complexes according to embodiment A18 or embodiment A19, wherein the populations of protein complexes each comprise a different combination of first effector moieties, second effector moieties and/or oligomeric core; or
a plurality of protein scaffolds according to any of embodiments A1 to A17, a plurality of first effector moieties that are able to bind specifically to the first binding site, and a plurality of second effector moieties that are able to bind specifically to the second binding site.

A21. A method for identifying a therapeutic drug or drug analog, the method comprising:
providing a protein complex according to embodiment A18 or embodiment A19;
contacting the protein complex with a biological system; and
measuring whether the protein complex induces a desired change in a property function of the biological system;
and optionally further comprising selecting a protein complex that induces a desired change in a property of the biological system.

A22. A method according to embodiment A21, further comprising:
synthesizing a therapeutic drug or drug candidate comprising the oligomeric core of the scaffold of the protein complex of the identified therapeutic drug or drug analog attached to the first and second effector moieties of said protein complex.

A23. A therapeutic drug or drug candidate obtainable or obtained according to the method of embodiment A21 or embodiment A22.

A24. A therapeutic drug or drug candidate, comprising:
(a) an oligomeric core comprising a plurality of subunit monomers attached to one or more first effector moieties and one or more second effector moieties; wherein said one or more first effector moieties and said one or more second effector moieties are positioned on the same face of the oligomeric core; and
wherein (i) said one or more first effector moieties comprise two or more first effector moieties and said one or more second effector moieties comprise two or more second effector moieties; and/or (ii) said oligomeric core does not comprise an antibody or antibody fragment; or
(b) a monomeric polypeptide attached to one or more first effector moieties and one or more second effector moieties; wherein said one or more first effector moieties and said one or more second effector moieties are positioned on the same face of the monomeric polypeptide; and
wherein (i) said one or more first effector moieties comprise two or more first effector moieties and said one or more second effector moieties comprise two or more second effector moieties; and/or (ii) said oligomeric core does not comprise an antibody or antibody fragment.

A25. The therapeutic drug or drug candidate of embodiment A24 (a), wherein the oligomeric core is as defined in any one of embodiments A1 to A17.

A26. The therapeutic drug or drug candidate of embodiment A24 or embodiment A25, wherein:
(a) each subunit monomer comprises a Collagen VIII NC1 (noncollagenous) domain, a Collagen X NC1 (noncollagenous) domain, a C1q head domain, a CutA1 protein, a Macrophage Migration Inhibitory Factor (MIF or MIF-2), a Tumor Necrosis Factor (TNF) or a homolog or paralog thereof; and/or (ii) each subunit monomer comprises a multimerising structural element comprising a polypeptide having at least 50% amino acid identity to SEQ ID NO: 1, SEQ ID NO: 2, SEQ ID NO: 3, SEQ ID NO: 19 or SEQ ID NO: 29, SEQ ID NO: 60, SEQ ID NO: 25, SEQ ID NO: 26, SEQ ID NO: 27, SEQ ID NO: 42, SEQ ID NO: 31, or SEQ ID NO: 58; or
(b) the monomeric polypeptide comprises Collagen VIII NC1 (noncollagenous) domain, a Collagen X NC1 (noncollagenous) domain, a C1q head domain, a CutA1 protein, a Macrophage Migration Inhibitory Factor (MIF or MIF-2), a Tumor Necrosis Factor (TNF) or a homolog or paralog thereof.

A27. A polypeptide comprising a first binding domain at the N terminus and a second binding domain at the C terminus, wherein the first and second binding domains are separated by a structural domain, and wherein the first binding domain and second binding domain are able to bind to their targets when expressed on a single cell or immobilised onto a plate or single bead.

A28. A polypeptide according to embodiment A27, wherein the structural domain is CutA1, an MIF or MIF-2, a TNF or TNF-like protein TL1A or CD40L, or an NC1 from Collagen VIII or Collagen X.

A29. A polypeptide according to embodiment A27 or embodiment A28, wherein the first binding domain and second binding domain are different antigen-binding domains, optionally wherein one or both antigen-binding domain is an antigen-binding fragment of an antibody, optionally an scFv or a Fab, or is a single domain antibody (sdAb), or other antibody mimetics or scaffolds selected to bind specific targets, or other proteins or peptides capable of specific binding with a biological molecule.

A30. A polypeptide according to embodiment A27 or embodiment A28, wherein the first binding domain and second binding domain are catcher domains each able to form an isopeptide linkage with a cognate peptide, optionally wherein the cognate peptide for the first binding domain is different from the cognate peptide for the second binding domain.

A31. A polypeptide according to embodiment A30 wherein each cognate peptide is attached to an antigen-binding domain, optionally wherein one or both cognate peptides are linked to the first and/or second catcher domain by an isopeptide bond.

A32. An oligomer comprising two or more polypeptides according to any of embodiment A27 to A 31.

A33. A polypeptide according to any of embodiments A27 to A31 or an oligomer according to embodiment A32, wherein the polypeptide or oligomer comprises the features of any of claims 1 to 26.

The following examples illustrate the invention. They do not however limit the invention in any way. In particular, there are many assays for protein binding and so a negative result in any specific assay is not determinative. The invention is defined according to the claims.

In summary, Example 1 details the production of constructs comprising a Collagen X NC1 structural domain and SnoopCatcher and SpyCatcher domains at the N and C termini, which are then covalently linked via isopeptide bonds to SpyTagged and SnoopTagged therapeutic polypeptides, and the isopeptide-bound constructs are then oligomerised to form a homotrimer. Example 2 provides the materials and methods used in the subsequent examples. Example 3 overviews the design of constructs according to the invention, identifies multiple components in suitable geometry, including C3 geometry, and demonstrates purification of SpC-PhCutA1-SnC (SEQ ID NO: 22). Example 4 illustrates how other assembly geometries can be modified to meet design criteria for preferred constructs of the invention. Example 5 highlights the exceptional stability of PhCutA1-derived components, as well as firm evidence for multimerization as predicted by its structure. Example 6 purifies SpyTagged/SnoopTagged components and decorates the resulting platform with tagged proteins. Example 7 demonstrates that after modular assembly, proteins can be cleaned up in a scalable fashion, herein utilizing the large size in solution via Dialysis against a 100-kDa membrane. Example 8 demonstrates that modular assembly enables rapid prototyping, including development of a HsCutA1-derived platform as a transition from PhCutA1-derived platform. Example 9 uses Alphafold to predict cis-orientation and contrast to non-cis IMX and Collagen XVIII NCI assemblies. Example 10 provides cell data showing that the assembled platform can be used for in vitro screening to elucidate the efficacy and downstream effects of ligands. Example 10 also shows that multi-domain polypeptide of PhCutA1 incorporating effector moieties can be readily produced. Expanding this screening platform, the Examples further show the generation of direct fusions of effector moieties to human CutA1, and the modification of human CutA1 by truncation and/or substitution of cysteine residues. These data demonstrate the successful production and biological function of such human CutA1-based constructs. Finally, the Examples demonstrate the utilization of cytokine-based direct fusions, granting the opportunity for development of drug candidates with additional physiological functionality.

### EXAMPLES

### Example 1

A polynucleotide sequence encoding a monomer of collagen NC1 attached by a linker at the N-terminus to one of SpyCatcher and SnoopCatcher and at the C-terminus to the other of SpyCatcher and SnoopCatcher is synthesized according to methods described in Sambrook et al., Molecular Cloning: A Laboratory Manual, 4th ed., Cold Spring Harbor Press, Plainsview, New York (2012). The polynucleotide sequence is expressed in a cellular expression system to produce the polypeptide fusion. The polypeptide fusion is allowed to oligomerise such that the SpyCatcher and SnoopCatcher binding sites are on the same face of the oligomerised trimeric construct.

Samples of the construct are contacted with a panel of different first and second therapeutic polypeptides each bound to SpyTag and SnoopTag, respectively, causing the Spy Tagged polypeptide to form a covalent isopeptide bond with each SpyCatcher moiety and the SnoopTagged polypeptide to form a covalent isopeptide bond with each SnoopCatcher moiety, thereby producing a library of collagen X NCI constructs, wherein each monomer in the construct is bound to each of two different therapeutic polypeptides, and the trimeric construct thus comprises three copies of each polypeptide. Each sample comprises a different combination of first and second therapeutic polypeptides.

Each sample in the library is assessed for its ability to trigger a biological reaction in a biological system, such as to cause cell death in a sample of cancer cells.

The samples from the library which are most effective in causing cancer cell death are noted. The combination of therapeutic polypeptides in such samples is noted.

A polynucleotide encoding a monomer of an oligomeric protein such as a monomer of collagen NC1, linked at the N-terminus to one of the therapeutic polypeptides and linked at the C-terminus to the other of the therapeutic peptides is synthesized as described in Sambrook et al., Molecular Cloning: A Laboratory Manual, 4^{th} ed., Cold Spring Harbor Press, Plainsview, New York (2012). The polynucleotide is expressed to produce a polypeptide monomer consisting of a fusion of the oligomeric protein monomer and the first and second therapeutic polypeptides. The monomer is allowed to oligomerise. The monomer is tested against the biological system. The biological reaction observed for the initial construct, e.g. the causation of cell death in a sample of cancer cells, is observed.

The Example can be performed with a polynucleotide sequence encoding a monomer of CutA1 attached by a linker at the N-terminus to one of SpyCatcher and SnoopCatcher and at the C-terminus to the other of SpyCatcher and SnoopCatcher.

### Example 2—Methods

Selection of scaffold protein components: Protein structures that meet the design criteria were selected from the Protein Data Bank (PDB). The use of adequate search filters (such as provided via http://www.rcsb.org/pdb/) enabled geometry-based prescreening, after which candidate structures were further inspected via protein structure visualization and by reference to biochemical properties described in relevant literature.

### Prediction of Assembled Protein Structures:

The multimeric 3D-structures of protein sequences were predicted using the ColabFold (Mirdita et al., 2022, Nature Methods) implementation of AlphaFold v2.0 (Jumper et al, 2021, Nature) either in Colab notebooks, or using the command-line program. The AlphaFold2-multimer-v2 modeltype was used, either with default flags, or by supplying appropriate custom templates with the—use-template and—custom-template-path flags. The highest-ranked model was visualized.

### Multiple Sequence Alignment:

Multiple sequence alignment was used to select Cys mutations. SEQ ID NO: 19 was used as the query for a HMMER (Eddy, 1998, Bioinformatics) search of the UniProtKB database (v.2021_04) (The UniProt Consortium, 2020, Nucleic Acids Research). An e-value cutoff of 0.0001 was used. Sequences with an alignment length shorter than 60% of SEQ ID NO: 19 were discarded. CD-HIT (Li and Godzik, 2006, Bioinformatics) was then used with a sequence identity threshold of 0.95 to remove redundant sequences. A multiple sequence alignment was then performed using MAFFT (Katoh et al, 2002, Nucleic Acids Research). The multiple sequence alignment from MAFFT was then analysed using the Biopython, NumPy and Pandas libraries in Python 3 to produce the frequency of each amino acid at each position in SEQ ID NO: 19. Gaps were excluded from this analysis.

Molecular cloning: Plasmids encoding recombinant proteins were provided by Twist Biosciences or ProteoGenix. DNA fragments and oligonucleotides were synthesised by Integrated DNA Technologies (IDT) and constructs were assembled through standard cloning procedures. To introduce synthesised DNA fragments into plasmid backbones, to introduce point mutations, or to make other adjustments to recombinant sequences, DNA was amplified using standard polymerase chain reaction (PCR) followed by standard cloning methods, including restriction cloning. Assembled constructs were transformed into E. coli NEB 5-alpha cells. Putative positive clones were grown overnight, and DNA was isolated from bacterial pellets via miniprep. Samples were sent for Sanger sequencing to Source Bioscience for sequence validation and alignment was performed using Benchling's molecular biology suite (www.benchling.com).

Protein purification: To obtain protein of SnT-L1 (16.1 kDa), L2-SpT (9.7 kDa), DgT-X3 (26.9 kDa), SpC-PhCutA1-SnC (39.0 kDa), SpC3-MIF2m-DgC (39.6 kDa) and SpC3-HsCutA1-DgC (40.5 kDa) DNA (synthesised by ProteoGenix, Twist Bioscience, or through standard cloning procedures) encoding for both proteins was transformed into BL21 (DE3) cells. Colonies were used to inoculate LB cultures with 50 μg/mL Kanamycin at 37° C. with 160-220 rpm shaking. Overnight cultures were diluted 1:100 into LB or 2×YT media supplemented with 50 μg/mL Kanamycin. Cultures were grown at 37° C. with 160-220 rpm shaking before induction of protein expression with 0.2-0.4 μM IPTG at OD 0.6-0.8 (LB) or 1.6-2.0 (2×YT). For platform proteins (SpC-PhCutA1-SnC, SpC3-MIF2m-DgC, SpC3-HsCutA1-DgC), samples were incubated for further 4 h at 37° C. with 160-200 rpm shaking. For tagged proteins (SnT-L1, L2-SpT, DgT-X3) samples were incubated for 16 h at 18° C. with 160-200 rpm shaking. For GST-SpC3, GST-DgC and Single-chain SpC3-HsCutA1-DgC, cultures were grown in Super-broth auto-induction media at 37° C. for 4 hours with 200 rpm shaking, then decreased to 30° C. and grown for a further 16 hours. Cells were collected by centrifugation at 5000×g for 15 min at 4° C. and pellets stored at −20° C. For protein purification, cell pellets were resuspended in Ni-NTA equilibration buffer (50 mM Tris, pH 7.8; 300 mM NaCl, 10 mM imidazole) supplemented with 1 mM PMSF, complete EDTA-free protease inhibitor cocktail and benzonase (5 U/mL). Samples were sonicated with an Ultrasonic Processor using a 20 mm probe and an amplitude of 20%, for 9-12 minutes pulsing on 2 seconds/off 4 seconds and spun down at 16,000×g for 30 min. Supernatants were retained for Ni-NTA chromatography.

Proteins were purified from cell lysates using pre-equilibrated HisPur Ni-NTA gravity-flow columns. Protein lysates were loaded to the resin. The resin was washed with 50 mM Tris, pH 7.8; 300 mM NaCl, 10 mM imidazole and subsequently with 50 mM Tris, pH 7.8; 300 mM NaCl, 30 mM imidazole until absorbance of the flow-through factions at 280 nm approached baseline. His-tagged proteins were eluted from the resin with two resin-bed volumes of Elution Buffer (50 mM Tris, pH 7.8; 300 mM NaCl, 200 mM imidazole) until the absorbance of the elution fractions at 280 nm approached baseline. Eluates were analysed by SDS-PAGE followed by Coomassie staining. Following Ni-NTA purification, samples in a high concentration of imidazole were dialysed into PBS using SnakeSkin™ Dialysis Tubing at 3K MWCO. An appropriate length of tubing was determined based on the total elution volume and was hydrated with Milli-Q water. Sample was transferred to the SnakeSkin™ Dialysis Tubing and placed in 5 L PBS overnight at 4° C. on a magnetic stirrer. After 16 h the buffer was replaced a further two times with 2 h incubations. Estimated protein concentrations were calculated from measurement of absorbance at 280 nm using an Implen NanoPhotometer N60 with reduced extinction coefficients predicted by ProtParam. L3-DgT was produced by Absolute Antibody.

To obtain proteins L1-L7, CC1-CC7 and DF1-DF6 (refer table below), DNA encoding these genes, in the pET28 expression vector, was transformed into BL21 (DE3) or SHuffle T7 express cells. Colonies were used to inoculate LB cultures with 50 μg/mL Kanamycin at 37° C. with 160-220 rpm shaking. Overnight cultures were diluted 1:100 into LB or 2× YT media supplemented with 50 μg/mL Kanamycin. Cultures were grown at 37° C. with 160-220 rpm shaking before induction of protein expression with 0.2-0.4 μM IPTG at OD 0.6-0.8 (LB) or 1.6-2.0 (2×YT). The cultures were incubated for further 4 hours at 37° C., or 16 hours at 18° C., with 160-200 rpm shaking. Cells were collected by centrifugation at 5000×g for 15 minutes at 4° C. and pellets stored at −80° C. prior to purification.

Cell pellets were resuspended in Ni-NTA equilibration buffer (50 mM Tris, pH 7.8; 300 mM NaCl, 10 mM imidazole) supplemented with 1 mM PMSF, 1× complete EDTA-free protease inhibitor cocktail and benzonase (5 U/mL). Samples were sonicated with an Ultrasonic Processor using a 20 mm probe and an amplitude of 20%, for 9-12 minutes pulsing on 2 seconds/off 4 seconds and spun down at 16,000×g for 30 min. Supernatants were retained for Ni-NTA chromatography.

The cleared lysate was loaded onto pre-equilibrated HisPur Ni-NTA gravity-flow columns. The resin was washed with 20 resin-bed volumes of Ni-NTA Wash Buffer 1 (50 mM Tris, pH 7.8; 300 mM NaCl, 10 mM imidazole) and subsequently with 10 resin-bed volumes of Ni-NTA Wash Buffer 2 (50 mM Tris, pH 7.8; 300 mM NaCl, 30 mM imidazole). His-tagged proteins were eluted from the resin with five resin-bed volumes of Ni-NTA Elution Buffer (50 mM Tris, pH 7.8; 300 mM NaCl, 200 mM imidazole) until the absorbance of the elution fractions at 280 nm approached baseline.

Eluates were analysed by SDS-PAGE followed by Coomassie staining. Proteins with a purity of <90% following Ni-NTA chromatography were further purified using and AKTA Pure with a Superdex 16/600 S75 or S200 column. Samples were purified at 4° C. using PBS buffer. The elution peak fractions were analysed by SDS-PAGE and the appropriate fractions were pooled. Proteins that were above 90% purity following Ni-NTA chromatography were dialysed against PBS using SnakeSkin™ Dialysis Tubing at 3K MWCO. Dialysed and AKTA purified proteins were then concentrated in a Pierce™ Protein Concentrator PES. Estimated protein concentrations were calculated from measurement of absorbance at 280 nm using an Implen NanoPhotometer N60 with reduced extinction coefficients predicted by ProtParam. Protein samples were adjusted to 100-300 μM and stored at −80° C.

For purification by anion exchange (AIEX) chromatography, cell pellets were resuspended in 50 mM Tris, pH 7.4 supplemented with 1 mM PMSF, complete EDTA-free protease inhibitor and benzonase (5 U/mL). Samples were sonicated with an Ultrasonic Processor using a 20 mm probe and an amplitude of 20%, for 9-12 minutes pulsing on 2 seconds/off 4 seconds and spun down at 16,000×g for 30 min. Supernatants were retained for AIEX chromatography.

AIEX was performed using an ÄKTA Pure 25 (Cytiva) with a HiTrap Q FF 1 mL column at 4° C. Prior to loading the system lines were primed with Buffer A (50 mM Tris, pH 7.8) and Buffer B (50 mM Tris, 1 M NaCl, pH 7.8). The column was first equilibrated with 5 CV of Buffer A. The lysate was loaded onto the column with the sample pump using a flow rate of 1 mL min⁻¹. The column was washed with 5 CV of Buffer A. The sample was then eluted using a linear gradient of 0-1 M NaCl. The column was washed with 5 CV Buffer B and equilibrated with 5 CV Buffer A. 5 μl samples were taken from each fraction, corresponding to major elution peaks for SDS-PAGE analysis. Fractions corresponding to the peak for the protein of interest were pooled and used in downstream applications or stored at −20° C.

Catcher-Core designs

| Name | Core | Mutation | Linker | SEQ ID NO: |
|---|---|---|---|---|
| CC1 | HsCutA1₄₄₋₁₇₉ | [C75, C96] | G4S | 24 |
| CC2 | HsCutA1₄₄₋₁₇₉ | [C75, C96] | (G4S)3 | 71 |
| CC3 | HsCutA1₄₄₋₁₇₉ | [C75A, C96A] | (G4S)3 | 72 |
| CC4 | HsCutA1₄₄₋₁₇₉ | [C75V, C96S] | (G4S)3 | 73 |
| CC5 | HsCutA1₆₀₋₁₇₁ | [C75, C96] | (G4S)3 | 74 |
| CC6 | HsCutA1₆₀₋₁₇₁ | [C75A, C96A] | (G4S)3 | 75 |
| CC7 | HsCutA1₆₀₋₁₇₁ | [C75V, C96S] | (G4S)3 | 76 |

Direct fusion designs

| Name | Description |
|---|---|
| DF1 | L7-(G4S)5-[C75V, C96S]HsCutA1₄₄₋₁₇₉ |
| DF2 | [C75V, C96S]HsCutA1₄₄₋₁₇₉-(G4S)8-L7 |
| DF3 | L7-(G4S)5-[C75V, C96S]HsCutA1₄₄₋₁₇₉-(G4S)8-L7 |
| DF4 | L7-(G4S)5-[C75V, C96S]HsCutA1₆₀₋₁₇₁-(G4S)8-L7 |
| DF5 | L7-(G4S)5-CD40L₁₂₀₋₂₆₁-(G4S)8-L7 |
| DF6 | L7-(G4S)5-TNF₇₇₋₂₃₃-(G4S)8-L7 |
| DF7 | L7-(G4S)5-CD40L₅₈₋₁₈₃-(G4S)8-L7 |

Size exclusion chromatography: Following Ni-NTA purification, proteins were optionally purified further using size exclusion chromatography, as shown in the Figures. This was performed using an ÄKTA Pure 25 (Cytiva) with a HiLoad Superdex 75 pg (SnT-L1 and L2-SpT) or a HiLoad Superdex 200 pg (SpC-PhCutA1-SnC and L1-PhCutA1-L2) column. The column was first equilibrated with one column volume of PBS. Prior to loading, the protein sample was concentrated to ˜1 mL and injected onto the column using a 2 mL injection loop. The sample was then separated using a flow rate of 1 mL min⁻¹ with 2 mL fraction size collection. 20 μl samples were taken from each fraction, corresponding to major elution peaks for SDS-PAGE analysis. Fractions corresponding to the peak for the protein of interest were pooled and used in downstream applications or stored at −20° C.

HsCutA1 assemblies were purified using a Superose 6 Increase 5/150 column. The column was first equilibrated with one column volume of PBS. Prior to loading, the protein sample was prepared to ˜100 μL and injected onto the column via a Hamilton 700 Microliter Syringe. The sample was then separated using a flow rate of 0.3 mL min⁻¹ with 0.1 mL fraction size collection. 10 μL samples were taken from each fraction, corresponding to major elution peaks for SDS-PAGE analysis. Fractions corresponding to the peak for the protein of interest were pooled and used in downstream applications or stored at −20° C.

Protein quantification by BCA or Bradford assay: Prior to conjugation, samples were quantified using a BCA Protein Assay kit (ThermoFisher) or Bradford Plus Protein Kit (ThermoFisher), according to the manufacturer's instructions. BSA standards were diluted in 1×PBS. Purified protein was diluted 1/5, 1/10 and 1/20 in 1×PBS to ensure concentration was within the linear range of the assay. Following incubation with the BCA or Bradford reagent, absorbance at 562 nm or 595 nm, respectively, was measured on a BMG FluoSTAR Omega plate reader. Concentration in mg/mL was interpolated based on the standard curve.

Catcher-based protein conjugation: Platform conjugation with relevant ligands were performed between 1:1:1-1:2:2 of platform: ligand: ligand ratios, according to the specific assays as detailed in the relevant figures. The conjugation reactions proceeded at 25° C. for 16 h, 24 h or 64 h, according to relevant assays, and samples were analysed by SDS-PAGE (8%, 16%) followed by Coomassie staining. Confirmatory dialysis of conjugated assembly H6-SpC-PhCutA1-SnC: SnT-L1:L2-SpT to remove excess substrates was performed using an HTDialysis 12-well block with a 100 kDa MWCO cellulose membrane at room temperature. Prior to dialysis, the membrane was hydrated for 60 min in sterile Milli-Q, replaced by 20% ethanol for 20 min and washed in sterile Milli-Q water twice before use. Both sample and dialysate contained 1×PMSF. Samples of the assembled platform and dialysate were taken at 2 h, 4 h, 8 h and 16 h during dialysis and analysed by SDS-PAGE (14%) followed by Coomassie staining.

Preparatory dialysis of conjugated assemblies as input for cell assays were done as above, but with the following modifications: no PMSF was added, and the dialysis was performed at room temperature overnight.

Another variation of the conjugation process is as follows. Protein samples were first quantified using the Coomassie Plus Bradford assay (ThermoFisher). Conjugation samples of SpT, SpT3, DgT and SnT binder proteins to CutA1-based cores fused with SpyCatcher003 and DogCatcher were prepared at 20 μM with a 1:1.2:1.2 binder excess of platform: ligand: ligand ratios. The reactions were prepared in PBS buffer supplemented with 1× cOmplete EDTA-free protease inhibitor and 50 μg/mL Penicillin-Streptomycin. Samples were incubated at 25° C. for 1 hour for SpT3 and DgT binders and 16 hours for SpT and SnT proteins. Excess binders were removed by dialysis using a 100 kDa MWCO cellulose membrane.

Another variation of the conjugation process is as follows. Conjugation reactions were performed as above, except no complete EDTA-free protease inhibitor was added. Assemblies were then purified as described in Example 7. In brief, A MagneGST resin column pre-loaded with GST-Catcher was utilised to capture excess ligands from an assembly mixture upon incubation with the resin. To pre-load the resin, GST-Catchers were mixed in PBS to give a final concentration of 2.5 mg of GST-SpyCatcher003 and 2.5 mg of GST-DogCatcher. An appropriate amount of MagneGST resin was added to achieve full capture of both proteins, assuming a binding capacity of 5 mg/mL of resin. The suspension was washed repeatedly with PBS until the A280 of the supernatant approached baseline. The suspension was kept on ice as a 50% slurry with PBS until used in the assembly purification. An appropriate volume of MagneGST resin bound GST-DogCatcher and GST-SpyCatcher003 suspension was added to the conjugation reaction to give a final concentration of 16 μM for each GST-Catcher. The suspension was then incubated at room temperature for 4 hours with 600 rpm shaking. The resin was sedimented using and Opentron magnetic module engaged for 5 minutes. The supernatant was then transferred to a new 96 well plate. The post-purification conjugates were then quantified via Bradford assay and analysed by SDS-PAGE followed by Coomassie staining.

HsCutA1 crosslinking: To crosslink H6-SpC3-HsCutA1-DgC, 10 μM of the protein was incubated with 0.1% glutaraldehyde in PBS for 20 min at 37° C. Samples were taken at the indicated intervals during the reaction and 100 mM Tris pH 8.8 was added to stop the reaction. Samples were analysed by SDS-PAGE (12%) followed by Coomassie staining.

Cell culture: NCI-N87 (CRL-5822) and A-431 (CRL-1555) cells were obtained from ATCC and routinely cultured in RPMI and DMEM, respectively, supplemented with 10% FCS and 5% Penicillin/Streptomycin. Colo 205 (CCL-222) was obtained from ATCC and routinely cultured in RPMI, supplemented with 10% FCS and 5% Penicillin/Streptomycin.

Cell viability assay: 2000 NCI-N87 cells/well were seeded into 96-well plates and grown in DMEM supplemented with 10% FCS for 24 h before starvation in DMEM medium containing 0.2% FCS for 24 h. Cells were then treated or mock-treated with various concentrations (0.01-100 nM) of protein assemblies with two ligands (H6-SpC-PhCutA1-SnC: SnT-L1:L2-SpT) or one ligand only (H6-SpC-PhCutA1-SnC: SnT-L1, H6-SpC-PhCutA1-SnC:L2-SpT). Scaffold only (H6-SpC-PhCutA1-SnC), ligands only (SnT-L1, L2-SpT, SnT-L1+L2-SpT) and monoclonal control antibodies against both ligands were used as controls. All antibodies were diluted in starvation medium. 1 h after the start of the treatment, assay-relevant growth factors were added to a final concentration of 30 ng/mL with a final FCS concentration of 2%. Cells were then grown for 7 days, and surviving fractions were measured using the MTT assay. 20 μL of 5 mg/mL 3-(4,5-Dimethylthiazol-2-yl)-2,5-diphenyltetrazolium bromide (MTT) in PBS were added to each well containing 200 μL medium. Incubation for 3 h was followed by media aspiration, formazan dissolution in 100% DMSO and absorbance reading at 570 nm. Surviving fractions were normalised to mock-treated controls. For the bispecific panel screening, 2,000 Colo 205 or NCI-N87 cells/well were seeded into 96-well plates and grown in normal growth medium for 24 h. Cells were then treated or mock-treated with various concentrations (0.0064-100 nM) of protein assemblies comprising a scaffold and two ligands or a scaffold and one ligand only. Cells were grown for further 5 (Colo 205) or 7 days (NCI-N87), and surviving fractions were measured using the Cell Titer Glo Assay (Promega) for Colo 205 and the MTT assay for NCI-N87 cells. Surviving fractions were normalised to mock-treated controls. To compare toxicity of L7 assemblies comprising different valencies, 2,000 Colo 205 or 1,000 HCT116 cells/well were seeded into 96-well plates and grown in normal growth medium for 24 h. Cells were then treated or mock-treated with various concentrations (0.0008-2 nM) of protein assemblies comprising a monomeric or trimeric scaffold or a linker assembled with one or two L7 ligands, resulting in bivalent, trivalent or hexavalent L7 assemblies. Cells were grown for further 5 (Colo 205) or 4 days (HCT116), and surviving fractions were measured using the MTT assay. Surviving fractions were normalised to mock-treated controls. To compare toxicity of nanobody based DgT-L7 assemblies with SpT-binders consisting of multi-domain ligands, 2,000 Colo 205 cells/well were seeded into 96-well plates and grown in normal growth medium for 24 h. Cells were then treated or mock-treated with various concentrations (0.0008-2 nM) of protein assemblies comprising a SpT multi-domain binder in scFv or Fab format and DgT-L7. The SpT-L7: HsCutA1:L7-DgT assembly was used as a comparison. Cells were grown for further 5 days, and surviving fractions were measured using the MTT assay. Surviving fractions were normalised to mock-treated controls. To compare toxicity of a tetrameric L7-nanobody-based failed clinical candidate and a proprietary multivalent bispecific construct consisting of L7 and L11 binders fused to [C75A, C96A]HsCutA₆₀₋₁₇₀. 1,000 HCT116 cells/well were seeded into 96-well plates and grown in normal growth medium for 24 h. Cells were then treated or mock-treated with various concentrations (0.000256-0.8 nM) of the failed tetrameric clinical candidate TAS266 and the bispecific multivalent HsCutA1-based direct fusion. The TAS266 nanobody L7 and HsCutA 1 core were used as controls. Cells were grown for further 5 days, and surviving fractions were measured using the MTT assay. Surviving fractions were normalised to mock-treated controls.

Western blotting: Stability assay: H6-SpC-PhCutA1-SnC and H6-SpC-PhCutA1-SnC: SnT-L1:L2-SpT (purified using size exclusion chromatography) were incubated in PBS and complete media containing 10% FCS for 4 days at 4° C. or 37° C. Samples were run on 4-20% Tris-Glycine SDS-Page gels and transferred to nitrocellulose membranes using the iBlot 2 system (Thermo Fisher). Membranes were probed with an antibody against anti-polyHistidine (Sigma-Aldrich, cat no. A7058, 1:2000) and signals were detected using ECL. Akt/ERK signalling: 1.5×10⁶ NCI-N87 cells were seeded into T25 flasks and grown in DMEM supplemented with 10% FCS and 5% Penicillin/Streptomycin medium for 24 h before starvation in medium containing 0.2% FCS for 24 h. Cells were then treated or mock-treated with 25 nM of protein assemblies containing both (H6-SpC-PhCutA1-SnC: SnT-L1:L2-SpT) or one ligand (H6-SpC-PhCutA1-SnC: SnT-L1, H6-SpC-PhCutA1-SnC:L2-SpT). Scaffold only (H6-SpC-PhCutA1-SnC), ligands only (SnT-L1, L2-SpT, SnT-L1+L2-SpT) and commercially available monoclonal antibodies against the two target proteins were used as controls. 1 h after the start of the treatment, cells were stimulated by adding assay-relevant growth factors to a final concentration of 50 ng/ml and incubated for 1 h with FCS at 0.2% throughout the treatment. Whole cells extracts were prepared using RIPA buffer supplemented with phosphatase and protease inhibitors. 25 μg of protein per lane were separated on 4-12% Bis-Tris gels and transferred to nitrocellulose membranes using the iBlot2 system according to manufacturer's instructions. Membranes were probed with antibodies against p-Akt (Cell Signalling, cat no. 4060, 1:2000), Akt (Cell Signalling, cat no. 2920, 1:2000), p-ERK 1/2 (Cell Signalling, cat no. 9101, 1:1000), ERK1/2 (Cell Signalling, cat no. 4695S, 1:1000). Signals were detected using ECL.

Cell killing assay: To investigate protein assemblies targeting L1 and L3, NCI-N87 cells (30,000 cells/well) were seeded into 96-well plates and grown in complete media (DMEM, supplemented with 10% FCS and 5% Penicillin/Streptomycin) for 24 h, followed by starvation in medium containing 0.2% FCS and 5% Penicillin/Streptomycin for 24 h. Cells were then treated with various concentrations (0.01-100 nM) of assembled proteins H6-SpC3-HsCutA1-DgC:L1-SpT:L3-DgT, H6-SpC3-HsCutA1-DgC:L1-SpT and H6-SpC3-HsCutA1-DgC:L3-DgT scaffold only (H6-SpC3-HsCutA1-DgC) and ligands only (L1-SpT, L3-DgT) and incubated for 40 h. Surviving fractions were measured by MTT assay and normalised to mock-treated control cells.

Apoptosis assay: 10,000 Colo 205 cells were seeded into 96-well plates and grown in normal growth medium for 24 h. Cells were then treated with increasing concentrations (0.0008-10 nM) of direct fusions or protein assemblies comprising a scaffold and two ligands, or a scaffold and one ligand only. Cells were grown for further 48 h and surviving fractions were measured using the Cell Titer Glo Assay. Surviving fractions were normalised to mock-treated controls.

### Example 3—Suitable Selection of Recombinant Protein Components Enables Simple Preparation of Protein Complexes Featuring Binding in Cis-Oriented Geometry

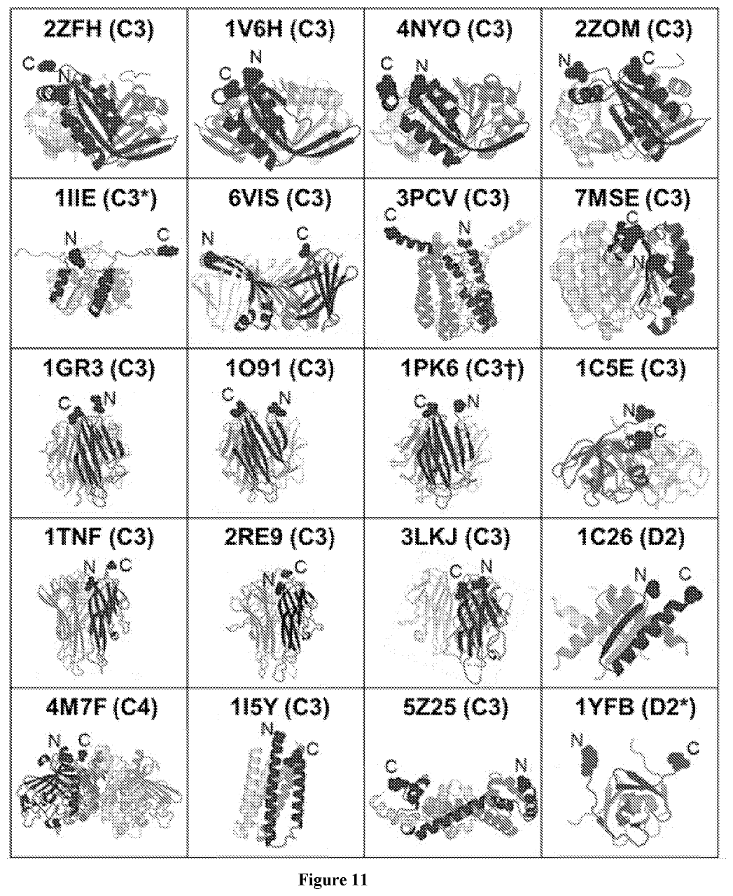

The inventors recognised that a multimeric protein component in which each monomer features a C-terminus and N-terminus that are in proximity to each other or to the termini of other monomers in the same complex can be utilised to project binding sites towards a single binding surface in “cis-orientation”. Publicly available protein structures were filtered for keywords and/or geometric parameters to identify protein structures with suitable geometry to arrive at multimeric protein complexes in “cis-orientation” via recombinant fusion of binding sites at monomer termini, or ligands to such a protein complex (FIG. 11).

The inventors identified a number of suitable domains, including:

Collagen X NCI domain (PDB ID: 1GR3), Collagen VIII NC1 domain (PDB ID: 1091), CutA1 (copper tolerance A) proteins from various species (such as the CutA1 proteins from Pyrococcus horikoshii (PDB ID: 4YNO), Homo sapiens (PDB ID: 2ZFH), Thermus thermophiles (PDB ID: 1V6H); Oryza sativa (PDB ID: 2ZOM); or Shewanella sp. SIBI (PDB ID: 3AHP), C1q head domain (PDB ID: 1PK6), TNF-like protein TL1A (PDB ID: 2RE9), TNF (PDB ID: 1TNF), MIF (PDB ID: 1CA7), MIF2 (PDB ID: 7MSE), and other protein structures described herein or depicted in FIG. 11.

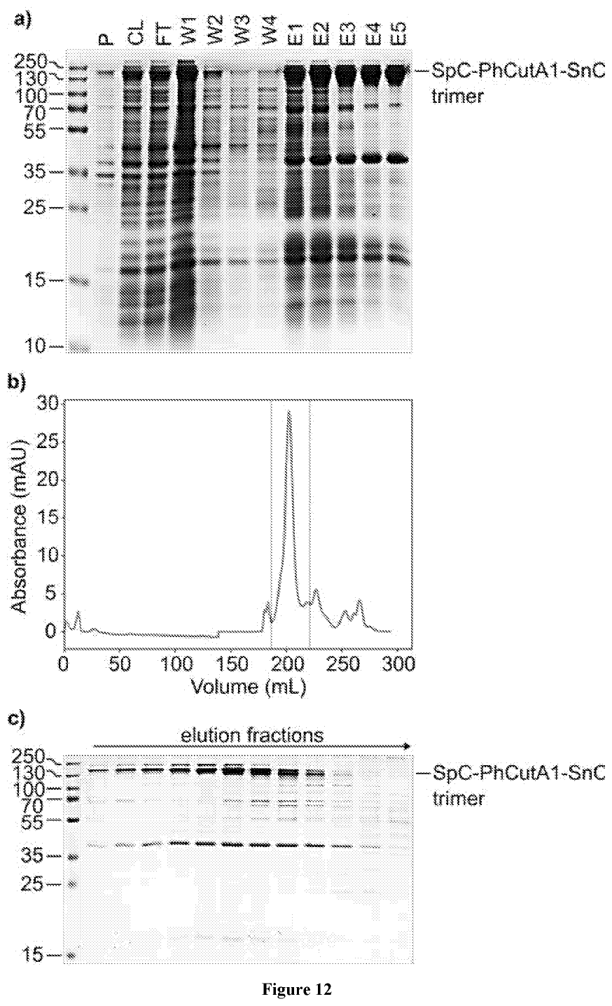

The inventors were able to readily express and prepare PhCutA1 from Pyrococcus horikoshii (SEQ ID NO: 1) fused to SpyCatcher (SEQ ID NO: 4)N-terminally (via a GSGS linker) and SnoopCatcher (SEQ ID NO: 12)C-terminally (via a GSGS linker) recombinantly in E. coli, further featuring a His-tag N-terminal of SpyCatcher (SEQ ID NO: 21, FIG. 12). Notably, this construct “H6-SpyCatcher-PhCutA1-SnoopCatcher” or “SpC-PhCutA1-SnC” featured hyper-thermostable trimerisation as apparent in resistance to boiling in denaturing SDS-loading buffer, a property characteristic to intact PhCutA1 (Tanaka et al., 2006, FEBS Letters, 580 (17), pp. 4224-4230). Therefore, multimerisation properties of the core protein PhCutA1 were successfully imparted onto the SpC-PhCutA1-SnC scaffold.

### Example 4—A Protein Complex Suitable for “Cis-Oriented” Display Via Recombinant Fusion N-Terminal and C-Terminal of Monomer Proteins can be Derived from Heteromeric Protein Complexes or Dihedral Protein Assemblies

In addition to protein structures or domains already featuring geometry suitable for “cis-oriented” display by recombinant fusion at N-terminal and C-terminal sites, the inventors also identified proteins from which such components could be derived.

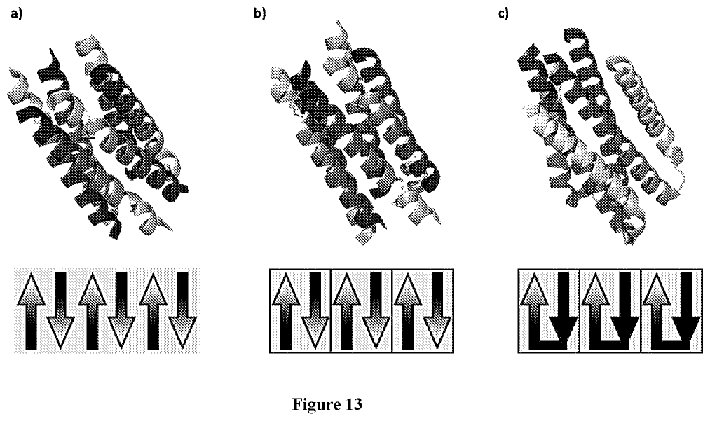

With a suitable linker between C-terminus and N-terminus of antiparallel coiled-coils in PDB ID 5w0j (Spencer & Hochbaum, 2017, Biochemistry, 56 (40), pp. 5300-5308), the structure mimics the structure of circularly symmetric HIV GP41 (PDB ID 1i5y) (FIG. 13 a,c). Herein, a heteromeric antiparallel coiled-coil assembly (PDB ID 5vte) derived in the same publication as PDB ID 5w0j (Spencer & Hochbaum, 2017) provides an example of how simple (rational) mutagenesis could mitigate inversion of the termini by encouraging uniform assembly. Coiled-coil proteins are easy to design and can benefit from designed properties such as pH-sensitivity (Nagarkar et al., 2020, Peptide Science, 112 (5), e24180) or as bioactive protein switches (Langan et al., 2019, Nature, 572 (7768), pp. 205-210).

### Example 5—CutA1 Proteins Retain Trimeric Structure after Recombinant Fusion to Catcher Proteins

PhCutA1 is a highly thermostable protein that retains trimeric structure even after boiling in denaturing SDS-loading buffer. As shown in FIG. 14, a protein band was observed between 130-250 kDa indicating stable trimerisation of PhCutA1 even after recombinant fusion to SpC and SnC, confirming that the overall complex assembles according to our structure-based design. To further validate that this observed band was dependent on correct PhCutA1 assembly, we used harsh denaturing conditions, upon which we observed partial monomerisation of SpC-PhCutA1-SnC.

### Example 6: The Use of Modular Components Enables Rapid Assembly of Ligand Proteins And Other Effectors onto Scaffold Proteins of Choice to Impart Complex Geometry

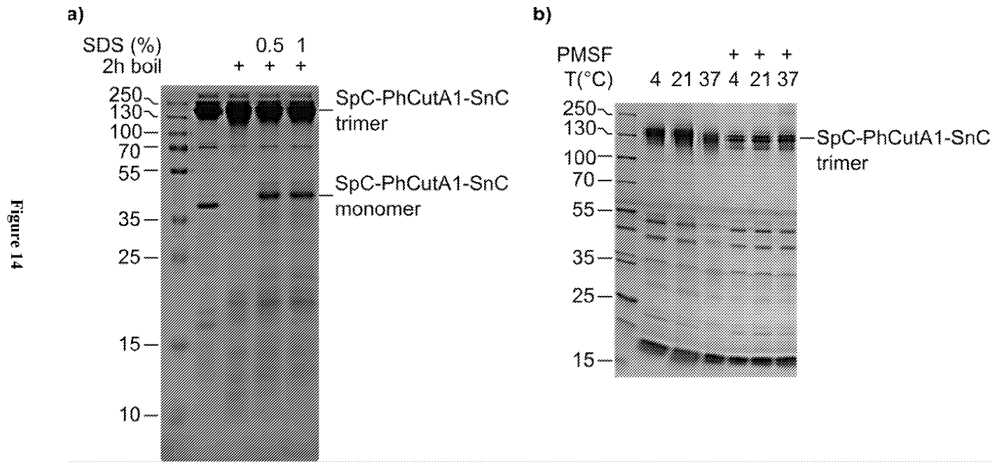

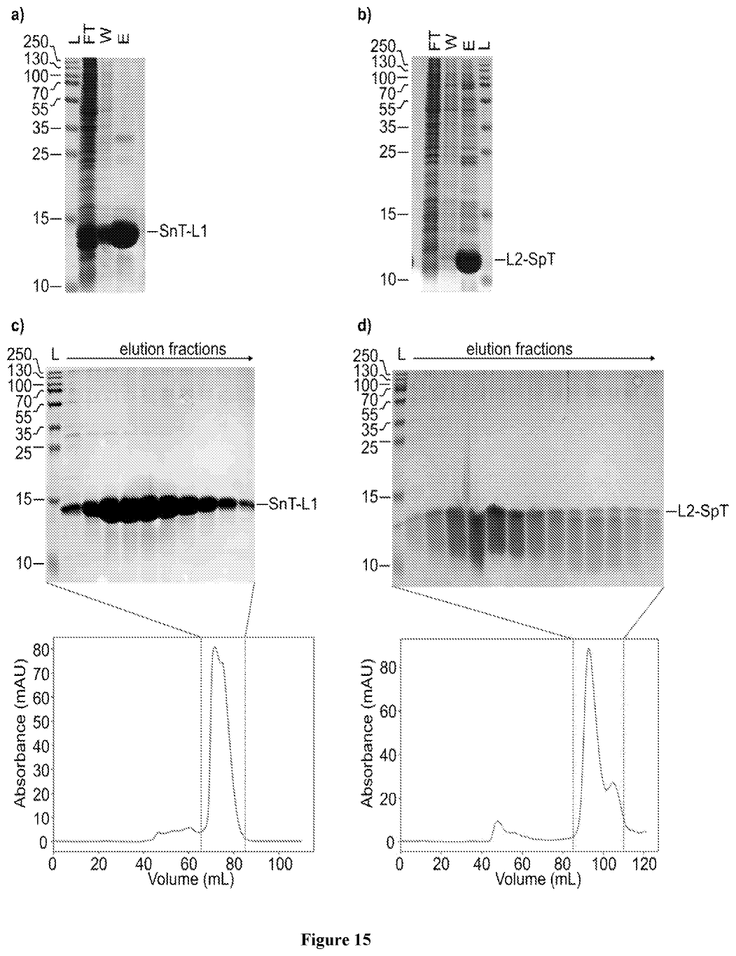

To demonstrate the feasibility of modular component assembly, Spy Tagged, SnoopTagged and DogTagged ligands were designed and expressed. Spy/Snoop-tagged protein components SnT-L1 and L2-SpT are ligands against common cellular antigens. Ni-NTA purification resulted in clean-up of both ligand proteins (FIG. 15 a-b), which was optionally followed by size exclusion chromatography (FIG. 15 c-d). These components were used to confirm that conjugation of tagged ligands to modular platforms containing Catcher proteins yields fully assembled platforms with ligands attached.

After incubation of SnT-L1 and/or L2-SpT with SpC-PhCutA1-SnC or control protein SpC-PC-SnC, we observed that conjugation was able to go to completion for all samples (FIG. 16a-c). Notably, hyper-thermostable trimerisation of SpC-PhCutA1-SnC was retained even during decoration with both SnT-L1 and L2-SpT. Conjugation of H6-SpC-PhCutA1-SnC (117 kDa as trimer) and H6-SpC-PC-SnC (31 kDa) with SnT-L1 and with L2-SpT resulted in a band shift corresponding to the added molecular weight (16.1 kDa per monomer SnT-L1, 9.7 kDa per monomer L2-SpT). Simultaneous conjugation of both ligands to either platform resulted in a significant band shift (expected 25.8 kDa per monomer) (FIG. 15 c-d). Furthermore, the consumption of ligands was observed in a reduction of ligand intensity (added in excess of platform).

### Example 7: Integration of Modular Assembly with Simple Post-Assembly Clean-Up Enables the Manufacture of Uniform Drug Candidates for Downstream Analysis

To validate a simple and effective method for the purification of assembled drug candidates in an automation-compatible manner, the inventors investigated the suitability of a reusable, 96- and 12-well high-throughput dialysis device for drug candidate purification. The inventors have demonstrated that the modular assembly of SpC-PhCutA1-SnC with SnT-L1 and L2-SpT can be purified via high-throughput dialysis for 16 h with regular buffer changes. For this, assembly was performed with a protein component ratio of 1:1:1 and samples were incubated at 25° C. for 16 h. Dialysis was performed using a 12-well high-throughput dialysis block with a 100 kDa MWCO cellulose membrane at room temperature.

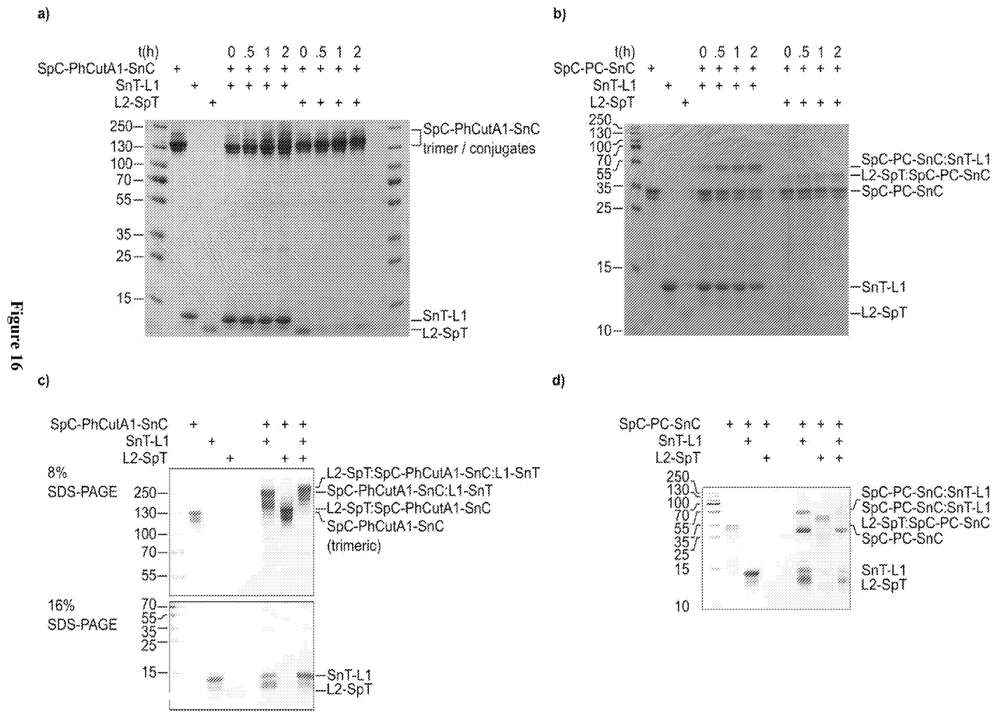

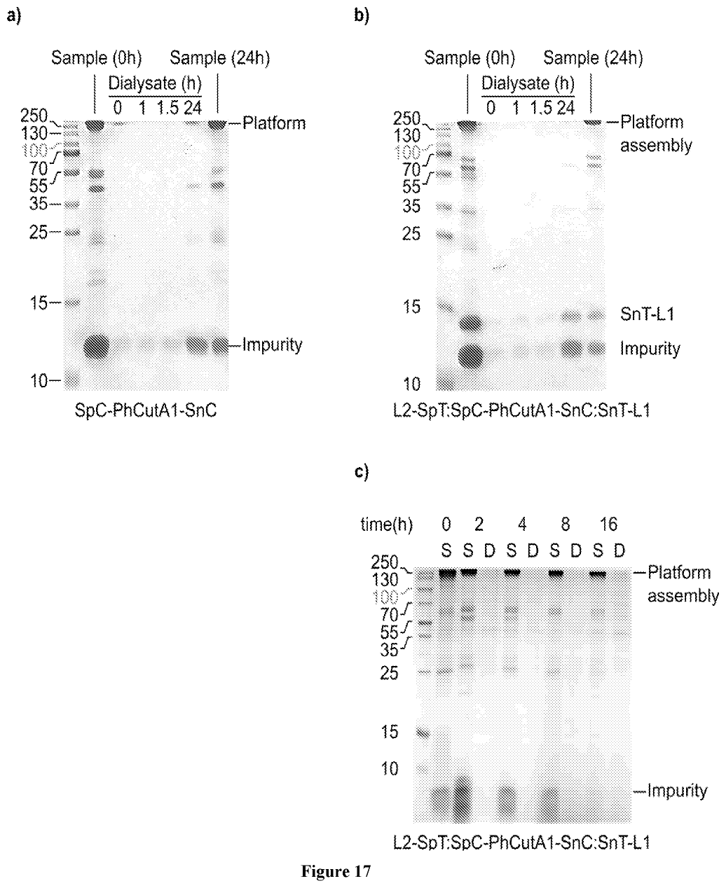

Both sample and dialysate contained 1×PMSF to avoid protein degradation during dialysis. Samples of the assembled platform and dialysate were taken at 2 h, 4 h, 8 h and 16 h during dialysis and analysed by SDS-PAGE to demonstrate the removal of excess ligands and low molecular impurities over time (FIG. 17).

This demonstrates that a large molecular weight assembly is present and stable in solution. Its properties enable a simple purification protocol. Furthermore, this methodology is scalable and automation-compatible for final protein assembly workflow, which can then be used for downstream in vitro analysis.

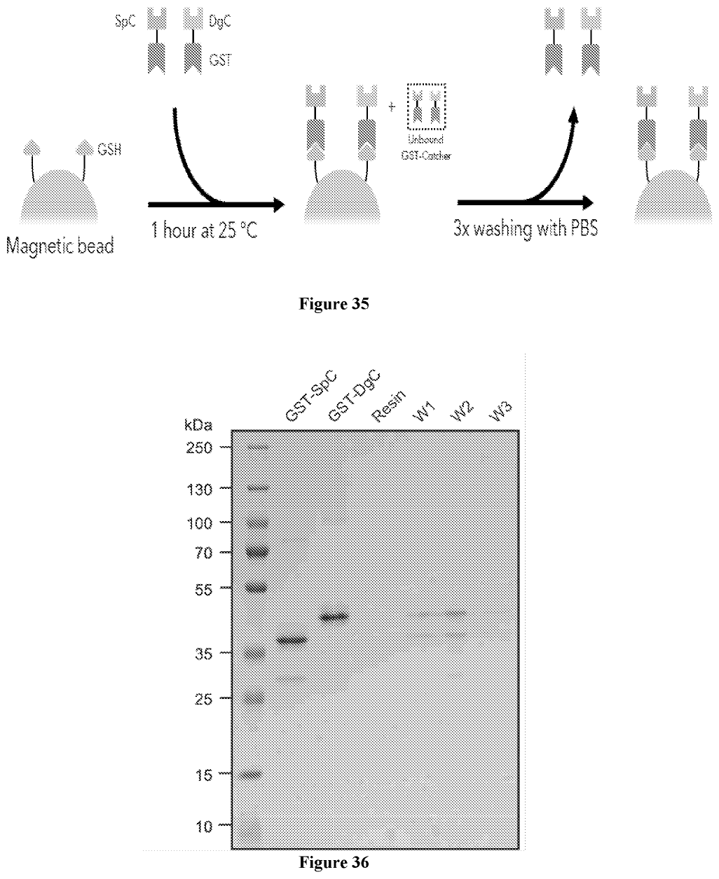

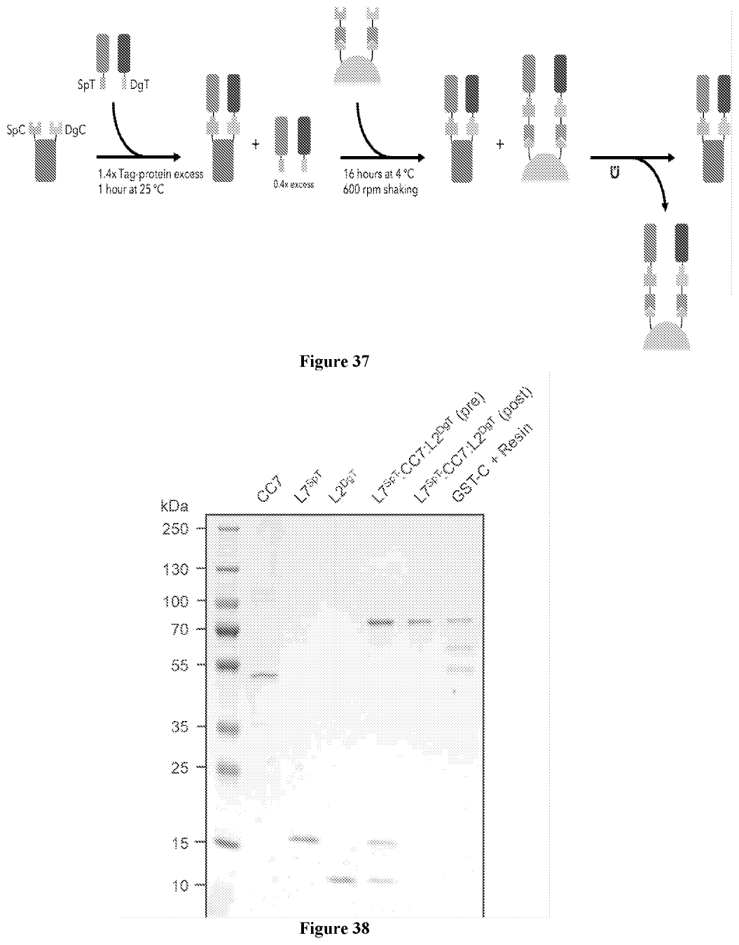

An alternative automation compatible method was developed using GST-SpyCatcher003 (“GST-SpC3”, SEQ ID NO: 104) and GST-DogCatcher (“GST-DgC”, SEQ ID NO: 105) bound to commercially available paramagnetic beads to quench unconjugated tagged proteins from an assembly of SpT and DgT binders to a Catcher-core (FIG. 37). This allows for cost-effective, rapid purification of assemblies that is easily scalable. It is suitable for low-volume plate-based formats that can be automated through the use of a liquid handling robot. The GST-Catchers are loaded onto the beads (FIG. 35) and the unbound excess removed via washing with PBS (FIG. 36). The GST-Catcher bound beads are added to each conjugation reaction to give a 2×excess of GST-Catcher to the corresponded unconjugated L2-DgT and L7-SpT. This can then be removed via magnetic capture of the particles, leaving a supernatant that contains only the fully assembled molecule (FIG. 38). This method is advantageous, relative to covalent-conjugation of catchers to beads, as it can be performed on-demand at a variety of scales. There are also no complex chemical processes required or reducing conditions. Furthermore, the design of the catcher constructs, the ratio of catcher proteins, or resin can easily be changed as demand requires.

### Example 8: Modular Component Design Enables Rapid Prototyping and a Gradual Transition of Assembly Platforms Optimised for Early Screening to Assembly Platforms Optimised for More Downstream Therapeutic Validation

With PhCutA1, the inventors have demonstrated the design, production, assembly and purification of a bacterial scaffold protein for use in in vitro screening (FIG. 11, FIG. 12). To further demonstrate the ability to rapidly change platform components, including for potential drug utility, we have also identified, cloned, expressed and purified scaffolds based on the human homologue of the CutA1 protein, HsCutA1 (FIG. 11) and a mutant of the human Macrophage Migration Inhibitory Factor 2 (MIF2), MIF2m (FIG. 11). The SpC3-HsCutA1-DgC (SEQ ID NO: 24) platform features SpyCatcher003 (SEQ ID NO: 8) and DogCatcher (SEQ ID NO: 23) for seamless modular conjugation to tagged-ligands fused to HsCutA1 (SEQ ID NO: 29, truncated to retain some natural amino acids beyond the oligomeric core as linkers), representing a human variant for in vitro validation and downstream therapeutic validation. SpC3-MIF2m-DgC (SEQ ID NO: 28) features a different scaffold with similar C3 geometry and with longer linkers (GGGGSGGGGSGGGGS) compared to SpC-PhCutA1-SnC (GSGS) and Sp3-HsCutA1-DgC (GGGGS). To prepare SpC3-HsCutA1-DgC and SpC3-MIF2-DgC, BL21 (DE3) cells were used for protein expression, followed by Ni-NTA gravity flow column purification (FIG. 18a). As with PhCutA1, platforms derived from HsCutA1 and MIF2m were readily prepared to be available for ligand assembly.

HsCutA1 is a stable protein with a near identical fold as PhCutA1 (FIG. 11), however it is readily denatured to a monomer during boiling in SDS-loading buffer (FIG. 18). Upon incubation with the crosslinking agent glutaraldehyde, we observed covalent crosslinking of monomeric subunits of SpC3-HsCutA1-DgC with an approximately threefold increase in apparent molecular weight, confirming that the protein is a trimer in solution (FIG. 18c) and correctly assembles as predicted from the protein structure. Other ligands were also conjugated to SpC3-HsCutA1-DgC, and the different samples of unconjugated scaffold only, conjugated scaffold+one ligand, and conjugated scaffold+two ligands were then subjected to size exclusion chromatography to show the increase in hydrodynamic radii as the assembly complexes increase in size and removal of excess ligands.

### Example 9: The Capacity of Platform Designs to Produce “Cis-Orientation” can be Explored Computationally

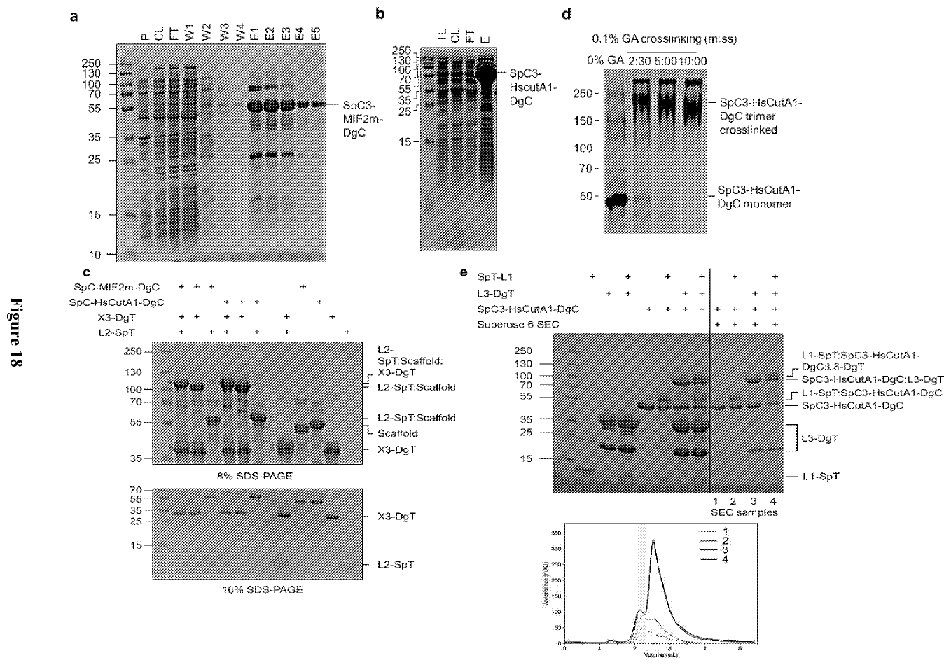

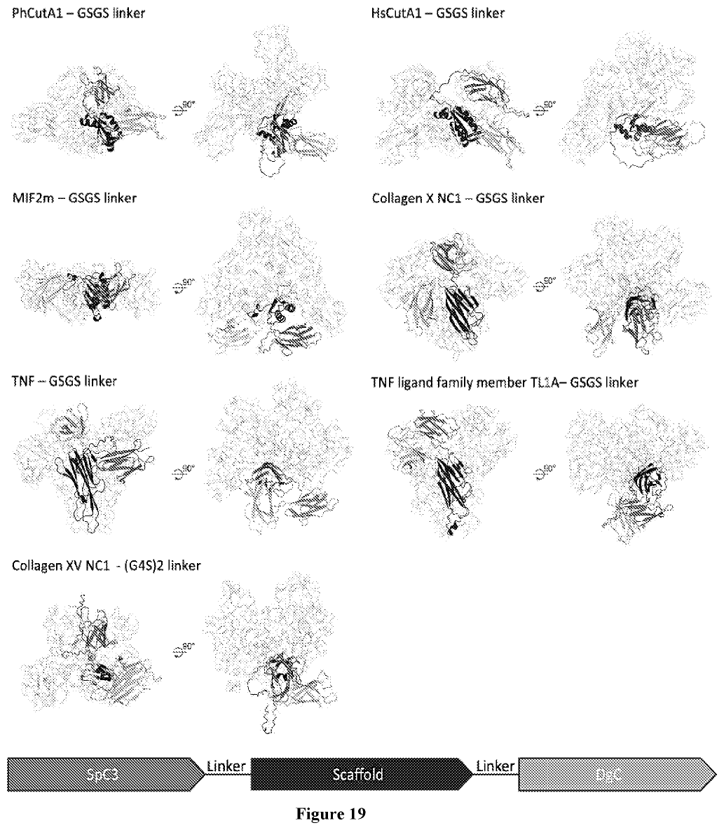

Despite preselection of core components with suitable arrangement of N-terminal and C-terminal fusion sites, the introduction of effector or ligand proteins as well as the connection of such domains via linkers of varying length and flexibility can affect fusion protein geometry. The inventors used an implementation of Alphafold v2.0 optimized for multivalent protein assembly to predict the orientation of varying Catcher components conjugated onto different core proteins. Herein, PhCutA1, HsCutA1, Col X NC1, TNF and TL1A were predicted to assume cis-oriented display of Catcher components as a stable trimer (FIG. 19). For PhCutA1, this confirms the crystal structure of PhCutA1 alone (FIG. 11) and various experiments (FIG. 12, FIG. 14). For comparison, we also tested core proteins which were previously utilized in “trans-oriented” designs, namely IMX313 (Brune et al., 2017, Bioconjugate Chemistry, 28 (5), pp. 1544-1551) and the NCI domain of Collagen XV (Lobo et al., 2006, International Journal of Cancer, 119 (2), pp. 455-462). Herein, SpC3-IMX-DgC featuring GSGS linkers (SEQ ID 35) was not able to produce an intact core structure. Such a display may result in steric clashes upon conjugation with SpyTagged/DogTagged proteins; notably, Brune et al introduced a prolonged, rigid linker between IMX and SnoopCatcher, separating the orthogonal SpyCatcher proteins. Like IMX, SpC3-Collagen XV NC1-DgC featuring GSGS linkers (SEQ ID 33) was not able to produce an intact core structure. Upon replacing GSGS linkers (as in SEQ ID 33) with longer (G4S) 2 linkers, we observed a staggered confirmation of catchers in SpC3-Collagen XV NC1-DgC.

### Example 10: The Assembled Platform can be Used for In Vitro Screening to Elucidate on the Efficacy of Ligands

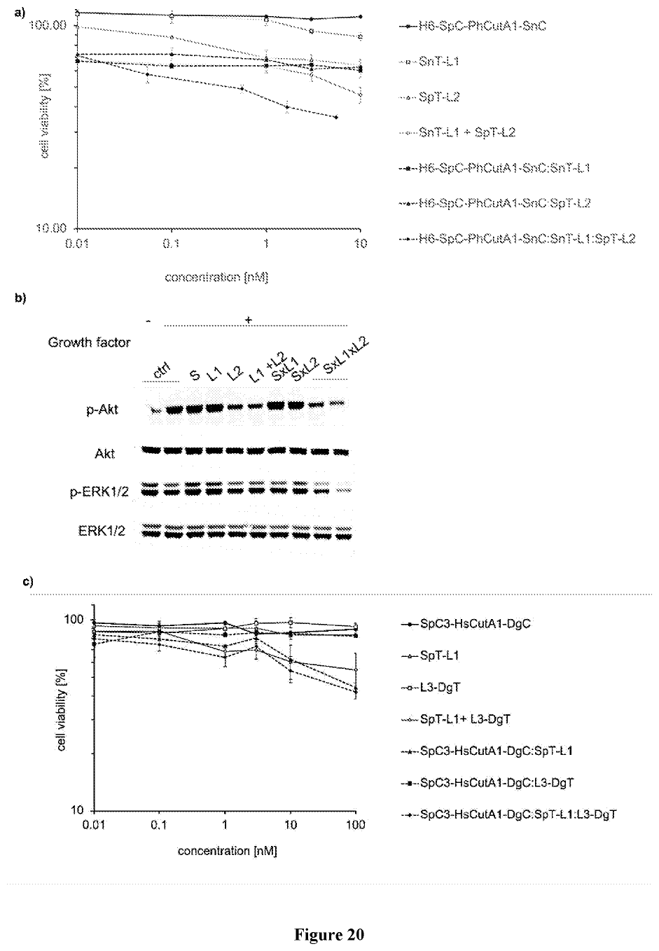

We tested if PhCutA1 fully conjugated with ligands against two different targets involved in cell proliferation is able to inhibit growth factor-induced cell growth (FIG. 20a). NCI-N87 cells were treated or mock-treated with indicated concentrations of protein assemblies containing two ligands (H6-SpC-PhCutA1-SnC: SnT-L1:L2-SpT) or one ligand only (H6-SpC-PhCutA1-SnC: SnT-L1, H6-SpC-PhCutA1-SnC:L2-SpT). Scaffold only (H6-SpC-PhCutA1-SnC), ligands only (SnT-L1, L2-SpT, SnT-L1+L2-SpT) and monoclonal control antibodies against receptor targets of L1 or L2 were used as controls. 1 h after the start of the treatment, cells were stimulated by addition of a relevant growth factor. Cell viability was measured using the MTT assay 7 days later. Surviving fractions were normalised to mock-treated control cells. Treatment with only scaffold or ligands has no significant impact on cell viability while treatment with the conjugated assembly with only one ligand component results in a modest reduction of cell survival (around 60% cell viability at 10 nM of either protein conjugate). Treatment with the fully assembled assembly resulted in significantly decreased cell numbers, with only 35% of viable cells present after growth in the presence of 10 nM of the full assembly. This was very similar to the survival rate we observed after treatment with a combination of the monoclonal control antibodies at the same concentrations (10 nM). This suggests that the constructs are blocking both targets on the same cell. To further confirm this, a control consisting of H6-SpC-PhCutA1-SnC: SnT-L1, H6-SpC-PhCutA1-SnC:L2-SpT in the same treatment sample could be added for future assays.

We further investigated if the fully-conjugated assembly represses downstream activation of Akt and Erk1/2 (FIG. 20b). After starving NCI-N87 cells for 24 h in medium containing 0.2% FCS, they were treated with Catcher Core only or conjugated assembly of proteins SnT-L1 and L2-SpT for 1 h and then stimulated with growth factors for 1 h before preparation of whole cell extracts. Binding of the two used ligands to their receptors results in activation of two downstream signalling pathways and in the phosphorylation of their main effectors, Akt and Erk1/2. Phosphorylation levels of these two proteins were analysed by Western Blotting. Erk1 is constitutively phosphorylated in NCI-N87 cells even in the absence of growth factor stimulation. However, fully-conjugated assembly (H6-SpC-PhCutA1-SnC: SnT-L1:L2-SpT) significantly represses Erk phosphorylation. Growth factor-induced activation of Akt is prevented by unbound L2 at this concentration. Importantly, phosphorylation levels decrease further in the presence of the full assembly.

Once targets for drug development are confirmed, direct fusion of the ligands to the core protein as a multi-domain polypeptide can also be achieved without the usage of Tag and Catcher components for drugs that are validated for clinical testing (FIG. 21).

We also investigated whether a different scaffold, SpC-HsCutA1-SnC, conjugated to another pair of ligands (SpT-L1, L3-DgT) causes apoptosis within 2 days. (FIG. 20c). After starving NCI-N87 cells for 24 h in medium containing 0.2% FCS, they were treated with scaffold only, ligand only or conjugated assembly of proteins SpT-L1 and L3-DgT for 48 h in starving medium. Cell viability was measured by MTT assay and surviving fractions were normalised to mock-treated control cells. Treatment with scaffold or either ligand only did not result in reduced cell viability. The highest decrease in cell survival was observed after treatment with the fully assembled scaffold SpC-HsCutA1-DgC: SpT-L1:L3-DgT.

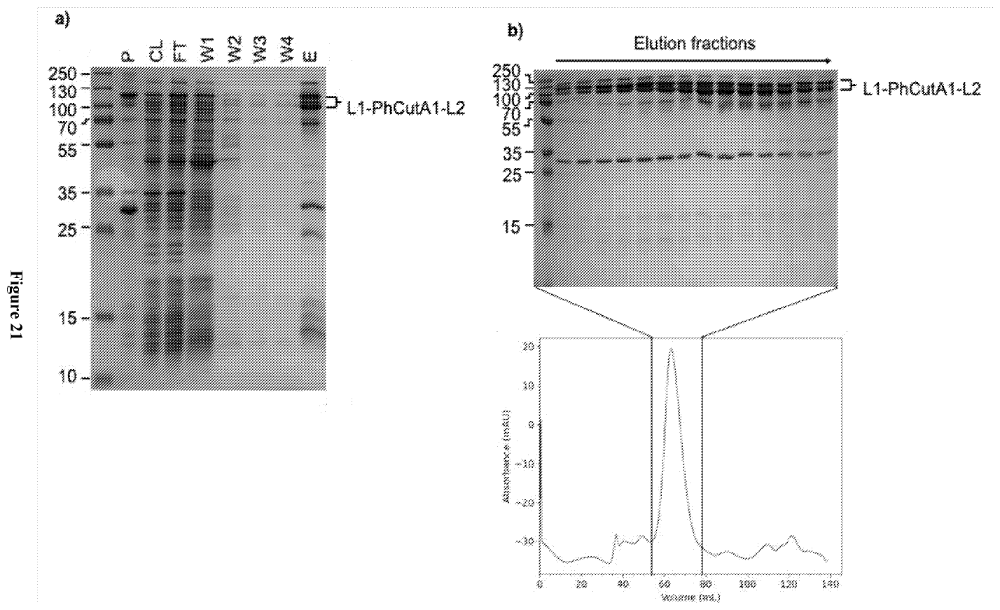

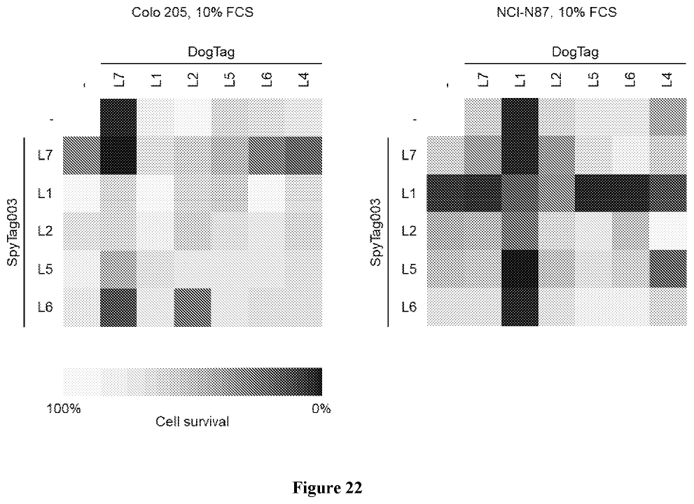

We then expanded the ligand library. Five Spy- and six Dog-Tagged ligands were used to produce 11 single assemblies (SpyTag-ligand or DogTag-ligand only on a scaffold) and 30 fully conjugated assemblies (both SpyTag- and DogTag-ligands on a scaffold) and cytotoxicity was determined in 2 cell lines. Tagged ligands were assembled onto SpC3-HsCutA1-DgC (SEQ ID NO: 24) using a liquid handling machine, and any excess reactants were removed. Colo 205 and NCI-N87 cells were treated or mock-treated with increasing concentrations of the 41 assemblies and surviving fractions were determined as described. Cell survival at the highest concentrations used is presented in a matrix showing the cellular responses to all combinations. Such matrices can be used to select hits for downstream drug development (FIG. 22).

To demonstrate that the ligand library can also consist of binders containing multiple domains, Spy-Tagged scFv and Fab binders have been used as proof-of-principle for protein assembly using SpC3-GGGGS-[C75A, C96A, K82C]HsCutA₄₄₋₁₇₉-GGGGS-DgC (SEQ ID NO: 93). The nanobody-based L7 hexavalent assembly has been used as a control to compare toxicity profiles of binders consisting of single and multi domains. Depending on the binder and target, different binder formatting such as Fab and scFv can impact the toxicity profile of protein assemblies, such as L10-based scFv and Fab containing assemblies (FIG. 33).

### Example 11: Ligands can be Recombinantly Fused Directly to the Core Proteins without the Catcher Modules as a Clinically Relevant Format

Once a hit is identified from a screening panel, the bispecific assembly will need to be reformatted to remove the bacteria-derived Catcher modules to reduce the possibility of inducing unintended and unexpected immune response in a clinical setting. The hit will be reformatted as a direct fusion of the ligand proteins directly to the multimerising core, yielding e.g. L1-HsCutA1-L2. The ligands are genetically fused to the HsCutA 1 via linkers; we hypothesised that these linkers should approximate the length of the removed Catcher modules depending on the ligand position relative to the Tag, such as by using different α-helical linkers or flexible linkers (FIG. 23). We determined cytotoxicity of the direct fusion version of L5-GGGGS-HsCutA1₄₄₋₁₇₉-GGGGS-L4, L1-(GGGGS)3-HsCutA1₄₄₋₁₇₉-(GGGGS)3-L2, L2-(GGGGS)4-HsCutA1₄₄₋₁₇₉-(GGGGS)4-L4 and their respective assemblies with SpC3-HsCutA1₄₄₋₁₇₉-DgC (SEQ ID NO: 24) and tagged L1, L2, L4, and L5 in NCI-N87 cells. Cytotoxicity of the direct fusions was lower compared to their fully assembled counterparts (L5/L4 and L1/L2), suggesting that more accurate linker design, also considering linkers present in the modular HsCutA1 scaffold and/or between modular tags and binder proteins, was necessary (FIG. 23b).

L7 targets Death Receptor 5, a receptor relying on multimerisation to trigger apoptosis and featuring a trimeric protein as its natural ligand (TRAIL). Cytotoxicity was induced by trivalent assemblies consisting of a HsCutA1 Catcher core and either Dog-tagged or Spy003-tagged L7 binders, (FIG. 22, FIG. 23c). Toxicity further increased when L7 was assembled with HsCutA1 via both DogTag and SpyTag003, giving rise to hexavalent assemblies (FIG. 22, FIG. 23d). We next compared cytotoxicity in Colo205 cells induced by hexavalent direct fusions consisting of L7 and HsCutA1 separated via flexible linkers of different lengths {L7-(GGGGS)5-[C75V, C96S]HsCutA1₄₄₋₁₇₉-(GGGGS)8-L7, L7-(GGGGS)5-[C75V, C96S]HsCutA1₄₄₋₁₇₉, [C75V, C96S]HsCutA1₄₄₋₁₇₉-(GGGGS)8-L7, L7-(GGGGS)5-[C75V, C96S]HsCutA1₆₀₋₁₇₁-(GGGGS)8-L7} and by their respective assemblies {(SEQ ID NO: 73 SpC3-[C75V, C96S]HsCutA1₄₄₋₁₇₉-DgC or SEQ ID NO: 76 SpC3-[C75V, C96S]HsCutA1₆₀₋₁₇₁-DgC and tagged L7)}. Increased levels of cytotoxicity conferred by the direct fusions compared to their respective assemblies were noted (FIG. 23d). Differences may be attributable to the effect of linker lengths and positioning of the ligands with respect to the core termini. Iterative rounds of linker optimisation may result in more efficient translation from assembly to direct fusion format.

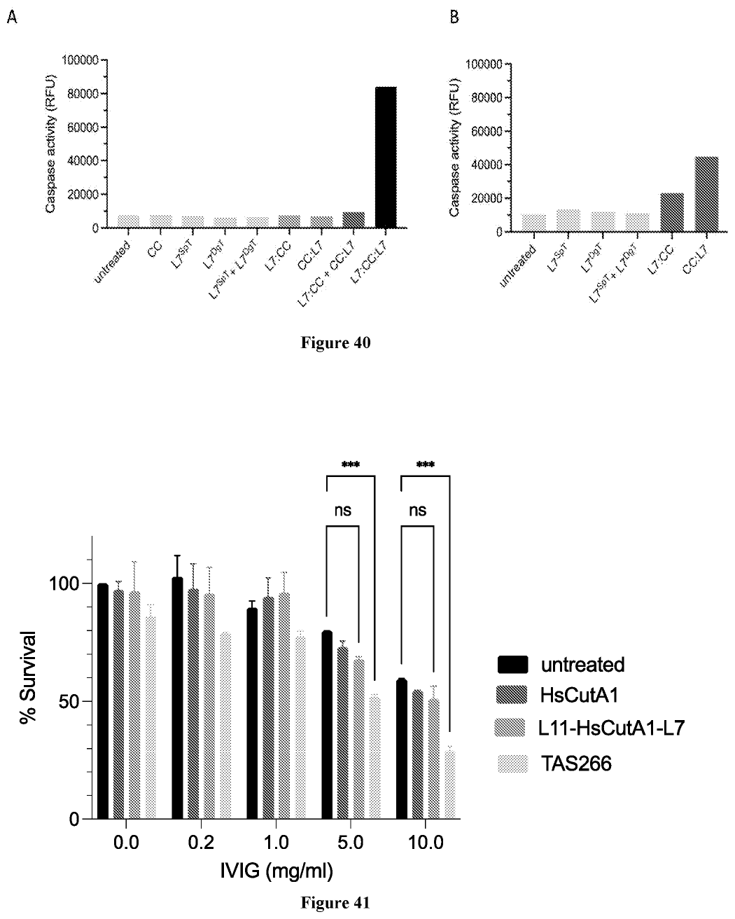

Increased Caspase 3/7 activity after treatment of Colo205 cells with assemblies consisting of Dog-tagged and/or Spy003-tagged L7 and SpC3-HsCutA1₄₄₋₁₇₉-DgC (SEQ ID NO: 24) is consistent with induction of apoptosis (FIG. 40). This demonstrates, for instance, the suitability of CutA1 as a core component of protein complexes (such as assemblies) which function as multivalency-dependent receptor agonists, including agonists of members of the TNF receptor superfamily.

Treatment of Colo205 cells with direct fusions consisting of L7 and three additional cores, OX40L (SEQ ID NO: 78), CD40L (SEQ ID NO: 79), and TNF (SEQ ID NO: 80), resulted in comparable levels of cytotoxicity to those induced by HsCutA1-based fusions [C75V, C96S]HsCutA1₄₄₋₁₇₉ and [C75V, C96S]HsCutA1₆₀₋₁₇₁. We also investigated the potential of a multivalent bispecific HsCutA1-based protein assembly to serve as drug candidate by comparing its cytotoxicity to that of the failed clinical candidate TAS266, a tetrameric L7-nanobody-based molecule. We fused L7 (as used in TAS266) and L11 to [C75A, C96A]HsCutA1₆₀₋₁₇₀, resulting in a proprietary multivalent bispecific construct. L11-HsCutA1-L7 and TAS266 elicit comparable survival profiles in the colorectal cancer cell line HCT116. This effect is specific to L11-HsCutA1-L7, and monovalent L7-binder or HsCutA1 have no effect on cell viability (FIG. 34).

It was hypothesised that the severe hepatotoxicity which lead to clinical failure of TAS266 was a result of antidrug antibody-dependent hyperclustering of DR5 and consequent overinduction of in liver apoptosis cells (Inhibrx, https://patents.google.com/patent/WO2017011837A2/en). Such antibodies are present in commercially available intravenous immunoglobulin (IVIG) solutions (Inhibrx, https://patents.google.com/patent/WQ2017011837A2/en). In contrast to TAS266, L11-HsCutA1-L7 did not increase IVIG-dependent toxicity in the HepG2 hepatocyte line (FIG. 41).

In conclusion, decreased valency of the DR5 targeting binder from four to three and simultaneous introduction of bi-specificity may allow L11-HsCutA1-L7 to elicit less severe adverse effects and display increased tumour-specificity.

### Example 12: Rational Engineering of CutA1 Core Protein for Termini Truncation and Native Cysteine Removal

CutA1 has multiple advantageous features to be utilised as a core protein in a bispecific molecule, as detailed above. Nonetheless, it may be beneficial to modify the protein to have better compatibility with the workflow described elsewhere herein. Such modifications include truncating the termini of HsCutA1 to remove regions which are unresolved in the crystal structure (PDB ID: 2ZFH) and to enforce a similar distance of the N- and C-terminal core residues relative to the surface of the core, and to remove native unpaired cysteines from the HsCutA1 sequence as they may interfere with downstream processes.

Described elsewhere herein are HsCutA1 full length including the signal peptide (SEQ ID NO: 19), removal of 43 N-terminal residues to yield HsCutA1₄₄₋₁₇₉ (SEQ ID NO: 24, 29, 54), and removal of 59 N-terminal residues and 11 C-terminal residues to yield HsCutA1₆₀₋₁₇₁ (SEQ ID NO: 60). Removing 59 N-terminal residues and 8 C-terminal residues to yield HsCutA1₆₀₋₁₇₁ (SEQ ID NO: 66) can enable similar distance of residues that project away from the HsCutA1 core (FIG. 24). Protein production between the two molecules show no drastic difference in expression, which suggests the truncations do not heavily impact HsCutA1 (FIG. 24). Trends in cytotoxicity in Colo 205 cells upon assembly with tagged L7 was largely comparable between the two molecules, suggesting imparted function is not affected (FIG. 24).

HsCutA1 has two native unpaired cysteines at positions 75 and 96 (FIG. 25). Alanine scanning mutagenesis is a common strategy to substitute unpaired cysteine residues. Further, a multiple sequence alignment was produced using the HsCutA1 sequence to identify preferred amino acid frequencies at positions C75 and C96 across 406 homologs. Thereafter, these residues were mutated to either alanine for both positions yielding [C75A, C96A]HsCutA1 mutant or to valine at position 75 and to serine at position 96, yielding [C75V, C96S]HsCutA1. Protein production between the molecules show no drastic difference in expression, which suggests that cysteine removal do not heavily impact HsCutA1 (FIG. 25). Cell killing effects in Colo 205 cells upon assembly with tagged L7 were largely comparable, suggesting imparted function is not affected (FIG. 25).

### Example 13: Mutagenesis of CutA1 as a Core Protein for Multivalent Bispecific Drug Conjugation

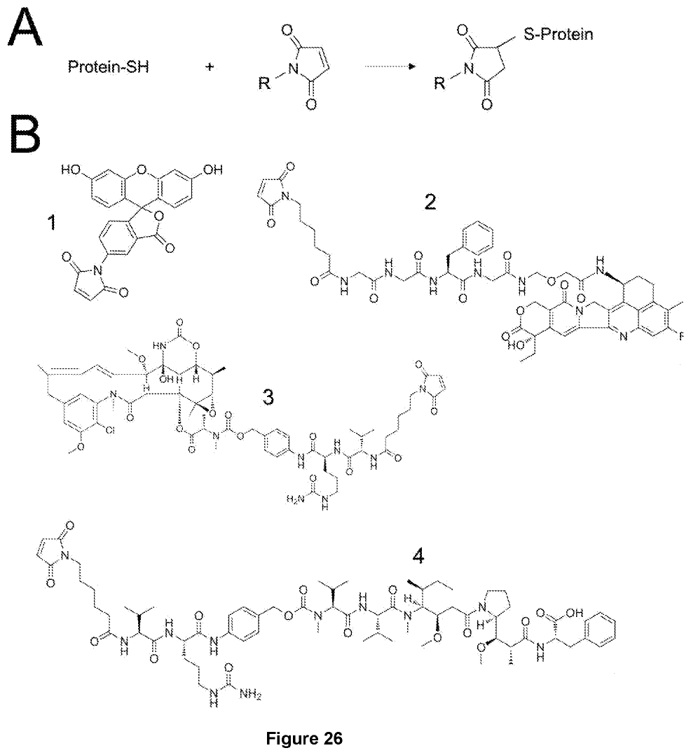

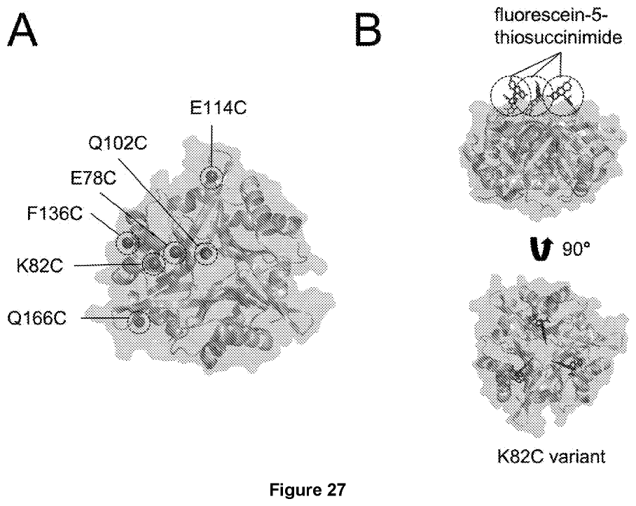

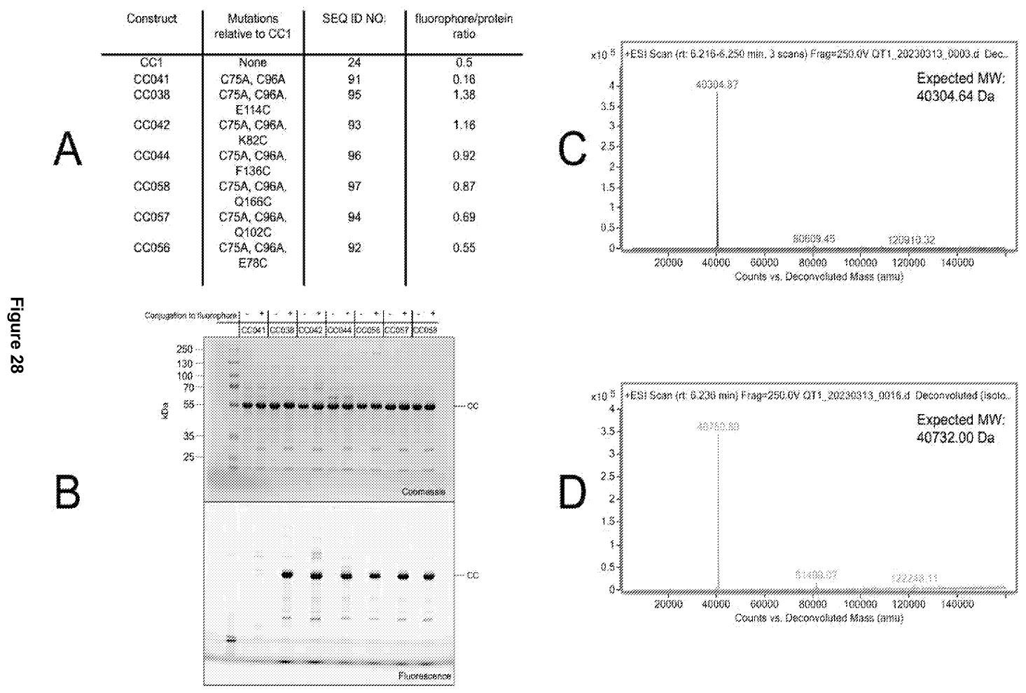

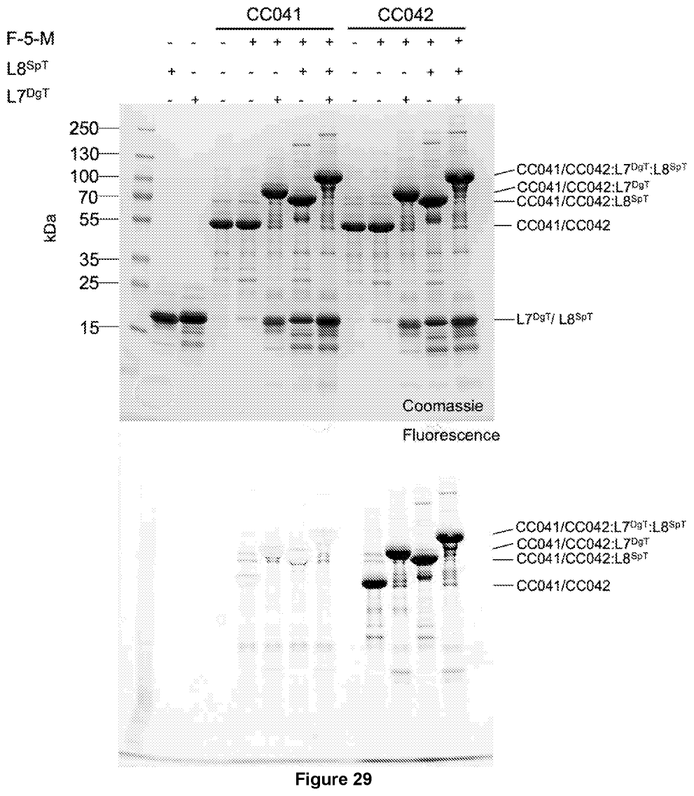

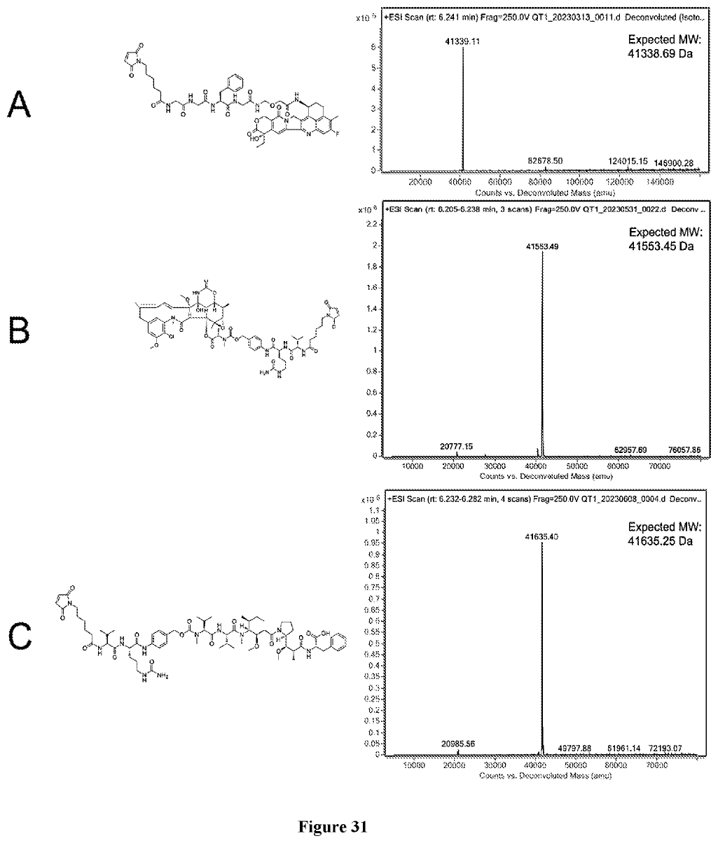

To enable site-specific conjugation of payloads to the CutA1 Catcher-core, 6 amino acid positions in CutA1 [44-179, C75A, C96A] (SEQ ID NO: 67) were mutated to Cys (see Methods: Cysteine mutant design) based on homology modelling or surface exposure. The thiol groups of Cys residues could then be reacted with the maleimide moiety of linker-molecule-of-interest compounds such as fluorescein-5-maleimide (FIG. 26A, B). The positions of the mutations on the CutA1 structure are shown in FIG. 27A. A comparison of conjugation efficiency (dye/protein ratio) between the different CutA1 Catcher core mutants (SEQ ID NO: 92, 93, 94, 95, 96, 97), a non-Cys-containing negative control, CC041 (SEQ ID NO: 91), and the wild-type (SEQ ID NO: 24) is shown in FIG. 28A. All Cys mutants featured a higher conjugation efficiency compared to wild-type and the negative control. CC041 (SEQ ID NO: 91) displays a low background level of non-specific fluorescein-5-maleimide labelling. Mass spectra of the CC042 (SEQ ID NO: 93) conjugated to fluorescein-5-maleimide (FIG. 28C, D) and three different linker-drug compounds (FIG. 31A, B, C) are consistent with complete conjugation of a single linker-payload compound to each CutA1 monomer. This is equivalent to a ‘drug-to-antibody’ ratio of 3:1 as CutA1 is trimeric. FIG. 29 shows conjugation of CC042 (SEQ ID NO: 93) with fluorescein-5-maleimide, as well as assembly of fluorescein-conjugated CC042 with SpyTagged- and DogTagged-ligands. In contrast to CC042, the negative control CC041 (SEQ ID NO: 91) only displays a low level of non-specific labelling. Both CC041 and CC042 assemble with ligands. SDS-PAGE followed by Coomassie staining showed that intensity of bands corresponding to the predicted molecular weight of assemblies are very similar irrespective of the Catcher-core being efficiently fluorescein-labelled (CC042) or not (CC041) (FIG. 29). In summary, FIG. 29 suggests that maleimide conjugation does not compromise the ability of the Catcher-core protein to assemble with tagged ligands.

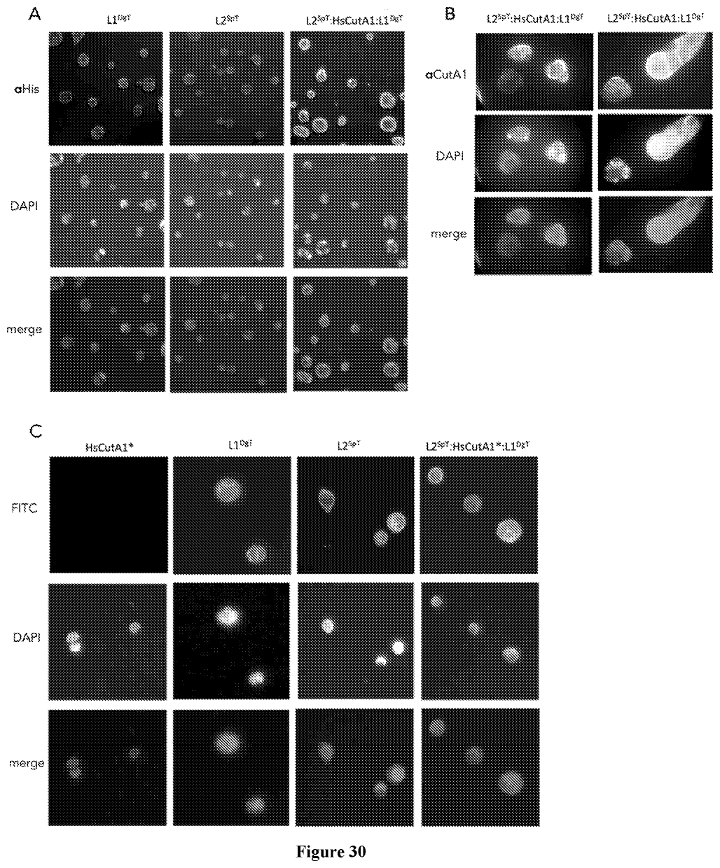

The Example also demonstrates the generation of dye-conjugated bispecifics, enabling rapid imaging without the need for antibody-based staining. FIG. 30C depicts fluorescein-conjugated CutA1-core which has been assembled with one or two binders to generate multivalent and multivalent bispecific fluorescein-labelled assemblies. This system allows the generation of a library of multivalent bispecific fluorescein-labelled drug conjugates at scale. These assemblies can then be directly used for imaging without the need to use fluorescently-labelled antibodies, e.g. against the Catcher Core or binders. In addition, this system can be used for high-throughput imaging applications, e.g. to screen for high binding/internalising binder clones using high-throughput imaging applications.

### Sequences Used in this Example

CC1
MGSSHHHHHHSSGVTTLSGLSGEQGPSGDMTTEEDSATHIKFSKRDEDGR
ELAGATMELRDSSGKTISTWISDGHVKDFYLYPGKYTFVETAAPDGYEVA
TPIEFTVNEDGQVTVDGEATEGDAHTGGGGSMASGSPPTQPSPASDSGSG
YVPGSVSAAFVTCPNEKVAKEIARAVVEKRLAACVNLIPQITSIYEWKGK
IEEDSEVLMMIKTQSSLVPALTDFVRSVHPYEVAEVIALPVEQGNFPYLQ
WVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYK
PVQNKPIVSFRIVDGEVRDVTSIVPQ
CC038
MGSSHHHHHHSSGVTTLSGLSGEQGPSGDMTTEEDSATHIKFSKRDEDGR
ELAGATMELRDSSGKTISTWISDGHVKDFYLYPGKYTFVETAAPDGYEVA
TPIEFTVNEDGQVTVDGEATEGDAHTGGGGSMASGSPPTQPSPASDSGSG
YVPGSVSAAFVTAPNEKVAKEIARAVVEKRLAAAVNLIPQITSIYEWKGK
ICEDSEVLMMIKTQSSLVPALTDFVRSVHPYEVAEVIALPVEQGNFPYLQ
WVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYK
PVQNKPIVSFRIVDGEVRDVTSIVPQ
CC041
MGSSHHHHHHSSGVTTLSGLSGEQGPSGDMTTEEDSATHIKFSKRDEDGR
ELAGATMELRDSSGKTISTWISDGHVKDFYLYPGKYTFVETAAPDGYEVA
TPIEFTVNEDGQVTVDGEATEGDAHTGGGGSMASGSPPTQPSPASDSGSG
YVPGSVSAAFVTAPNEKVAKEIARAVVEKRLAAAVNLIPQITSIYEWKGK
IEEDSEVLMMIKTQSSLVPALTDFVRSVHPYEVAEVIALPVEQGNFPYLQ
WVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYK
PVQNKPIVSFRIVDGEVRDVTSIVPQ
CC042
MGSSHHHHHHSSGVTTLSGLSGEQGPSGDMTTEEDSATHIKFSKRDEDGR
ELAGATMELRDSSGKTISTWISDGHVKDFYLYPGKYTFVETAAPDGYEVA
TPIEFTVNEDGQVTVDGEATEGDAHTGGGGSMASGSPPTQPSPASDSGSG
YVPGSVSAAFVTAPNEKVACEIARAVVEKRLAAAVNLIPQITSIYEWKGK
IEEDSEVLMMIKTQSSLVPALTDFVRSVHPYEVAEVIALPVEQGNFPYLQ
WVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYK
PVQNKPIVSFRIVDGEVRDVTSIVPQ
CC044
MGSSHHHHHHSSGVTTLSGLSGEQGPSGDMTTEEDSATHIKFSKRDEDGR
ELAGATMELRDSSGKTISTWISDGHVKDFYLYPGKYTFVETAAPDGYEVA
TPIEFTVNEDGQVTVDGEATEGDAHTGGGGSMASGSPPTQPSPASDSGSG
YVPGSVSAAFVTAPNEKVAKEIARAVVEKRLAAAVNLIPQITSIYEWKGK
IEEDSEVLMMIKTQSSLVPALTDCVRSVHPYEVAEVIALPVEQGNFPYLQ
WVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYK
PVQNKPIVSFRIVDGEVRDVTSIVPQ
CC056
MGSSHHHHHHSSGVTTLSGLSGEQGPSGDMTTEEDSATHIKFSKRDEDGR
ELAGATMELRDSSGKTISTWISDGHVKDFYLYPGKYTFVETAAPDGYEVA
TPIEFTVNEDGQVTVDGEATEGDAHTGGGGSMASGSPPTQPSPASDSGSG
YVPGSVSAAFVTAPNCKVAKEIARAVVEKRLAAAVNLIPQITSIYEWKGK
IEEDSEVLMMIKTQSSLVPALTDFVRSVHPYEVAEVIALPVEQGNFPYLQ
WVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYK
PVQNKPIVSFRIVDGEVRDVTSIVPQ
CC057
MGSSHHHHHHSSGVTTLSGLSGEQGPSGDMTTEEDSATHIKFSKRDEDGR
ELAGATMELRDSSGKTISTWISDGHVKDFYLYPGKYTFVETAAPDGYEVA
TPIEFTVNEDGQVTVDGEATEGDAHTGGGGSMASGSPPTQPSPASDSGSG
YVPGSVSAAFVTAPNEKVAKEIARAVVEKRLAAAVNLIPCITSIYEWKGK
IEEDSEVLMMIKTQSSLVPALTDFVRSVHPYEVAEVIALPVEQGNFPYLQ
WVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYK
PVQNKPIVSFRIVDGEVRDVTSIVPQ
CC058
MGSSHHHHHHSSGVTTLSGLSGEQGPSGDMTTEEDSATHIKFSKRDEDGR
ELAGATMELRDSSGKTISTWISDGHVKDFYLYPGKYTFVETAAPDGYEVA
TPIEFTVNEDGQVTVDGEATEGDAHTGGGGSMASGSPPTQPSPASDSGSG
YVPGSVSAAFVTAPNEKVAKEIARAVVEKRLAAAVNLIPQITSIYEWKGK
IEEDSEVLMMIKTQSSLVPALTDFVRSVHPYEVAEVIALPVEQGNFPYLQ
WVRCVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYK
PVQNKPIVSFRIVDGEVRDVTSIVPQ

### Alignment Showing Variation:

| | | |
|---|---|---|
| CC1 | MGSSHHHHHHSSGVTTLSGLSGEQGPSGDMTTEEDSATHIKFSKRDEDGRELAGATMELR | 60 |
| CC058 | MGSSHHHHHHSSGVTTLSGLSGEQGPSGDMTTEEDSATHIKFSKRDEDGRELAGATMELR | 60 |
| CC057 | MGSSHHHHHHSSGVTTLSGLSGEQGPSGDMTTEEDSATHIKFSKRDEDGRELAGATMELR | 60 |
| CC056 | MGSSHHHHHHSSGVTTLSGLSGEQGPSGDMTTEEDSATHIKFSKRDEDGRELAGATMELR | 60 |
| CC044 | MGSSHHHHHHSSGVTTLSGLSGEQGPSGDMTTEEDSATHIKFSKRDEDGRELAGATMELR | 60 |
| CC042 | MGSSHHHHHHSSGVTTLSGLSGEQGPSGDMTTEEDSATHIKFSKRDEDGRELAGATMELR | 60 |
| CC038 | MGSSHHHHHHSSGVTTLSGLSGEQGPSGDMTTEEDSATHIKFSKRDEDGRELAGATMELR | 60 |
| CC041 | MGSSHHHHHHSSGVTTLSGLSGEQGPSGDMTTEEDSATHIKFSKRDEDGRELAGATMELR | 60 |
| | ***************************F******************************** | |
| CC1 | DSSGKTISTWISDGHVKDFYLYPGKYTFVETAAPDGYEVATPIEFTVNEDGQVTVDGEAT | 120 |
| CC058 | DSSGKTISTWISDGHVKDFYLYPGKYTFVETAAPDGYEVATPIEFTVNEDGQVTVDGEAT | 120 |
| CC057 | DSSGKTISTWISDGHVKDFYLYPGKYTFVETAAPDGYEVATPIEFTVNEDGQVTVDGEAT | 120 |
| CC056 | DSSGKTISTWISDGHVKDFYLYPGKYTFVETAAPDGYEVATPIEFTVNEDGQVTVDGEAT | 120 |
| CC044 | DSSGKTISTWISDGHVKDFYLYPGKYTFVETAAPDGYEVATPIEFTVNEDGQVTVDGEAT | 120 |
| CC042 | DSSGKTISTWISDGHVKDFYLYPGKYTFVETAAPDGYEVATPIEFTVNEDGQVTVDGEAT | 120 |
| CC038 | DSSGKTISTWISDGHVKDFYLYPGKYTFVETAAPDGYEVATPIEFTVNEDGQVTVDGEAT | 120 |
| CC041 | DSSGKTISTWISDGHVKDFYLYPGKYTFVETAAPDGYEVATPIEFTVNEDGQVTVDGEAT | 120 |
| | ************************************************************ | |
| CC1 | EGDAHTGGGGSMASGSPPTQPSPASDSGSGYVPGSVSAAFVTCPNEKVAKEIARAVVEKR | 180 |
| CC058 | EGDAHTGGGGSMASGSPPTQPSPASDSGSGYVPGSVSAAFVTAPNEKVAKEIARAVVEKR | 180 |
| CC057 | EGDAHTGGGGSMASGSPPTQPSPASDSGSGYVPGSVSAAFVTAPNEKVAKEIARAVVEKR | 180 |
| CC056 | EGDAHTGGGGSMASGSPPTQPSPASDSGSGYVPGSVSAAFVTAPNCKVAKEIARAVVEKR | 180 |
| CC044 | EGDAHTGGGGSMASGSPPTQPSPASDSGSGYVPGSVSAAFVTAPNEKVAKEIARAVVEKR | 180 |
| CC042 | EGDAHTGGGGSMASGSPPTQPSPASDSGSGYVPGSVSAAFVTAPNEKVACEIARAVVEKR | 180 |
| CC038 | EGDAHTGGGGSMASGSPPTQPSPASDSGSGYVPGSVSAAFVTAPNEKVAKEIARAVVEKR | 180 |
| CC041 | EGDAHTGGGGSMASGSPPTQPSPASDSGSGYVPGSVSAAFVTAPNEKVAKEIARAVVEKR | 180 |
| | ************************************************************ | |
| CC1 | LAACVNLIPQITSIYEWKGKIEEDSEVIMMIKTQSSLVPALTDFVRSVHPYEVAEVIALP | 240 |
| CC058 | LAAAVNLIPQITSIYEWKGKIEEDSEVLMMIKTQSSLVPALTDFVRSVHPYEVAEVIALP | 240 |
| CC057 | LAAAVNLIPCITSIYEWKGKIEEDSEVLMMIKTQSSLVPALTDFVRSVHPYEVAEVIALP | 240 |
| CC056 | LAAAVNLIPQITSIYEWKGKIEEDSEVLMMIKTQSSLVPALTDFVRSVHPYEVAEVIALP | 240 |
| CC044 | LAAAVNLIPQITSIYEWKGKIEEDSEVLMMIKTQSSLVPALTDCVRSVHPYEVAEVIALP | 240 |
| CC042 | LAAAVNLIPQITSIYEWKGKIEEDSEVIMMIKTQSSLVPALTDFVRSVHPYEVAEVIALP | 240 |
| CC038 | LAAAVNLIPQITSIYEWKGKICEDSEVIMMIKTQSSLVPALTDFVRSVHPYEVAEVIALP | 240 |
| CC041 | LAAAVNLIPQITSIYEWKGKIEEDSEVLMMIKTQSSLVPALTDFVRSVHPYEVAEVIALP | 240 |
| | ************************************************************ | |
| CC1 | VEQGNFPYLQWVRQVTESVSDSITVLPGGGGSKLGEIEFIKVDKTDKKPLRGAVFSLQKQ | 300 |
| CC058 | VEQGNFPYLQWVRCVTESVSDSITVLPGGGGSKLGEIEFIKVDKTDKKPLRGAVFSLQKQ | 300 |
| CC057 | VEQGNFPYLQWVRQVTESVSDSITVLPGGGGSKLGEIEFIKVDKTDKKPLRGAVFSLQKQ | 300 |
| CC056 | VEQGNFPYLQWVRQVTESVSDSITVLPGGGGSKLGEIEFIKVDKIDKKPLRGAVFSLQKQ | 300 |
| CC044 | VEQGNFPYLQWVRQVTESVSDSITVLPGGGGSKLGEIEFIKVDKTDKKPLRGAVFSLQKQ | 300 |
| CC042 | VEQGNFPYLQWVRQVTESVSDSITVLPGGGGSKLGEIEFIKVDKTDKKPLRGAVFSLQKQ | 300 |
| CC038 | VEQGNFPYLQWVRQVTESVSDSITVLPGGGGSKLGEIEFIKVDKTDKKPLRGAVFSLQKQ | 300 |
| CC041 | VEQGNFPYLQWVRQVTESVSDSITVLPGGGGSKLGEIEFIKVDKTDKKPLRGAVFSLQKQ | 300 |
| | ************************************************************ | |
| CC1 | HPDYPDIYGAIDQNGTYQDVRTGEDGKLTFTNLSDGKYRLIENSEPPGYKPVQNKPIVSF | 360 |
| CC058 | HPDYPDIYGAIDQNGTYQDVRTGEDGKLTFTNLSDGKYRLIENSEPPGYKPVQNKPIVSF | 360 |
| CC057 | HPDYPDIYGAIDQNGTYQDVRTGEDGKLTFTNLSDGKYRLIENSEPPGYKPVQNKPIVSF | 360 |
| CC056 | HPDYPDIYGAIDQNGTYQDVRTGEDGKLIFTNLSDGKYRLIENSEPPGYKPVQNKPIVSF | 360 |
| CC044 | HPDYPDIYGAIDQNGTYQDVRTGEDGKLTFTNLSDGKYRLIENSEPPGYKPVQNKPIVSF | 360 |
| CC042 | HPDYPDIYGAIDQNGTYQDVRTGEDGKLTFTNLSDGKYRLIENSEPPGYKPVQNKPIVSF | 360 |
| CC038 | HPDYPDIYGAIDQNGTYQDVRTGEDGKLTFTNLSDGKYRLIENSEPPGYKPVQNKPIVSF | 360 |
| CC041 | HPDYPDIYGAIDQNGTYQDVRTGEDGKLTFTNLSDGKYRLIENSEPPGYKPVQNKPIVSF | 360 |
| | ************************************************************ | |
| CC1 | RIVDGEVRDVTSIVPQ | 376 |
| CC058 | RIVDGEVRDVTSIVPQ | 376 |
| CC057 | RIVDGEVRDVTSIVPQ | 376 |
| CC056 | RIVDGEVRDVTSIVPQ | 376 |
| CC044 | RIVDGEVRDVTSIVPQ | 376 |
| CC042 | RIVDGEVRDVTSIVPQ | 376 |
| CC038 | RIVDGEVRDVTSIVPQ | 376 |
| CC041 | RIVDGEVRDVTSIVPQ | 376 |
| | **************** | |

### Methods

### Cysteine Mutant Design

### Cysteine Mutants K82C, E114C, F136C

The human CutA1 crystal structure (PDB ID 2ZFH) was aligned against homologous structures using FATCAT (Ye, Y. & Godzik, A. FATCAT: a web server for flexible structure comparison and structure similarity searching. Nucleic Acids Res. 32, W582-585 (2004)). Positions with corresponding surface facing Cys residues in homologues were selected, leading to the following mutations: K82C, E114C, F136C (SEQ ID NO: 86, 88, 89)

### Cysteine Mutants E78C, Q102C, Q166C

The human CutA1 crystal structure (PDB ID 2ZFH) was minimised using the EvoEF2 (Huang, X., Pearce, R. & Zhang, Y. EvoEF2: accurate and fast energy function for computational protein design. Bioinforma. Oxf. Engl. 36, 1135-1142 (2020)) force field with the RepairStructure command. Then, Cys 75 and Cys 96 were mutated to Ala using the BuildMutant command. Subsequently using this model with Cys to Ala mutations, structural models with a single Cys mutation at positions V64, E78, K79, E83, K91, Q102, K110, S139, F158 and Q166 were generated. The solvent accessibility of the in silico Cys mutant models was evaluated using PDBePISA (Krissinel, E. & Henrick, K. Inference of Macromolecular Assemblies from Crystalline State. J. Mol. Biol. 372, 774-797 (2007) by averaging the solvent-accessible surface area value for the mutated residue over the three chains of CutA1. Three mutations were selected across a range of solvent accessibility: E78C, Q102C, Q166C (SEQ ID NO: 85, 87, 90)

| | Average solvent-accessible surface area |
|---|---|
| Mutation | (Å²) |
| E78C | 59.96 |
| Q102C | 121.83 |
| Q166C | 80.01 |

All of the above Cys mutations were introduced individually into the SpC3-G4S-HsCutA1 [44-179, C75A, C96A]-G4S-DgC construct (SEQ ID NO: 91).

### Protein Expression

To obtain proteins CC041, CC038, CC042, CC044, CC056, CC057, and CC058, DNA encoding these genes, in the pET28 expression vector, was transformed into BL21 (DE3) or SHuffle T7 express cells. Colonies were used to inoculate LB cultures with 50 μg/mL Kanamycin at 37° C. with 160-220 rpm shaking. Overnight cultures were diluted 1:100 into LB or 2×YT media supplemented with 50 μg/mL Kanamycin. Cultures were grown at 37° C. with 160-220 rpm shaking before induction of protein expression with 0.2-0.4 μM IPTG at OD 0.6-0.8 (LB) or 1.6-2.0 (2×YT). The cultures were incubated for a further 4 hours at 37° C., or 16 hours at 18° C., with 160-200 rpm shaking.

Alternatively, overnight cultures were diluted 1:100 into SB autoinduction media supplemented with 50 μg/mL Kanamycin. Cultures were grown at 37° C. with 160-220 rpm shaking for 4 hours. The cultures were then incubated for a further 16 hours at 30° C. with 160-200 rpm shaking.

Cells were collected by centrifugation at 5000×g for 15 minutes at 4° C. and pellets stored at −80° C. prior to purification.

### Protein Purification

### Ni-NTA

For proteins CC038, CC042, CC044, CC056, CC057, and CC058, bacterial cell pellets were resuspended in Ni-NTA equilibration buffer (50 mM Tris, pH 7.8; 300 mM 25 NaCl, 10 mM imidazole) supplemented with 1 mM PMSF, 1×complete EDTA-free protease inhibitor cocktail, benzonase (5 U/mL) and 5 mM 2-mercaptoethanol. Samples were sonicated with an Ultrasonic Processor using a 20 mm probe and an amplitude of 20%, for 9-12 minutes pulsing on 2 seconds/off 4 seconds.

Alternatively, bacterial cell pellets were resuspended in B-PER chemical lysis buffer (ThermoFisher) supplemented with 1 mM PMSF, 1× complete EDTA-free protease inhibitor cocktail, benzonase (5 U/mL), 1 μg/ml lysozyme and 5 mM 2-mercaptoethanol.

The samples were then centrifuged at 16,000×g for 30 min. Supernatants were retained for Ni-NTA chromatography.

The supernatant was loaded onto pre-equilibrated HisPur Ni-NTA gravity-flow columns. The resin was washed with 20 resin-bed volumes of Ni-NTA Wash Buffer 1 (50 mM Tris, pH 7.8; 300 mM NaCl, 10 mM imidazole) supplemented with 5 mM 2-mercaptoethanol and subsequently with 10 resin-bed volumes of Ni-NTA Wash Buffer 2 (50 mM Tris, pH 7.8; 300 mM NaCl, 30 mM imidazole) supplemented with 5 mM 2-mercaptoethanol. His-tagged proteins were eluted from the resin with five resin-bed volumes of Ni-NTA Elution Buffer (50 mM Tris, pH 7.8; 300 mM NaCl, 200 mM imidazole) supplemented with 5 mM 2-mercaptoethanol until the absorbance of the elution fractions at 280 nm approached baseline.

For protein CC041, the same protocols were used but without supplementing buffers with 5 mM 2-mercaptoethanol.

### SEC

For CC038, CC042, CC044, CC056, CC057, CC058, proteins that were above 90% purity following Ni-NTA chromatography were dialysed against PBS supplemented with 5 mM 2-mercaptoethanol using SnakeSkin™ Dialysis Tubing at 3K MWCO. The proteins were then concentrated in a Pierce™ Protein Concentrator PES. The proteins were then further purified using an AKTA Pure FPLC with a Superdex 16/600 S200 column. Samples were purified at 4° C. using PBS buffer supplemented with 2 mM TCEP. The elution peak fractions were analysed by SDS-PAGE and the appropriate fractions were pooled. Estimated protein concentrations were calculated from measurement of absorbance at 280 nm using an Implen NanoPhotometer N60 with reduced extinction coefficients predicted by ProtParam. Protein samples were adjusted to 100-300 μM and stored at −80° C.

For CC041 the same protocol was used but without supplementing buffers with 2-mercaptoethanol and TCEP.

### Cysteine-Maleimide Conjugation

### Catcher-Core Protein Reduction

Catcher-core protein variants containing single Cys residues were prepared at a concentration of 50 μM in PBS buffer pH 7.4 (Formedium) with 50 mM EDTA (Formedium). To reduce cysteine residues, 100 molar equivalents (relative to protein thiols) of 40 mM TCEP (Melford) in PBS buffer at pH 7.0 were added to the Catcher-core protein and the mixture was incubated for either a) 16 hours at 37° C., or b) 2 hours at 25° C. on a microplate shaker (Fisher scientific) with shaking at 600 rpm.

### Maleimide Conjugation

10 to 15 molar equivalents (relative to protein thiols) of fluorescein-5-maleimide, deruxtecan, MC-Val-Cit-PAB-DM1 or MC-Val-Cit-PAB-MMAF (MedChemExpress, dissolved in DMSO) were added and the mixture was incubated for 1 hour at 25° C. Finally, molar equivalents (relative to maleimide) of L-cysteine (Melford) (dissolved in PBS pH 7.4) were added and the mixture was incubated for 15 minutes to terminate the reaction.

### Dye-Protein Conjugate Purification

The reaction mixture was centrifuged for 10 minutes at 17,000 g to remove precipitate. Subsequently the reaction mixture was loaded on a PD-10 gravity flow desalting column (Cytiva), followed by up to 2.5 mL of PBS buffer. The conjugated protein was eluted with an additional 3.5 mL of PBS buffer. 250 μL elution fractions with a high protein concentration, as determined by UV/Vis spectrophotometry using an IMPLEN Nanophotometer at wavelengths of 280 nm and 495 nm, were selected and concentrated using a 0.5 mL Pierce™ protein concentrator (ThermoFisher) with either a 3 or 10 kDa MWCO filter to a final volume of approximately 100-400 μL. Protein concentration was adjusted for the contribution of the dye to absorbance at 280 nm using the following formula:

$${C}_{prot} = \frac{{A}_{280} - 0.3 \times {A}_{495}}{{ε}_{prot}}$$

A₂₈₀ denotes the absorbance of the payload-protein conjugate at 280 nm, A495 denotes the absorbance at 495 nm and εₚᵣₒₜ denotes the extinction coefficient of the reduced protein at 280 nm as calculated using ExPASy ProtParam.

### Dye/Protein Ratio Estimation

Final dye/protein ratio after concentration was estimated using the following formula:

$$dye / protein ratio = \frac{{A}_{495}}{{ε}_{dye} \times {C}_{prot}}$$

A₄₉₅ denotes the absorbance of the payload-protein conjugate at 495 nm, ε_{dye} denotes the extinction coefficient of the dye at 495 nm (68,000 M⁻¹cm⁻¹), Cₚᵣₒₜ denotes the adjusted protein concentration according to the above equation.

### Drug-Protein Conjugate Purification

The reaction mixture was centrifuged for 10 minutes at 17,000 g to remove precipitates. Subsequently the reaction mixture was loaded on a PD-10 gravity flow desalting column (Cytiva), followed by up to 2.5 mL of PBS buffer. The conjugated protein was eluted with an additional 3.5 mL PBS buffer. Elution fractions with a high protein concentration, as determined by UV/Vis spectrophotometry using an IMPLEN Nanophotometer at wavelengths of 280 nm and a wavelength with high absorbance from the drug, λ_{drug}, were selected and concentrated using a 0.5 mL Pierce™ protein concentrator (ThermoFisher) with either a 10 kDa MWCO filter to a final volume of approximately 100-400 μL.

| | | ε_{drug}^{λdrug} - | ε_{drug}²⁸⁰ - |
|---|---|---|---|
| | | extinction | extinction |
| Linker-drug | | coefficient of the | coefficient of the |
| compound | λ_{drug} | drug at λdrug | drug at 280 nm |
| deruxtecan | 370 nm | 20,982 M⁻¹cm⁻¹ | 10,764 M⁻¹cm⁻¹ |
| MC-Val-Cit-PAB- | 252 nm | 26,350 M⁻¹cm⁻¹ | 5,456 M⁻¹cm⁻¹ |
| DM1 | | | |
| MC-Val-Cit-PAB- | 214 nm | — | — |
| MMAF* | | | |

For deruxtecan, extinction coefficients at 280 nm (10,764 M⁻¹cm⁻¹) and 370 nm (20,982 M⁻¹cm⁻¹) for a close deruxtecan analogue (compound 21a⁶) were used. For DM1, extinction coefficients at 280 nm and 252 nm were used as determined in (Fishkin, N. Maytansinoid-BODIPY Conjugates: Application to Microscale Determination of Drug Extinction Coefficients and for Quantification of Maytansinoid Analytes. Mol. Pharmaceutics 12, 1745-1751 (2015)). * Absorbance at 214 nm for MMAF was only used to determine the fractions at which free MC-Val-Cit-PAB-MMAF begins to elute as MMAF absorbance overlaps with protein peptide bond absorbance.

Protein concentration was adjusted for the contribution of the drug to absorbance at 280 nm using the following formula from (Chen, Y. Drug-to-Antibody Ratio (DAR) by UV/Vis Spectroscopy. in Antibody-Drug Conjugates (ed. Ducry, L.) 267-273 (Humana Press, 2013)):

$${C}_{prot} = \frac{{A}_{280} \times {ε}_{drug}^{λ drug} - {A}_{λ drug} \times {ε}_{drug}^{280}}{( {ε}_{prot}^{280} \times {ε}_{drug}^{λ drug} - {ε}_{drug}^{280} - {ε}_{prot}^{λ drug} ) \times l}$$

A₂₈₀ denotes the absorbance of the linker-drug-protein conjugate at 280 nm, A_{λdrug} denotes the absorbance of the linker-drug-protein conjugate at a wavelength specific to the drug,

$${ε}_{drug}^{λ drug}$$

denotes the extinction coefficient of the drug at λ_{drug},

$${ε}_{drug}^{280}$$

denotes the extinction coefficient of the drug at 280 nm,

$${ε}_{prot}^{280}$$

denotes the extinction coefficient of the reduced protein at 280 nm as calculated using ExPASy ProtParam and

$${ε}_{prot}^{λ drug}$$

denotes the extinction coefficient of the protein at λ_{drug} as estimated by Nanophotometer measurements at 280 nm and λ_{drug} of the unconjugated protein, and l denotes the optical path length.

Subsequently, the concentrated sample was further purified using a Slide-A-Lyzer® mini dialysis device (Thermo scientific, 0.5 ml sample volume, 20 kDa MWCO cut off filter) for eight rounds of dialysis into 14 ml of PBS buffer, each of at least two hours. Final linker-drug-protein conjugate concentrations were determined using the Coomassie Plus Bradford assay (ThermoFisher).

### Payload Protein Ratio Estimation

The final drug concentration after dialysis was estimated using the following formula from (Chen, Y. Drug-to-Antibody Ratio (DAR) by UV/Vis Spectroscopy. in Antibody-Drug Conjugates (ed. Ducry, L.) 267-273 (Humana Press, 2013)):

$${C}_{drug} = \frac{{A}_{280} \times {ε}_{prot}^{λ drug} - {A}_{λ drug} \times {ε}_{prot}^{280}}{( {ε}_{drug}^{280} \times {ε}_{prot}^{λ drug} - {ε}_{prot}^{280} - {ε}_{drug}^{λ drug} ) \times l}$$

The final drug-to-protein molar ratio was then estimated using:

$$drug / protein ratio = \frac{{C}_{drug}}{{C}_{prot}}$$

### LC-MS

The molecular weight of the dye-protein and drug-protein conjugates of SpyCatcher3-G4S-HsCutA1 [44-179, C75A, C96A, K82C]-G4S-DogCatcher, or CC042 (SEQ ID NO: 93) was analysed using LC-MS. An Agilent 1290 UPLC and a 6550 ESI Q-TOF mass spectrometer were used. LC buffer A was 0.1% formic acid in water, LC buffer B was 0.1% formic acid in acetonitrile. An Agilent PLRP-S 1000A (5 μm 50×2.1 mm) column was used at 60C, flow rate of 0.4 ml/min and 0.2 μL sample injection volume. Solvent composition was held at 20% B for 5 minutes, increased to 75% B in 1 minute then to 100% B in 4 minutes and held for 2 minutes. Positive ion spectra were acquired over the m/z range 300-3200 at the rate of 1 spectrum per second.

### Catcher-Based Conjugation

### SpyTag DogTag-Ligand Assembly Test

Reaction mixtures of fluorescein-5-maleimide-conjugated CutA1-based cores fused with SpyCatcher003 and DogCatcher and SpT3 and DgT binder proteins were prepared at 10 μM Catcher-core with a 2:1 molar ratio of each binder over the Catcher-core. The reactions were prepared in PBS buffer and incubated on a plate shaker at 600 rpm at 25° C. for 1 hour. Subsequently, 6×SDS loading buffer was added and the samples were heated at 95° C. for 5 minutes to terminate the reaction and prepare the samples for visualisation by SDS-PAGE.

Another variation of the conjugation process is as follows. Protein samples were first quantified using the Coomassie Plus Bradford assay (ThermoFisher). Conjugation samples of SpT3, and DgT binder proteins to CutA1-based cores fused with SpyCatcher003 and DogCatcher were prepared at 20 μM with a 1:1.2:1.2 binder excess of platform: ligand: ligand ratios. The reactions were prepared in PBS buffer supplemented with 1×complete EDTA-free protease inhibitor and 50 μg/mL Penicillin-Streptomycin. Samples were incubated at 25° C. for 1 hour for SpT3 and DgT binders.

Excess binders were removed by loading and washing a resin column. (The resin column was loaded with GST-Catchers, washed, followed by loading of the assembly mixture.) The assemblies were visualised by SDS-PAGE to validate binder removal. This procedure is described in more detail in Example 2: Methods (under Catcher-based protein conjugation).

### Cell Bio Imaging

### Anti-his & Anti-CutA1 Imaging:

8-well chamber slides (Lab-Tek) were coated with 200 μL Poly-L-lysine for 1 h at RT under mild shaking. Slides were washed 3× with 500 μL filtered MilliQ water and dried for 2 h at RT. NCI-N87 cells were seeded at 1.5×10⁴ cells/well into 8-well chamber slides in 300 μL RPMI-1640 medium supplemented with 10% fetal calf serum and incubated at 37° C. 5% CO₂. After 24 h cells were treated with binders or full assembly at 200 nM concentrations and incubated for 2 h45 mins at 37° C. 5% CO₂. Cells were washed 2× with DPBS and fixed with 4% paraformaldehyde in PBS for 15 mins at RT. Then, cells were washed 2×5 mins with DPBS-T (0.1% Triton X-100) and blocked in DPBS-T, 5% FCS (blocking buffer) for 1 h at RT. For immunofluorescent staining cells were treated with a primary antibody (a. anti-His antibody, proteintech 66005, b. anti-CutA1 antibody, abcam ab192236), 1:500 dilution in blocking buffer at 4° C. overnight. Cells were washed 3×5 mins with DPBS-T at RT and stained with a secondary antibody (a. goat anti-mouse IgG H&L DL488 b. goat anti-rabbit IgG H&L DL488, Abbexa), washed 3×5 mins with DPBS-T at RT and mounted using Fluoroshild mounting medium with DAPI (Abcam). Cells were imaged on the Nikon Ti2 inverted confocal microscope and images were analysed using Fiji.

### Fluorescein-Imaging:

8-well chamber slides (Lab-Tek) were coated with 200 μL Poly-L-lysine for 1 h at RT under mild shaking. Slides were washed 3× with 500 μL filtered MilliQ water and dried for 2 h at RT. NCI-N87 cells were seeded at 2.5×10⁴ cells/well into 8-well chamber slides in 300 μL RPMI-1640 medium supplemented with 10% fetal calf serum and incubated at 37° C. 5% CO₂. After 24 h cells were treated with fluorescein-5-maleimide-labelled catcher core CC042 (HsCutA1*, 100 nM), single assemblies (HsCutA1*:L1, 100 nM; L2: HsCutA1*, 300 nM) or full assembly (L2: HsCutA1*:L1, 100 nM) or full assembly and incubated for 4 h at 37° C. 5% CO₂. Cells were washed 2× with complete medium followed by 2 washes in DPBS and fixation with 4% paraformaldehyde in PBS for 15 mins at RT. Then, cells were washed 3×5 mins with DPBS-T (0.05% Tween 20) and mounted using Fluoroshield mounting medium with DAPI (Abcam). Cells were imaged on the Nikon Ti2 inverted confocal microscope and images were analysed using Fiji.

### Example 14: Single-Chain CutA1 for 1+1 Stoichiometry Controls at Fixed Geometry

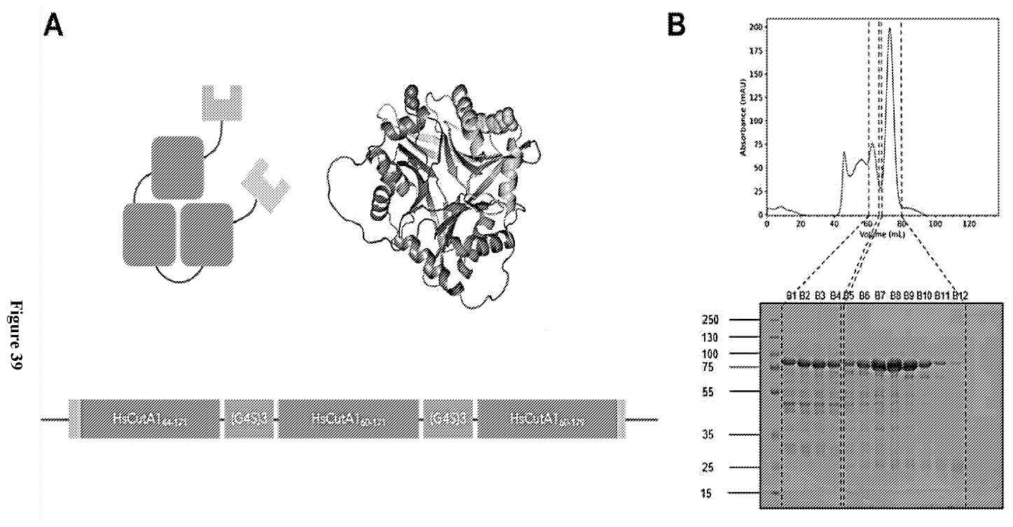

In order to allow for the separate control of bispecificity and multivalency, a single-chain HsCutA1 protein (scHsCutA1 as in SEQ ID NO: 101) was designed that results in 1+1 format in the final assembled molecules while containing the same geometry. The construct was designed to contain a single N-terminal SpyCatcher003 and C-terminal DogCatcher, with each HsCutA1 fused via a (GGGGS)3 linker (FIG. 39).

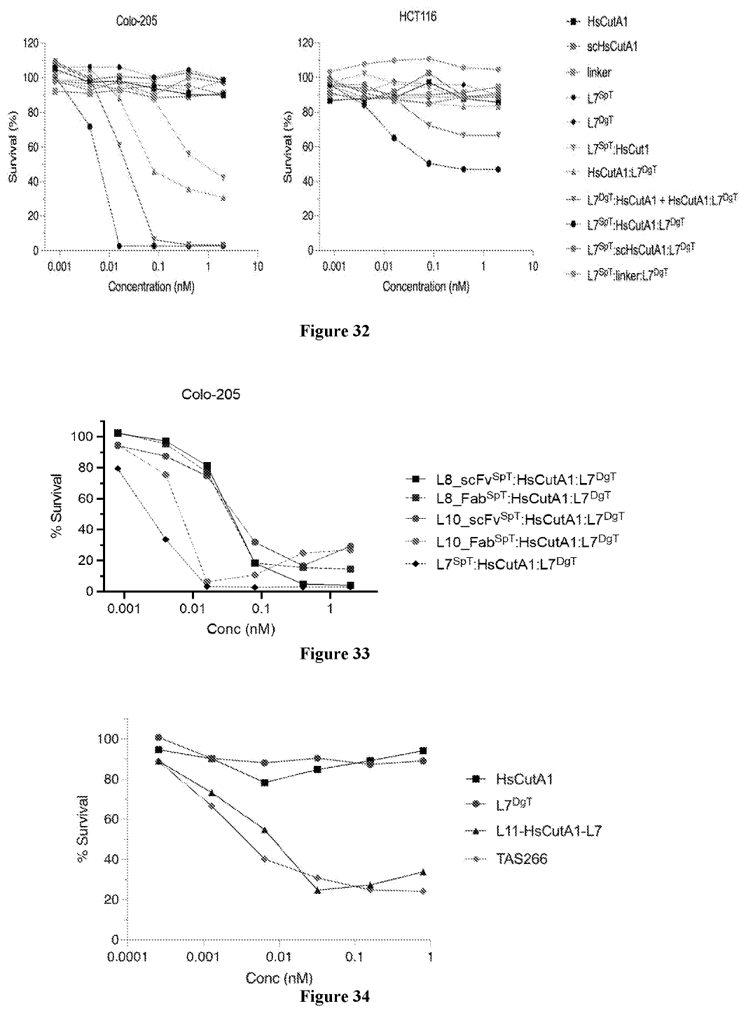

Its applicability has been demonstrated during the assessment of HsCutA1-induced agonism of DR5 targeting protein assemblies (FIG. 32). To demonstrate multivalency as the mechanism of action, HsCutA1, scHsCutA1 and ribosomal linker-based protein assemblies have been used to treat the trail-sensitive cell line Colo-205 and the semitrail-sensitive cell line HCT116 followed by cell viability measurements. In both cell lines the hexavalent HsCutA1 assembly resulted in the relatively lowest about of cell survival in both cell lines. The trivalent HsCutA1:L7-DgT assembly has a moderate effect in the trail-sensitive cell line, but no effect on cell survival in the semi-sensitive cell line. The scHsCutA1 and linker-based assemblies both showed no effect on cell viability in either cell line, demonstrating multivalent formatting of DR5 is required to induce cytotoxicity in either cell line with hexavalency showing higher potency to trivalency in both models. The scCutA1 assembly serves as a 1+1 control with fixed geometry comparable to the trimer HsCutA1, whereas the ribosomal linker L9 is a more generic 1+1 control.

ScCutA1 assemblies have the potential to be used in further in vitro and in vivo applications, such as increasing the drug-to-antibody ratio (DAR) of drug candidates with a preferred 1+1 format with the same geometry as trimeric HsCutA1.

### Brief Description of the Informal Sequence Listing

SEQ ID NO: 1 shows the amino acid sequence of a monomer of the CutA1 protein from Pyrococcus horikoshii (PhCutA1).

SEQ ID NO: 2 shows the amino acid sequence of a monomer of the Collagen X NC1 protein domain.

SEQ ID NO: 3 shows the amino acid sequence of a monomer of the Collagen VIII protein.

SEQ ID NO: 4 shows the amino acid sequence of “SpyCatcher”. This is also referred to herein as “SpyCatcher 001”.

SEQ ID NO: 5 shows the amino acid sequence of “SpyTag”. This is also referred to herein as “SpyTag 001”.

SEQ ID NO: 6 shows the amino acid sequence of “SpyCatcher 002”.

SEQ ID NO: 7 shows the amino acid sequence of “SpyTag 002”.

SEQ ID NO: 8 shows the amino acid sequence of “SpyCatcher 003”.

SEQ ID NO: 9 shows the amino acid sequence of “SpyTag 003”.

SEQ ID NO: 10 shows the amino acid sequence of “SpyLigase”.

SEQ ID NO: 11 shows the amino acid sequence of “K-Tag”.

SEQ ID NO: 12 shows the amino acid sequence of “SnoopCatcher”.

SEQ ID NO: 13 shows the amino acid sequence of “SnoopTag”.

SEQ ID NO: 14 shows the amino acid sequence of “SnoopLigase”.

SEQ ID NO: 15 shows the amino acid sequence of “SnoopTagJr”.

SEQ ID NO: 16 shows the amino acid sequence of “DogTag”.

SEQ ID NO: 17 shows the amino acid sequence of Pilin-C.

SEQ ID NO: 18 shows the amino acid sequence of “Isopeptag”.

SEQ ID NO: 19 shows the amino acid sequence of a monomer of the human CutA1 protein.

SEQ ID NO: 20 shows the amino acid sequence of a monomer of the his-tagged construct H6-SpyCatcher-NC1-SnoopCatcher.

SEQ ID NO: 21 shows the amino acid sequence of a monomer of the his-tagged construct H6-SpyCatcher-PhCutA1-SnoopCatcher.

SEQ ID NO: 22 shows the amino acid sequence of a H6-SpyCatcher-αH_Linker-SnoopCatcher construct.

SEQ ID NO: 23 shows the amino acid sequence of “DogCatcher”.

SEQ ID NO: 24 shows the amino acid sequence of a monomer of the His-tagged construct H6-SpyCatcher003-HsCutA1-DogCatcher, with HsCutA1 truncated as in SEQ ID NO: 29.

SEQ ID NO: 25 shows the amino acid sequence of a monomer of macrophage migration inhibitory factor (MIF).

SEQ ID NO: 26 shows the amino acid sequence of a monomer of human macrophage migration inhibitory factor 2 (MIF2).

SEQ ID NO: 27 shows the amino acid sequence of a monomer of human macrophage migration inhibitory factor 2 (MIF2) with mutations S62A and F99A of MIF (MIF2m).

SEQ ID NO: 28 shows the amino acid sequence of a monomer of the His-tagged construct H6-SpyCatcher003-MIF2m-DogCatcher with mutations S62A and F99A of MIF2 (MIF2m).

SEQ ID NO: 29 shows the amino acid sequence of a monomer of the human CutA1 truncated based on the resolved structure in PDB ID: 2zfh, representing an intermediate truncation between SEQ ID NO: 19 and SEQ ID NO: 60.

SEQ ID NO: 30 shows the amino acid sequence of the His-tagged fusion of DogTag to a variant (first described in Grünberg et al, 2013) of mCitrine fluorescent protein DgT-X3.

SEQ ID NO: 31 shows the amino acid sequence of a monomer of the TNF-like protein TL1A.

SEQ ID NO: 32 shows the amino acid sequence of a monomer of the construct SpyCatcher003-TL1A-DogCatcher as used in structural prediction.

SEQ ID NO: 33 shows the amino acid sequence of a monomer of the construct SpyCatcher003-Col XV NC1-DogCatcher as used in structural prediction.

SEQ ID NO: 34 shows the amino acid sequence of a monomer of the construct SpyCatcher003-MIF2m-DogCatcher as used in structural prediction with mutations S62A and F99A of MIF2.

SEQ ID NO: 35 shows the amino acid sequence of a monomer of the construct SpyCatcher003-IMX-DogCatcher as used in structural prediction.

SEQ ID NO: 36 shows the amino acid sequence of Chain A of heterotrimeric C1q head domain.

SEQ ID NO: 37 shows the amino acid sequence of Chain B of heterotrimeric C1q head domain.

SEQ ID NO: 38 shows the amino acid sequence of Chain C of heterotrimeric C1q head domain.

SEQ ID NO: 39 shows the amino acid sequence of a monomer of the CutA1 from Thermus Thermophilus HB8.

SEQ ID NO: 40 shows the amino acid sequence of a monomer of the CutA1 from Oryza sativa.

SEQ ID NO: 41 shows the amino acid sequence of a monomer of the CutA1 from Shewanella sp. SIB1.

SEQ ID NO: 42 shows the amino acid sequence of a monomer of soluble human tumor necrosis factor alpha (TNF-α or TNF) as resolved in PDB ID 1TNF.

SEQ ID NO: 43 shows the amino acid sequence of a monomer of the antiparallel coiled coil hexamer.

SEQ ID NO: 44 shows the amino acid sequence of a monomer of the HIV-1 GP41 core.

SEQ ID NO: 45 shows the amino acid sequence of a monomer of a circular permutant of cytochrome c555.

SEQ ID NO: 46 shows the amino acid sequence of a monomer of the MHC class II-associated invariant chain.

SEQ ID NO: 47 shows the amino acid sequence of a monomer of the p53.

SEQ ID NO: 48 shows the amino acid sequence of a monomer of a fibrinogen-like domain.

SEQ ID NO: 49 shows the amino acid sequence of a monomer of the Collagen IV NC1 domain.

SEQ ID NO: 50 shows the amino acid sequence of a monomer of the Bacillus subtilis ArbB.

SEQ ID NO: 51 shows the amino acid sequence of a monomer of the phage lambda head protein D.

SEQ ID NO: 52 shows the amino acid sequence of a monomer of a domain-swapped trimer variant of HCRBPII.

SEQ ID NO: 53 shows the amino acid sequence of a monomer of the T1L reovirus attachment protein signal.

SEQ ID NO: 54 shows the amino acid sequence of a monomer of the construct SpyCatcher003-HsCutA1-DogCatcher as used in structural prediction.

SEQ ID NO: 55 shows the amino acid sequence of a monomer of the construct SpyCatcher003-PhCutA1-DogCatcher as used in structural prediction.

SEQ ID NO: 56 shows the amino acid sequence of a monomer of the construct SpyCatcher003-Col X NC1-DogCatcher as used in structural prediction.

SEQ ID NO: 57 shows the amino acid sequence of a monomer of the construct SpyCatcher003-TNF-DogCatcher as used in structural prediction.

SEQ ID NO: 58 shows the amino acid sequence of a monomer of the TNF family protein CD40 ligand (CD40L).

SEQ ID NO: 59 shows the amino acid sequence of a monomer of human leukotriene C4 synthase.

SEQ ID NO: 60 shows the amino acid sequence of a monomer of the human CutA1 as resolved in PDB ID: 2zfh, representing a truncation of SEQ ID NO: 19.

SEQ ID NO: 61 shows the amino acid sequence of a GGGGS linker fused to HsCutA1 to create a direct fusion construct without SpyCatcher, DogCatcher, or SnoopCatcher modules.

SEQ ID NO: 62 shows the amino acid sequence of a (GGGGS)3 linker fused to HsCutA1 to create a direct fusion construct without SpyCatcher, DogCatcher, or SnoopCatcher modules.

SEQ ID NO: 63 shows the amino acid sequence of a (GGGGS)4 linker fused to HsCutA1 to create a direct fusion construct without SpyCatcher, DogCatcher, or SnoopCatcher modules.

SEQ ID NO: 64 shows the amino acid sequence of a (EAAAK)₇ linker fused to HsCutA1 to create a direct fusion construct without SpyCatcher, DogCatcher, or SnoopCatcher modules.

SEQ ID NO: 65 shows the amino acid sequence of a Ribosomal L9 protein α-helical linker fused to HsCutA1 to create a direct fusion construct without SpyCatcher, DogCatcher, or SnoopCatcher modules.

SEQ ID NO: 66 shows the amino acid sequence of a monomer of human CutA1 truncated at both termini to HsCutA1₆₀₋₁₇₁.

SEQ ID NO: 67 shows the amino acid sequence of a monomer of human CutA1 as in SEQ ID NO: 29 with C75A and C96A mutation.

SEQ ID NO: 68 shows the amino acid sequence of a monomer of human CutA1 as in SEQ ID NO: 29 with C75V and C96S mutation.

SEQ ID NO: 69 shows the amino acid sequence of a monomer of human CutA1 as in SEQ ID NO: 66 with C75A and C96A mutation.

SEQ ID NO: 70 shows the amino acid sequence of a monomer of human CutA1 as in SEQ ID NO: 66 with C75V and C96S mutation.

SEQ ID NO: 71 shows the amino acid sequence of a monomer of the His-tagged construct H6-SpyCatcher003-(GGGGS)3-HsCutA1₄₄₋₁₇₉-(GGGGS)3-DogCatcher, with HsCutA1 truncated as in SEQ ID NO: 29 and with linker as in SEQ ID NO: 62.

SEQ ID NO: 72 shows the amino acid sequence of a monomer of the His-tagged construct H6-SpyCatcher003-(GGGGS)3-[C75A, C96A]HsCutA1₄₄₋₁₇₉-(GGGGS)3-DogCatcher, with HsCutA1 truncated as in SEQ ID NO: 67 and with linker as in SEQ ID NO: 62.

SEQ ID NO: 73 shows the amino acid sequence of a monomer of the His-tagged construct H6-SpyCatcher003-(GGGGS)3-[C75V, C96S]HsCutA1₄₄₋₁₇₉-(GGGGS)3-DogCatcher, with HsCutA1 truncated as in SEQ ID NO: 68 and with linker as in SEQ ID NO: 62.

SEQ ID NO: 74 shows the amino acid sequence of a monomer of the His-tagged construct H6-SpyCatcher003-(GGGGS)3-HsCutA1₆₀₋₁₇₁-(GGGGS)3-DogCatcher, with HsCutA1 truncated as in SEQ ID NO: 66 and with linker as in SEQ ID NO: 62.

SEQ ID NO: 75 shows the amino acid sequence of a monomer of the His-tagged construct H6-SpyCatcher003-(GGGGS)3-[C75A, C96A]HsCutA1₆₀₋₁₇₁-(GGGGS)3-DogCatcher, with HsCutA1 truncated as in SEQ ID NO: 69 and with linker as in SEQ ID NO: 62.

SEQ ID NO: 76 shows the amino acid sequence of a monomer of the His-tagged construct H6-SpyCatcher003-(GGGGS)3-[C75V, C96S]HsCutA1₆₀₋₁₇₁-(GGGGS)3-DogCatcher, with HsCutA1 truncated as in SEQ ID NO: 70 and with linker as in SEQ ID NO: 62.

SEQ ID NO: 77 shows the amino acid sequence of a monomer of human CutA1 truncated at both termini to HsCutA1₆₇₋₁₇₁ with C75A and C96A mutation.

SEQ ID NO: 78 shows the amino acid sequence of a monomer of human OX40L truncated to OX40L₅₈₋₁₈₃.

SEQ ID NO: 79 shows the amino acid sequence of a monomer of human CD40L truncated to CD40L₁₂₀₋₂₆₁, representing a truncation of SEQ ID: 58.

SEQ ID NO: 80 shows the amino acid sequence of a monomer of soluble human tumor necrosis factor alpha (TNF-α or TNF), as crystallized in PDB ID 1TNF and as demonstrated in FIG. 23e, fully incorporating SEQ ID NO: 42 and also referred to herein as TNF₇₇₋₂₃₃.

SEQ ID NO: 81 shows the amino acid sequence of a monomer of the PII family protein GlnB from Synechococcus elongatus PCC 7942.

SEQ ID NO: 82 shows the amino acid sequence of a monomer of the PII family protein GlnK from Escherichia coli.

SEQ ID NO: 83 shows the amino acid sequence of the PII-like domain (YqfO₁₃₅₋₂₃₉) of a monomer of the YqfO protein from Bacillus cereus ATCC 14579.

SEQ ID NO: 84 shows the amino acid sequence of the PII-like domain (SA1388₁₂₆₋₂₃₆) of a monomer of the SA1388 protein from Staphylococcus aureus subsp. aureus N315.

SEQ ID NO: 85 shows the amino acid sequence of a monomer of human CutA1 as in SEQ ID NO: 67 with a E78C mutation.

SEQ ID NO: 86 shows the amino acid sequence of a monomer of human CutA1 as in SEQ ID NO: 67 with a K82C mutation.

SEQ ID NO: 87 shows the amino acid sequence of a monomer of human CutA1 as in SEQ ID NO: 67 with a Q102C mutation.

SEQ ID NO: 88 shows the amino acid sequence of a monomer of human CutA1 as in SEQ ID NO: 67 with a E114C mutation.

SEQ ID NO: 89 shows the amino acid sequence of a monomer of human CutA1 as in SEQ ID NO: 67 with a F136C mutation.

SEQ ID NO: 90 shows the amino acid sequence of a monomer of human CutA1 as in SEQ ID NO: 67 with a Q166C mutation.

SEQ ID NO: 91 shows the amino acid sequence of the His-tagged construct H6-SpyCatcher003-GGGGS-[C75A, C96A]HsCutA1₄₄₋₁₇₉-GGGGS-DogCatcher, with HsCutA1 truncated as in SEQ ID NO: 67 and with linker as in SEQ ID NO: 61.

SEQ ID NO: 92 shows the amino acid sequence of the His-tagged construct H6-SpyCatcher003-GGGGS-[C75A, C96A, E78C]HsCutA1₄₄₋₁₇₉-GGGGS-DogCatcher, with HsCutA1 truncated as in SEQ ID NO: 85 and with linker as in SEQ ID NO: 61.

SEQ ID NO: 93 shows the amino acid sequence of the His-tagged construct H6-SpyCatcher003-GGGGS-[C75A, C96A, K82C]HsCutA1₄₄₋₁₇₉-GGGGS-DogCatcher, with HsCutA1 truncated as in SEQ ID NO: 86 and with linker as in SEQ ID NO: 61.

SEQ ID NO: 94 shows the amino acid sequence of the His-tagged construct H6-SpyCatcher003-GGGGS-[C75A, C96A, Q102C]HsCutA1₄₄₋₁₇₉-GGGGS-DogCatcher, with HsCutA1 truncated as in SEQ ID NO: 87 and with linker as in SEQ ID NO: 61.

SEQ ID NO: 95 shows the amino acid sequence of the His-tagged construct H6-SpyCatcher003-GGGGS-[C75A, C96A, E114C]HsCutA1₄₄₋₁₇₉-GGGGS-DogCatcher, with HsCutA1 truncated as in SEQ ID NO: 88 and with linker as in SEQ ID NO: 61.

SEQ ID NO: 96 shows the amino acid sequence of the His-tagged construct H6-SpyCatcher003-GGGGS-[C75A, C96A, F136C]HsCutA1₄₄₋₁₇₉-GGGGS-DogCatcher, with HsCutA1 truncated as in SEQ ID NO: 89 and with linker as in SEQ ID NO: 61.

SEQ ID NO: 97 shows the amino acid sequence of the His-tagged construct H6-SpyCatcher003-GGGGS-[C75A, C96A, Q166C]HsCutA1₄₄₋₁₇₉-GGGGS-DogCatcher, with HsCutA1 truncated as in SEQ ID NO: 90 and with linker as in SEQ ID NO: 61.

SEQ ID NO: 98 shows the amino acid sequence of the His-tagged construct H6-SpyCatcher003-(GGGGS)3-L9-(GGGGS)3-DogCatcher, with L9 as in SEQ ID NO: 65 and with linker as in SEQ ID NO: 62.

SEQ ID NO: 99 shows the amino acid sequence of the His-tagged construct H6-SpyCatcher003-(GGGGS)-scHsCutA1-(GGGGS)-DogCatcher, with scHsCutA1 as in SEQ ID NO: 101 and with linker as in SEQ ID NO: 61.

SEQ ID NO: 100 shows the amino acid sequence of a monomer of human CutA1 as in SEQ ID NO: 19 with C75V and C96S mutation.

SEQ ID NO: 101: shows the amino acid sequence of single chain human CutA1 (scHsCutA1) construct HsCutA1₄₄₋₁₇₁-(GGGGS)3-HsCutA1₆₀₋₁₇₁-(GGGGS)3-HsCutA160-171, with HsCutA1₄₄₋₁₇₁ as a truncation of SEQ ID NO: 100, HsCutA1₆₀₋₁₇₁ truncated as in

SEQ ID NO: 69, HsCutA1₆₀₋₁₇₁ as a truncation of SEQ ID NO: 100 and with linker as in SEQ ID NO: 62.

SEQ ID NO: 102 shows the amino acid sequence of the His-tagged construct HsCutA160-171-(GGGGS)5-L7, with HsCutA1 as in SEQ ID NO: 69, L7 as VHH (TAS266, SEQ ID NO: 106) and with linker as in SEQ ID NO: 103, representing a partial sequence of L11-HsCutA1-L7 as used in FIG. 41.

SEQ ID NO: 103 shows the amino acid sequence of a (GGGGS)5 linker fused to HsCutA1 to create a direct fusion construct without SpyCatcher, DogCatcher, or SnoopCatcher modules.

SEQ ID NO: 104 shows the amino acid sequence of His-GST-(GGGGS)3-SpyCatcher 003.

SEQ ID NO: 105 shows the amino acid sequence of His-GST-(GGGGS)3-DogCatcher.

SEQ ID NO. 106 shows the amino acid sequence of a single anti-DR5 VHH domain of the multivalent DR5 agonist TAS266.

SEQ ID NO: 1
MIIVYTTFPDWESAEKVVKTLLKERLIACANLREHRAFYWWEGKIEEDKEVGAILKTRED
LWEELKERIKELHPYDVPAIIRIDVDDVNEDYLKWLIEETKK
SEQ ID NO: 2
TGMPVSAFTVILSKAYPAIGTPIPFDKILYNRQQHYDPRTGIFTCQIPGIYYFSYHVHVK
GTHVWVGLYKNGTPVMYTYDEYTKGYLDQASGSAIIDLTENDQVWLQLPNAESNGLYSSF
YVHSSFSGFLVAPM
SEQ ID NO: 3
EMPAFTAELTVPFPPVGAPVKFDKLLYNGRQNYNPQTGIFTCEVPGVYYFAYHVHCKGGN
VWVALFKNNEPMMYTYDEYKKGFLDQASGSAVLLLRPGDQVFLQMPSEQAAGLYAGQYVH
SSFSGYLLYPM
SEQ ID NO: 4
VDTLSGLSSEQGQSGDMTIEEDSATHIKFSKRDEDGKELAGATMELRDSSGKTISTWISD
GQVKDFYLYPGKYTFVETAAPDGYEVATAITFTVNEQGQVTVNGKATKGDAHI
SEQ ID NO: 5
AHIVMVDAYKPTK
SEQ ID NO: 6
VTTLSGLSGEQGPSGDMTTEEDSATHIKFSKRDEDGRELAGATMELRDSSGKTISTWISD
GHVKDFYLYPGKYTFVETAAPDGYEVATAITFTVNEQGQVTVNGEATKGDAHT
SEQ ID NO: 7
VPTIVMVDAYKRYK
SEQ ID NO: 8
VTTLSGLSGEQGPSGDMTTEEDSATHIKFSKRDEDGRELAGATMELRDSSGKTISTWISD
GHVKDFYLYPGKYTFVETAAPDGYEVATPIEFTVNEDGQVTVDGEATEGDAHT
SEQ ID NO: 9
RGVPHIVMVDAYKRYK
SEQ ID NO: 10
GQSGDGKELAGATMELRDSSGKTISTWISDGQVKDFYLYPGKYTFVETAAPDGYEVATAI
TFTVNEQGQVTVNGKATKGGSGGSGGSGEDSATHI
SEQ ID NO: 11
ATHIKFSKRD
SEQ ID NO: 12
KPLRGAVFSLQKQHPDYPDIYGAIDQNGTYQNVRTGEDGKLTFKNLSDGKYRLFENSEPA
GYKPVQNKPIVAFQIVNGEVRDVTSIVPQDIPATYEFTNGKHYITNEPIPPK
SEQ ID NO: 13
KLGDIEFIKVNK
SEQ ID NO: 14
VNKNDKKPLRGAVFSLQKQHPDYPDIYGAIDQNGTYQNVRTGEDGKLTFKNLSDGKYRLF
ENSEPPGYKPVQNKPIVAFQIVNGEVRDVTSIVPPGVPATYEFT
SEQ ID NO: 15
KLGSIEFIKVNK
SEQ ID NO: 16
DIPATYEFTDGKHYITNEPIPPK
SEQ ID NO: 17
ATTVHGETVVNGAKLTVTKNLDLVNSNALIPNTDFTFKIEPDTTVNEDGNKFKGVALNTP
MTKVTYTNSDKGGSNTKTAEFDFSEVTFEKPGVYYYKVTEEKIDKVPGVSYDTTSYTVQV
HVLWNEEQQKPVATYIVGYKEGSKVPIQFKNSLDSTTLTVKKKVSGTGGDRSKDFNFGLT
LKANQYYKASEKVMIEKTTKGGQAPVQTEASIDQLYHFTLKDGESIKVTNLPVGVDYVVT
EDDYKSEKYTTNVEVSPQDGAVKNIAGNSTEQETSTDKDMTI
SEQ ID NO: 18
TDKDMTITFTNKKDAE
SEQ ID NO: 19
MSGGRAPAVLLGGVASLLLSFVWMPALLPVASRLLLLPRVLLTMASGSPPTQPSPASDSG
SGYVPGSVSAAFVTCPNEKVAKEIARAVVEKRLAACVNLIPQITSIYEWKGKIEEDSEVL
MMIKTQSSLVPALTDFVRSVHPYEVAEVIALPVEQGNFPYLQWVRQVTESVSDSITVLP
SEQ ID NO: 20
MGSSHHHHHHSSGVDTLSGLSSEQGQSGDMTIEEDSATHIKFSKRDEDGKELAGATMELR
DSSGKTISTWISDGQVKDFYLYPGKYTFVETAAPDGYEVATAITFTVNEQGQVTVNGKAT
KGDAHIGSGSTGMPVSAFTVILSKAYPAIGTPIPFDKILYNRQQHYDPRTGIFTCQIPGI
YYFSYHVHVKGTHVWVGLYKNGTPVMYTYDEYTKGYLDQASGSAIIDLTENDQVWLQLPN
AESNGLYSSEYVHSSFSGFLVAPMGSGSKPLRGAVFSLQKQHPDYPDIYGAIDQNGTYQN
VRTGEDGKLTFKNLSDGKYRLFENSEPAGYKPVQNKPIVAFQIVNGEVRDVTSIVPQDIP
ATYEFTNGKHYITNEPIPPK
SEQ ID NO: 21
MGSSHHHHHHSSGVDTLSGLSSEQGQSGDMTIEEDSATHIKFSKRDEDGKELAGATMELR
DSSGKTISTWISDGQVKDFYLYPGKYTFVETAAPDGYEVATAITFTVNEQGQVTVNGKAT
KGDAHIGSGSMIIVYTTFPDWESAEKVVKTLLKERLIACANLREHRAFYWWEGKIEEDKE
VGAILKTREDLWEELKERIKELHPYDVPAIIRIDVDDVNEDYLKWLIEETKKGSGSKPLR
GAVFSLQKQHPDYPDIYGAIDQNGTYQNVRTGEDGKLTFKNLSDGKYRLFENSEPAGYKP
VQNKPIVAFQIVNGEVRDVTSIVPQDIPATYEFTNGKHYITNEPIPPK
SEQ ID NO: 22
MGSSHHHHHHSSGVDTLSGLSSEQGQSGDMTIEEDSATHIKFSKRDEDGKELAGATMELR
DSSGKTISTWISDGQVKDFYLYPGKYTFVETAAPDGYEVATAITFTVNEQGQVTVNGKAT
KGDAHIGSGSPANLKALEAQKQKEQRQAAEELANAKKLKEQLEKGSGSKPLRGAVFSLQK
QHPDYPDIYGAIDQNGTYQNVRTGEDGKLTFKNLSDGKYRLFENSEPAGYKPVQNKPIVA
FQIVNGEVRDVTSIVPQDIPATYEFTNGKHYITNEPIPPK
SEQ ID NO 23
KLGEIEFIKVDKTDKKPLRGAVFSLQKQHPDYPDIYGAIDQNGTYQDVRTGEDGKLTFTN
LSDGKYRLIENSEPPGYKPVQNKPIVSFRIVDGEVRDVTSIVPQ
SEQ ID NO 24
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSMASGSPPTQPSPASDSGSGYVPGSVSAAFVTCPNEKVAKEIARAVVEKR
LAACVNLIPQITSIYEWKGKIEEDSEVLMMIKTQSSLVPALTDFVRSVHPYEVAEVIALP
VEQGNFPYLQWVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYKPVQNKPIVSF
RIVDGEVRDVTSIVPQ
SEQ ID NO: 25
PMFIVNTNVPRASVPDGFLSELTQQLAQATGKPPQYIAVHVVPDQLMAFGGSSEPCALCS
LHSIGKIGGAQNRSYSKLLCGLLAERLRISPDRVYINYYDMNAANVGWNNSTFA
SEQ ID NO: 26
PFLELDTNLPANRVPAGLEKRLCAAAASILGKPADRVNVTVRPGLAMALSGSTEPCAQLS
ISSIGVVGTAEDNRSHSAHFFEFLTKELALGQDRILIRFFPLESWQIGKIGTVMTFL
SEQ ID NO: 27
PFLELDTNLPANRVPAGLEKRLCAAAASILGKPADRVNVTVRPGLAMALSGSTEPCAQLS
IASIGVVGTAEDNRSHSAHFFEFLTKELALGQDRILIRFAPLESWQIGKIGTVMTFL
SEQ ID NO: 28
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSGGGGSGGGGSPFLELDTNLPANRVPAGLEKRLCAAAASILGKPADRVNV
TVRPGLAMALSGSTEPCAQLSIASIGVVGTAEDNRSHSAHFFEFLTKELALGQDRILIRF
APLESWQIGKIGTVMTFLGGGGSGGGGSGGGGSKLGEIEFIKVDKTDKKPLRGAVFSLQK
QHPDYPDIYGAIDQNGTYQDVRTGEDGKLTFTNLSDGKYRLIENSEPPGYKPVQNKPIVS
FRIVDGEVRDVTSIVPQ
SEQ ID NO: 29
MASGSPPTQPSPASDSGSGYVPGSVSAAFVTCPNEKVAKEIARAVVEKRLAACVNLIPQI
TSIYEWKGKIEEDSEVLMMIKTQSSLVPALTDFVRSVHPYEVAEVIALPVEQGNFPYLQW
VRQVTESVSDSITVLP
SEQ ID NO: 30
MSHHHHHHGSTGLEVLFQGPTGSSDIPATYEFTDGKHYITNEPIPPKGGGGSGGGGSVSK
GEELFTGVVPILVELDGDVNGHKFSVSGEGEGDATYGKLELKFICTTGKLPVPWPTLVTT
FGYGLMCFARYPDHMKQHDFFKSAMPEGYVQERTIFFKDDGNYKTRAEVKFEGDTLVNRI
ELKGIDFKEDGNILGHKLEYDYNSHNVYIMADKQKNGIKVNFKIRHNIEDGSVQLADHYQ
QNTPIGDGPVLLPDNHYLEYQSALSKDPNEKRDHMVLAEFVTAEGITLGMDELYK
SEQ ID NO: 31
LRADGDKPRAHLTVVRQTPTQHFKNQFPALHWEHELGLAFTKNRMNYTNKFLLIPESGDY
FIYSQVTFRGMTSECSEIRQAGRPNKPDSITVVITKVTDSYPEPTQLLMGTKSVCEVGSN
WFQPIYLGAMESLQEGDKLMVNVSDISLVDYTKEDKTFFGAFLL
SEQ ID NO: 32
VTTLSGLSGEQGPSGDMTTEEDSATHIKFSKRDEDGRELAGATMELRDSSGKTISTWISD
GHVKDFYLYPGKYTFVETAAPDGYEVATPIEFTVNEDGQVTVDGEATEGDAHTGSGSLRA
DGDKPRAHLTVVRQTPTQHFKNQFPALHWEHELGLAFTKNRMNYTNKFLLIPESGDYFIY
SQVTFRGMTSECSEIRQAGRPNKPDSITVVITKVTDSYPEPTQLLMGTKSVCEVGSNWFQ
PIYLGAMFSLQEGDKLMVNVSDISLVDYTKEDKTFFGAFLLGSGSKLGEIEFIKVDKTDK
KPLRGAVFSLQKQHPDYPDIYGAIDQNGTYQDVRTGEDGKLTFTNLSDGKYRLIENSEPP
GYKPVQNKPIVSFRIVDGEVRDVTSIVPQ
SEQ ID NO: 33
VTTLSGLSGEQGPSGDMTTEEDSATHIKFSKRDEDGRELAGATMELRDSSGKTISTWISD
GHVKDFYLYPGKYTFVETAAPDGYEVATPIEFTVNEDGQVTVDGEATEGDAGSGSVTAFS
NMDDMLQKAHLVIEGTFIYLRDSTEFFIRVRDGWKKLQLGELIPIPADSPPPPALSSNPG
SGSKLGEIEFIKVDKTDKKPLRGAVFSLQKQHPDYPDIYGAIDQNGTYQDVRTGEDGKLT
FTNLSDGKYRLIENSEPPGYKPVQNKPIVSFRIVDGEVRDVTSIVPQ
SEQ ID NO: 34
VTTLSGLSGEQGPSGDMTTEEDSATHIKESKRDEDGRELAGATMELRDSSGKTISTWISD
GHVKDFYLYPGKYTFVETAAPDGYEVATPIEFTVNEDGQVTVDGEATEGDAHTGSGSPFL
ELDTNLPANRVPAGLEKRLCAAAASILGKPADRVNVTVRPGLAMALSGSTEPCAQLSIAS
IGVVGTAEDNRSHSAHFFEFLTKELALGQDRILIRFAPLESWQIGKIGTVMTFLGSGSKL
GEIEFIKVDKTDKKPLRGAVFSLQKQHPDYPDIYGAIDQNGTYQDVRTGEDGKLTFTNLS
DGKYRLIENSEPPGYKPVQNKPIVSFRIVDGEVRDVTSIVPQ
SEQ ID NO: 35
VTTLSGLSGEQGPSGDMTTEEDSATHIKFSKRDEDGRELAGATMELRDSSGKTISTWISD
GHVKDFYLYPGKYTFVETAAPDGYEVATPIEFTVNEDGQVTVDGEATEGDAHTGSGSKKQ
GDADVCGEVAYIQSVVSDCHVPTAELRTLLEIRKLFLEIQKLKVELQGLSKEGSGSKLGE
IEFIKVDKTDKKPLRGAVFSLQKQHPDYPDIYGAIDQNGTYQDVRTGEDGKLTFTNLSDG
KYRLIENSEPPGYKPVQNKPIVSFRIVDGEVRDVTSIVPQ
SEQ ID NO: 36
QPRPAFSAIRRNPPMGGNVVIFDTVITNQEEPYQNHSGRFVCTVPGYYYFTFQVLSQWEI
CLSIVSSSRGQVRRSLGFCDTTNKGLFQVVSGGMVLQLQQGDQVWVEKDPKKGHIYQGSE
ADSVFSGFLIFPS
SEQ ID NO: 37
TQKIAFSATRTINVPLRRDQTIRFDHVITNMNNNYEPRSGKFTCKVPGLYYFTYHASSRG
NLCVNLMRGRERAQKVVTFCDYAYNTFQVTTGGMVLKLEQGENVFLQATDKNSLLGMEGA
NSIFSGFLLFPD
SEQ ID NO: 38
KFQSVFTVTRQTHQPPAPNSLIRFNAVLTNPQGDYDTSTGKFTCKVPGLYYFVYHASHTA
NLCVLLYRSGVKVVTFCGHTSKTNQVNSGGVLLRLQVGEEVWLAVNDYYDMVGIQGSDSV
FSGFLLFPD
SEQ ID NO: 39
MEEVVLITVPSEEVARTIAKALVEERLAACVNIVPGLTSIYRWQGEVVEDQELLLLVKTT
THAFPKLKERVKALHPYTVPEIVALPIAEGNREYLDWLRENTG
SEQ ID NO: 40
STTVPSIVVYVTVPNKEAGKRLAGSIISEKLAACVNIVPGIESVYWWEGKVQTDAEELLI
IKTRESLLDALTEHVKANHEYDVPEVIALPIKGGNLKYLEWLKNSTR
SEQ ID NO: 41
KPEQLLIFTTCPDADIACRIATALVEAKLAACVQIGQAVESIYQWDNNICQSHEVPMQIK
CMTTDYPAIEQLVITMHPYEVPEFIATPIIGGFGPYLQWIKDNSPS
SEQ ID NO: 42
RTPSDKPVAHVVANPQAEGQLQWLNRRANALLANGVELRDNQLVVPSEGLYLIYSQVLFK
GQGCPSTHVLLTHTISRIAVSYQTKVNLLSAIKSPCQRETPEGAEAKPWYEPIYLGGVFQ
LEKGDRLSAEINRPDYLLFAESGQVYFGIIAL
SEQ ID NO: 43
ELAQAFKEIAKAFKEIAKAFEFIAQAIE
SEQ ID NO: 44
IVQQQNNLLRAIEAQQHLLQLTVWAIKQLQARSGGRGGWMEWDREINNYTSLIHSLIEES
Q
SEQ ID NO: 45
VDPAKEAIMKPQLTMLKGLSDAELKALADFILRIAKQAQEKQQQDVAKAIFQQKGCGSCH
QANVDTVGPSLAKIAQAYAGKEDQLIKFLKGEAPAI
SEQ ID NO: 46
YGNMTEDHVMHLLQNADPLKVYPPLKGSFPENLRHLKNTMETIDWKVFESWMHHWLLFEM
SRHSLEQKPTDAPPK
SEQ ID NO: 47
GEYFTLQIRGRERFEMFRELNEALELKDAQAG
SEQ ID NO: 48
SRPRDCLDVLLSGQQDDGVYSVFPTHYPAGFQVYCDMRTDGGGWTVFQRREDGSVNFFRG
WDAYRDGFGRLTGEHWLGLKRIHALTTQAAYELHVDLEDFENGTAYARYGSFGVGLFSVD
PEEDGYPLTVADYSGTAGDSLLKHSGMRFTTKDRDSDHSENNCAAFYRGAWWYRNCHTSN
LNGQYLRGAHASYADGVEWSSWTGWQYSLKFSEMKIRPVR
SEQ ID NO: 49
VDHGFLVTRHSQTIDDPQCPSGTKILYHGYSLLYVQGNERAHGQDLGTAGSCLRKFSTMP
FLFCNINNVCNFASRNDYSYWLSTPEPMPMSMAPITGENIRPFISRCAVCEAPAMVMAVH
SQTIQIPPCPSGWSSLWIGYSFVMHTSAGAEGSGQALASPGSCLEEFRSAPFIECHGRGT
CNYYANAYSFWLATIERSEMFKKPTPSTLKAGELRTHVSRCQVCMRRT
SEQ ID NO: 50
FMKSTGIVRKVDELGRVVIPIELRRTLGIAEKDALEIYVDDEKIILKKYKPN
SEQ ID NO: 51
SDPAHTATAPGGLSAKAPAMTPLMLDTSSRKLVAWDGTTDGAAVGILAVAADQTSTTLTF
YKSGTFRYEDVLWPEAASDETKKRTAFAGTAISIV
SEQ ID NO: 52
TRDFNGTWEMESNENFEGYMKALDIDFATRKIAVRLTFTDVIDQDGDNFKTKATSTFLNY
DEDFTVGVEFDEYTKSLDNRHVKALVTWEGDVLVCVQKGEKENRGWKKWIEGDKLYLELT
CGDQVCRQVFKKK
SEQ ID NO: 53
LPTYRYPLELDTANNRVQVADRFGMRTGTWTGQLQYQHPQLSWRANVTLNLMKVDDWLVL
SFSQMTTNSIMADGKFVINFVSGLSSGWQTGDTEPSSTIDPLSTTFAAVQFLNNGQRIDA
FRIMGVSEWTDGELEIKNYGGTYTGHTQVYWAPWTIMYPCNV
SEQ ID NO: 54
VTTLSGLSGEQGPSGDMTTEEDSATHIKFSKRDEDGRELAGATMELRDSSGKTISTWISD
GHVKDFYLYPGKYTFVETAAPDGYEVATPIEFTVNEDGQVTVDGEATEGDAHTGSGSMAS
GSPPTQPSPASDSGSGYVPGSVSAAFVTCPNEKVAKEIARAVVEKRLAACVNLIPQITSI
YEWKGKIEEDSEVLMMIKTQSSLVPALTDFVRSVHPYEVAEVIALPVEQGNFPYLQWVRQ
VTESVSDSITVLPGSGSKLGEIEFIKVDKTDKKPLRGAVFSLQKQHPDYPDIYGAIDQNG
TYQDVRTGEDGKLTFTNLSDGKYRLIENSEPPGYKPVQNKPIVSFRIVDGEVRDVTSIVP
Q
SEQ ID NO: 55
VTTLSGLSGEQGPSGDMTTEEDSATHIKFSKRDEDGRELAGATMELRDSSGKTISTWISD
GHVKDFYLYPGKYTFVETAAPDGYEVATPIEFTVNEDGQVTVDGEATEGDAHTGSGSMII
VYTTFPDWESAEKVVKTLLKERLIACANLREHRAFYWWEGKIEEDKEVGAILKTREDLWE
ELKERIKELHPYDVPAIIRIDVDDVNEDYLKWLIEETKKGSGSKLGEIEFIKVDKTDKKP
LRGAVFSLQKQHPDYPDIYGAIDQNGTYQDVRTGEDGKLTFTNLSDGKYRLIENSEPPGY
KPVQNKPIVSFRIVDGEVRDVTSIVPQ
SEQ ID NO: 56
VTTLSGLSGEQGPSGDMTTEEDSATHIKFSKRDEDGRELAGATMELRDSSGKTISTWISD
GHVKDFYLYPGKYTFVETAAPDGYEVATPIEFTVNEDGQVTVDGEATEGDAHTGSGSTGM
PVSAFTVILSKAYPAIGTPIPFDKILYNRQQHYDPRTGIFTCQIPGIYYFSYHVHVKGTH
VWVGLYKNGTPVMYTYDEYTKGYLDQASGSAIIDLTENDQVWLQLPNAESNGLYSSEYVH
SSFSGFLVAPMGSGSKLGEIEFIKVDKTDKKPLRGAVFSLQKQHPDYPDIYGAIDQNGTY
QDVRTGEDGKLTFTNLSDGKYRLIENSEPPGYKPVQNKPIVSFRIVDGEVRDVTSIVPQ
SEQ ID NO: 57
VTTLSGLSGEQGPSGDMTTEEDSATHIKFSKRDEDGRELAGATMELRDSSGKTISTWISD
GHVKDFYLYPGKYTFVETAAPDGYEVATPIEFTVNEDGQVTVDGEATEGDAHTGSGSRTP
SDKPVAHVVANPQAEGQLQWLNRRANALLANGVELRDNQLVVPSEGLYLIYSQVLFKGQG
CPSTHVLLTHTISRIAVSYQTKVNLLSAIKSPCQRETPEGAEAKPWYEPIYLGGVFQLEK
GDRLSAEINRPDYLLFAESGQVYFGIIALGSGSKLGEIEFIKVDKTDKKPLRGAVFSLQK
QHPDYPDIYGAIDQNGTYQDVRTGEDGKLTFTNLSDGKYRLIENSEPPGYKPVQNKPIVS
FRIVDGEVRDVTSIVPQ
SEQ ID NO: 58
QIAAHVISEASSKTTSVLQWAEKGYYTMSNNLVTLENGKQLTVKRQGLYYIYAQVTFCSN
REASSQAPFIASLCLKSPGRFERILLRAANTHSSAKPCGQQSIHLGGVFELQPGASVFVN
VTDPSQVSHGTGFTSFGLLKL
SEQ ID NO: 59
MKDEVALLAAVTLLGVLLQAYFSLQVISARRAFRVSPPLTTGPPEFERVYRAQVNCSEYF
PLFLATLWVAGIFFHEGAAALCGLVYLFARLRYFQGYARSAQLRLAPLYASARALWLLVA
LAALGLLAHFLPAALRAALLGRLRTL
SEQ ID NO: 60
SGYVPGSVSAAFVTCPNEKVAKEIARAVVEKRLAACVNLIPQITSIYEWKGKIEEDSEVL
MMIKTQSSLVPALTDFVRSVHPYEVAEVIALPVEQGNFPYLQWVRQVT
SEQ ID NO: 61
GGGGS
SEQ ID NO: 62
GGGGSGGGGSGGGGS
SEQ ID NO: 63
GGGGSGGGGSGGGGSGGGGS
SEQ ID NO: 64
EAAAKEAAAKEAAAKEAAAKEAAAKEAAAKEAAAK
SEQ ID NO: 65
PANLKALEAQKQKEQRQAAEELANAKKLKEQLEK
SEQ ID NO: 66
GSGYVPGSVSAAFVTCPNEKVAKEIARAVVEKRLAACVNLIPQITSIYEWKGKIEEDSEV
LMMIKTQSSLVPALTDFVRSVHPYEVAEVIALPVEQGNFPYLQWVRQVTESV
SEQ ID NO: 67
MASGSPPTQPSPASDSGSGYVPGSVSAAFVTAPNEKVAKEIARAVVEKRLAAAVNLIPQI
TSIYEWKGKIEEDSEVLMMIKTQSSLVPALTDFVRSVHPYEVAEVIALPVEQGNFPYLQW
VRQVTESVSDSITVLP
SEQ ID NO: 68
MASGSPPTQPSPASDSGSGYVPGSVSAAFVTVPNEKVAKEIARAVVEKRLAASVNLIPQI
TSIYEWKGKIEEDSEVLMMIKTQSSLVPALTDFVRSVHPYEVAEVIALPVEQGNFPYLQW
VRQVTESVSDSITVLP
SEQ ID NO: 69
GSGYVPGSVSAAFVTAPNEKVAKEIARAVVEKRLAAAVNLIPQITSIYEWKGKIEEDSEV
LMMIKTQSSLVPALTDFVRSVHPYEVAEVIALPVEQGNFPYLQWVRQVTESV
SEQ ID NO: 70
GSGYVPGSVSAAFVTVPNEKVAKEIARAVVEKRLAASVNLIPQITSIYEWKGKIEEDSEV
LMMIKTQSSLVPALTDFVRSVHPYEVAEVIALPVEQGNFPYLQWVRQVTESV
SEQ ID NO: 71
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSGGGGSGGGGSMASGSPPTQPSPASDSGSGYVPGSVSAAFVTCPNEKVAK
EIARAVVEKRLAACVNLIPQITSIYEWKGKIEEDSEVLMMIKTQSSLVPALTDFVRSVHP
YEVAEVIALPVEQGNFPYLQWVRQVTESVSDSITVLPGGGGSGGGGSGGGGSKLGEIEFI
KVDKTDKKPLRGAVFSLQKQHPDYPDIYGAIDQNGTYQDVRTGEDGKLTFTNLSDGKYRL
IENSEPPGYKPVQNKPIVSFRIVDGEVRDVTSIVPQ
SEQ ID NO: 72
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSGGGGSGGGGSMASGSPPTQPSPASDSGSGYVPGSVSAAFVTAPNEKVAK
EIARAVVEKRLAAAVNLIPQITSIYEWKGKIEEDSEVLMMIKTQSSLVPALTDFVRSVHP
YEVAEVIALPVEQGNFPYLQWVRQVTESVSDSITVLPGGGGSGGGGSGGGGSKLGEIEFI
KVDKTDKKPLRGAVFSLQKQHPDYPDIYGAIDQNGTYQDVRTGEDGKLTFTNLSDGKYRL
IENSEPPGYKPVQNKPIVSFRIVDGEVRDVTSIVPQ
SEQ ID NO: 73
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSGGGGSGGGGSMASGSPPTQPSPASDSGSGYVPGSVSAAFVTVPNEKVAK
EIARAVVEKRLAASVNLIPQITSIYEWKGKIEEDSEVLMMIKTQSSLVPALTDFVRSVHP
YEVAEVIALPVEQGNFPYLQWVRQVTESVSDSITVLPGGGGSGGGGSGGGGSKLGEIEFI
KVDKTDKKPLRGAVFSLQKQHPDYPDIYGAIDQNGTYQDVRTGEDGKLTFTNLSDGKYRL
IENSEPPGYKPVQNKPIVSFRIVDGEVRDVTSIVPQ
SEQ ID NO: 74
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSGGGGSGGGGSGSGYVPGSVSAAFVTCPNEKVAKIARAVVEKRLAACVNL
IPQITSIYEWEKGKIEEDSEVLMMIKTQSSLVPALTDFVRSVHPYEVAEVIALPVEQGNF
PYLQWVRQVTESVGGGGSGGGGSGGGGSKLGEIEFIKVDKTDKKPLRGAVFSLQKQHPDY
PDIYGAIDQNGTYQDVRTGEDGKLTFTNLSDGKYRLIENSEPPGYKPVQNKPIVSFRIVD
GEVRDVTSIVPQ
SEQ ID NO: 75
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSGGGGSGGGGSGSGYVPGSVSAAFVTAPNEKVAKEIARAVVEKRLAAAVN
LIPQITSIYEWKGKIEEDSEVLMMIKTQSSLVPALTDFVRSVHPYEVAEVIALPVEQGNF
PYLQWVRQVTESVGGGGSGGGGSGGGGSKLGEIEFIKVDKTDKKPLRGAVFSLQKQHPDY
PDIYGAIDQNGTYQDVRTGEDGKLTFTNLSDGKYRLIENSEPPGYKPVQNKPIVSFRIVD
GEVRDVTSIVPQ
SEQ ID NO: 76
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSGGGGSGGGGSGSGYVPGSVSAAFVTVPNEKVAKEIARAVVEKRLAASVN
LIPQITSIYEWKGKIEEDSEVLMMIKTQSSLVPALTDFVRSVHPYEVAEVIALPVEQGNF
PYLQWVRQVTESVGGGGSGGGGSGGGGSKLGEIEFIKVDKTDKKPLRGAVFSLQKQHPDY
PDIYGAIDQNGTYQDVRTGEDGKLTFTNLSDGKYRLIENSEPPGYKPVQNKPIVSFRIVD
GEVRDVTSIVPQ
SEQ ID NO: 77
SVSAAFVTAPNEKVAKEIARAVVEKRLAAAVNLIPQITSIYEWKGKIEEDSEVLMMIKTQ
SSLVPALTDFVRSVHPYEVAEVIALPVEQGNFPYLQWVRQVTESV
SEQ ID NO: 78
RIQSIKVQFTEYKKEKGFILTSQKEDEIMKVQDNSVIINCDGFYLISLKGYFSQEVDISL
HYQKDEEPLFQLKKVRSVNSLMVASLTYKDKVYLNVTTDNTSLDDFHVNGGELILIHQNP
GEFCVL
SEQ ID NO: 79
PQIAAHVISEASSKTTSVLQWAEKGYYTMSNNLVTLENGKQLTVKRQGLYYIYAQVTFCS
NREASSQAPFIASLCLKSPGRFERILLRAANTHSSAKPCGQQSIHLGGVFELQPGASVFV
NVTDPSQVSHGTGFTSFGLLKL
SEQ ID NO: 80
VRSSSRTPSDKPVAHVVANPQAEGQLQWLNRRANALLANGVELRDNQLVVPSEGLYLIYS
QVLFKGQGCPSTHVLLTHTISRIAVSYQTKVNLLSAIKSPCQRETPEGAEAKPWYEPIYL
GGVFQLEKGDRLSAEINRPDYLLFAESGQVYFGIIAL
SEQ ID NO: 81
MKKIEAIIRPFKLDEVKIALVNAGIVGMTVSEVRGFGRQKGQTERYRGAEYTVEFLQKLK
LEIVVEDAQVDTVIDKIVAAARTGEIGDGKIFVSPVDQTIRIRTGEKNADAI
SEQ ID NO: 82
MKLVTVIIKPFKLEDVREALSSIGIQGLTVTEVKGFGRQKGHAELYRGAEYSVNFLPKVK
IDVAIADDQLDEVIDIVSKAAYTGKIGDGKIFVAELQRVIRIRTGEADEAAL
SEQ ID NO: 83
EEMKKVVVFVPVTHAEEVRKALGDAGAGHIGNYSHCTFSSEGTGTFVPQEGINPYIGETG
QLERVEEVRIETIIPASLQRKVIKAMVTAHPYEEVAYDVYPLDNKG
SEQ ID NO: 84
SIINNQQDVYYKVQTYIPKDNVGPFKDKLSENGLAQEGNYEYCFFESEGRGQFKPVGEAN
PTIGQIDKIEDVDEVKIEFMIDAYQKSRAEQLIKQYHPYETPVEDFIEIKQ
SEQ ID NO: 85
MASGSPPTQPSPASDSGSGYVPGSVSAAFVTAPNCKVAKEIARAVVEKRLAAAVNLIPQI
TSIYEWKGKIEEDSEVIMMIKTQSSLVPALTDFVRSVHPYEVAEVIALPVEQGNFPYLQW
VRQVTESVSDSITVLP
SEQ ID NO: 86
MASGSPPTQPSPASDSGSGYVPGSVSAAFVTAPNEKVACEIARAVVEKRLAAAVNLIPQI
TSIYEWKGKIEEDSEVLMMIKTQSSLVPALTDFVRSVHPYEVAEVIALPVEQGNFPYLQW
VRQVTESVSDSITVLP
SEQ ID NO: 87
MASGSPPTQPSPASDSGSGYVPGSVSAAFVTAPNEKVAKEIARAVVEKRLAAAVNLIPCI
TSIYEWKGKIEEDSEVIMMIKTQSSLVPALTDFVRSVHPYEVAEVIALPVEQGNFPYLQW
VRQVTESVSDSITVLP
SEQ ID NO: 88
MASGSPPTQPSPASDSGSGYVPGSVSAAFVTAPNEKVAKEIARAVVEKRLAAAVNLIPQI
TSIYEWKGKICEDSEVLMMIKTQSSLVPALTDFVRSVHPYEVAEVIALPVEQGNFPYLQW
VRQVTESVSDSITVLP
SEQ ID NO: 89
MASGSPPTQPSPASDSGSGYVPGSVSAAFVTAPNEKVAKEIARAVVEKRLAAAVNLIPQI
TSIYEWKGKIEEDSEVIMMIKTQSSLVPALTDCVRSVHPYEVAEVIALPVEQGNFPYLQW
VRQVTESVSDSITVLP
SEQ ID NO: 90
MASGSPPTQPSPASDSGSGYVPGSVSAAFVTAPNEKVAKEIARAVVEKRLAAAVNLIPQI
TSIYEWKGKIEEDSEVIMMIKTQSSLVPALTDFVRSVHPYEVAEVIALPVEQGNFPYLQW
VRCVTESVSDSITVLP
SEQ ID NO: 91
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSMASGSPPTQPSPASDSGSGYVPGSVSAAFVTAPNEKVAKEIARAVVEKR
LAAAVNLIPQITSIYEWKGKIEEDSEVLMMIKTQSSLVPALTDFVRSVHPYEVAEVIALP
VEQGNFPYLQWVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYKPVQNKPIVSF
RIVDGEVRDVTSIVPQ
SEQ ID NO: 92
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSMASGSPPTQPSPASDSGSGYVPGSVSAAFVTAPNCKVAKEIARAVVEKR
LAAAVNLIPQITSIYEWKGKIEEDSEVLMMIKTQSSLVPALTDFVRSVHPYEVAEVIALP
VEQGNFPYLQWVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYKPVQNKPIVSF
RIVDGEVRDVTSIVPQ
SEQ ID NO: 93
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSMASGSPPTQPSPASDSGSGYVPGSVSAAFVTAPNEKVACEIARAVVEKR
LAAAVNLIPQITSIYEWKGKIEEDSEVLMMIKTQSSLVPALTDFVRSVHPYEVAEVIALP
VEQGNFPYLQWVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYKPVQNKPIVSF
RIVDGEVRDVTSIVPQ
SEQ ID NO: 94
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSMASGSPPTQPSPASDSGSGYVPGSVSAAFVTAPNEKVAKEIARAVVEKR
LAAAVNLIPCITSIYEWKGKIEEDSEVLMMIKTQSSLVPALTDFVRSVHPYEVAEVIALP
VEQGNFPYLQWVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYKPVQNKPIVSF
RIVDGEVRDVTSIVPQ
SEQ ID NO: 95
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSMASGSPPTQPSPASDSGSGYVPGSVSAAFVTAPNEKVAKEIARAVVEKR
LAAAVNLIPQITSIYEWKGKICEDSEVLMMIKTQSSLVPALTDFVRSVHPYEVAEVIALP
VEQGNFPYLQWVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYKPVQNKPIVSF
RIVDGEVRDVTSIVPQ
SEQ ID NO: 96
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSMASGSPPTQPSPASDSGSGYVPGSVSAAFVTAPNEKVAKEIARAVVEKR
LAAAVNLIPQITSIYEWKGKIEEDSEVLMMIKTQSSLVPALTDCVRSVHPYEVAEVIALP
VEQGNFPYLQWVRQVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYKPVQNKPIVSF
RIVDGEVRDVTSIVPQ
SEQ ID NO: 97
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSMASGSPPTQPSPASDSGSGYVPGSVSAAFVTAPNEKVAKEIARAVVEKR
LAAAVNLIPQITSIYEWKGKIEEDSEVLMMIKTQSSLVPALTDFVRSVHPYEVAEVIALP
VEQGNFPYLQWVRCVTESVSDSITVLPGGGGSKLGEIEFIKVDKTDKKPLRGAVFSLQKQ
HPDYPDIYGAIDQNGTYQDVRTGEDGKLTFTNLSDGKYRLIENSEPPGYKPVQNKPIVSF
RIVDGEVRDVTSIVPQ
SEQ ID NO: 98
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSGGGGSGGGGSPANLKALEAQKQKEQRQAAEELANAKKLKEQLEKGGGGS
GGGGSGGGGSKLGEIEFIKVDKTDKKPLRGAVFSLQKQHPDYPDIYGAIDQNGTYQDVRT
GEDGKLTFTNLSDGKYRLIENSEPPGYKPVQNKPIVSFRIVDGEVRDVTSIVPQ
SEQ ID NO: 99
MGSSHHHHHHSSGVTTLSGLSGEQGPSGDMTTEEDSATHIKFSKRDEDGRELAGATMELR
DSSGKTISTWISDGHVKDFYLYPGKYTFVETAAPDGYEVATPIEFTVNEDGQVTVDGEAT
EGDAHTGGGGSMASGSPPTQPSPASDSGSGYVPGSVSAAFVTVPNEKVAKEIARAVVEKR
LAASVNLIPQITSIYEWKGKIEEDSEVLMMIKTQSSLVPALTDFVRSVHPYEVAEVIALP
VEQGNFPYLQWVRQVTESVGGGGSGGGGSGGGGSGSGYVPGSVSAAFVTVPNEKVAKEIA
RAVVEKRLAASVNLIPQITSIYEWKGKIEEDSEVLMMIKTQSSLVPALTDFVRSVHPYEV
AEVIALPVEQGNFPYLQWVRQVTESVGGGGSGGGGSGGGGSGSGYVPGSVSAAFVTVPNE
KVAKEIARAVVEKRLAASVNLIPQITSIYEWKGKIEEDSEVLMMIKTQSSLVPALTDFVR
SVHPYEVAEVIALPVEQGNFPYLQWVRQVTESVSDSITVLPGGGGSKLGEIEFIKVDKTD
KKPLRGAVFSLQKQHPDYPDIYGAIDQNGTYQDVRTGEDGKLTFTNLSDGKYRLIENSEP
PGYKPVQNKPIVSFRIVDGEVRDVTSIVPQ
SEQ ID NO: 100
MSGGRAPAVLLGGVASLLLSFVWMPALLPVASRLLLLPRVLLTMASGSPPTQPSPASDSG
SGYVPGSVSAAFVTVPNEKVAKEIARAVVEKRLAASVNLIPQITSIYEWKGKIEEDSEVL
MMIKTQSSLVPALTDFVRSVHPYEVAEVIALPVEQGNFPYLQWVRQVTESVSDSITVLP
SEQ ID NO: 101
MASGSPPTQPSPASDSGSGYVPGSVSAAFVTVPNEKVAKEIARAVVEKRLAASVNLIPQI
TSIYEWKGKIEEDSEVLMMIKTQSSLVPALTDFVRSVHPYEVAEVIALPVEQGNFPYLQW
VRQVTESVGGGGSGGGGSGGGGSGSGYVPGSVSAAFVTVPNEKVAKEIARAVVEKRLAAS
VNLIPQITSIYEWKGKIEEDSEVLMMIKTQSSLVPALTDFVRSVHPYEVAEVIALPVEQG
NFPYLQWVRQVTESVGGGGSGGGGSGGGGSGSGYVPGSVSAAFVTVPNEKVAKEIARAVV
EKRLAASVNLIPQITSIYEWKGKIEEDSEVLMMIKTQSSLVPALTDFVRSVHPYEVAEVI
ALPVEQGNFPYLQWVRQVTESVSDSITVLP
SEQ ID NO: 102
GSGYVPGSVSAAFVTAPNEKVAKEIARAVVEKRLAAAVNLIPQITSIYEWKGKIEEDSEV
LMMIKTQSSLVPALTDFVRSVHPYEVAEVIALPVEQGNFPYLQWVRQVTESVGGGGSGGG
GSGGGGSGGGGSGGGGSGGGGSGGGGSGGGGSEVQLVESGGGSVQAGDSLRLSCAASGRT
FGSIRVGWFRQTPGKEREFVAAINRNDGTTYYADSVKGRFTISRDNAKNTVYMQMASLKP
EDTAVYYCAAGLQYNRSADRVPVGAVYWGQGTQVTVSSAAAGGGGSHHHHHH
SEQ ID NO: 103
GGGGSGGGGSGGGGSGGGGSGGGGS
SEQ ID NO: 104
MGSSHHHHHHSSGMSPILGYWKIKGLVQPTRLLLEYLEEKYEEHLYERDEGDKWRNKKFE
LGLEFPNLPYYIDGDVKLTQSMAIIRYIADKHNMLGGCPKERAEISMLEGAVLDIRYGVS
RIAYSKDFETLKVDFLSKLPEMLKMFEDRLCHKTYLNGDHVTHPDFMLYDALDVVLYMDP
MCLDAFPKLVCFKKRIEAIPQIDKYLKSSKYIAWPLQGWQATFGGGDHPPKGGGGSGGGG
SGGGGSVTTLSGLSGEQGPSGDMTTEEDSATHIKFSKRDEDGRELAGATMELRDSSGKTI
STWISDGHVKDFYLYPGKYTFVETAAPDGYEVATPIEFTVNEDGQVTVDGEATEGDAHT
SEQ ID NO: 105
MGSSHHHHHHSSGMSPILGYWKIKGLVQPTRLLLEYLEEKYEEHLYERDEGDKWRNKKFE
LGLEFPNLPYYIDGDVKLTQSMAIIRYIADKHNMLGGCPKERAEISMLEGAVLDIRYGVS
RIAYSKDFETLKVDFLSKLPEMLKMFEDRLCHKTYLNGDHVTHPDFMLYDALDVVLYMDP
MCLDAFPKLVCFKKRIEAIPQIDKYLKSSKYIAWPLQGWQATFGGGDHPPKGGGGSGGGG
SGGGGSKLGEIEFIKVDKTDKKPLRGAVFSLQKQHPDYPDIYGAIDQNGTYQDVRTGEDG
KLTFTNLSDGKYRLIENSEPPGYKPVQNKPIVSFRIVDGEVRDVTSIVPQ
SEQ ID NO: 106
VQAGDSLRLSCAASGRTFGSIRVGWFRQTPGKEREFVAAINRNDGTTYYADSVKGRFTIS
RDNAKNTVYMQMASLKPEDTAVYYCAAGLQYNRSADRVPVGAVYWGQGTQVTVSS

It is understood that the Examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

1. A polypeptide comprising a first binding domain at the N terminus and a second binding domain at the C terminus, wherein the first and second binding domains are separated by a CutA1 structural domain that contains one or more substitutions or deletions relative to wild-type CutA1, and wherein the first and second binding domains are the same or are different.

2. A polypeptide according to claim 1 or claim 2, wherein the CutA1 structural domain is human CutA1.

3. A polypeptide according to claim 1 or claim 2, wherein the substitution is of one or more cysteine residues, or wherein the deletion is of one or more residues at the N terminus and/or the C-terminus.

4. A polypeptide according to any of claims 1 to 3, wherein one or more cysteine residues in CutA1 are substituted with one or more Alanine, Valine or Serine residues.

5. A polypeptide according to any of claims 1 to 4, wherein the substitution comprises or consists of two cysteines substituted with two alanines.

6. A polypeptide according to any of claims 1 to 4, wherein the substitution comprises or consists of one cysteine substituted with a valine and one cysteine substituted with a serine.

7. A polypeptide according to any of claims 1 to 6, wherein cysteine residues at positions 75 and 96 of wild-type human CutA1 (SEQ ID NO: 19) are substituted for different residues.

8. A polypeptide according to claim 7, wherein the cysteine substitutions comprise or consist of (i) C75A, C96A or (ii) C75V, C96S.

9. A polypeptide according to any preceding claim, wherein the CutA1 structural domain comprises a cysteine residue at a position that is not a cysteine residue in the wild-type sequence.

10. A polypeptide according to claim 9, wherein one or more of the following residues of wild-type human CutA1 (SEQ ID NO: 19) are substituted for cysteine:
V64, E78, K79, K82, E83, K91, Q102, K110, E114, F136, S139, F158 and Q166.

11. A polypeptide according to any preceding claim, wherein 5 to 60 residues are deleted from the N terminus of CutA1, optionally wherein 10 to 59 residues are deleted.

12. A polypeptide according to any preceding claim, wherein 25, 26, 27, 28, 29, 30, 31, 32, 33, 34, 35, 36, 37, 38, 39, 40, 41, 42, 43, 44 or 45 residues are deleted from the N terminus of CutA1.

13. A polypeptide according to any preceding claim, wherein CutA1 is truncated human CutA1 beginning at residue 33 of SEQ ID NO: 19, or beginning at residue 44 of SEQ ID NO: 19, or beginning at residue 60 of SEQ ID NO: 19, or beginning at residue 24 of SEQ ID NO: 19.

14. A polypeptide according to any preceding claim, wherein 5 to 20 residues are deleted from the C terminus of CutA1, optionally wherein 6 to 12 residues are deleted.

15. A polypeptide according to any preceding claim, wherein the CutA1 is truncated CutA1 starting at any of residues 30 to 67 of SEQ ID NO: 19 and ending at any of residues 165 to 179.

16. A polypeptide according to any preceding claim, wherein the CutA1 is truncated CutA1 consisting of residues 44-179 or residues 60-171 of SEQ ID NO: 19.

17. A polypeptide according to any preceding claim, wherein:
the first binding domain and second binding domain are different antigen-binding domains, optionally wherein one or both antigen-binding domain is an antigen-binding fragment of an antibody, optionally an scFv or a Fab, or is a single domain antibody (sdAb), or other antibody mimetics or scaffolds selected to bind specific targets, or other proteins or peptides capable of specific binding with a biological molecule; and/or
wherein one or both of the first binding domain and the second binding domain is an agonist of a TNF Receptor superfamily member, optionally wherein the TNF Receptor superfamily member is a TRAIL receptor for example Death Receptor 5 (DR5).

18. A polypeptide according to any of claims 1 to 16, wherein the first binding domain and second binding domain are catcher domains each able to form an isopeptide linkage with a cognate peptide,
optionally wherein the cognate peptide for the first binding domain is different from the cognate peptide for the second binding domain, or
optionally wherein the cognate peptide for the first binding domain is the same as for the second binding domain, optionally wherein selective binding or conjugation is achieved by temporal or sequential control such as activation or inactivation of a second binding domain and/or by means of competitive binding.

19. A polypeptide according to claim 18 wherein each cognate peptide is attached to an antigen-binding domain, optionally wherein one or both cognate peptides are linked to the first and/or second catcher domain by an isopeptide bond.

20. A polypeptide according claim 18, wherein one or both of the cognate peptides is connected to an agonist of a TNF Receptor superfamily member, optionally wherein the TNF Receptor superfamily member is a TRAIL receptor for example Death Receptor 5 (DR5).

21. A polypeptide according to any of claims 1 to 20, wherein the first binding domain and second binding domain are able to bind to their targets when expressed on a single cell or immobilised onto a plate or single bead.

22. A polypeptide according to any preceding claim, comprising an effector molecule, optionally a drug molecule or dye molecule, optionally wherein the drug molecule comprises or consists of:
(a) an anticancer drug, optionally a cytotoxic drug;
(b) a tubulin inhibitor, optionally a maytansinoid, auristatin, or taxol derivative;
(c) monomethyl auristatin E (MMAE) or monomethyl auristatin F (MMAF);
(d) a compound derived from dolastatin 10;
(e) a tubulysin such as Tubulysin A;
(f) a DNA damaging agent;
(g) a duocarmycin;
(h) a calicheamicin;
(i) a pyrrolobenzodiazepine;
(j) SN-38 (active metabolite of irinotecan);
(k) an immunomodulator such as a TLR agonist or a STING agonist;
(l) deruxtecan;
(m) mertansine; or
(n) an immunotoxin, optionally Pseudomonas exotoxin A (PE).

23. A polypeptide according to claim 22, wherein the drug molecule or dye molecule is chemically conjugated to the polypeptide, optionally in the CutA1 domain and optionally to a cysteine residue in the CutA1 structural domain, optionally wherein the conjugated cysteine residue is not present in the native CutA1 sequence.

24. An oligomer comprising two or more polypeptides according to any preceding claim.

25. A polypeptide according to any of claims 1 to 23 or an oligomer according to claim 24, wherein the polypeptide or oligomer comprises the features of any of embodiments A1 to A26.

26. A polypeptide comprising:
a first binding domain at the N terminus and/or a second binding domain at the C terminus of a CutA1 structural domain or a cytokine structural domain, wherein when both the first and second binding domains are present they are separated by the CutA1 structural domain or a cytokine structural domain, optionally wherein the CutA1 structural domain or cytokine structural domain contains one or more substitutions or deletions relative to the respective wild-type CutA1 or cytokine, and wherein the first and second binding domains are the same or are different; or
a first binding domain connected to a second binding domain, wherein the second binding domain is connected to a CutA1 structural domain or a cytokine structural domain, optionally wherein the CutA1 structural domain or cytokine structural domain contains one or more substitutions or deletions relative to the respective wild-type CutA1 or cytokine, and wherein the first and second binding domains are the same or are different.

27. A polypeptide according to claim 26, wherein the cytokine is TNF, TL1A, OX40L or CD40L, SEQ ID NO: 80 or a modified version thereof, SEQ ID NO:31 or a modified version thereof, SEQ ID NO:78 or a modified version thereof, or SEQ ID NO:58 or a modified version thereof.

28. A CutA1 protein comprising one or more substitutions or deletions relative to wild-type CutA1, optionally connected by a peptide linkage as a fusion protein to a different polypeptide at the N-terminus and/or the C terminus of the CutA1 protein.

29. A CutA1 protein according to claim 28, wherein at least one native cysteine residue is substituted for a different amino acid residue, and/or wherein at least one native residue that it not cysteine is substituted for a cysteine residue.

30. A single polypeptide chain comprising multiple CutA1 sequences, optionally wherein a linker polypeptide between 3 and 30 amino acids in length lies between some or all of the CutA1 sequences, optionally wherein at least one and optionally all of the CutA1 sequences are is as defined in any of claims 1 to 16.

31. A single polypeptide according to claim 30, comprising three human CutA1 sequences each separated by a linker polypeptide, optionally comprising an isopeptide bond-forming domain at the N and/or C terminus.


SEQUENCE LISTING (143 sequences)

SEQ ID NO: 1 - protein, 102 aa, Pyrococcus horikoshii
MIIVYTTFPDWESAEKVVKTLLKERLIACANLREHRAFYWWEGKIEEDKEVGAILKTREDLWEELKERIKELHPYDVPAIIRIDVDDVNEDYLKWLIEETKK

SEQ ID NO: 2 - protein, 134 aa, Homo sapiens
TGMPVSAFTVILSKAYPAIGTPIPFDKILYNRQQHYDPRTGIFTCQIPGIYYFSYHVHVKGTHVWVGLYKNGTPVMYTYDEYTKGYLDQASGSAIIDLTENDQVWLQLPNAESNGLYSSEYVHSSFSGFLVAPM

SEQ ID NO: 3 - protein, 131 aa, Mus musculus [sequence omitted]

SEQ ID NO: 4 - protein, 113 aa, synthetic construct
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 5 - protein, 13 aa, synthetic construct
AHIVMVDAYKPTK

SEQ ID NO: 6 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 7 - protein, 14 aa, synthetic construct
VPTIVMVDAYKRYK

SEQ ID NO: 8 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 9 - protein, 16 aa, synthetic construct
RGVPHIVMVDAYKRYK

SEQ ID NO: 10 - protein, 95 aa, synthetic construct
GQSGDGKELAGATMELRDSSGKTISTWISDGQVKDFYLYPGKYTFVETAAPDGYEVATAITFTVNEQGQVTVNGKATKGGSGGSGGSGEDSATHI

SEQ ID NO: 11 - protein, 10 aa, synthetic construct
ATHIKFSKRD

SEQ ID NO: 12 - protein, 112 aa, synthetic construct
KPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 13 - protein, 12 aa, synthetic construct
KLGDIEFIKVNK

SEQ ID NO: 14 - protein, 104 aa, synthetic construct
VNKNDKKPLRGAVFSLQKQHPDYPDIYGAIDQNGTYQNVRTGEDGKLTFKNLSDGKYRLFENSEPPGYKPVQNKPIVAFQIVNGEVRDVTSIVPPGVPATYEFT

SEQ ID NO: 15 - protein, 12 aa, synthetic construct
KLGSIEFIKVNK

SEQ ID NO: 16 - protein, 23 aa, synthetic construct
DIPATYEFTDGKHYITNEPIPPK

SEQ ID NO: 17 - protein, 282 aa, synthetic construct
ATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTI

SEQ ID NO: 18 - protein, 16 aa, synthetic construct
TDKDMTITFTNKKDAE

SEQ ID NO: 19 - protein, 179 aa, Homo sapiens
MSGGRAPAVLLGGVASLLLSFVWMPALLPVASRLLLLPRVLLTMASGSPPTQPSPASDSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVSDSITVLP

SEQ ID NO: 20 - protein, 380 aa, synthetic construct
MGSSHHHHHHSSGVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGSGSTGMPVSAFTVILSKAYPAIGTPIPFDKILYNRQQHYDPRTGIFTCQIPGIYYFSYHVHVKGTHVWVGLYKNGTPVMYTYDEYTKGYLDQASGSAIIDLTENDQVWLQLPNAESNGLYSSEYVHSSFSGFLVAPMGSGSKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 21 - protein, 348 aa, synthetic construct
MGSSHHHHHHSSGVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGSGSMIIVYTTFPDWESAEKVVKTLLKERLIACANLREHRAFYWWEGKIEEDKEVGAILKTREDLWEELKERIKELHPYDVPAIIRIDVDDVNEDYLKWLIEETKKGSGSKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 22 - protein, 280 aa, synthetic construct
MGSSHHHHHHSSGVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGSGSPANLKALEAQKQKEQRQAAEELANAKKLKEQLEKGSGSKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 23 - protein, 104 aa, synthetic construct
KLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 24 - protein, 376 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSMASGSPPTQPSPASDSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVSDSITVLPGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 25 - protein, 114 aa, Homo sapiens
PMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 26 - protein, 117 aa, Homo sapiens
PFLELDTNLPANRVPAGLEKRLCAAAASILGKPADRVNVTVRPGLAMALSGSTEPCAQLSISSIGVVGTAEDNRSHSAHFFEFLTKELALGQDRILIRFFPLESWQIGKIGTVMTFL

SEQ ID NO: 27 - protein, 117 aa, synthetic construct
PFLELDTNLPANRVPAGLEKRLCAAAASILGKPADRVNVTVRPGLAMALSGSTEPCAQLSIASIGVVGTAEDNRSHSAHFFEFLTKELALGQDRILIRFAPLESWQIGKIGTVMTFL

SEQ ID NO: 28 - protein, 377 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSGGGGSGGGGSPFLELDTNLPANRVPAGLEKRLCAAAASILGKPADRVNVTVRPGLAMALSGSTEPCAQLSIASIGVVGTAEDNRSHSAHFFEFLTKELALGQDRILIRFAPLESWQIGKIGTVMTFLGGGGSGGGGSGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 29 - protein, 136 aa, synthetic construct
MASGSPPTQPSPASDSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVSDSITVLP

SEQ ID NO: 30 - protein, 295 aa, synthetic construct
MSHHHHHHGSTGLEVLFQGPTGSSDIPATYEFTDGKHYITNEPIPPKGGGGSGGGGSVSKGEELFTGVVPILVELDGDVNGHKFSVSGEGEGDATYGKLELKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYDYNSHNVYIMADKQKNGIKVNFKIRHNIEDGSVQLADHYQQNTPIGDGPVLLPDNHYLEYQSALSKDPNEKRDHMVLAEFVTAEGITLGMDELYK

SEQ ID NO: 31 - protein, 164 aa, Homo sapiens
LRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 32 - protein, 389 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSGSLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLLGSGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 33 - protein, 287 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAGSGSVTAFSNMDDMLQKAHLVIEGTFIYLRDSTEFFIRVRDGWKKLQLGELIPIPADSPPPPALSSNPGSGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 34 - protein, 342 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSGSPFLELDTNLPANRVPAGLEKRLCAAAASILGKPADRVNVTVRPGLAMALSGSTEPCAQLSIASIGVVGTAEDNRSHSAHFFEFLTKELALGQDRILIRFAPLESWQIGKIGTVMTFLGSGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 35 - protein, 280 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSGSKKQGDADVCGEVAYIQSVVSDCHVPTAELRTLLEIRKLFLEIQKLKVELQGLSKEGSGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 36 - protein, 133 aa, Homo sapiens
QPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPS

SEQ ID NO: 37 - protein, 132 aa, Homo sapiens
TQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPD

SEQ ID NO: 38 - protein, 129 aa, Homo sapiens
KFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 39 - protein, 103 aa, Thermus thermophilus
MEEVVLITVPSEEVARTIAKALVEERLAACVNIVPGLTSIYRWQGEVVEDQELLLLVKTTTHAFPKLKERVKALHPYTVPEIVALPIAEGNREYLDWLRENTG

SEQ ID NO: 40 - protein, 107 aa, Oryza sativa
STTVPSIVVYVTVPNKEAGKRLAGSIISEKLAACVNIVPGIESVYWWEGKVQTDAEELLIIKTRESLLDALTEHVKANHEYDVPEVIALPIKGGNLKYLEWLKNSTR

SEQ ID NO: 41 - protein, 106 aa, Shewanella sp.
KPEQLLIFTTCPDADIACRIATALVEAKLAACVQIGQAVESIYQWDNNICQSHEVPMQIKCMTTDYPAIEQLVITMHPYEVPEFIATPIIGGFGPYLQWIKDNSPS

SEQ ID NO: 42 - protein, 152 aa, Homo sapiens
RTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLLFAESGQVYFGIIAL

SEQ ID NO: 43 - protein, 28 aa, synthetic construct
ELAQAFKEIAKAFKEIAKAFEFIAQAIE

SEQ ID NO: 44 - protein, 61 aa, Human immunodeficiency virus 1
IVQQQNNLLRAIEAQQHLLQLTVWAIKQLQARSGGRGGWMEWDREINNYTSLIHSLIEESQ

SEQ ID NO: 45 - protein, 96 aa, synthetic construct
VDPAKEAIMKPQLTMLKGLSDAELKALADFILRIAKQAQEKQQQDVAKAIFQQKGCGSCHQANVDTVGPSLAKIAQAYAGKEDQLIKFLKGEAPAI

SEQ ID NO: 46 - protein, 75 aa, Homo sapiens
YGNMTEDHVMHLLQNADPLKVYPPLKGSFPENLRHLKNTMETIDWKVFESWMHHWLLFEMSRHSLEQKPTDAPPK

SEQ ID NO: 47 - protein, 32 aa, Homo sapiens
GEYFTLQIRGRERFEMFRELNEALELKDAQAG

SEQ ID NO: 48 - protein, 220 aa, Homo sapiens
SRPRDCLDVLLSGQQDDGVYSVFPTHYPAGFQVYCDMRTDGGGWTVFQRREDGSVNFFRGWDAYRDGFGRLTGEHWLGLKRIHALTTQAAYELHVDLEDFENGTAYARYGSFGVGLFSVDPEEDGYPLTVADYSGTAGDSLLKHSGMRFTTKDRDSDHSENNCAAFYRGAWWYRNCHTSNLNGQYLRGAHASYADGVEWSSWTGWQYSLKFSEMKIRPVR

SEQ ID NO: 49 - protein, 228 aa, Homo sapiens
VDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 50 - protein, 52 aa, Bacillus subtilis
FMKSTGIVRKVDELGRVVIPIELRRTLGIAEKDALEIYVDDEKIILKKYKPN

SEQ ID NO: 51 - protein, 95 aa, synthetic construct [sequence omitted]

SEQ ID NO: 52 - protein, 133 aa, Homo sapiens
TRDFNGTWEMESNENFEGYMKALDIDFATRKIAVRLTFTDVIDQDGDNFKTKATSTFLNYDEDFTVGVEFDEYTKSLDNRHVKALVTWEGDVLVCVQKGEKENRGWKKWIEGDKLYLELTCGDQVCRQVFKKK

SEQ ID NO: 53 - protein, 162 aa, Homo sapiens
LPTYRYPLELDTANNRVQVADRFGMRTGTWTGQLQYQHPQLSWRANVTLNLMKVDDWLVLSFSQMTTNSIMADGKFVINFVSGLSSGWQTGDTEPSSTIDPLSTTFAAVQFLNNGQRIDAFRIMGVSEWTDGELEIKNYGGTYTGHTQVYWAPWTIMYPCNV

SEQ ID NO: 54 - protein, 361 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSGSMASGSPPTQPSPASDSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVSDSITVLPGSGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 55 - protein, 327 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSGSMIIVYTTFPDWESAEKVVKTLLKERLIACANLREHRAFYWWEGKIEEDKEVGAILKTREDLWEELKERIKELHPYDVPAIIRIDVDDVNEDYLKWLIEETKKGSGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 56 - protein, 359 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSGSTGMPVSAFTVILSKAYPAIGTPIPFDKILYNRQQHYDPRTGIFTCQIPGIYYFSYHVHVKGTHVWVGLYKNGTPVMYTYDEYTKGYLDQASGSAIIDLTENDQVWLQLPNAESNGLYSSEYVHSSFSGFLVAPMGSGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 57 - protein, 377 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSGSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLLFAESGQVYFGIIALGSGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 58 - protein, 141 aa, synthetic construct
QIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 59 - protein, 146 aa, synthetic construct
MKDEVALLAAVTLLGVLLQAYFSLQVISARRAFRVSPPLTTGPPEFERVYRAQVNCSEYFPLFLATLWVAGIFFHEGAAALCGLVYLFARLRYFQGYARSAQLRLAPLYASARALWLLVALAALGLLAHFLPAALRAALLGRLRTL

SEQ ID NO: 60 - protein, 108 aa, synthetic construct
SGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVT

SEQ ID NO: 61 - protein, 5 aa, synthetic construct
GGGGS

SEQ ID NO: 62 - protein, 15 aa, synthetic construct
GGGGSGGGGSGGGGS

SEQ ID NO: 63 - protein, 20 aa, synthetic construct
GGGGSGGGGSGGGGSGGGGS

SEQ ID NO: 64 - protein, 35 aa, synthetic construct
EAAAKEAAAKEAAAKEAAAKEAAAKEAAAKEAAAK

SEQ ID NO: 65 - protein, 34 aa, synthetic construct
PANLKALEAQKQKEQRQAAEELANAKKLKEQLEK

SEQ ID NO: 66 - protein, 112 aa, synthetic construct
GSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESV

SEQ ID NO: 67 - protein, 136 aa, synthetic construct
MASGSPPTQPSPASDSGSGYVPGSVSAAFVTAPNEKVAKEIARAVVEKRLAAAVNLIPQITSIYEWKGKIEEDSEVLMMIKTQSSLVPALTDFVRSVHPYEVAEVIALPVEQGNFPYLQWVRQVTESVSDSITVLP

SEQ ID NO: 68 - protein, 136 aa, synthetic construct
MASGSPPTQPSPASDSGSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESVSDSITVLP

SEQ ID NO: 69 - protein, 112 aa, synthetic construct
GSGYVPGSVSAAFVTAPNEKVAKEIARAVVEKRLAAAVNLIPQITSIYEWKGKIEEDSEVLMMIKTQSSLVPALTDFVRSVHPYEVAEVIALPVEQGNFPYLQWVRQVTESV

SEQ ID NO: 70 - protein, 112 aa, synthetic construct
GSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESV

SEQ ID NO: 71 - protein, 396 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSGGGGSGGGGSMASGSPPTQPSPASDSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVSDSITVLPGGGGSGGGGSGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 72 - protein, 396 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSGGGGSGGGGSMASGSPPTQPSPASDSGSGYVPGSVSAAFVTAPNEKVAKEIARAVVEKRLAAAVNLIPQITSIYEWKGKIEEDSEVLMMIKTQSSLVPALTDFVRSVHPYEVAEVIALPVEQGNFPYLQWVRQVTESVSDSITVLPGGGGSGGGGSGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 73 - protein, 396 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSGGGGSGGGGSMASGSPPTQPSPASDSGSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESVSDSITVLPGGGGSGGGGSGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 74 - protein, 372 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSGGGGSGGGGSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVGGGGSGGGGSGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 75 - protein, 372 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSGGGGSGGGGSGSGYVPGSVSAAFVTAPNEKVAKEIARAVVEKRLAAAVNLIPQITSIYEWKGKIEEDSEVLMMIKTQSSLVPALTDFVRSVHPYEVAEVIALPVEQGNFPYLQWVRQVTESVGGGGSGGGGSGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 76 - protein, 372 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSGGGGSGGGGSGSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESVGGGGSGGGGSGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 77 - protein, 105 aa, synthetic construct
SVSAAFVTAPNEKVAKEIARAVVEKRLAAAVNLIPQITSIYEWKGKIEEDSEVLMMIKTQSSLVPALTDFVRSVHPYEVAEVIALPVEQGNFPYLQWVRQVTESV

SEQ ID NO: 78 - protein, 126 aa, synthetic construct
RIQSIKVQFTEYKKEKGFILTSQKEDEIMKVQDNSVIINCDGFYLISLKGYFSQEVDISLHYQKDEEPLFQLKKVRSVNSLMVASLTYKDKVYLNVTTDNTSLDDFHVNGGELILIHQNPGEFCVL

SEQ ID NO: 79 - protein, 142 aa, synthetic construct
PQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 80 - protein, 157 aa, synthetic construct
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLLFAESGQVYFGIIAL

SEQ ID NO: 81 - protein, 112 aa, Synechococcus elongatus
MKKIEAIIRPFKLDEVKIALVNAGIVGMTVSEVRGFGRQKGQTERYRGAEYTVEFLQKLKLEIVVEDAQVDTVIDKIVAAARTGEIGDGKIFVSPVDQTIRIRTGEKNADAI

SEQ ID NO: 82 - protein, 112 aa, Escherichia coli
MKLVTVIIKPFKLEDVREALSSIGIQGLTVTEVKGFGRQKGHAELYRGAEYSVNFLPKVKIDVAIADDQLDEVIDIVSKAAYTGKIGDGKIFVAELQRVIRIRTGEADEAAL

SEQ ID NO: 83 - protein, 106 aa, Bacillus cereus
EEMKKVVVFVPVTHAEEVRKALGDAGAGHIGNYSHCTFSSEGTGTFVPQEGTNPYIGETGQLERVEEVRIETIIPASLQRKVIKAMVTAHPYEEVAYDVYPLDNKG

SEQ ID NO: 84 - protein, 111 aa, Staphylococcus aureus
SIINNQQDVYYKVQTYIPKDNVGPFKDKLSENGLAQEGNYEYCFFESEGRGQFKPVGEANPTIGQIDKIEDVDEVKIEFMIDAYQKSRAEQLIKQYHPYETPVFDFIEIKQ

SEQ ID NO: 85 - protein, 136 aa, synthetic construct
MASGSPPTQPSPASDSGSGYVPGSVSAAFVTAPNCKVAKEIARAVVEKRLAAAVNLIPQITSIYEWKGKIEEDSEVLMMIKTQSSLVPALTDFVRSVHPYEVAEVIALPVEQGNFPYLQWVRQVTESVSDSITVLP

SEQ ID NO: 86 - protein, 136 aa, synthetic construct
MASGSPPTQPSPASDSGSGYVPGSVSAAFVTAPNEKVACEIARAVVEKRLAAAVNLIPQITSIYEWKGKIEEDSEVLMMIKTQSSLVPALTDFVRSVHPYEVAEVIALPVEQGNFPYLQWVRQVTESVSDSITVLP

SEQ ID NO: 87 - protein, 136 aa, synthetic construct
MASGSPPTQPSPASDSGSGYVPGSVSAAFVTAPNEKVAKEIARAVVEKRLAAAVNLIPCITSIYEWKGKIEEDSEVLMMIKTQSSLVPALTDFVRSVHPYEVAEVIALPVEQGNFPYLQWVRQVTESVSDSITVLP

SEQ ID NO: 88 - protein, 136 aa, synthetic construct
MASGSPPTQPSPASDSGSGYVPGSVSAAFVTAPNEKVAKEIARAVVEKRLAAAVNLIPQITSIYEWKGKICEDSEVLMMIKTQSSLVPALTDFVRSVHPYEVAEVIALPVEQGNFPYLQWVRQVTESVSDSITVLP

SEQ ID NO: 89 - protein, 136 aa, synthetic construct
MASGSPPTQPSPASDSGSGYVPGSVSAAFVTAPNEKVAKEIARAVVEKRLAAAVNLIPQITSIYEWKGKIEEDSEVLMMIKTQSSLVPALTDCVRSVHPYEVAEVIALPVEQGNFPYLQWVRQVTESVSDSITVLP

SEQ ID NO: 90 - protein, 136 aa, synthetic construct
MASGSPPTQPSPASDSGSGYVPGSVSAAFVTAPNEKVAKEIARAVVEKRLAAAVNLIPQITSIYEWKGKIEEDSEVLMMIKTQSSLVPALTDFVRSVHPYEVAEVIALPVEQGNFPYLQWVRCVTESVSDSITVLP

SEQ ID NO: 91 - protein, 376 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSMASGSPPTQPSPASDSGSGYVPGSVSAAFVTAPNEKVAKEIARAVVEKRLAAAVNLIPQITSIYEWKGKIEEDSEVLMMIKTQSSLVPALTDFVRSVHPYEVAEVIALPVEQGNFPYLQWVRQVTESVSDSITVLPGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 92 - protein, 376 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSMASGSPPTQPSPASDSGSGYVPGSVSAAFVTAPNCKVAKEIARAVVEKRLAAAVNLIPQITSIYEWKGKIEEDSEVLMMIKTQSSLVPALTDFVRSVHPYEVAEVIALPVEQGNFPYLQWVRQVTESVSDSITVLPGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 93 - protein, 376 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSMASGSPPTQPSPASDSGSGYVPGSVSAAFVTAPNEKVACEIARAVVEKRLAAAVNLIPQITSIYEWKGKIEEDSEVLMMIKTQSSLVPALTDFVRSVHPYEVAEVIALPVEQGNFPYLQWVRQVTESVSDSITVLPGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 94 - protein, 376 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSMASGSPPTQPSPASDSGSGYVPGSVSAAFVTAPNEKVAKEIARAVVEKRLAAAVNLIPCITSIYEWKGKIEEDSEVLMMIKTQSSLVPALTDFVRSVHPYEVAEVIALPVEQGNFPYLQWVRQVTESVSDSITVLPGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 95 - protein, 376 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSMASGSPPTQPSPASDSGSGYVPGSVSAAFVTAPNEKVAKEIARAVVEKRLAAAVNLIPQITSIYEWKGKICEDSEVLMMIKTQSSLVPALTDFVRSVHPYEVAEVIALPVEQGNFPYLQWVRQVTESVSDSITVLPGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 96 - protein, 376 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSMASGSPPTQPSPASDSGSGYVPGSVSAAFVTAPNEKVAKEIARAVVEKRLAAAVNLIPQITSIYEWKGKIEEDSEVLMMIKTQSSLVPALTDCVRSVHPYEVAEVIALPVEQGNFPYLQWVRQVTESVSDSITVLPGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 97 - protein, 376 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSMASGSPPTQPSPASDSGSGYVPGSVSAAFVTAPNEKVAKEIARAVVEKRLAAAVNLIPQITSIYEWKGKIEEDSEVLMMIKTQSSLVPALTDFVRSVHPYEVAEVIALPVEQGNFPYLQWVRCVTESVSDSITVLPGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 98 - protein, 294 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSGGGGSGGGGSPANLKALEAQKQKEQRQAAEELANAKKLKEQLEKGGGGSGGGGSGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 99 - protein, 630 aa, synthetic construct
MGSSHHHHHHSSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSMASGSPPTQPSPASDSGSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESVGGGGSGGGGSGGGGSGSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESVGGGGSGGGGSGGGGSGSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESVSDSITVLPGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 100 - protein, 179 aa, synthetic construct
MSGGRAPAVLLGGVASLLLSFVWMPALLPVASRLLLLPRVLLTMASGSPPTQPSPASDSGSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESVSDSITVLP

SEQ ID NO: 101 - protein, 390 aa, synthetic construct
MASGSPPTQPSPASDSGSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESVGGGGSGGGGSGGGGSGSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESVGGGGSGGGGSGGGGSGSGYVPGSVSAAFVTVPNEKVAKEIARAVVEKRLAASVNLIPQITSIYEWKGKIEEDSEVLMMIKTQSSLVPALTDFVRSVHPYEVAEVIALPVEQGNFPYLQWVRQVTESVSDSITVLP

SEQ ID NO: 102 - protein, 292 aa, synthetic construct
GSGYVPGSVSAAFVTAPNEKVAKEIARAVVEKRLAAAVNLIPQITSIYEWKGKIEEDSEVLMMIKTQSSLVPALTDFVRSVHPYEVAEVIALPVEQGNFPYLQWVRQVTESVGGGGSGGGGSGGGGSGGGGSGGGGSGGGGSGGGGSGGGGSEVQLVESGGGSVQAGDSLRLSCAASGRTFGSIRVGWFRQTPGKEREFVAAINRNDGTTYYADSVKGRFTISRDNAKNTVYMQMASLKPEDTAVYYCAAGLQYNRSADRVPVGAVYWGQGTQVTVSSAAAGGGGSHHHHHH

SEQ ID NO: 103 - protein, 25 aa, synthetic construct
GGGGSGGGGSGGGGSGGGGSGGGGS

SEQ ID NO: 104 - protein, 359 aa, synthetic construct
MGSSHHHHHHSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGGGGSGGGGSGGGGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 105 - protein, 350 aa, synthetic construct
MGSSHHHHHHSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGGGGSGGGGSGGGGSKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 106 - protein, 115 aa, synthetic construct
VQAGDSLRLSCAASGRTFGSIRVGWFRQTPGKEREFVAAINRNDGTTYYADSVKGRFTISRDNAKNTVYMQMASLKPEDTAVYYCAAGLQYNRSADRVPVGAVYWGQGTQVTVSS

SEQ ID NO: 107 - protein, 147 aa, synthetic construct
RLLLLPRVLLTMASGSPPTQPSPASDSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVSDSITVLP

SEQ ID NO: 108 - protein, 15 aa, synthetic construct
GGGGSGGGGSGGGGS

SEQ ID NO: 109 - protein, 5 aa, synthetic construct
GGGGS

SEQ ID NO: 110 - protein, 4 aa, synthetic construct
GSGS

SEQ ID NO: 111 - protein, 10 aa, synthetic construct
GGGGSGGGGS

SEQ ID NO: 112 - protein, 5 aa, synthetic construct
EAAAK

SEQ ID NO: 113 - protein, 10 aa, synthetic construct
EAAAKEAAAK

SEQ ID NO: 114 - protein, 15 aa, synthetic construct
EAAAKEAAAKEAAAK

SEQ ID NO: 115 - protein, 25 aa, synthetic construct
EAAAKEAAAKEAAAKEAAAKEAAAK

SEQ ID NO: 116 - protein, 5 aa, synthetic construct
PAPAP

SEQ ID NO: 117 - protein, 17 aa, synthetic construct
VSQTSKLTRAETVFPDV

SEQ ID NO: 118 - protein, 6 aa, synthetic construct
PLGLWA

SEQ ID NO: 119 - protein, 6 aa, synthetic construct
RVLAEA

SEQ ID NO: 120 - protein, 10 aa, synthetic construct
EDVVCCSMSY

SEQ ID NO: 121 - protein, 8 aa, synthetic construct
GGIEGRGS

SEQ ID NO: 122 - protein, 10 aa, synthetic construct
TRHRQPRGWE

SEQ ID NO: 123 - protein, 10 aa, synthetic construct
AGNRVRRSVG

SEQ ID NO: 124 - protein, 5 aa, synthetic construct
RRRRR

SEQ ID NO: 125 - protein, 9 aa, synthetic construct
RRRRRRRRR

SEQ ID NO: 126 - protein, 4 aa, synthetic construct
GFLG

SEQ ID NO: 127 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 128 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 129 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 4 aa, synthetic construct
GGGS

SEQ ID NO: 131 - protein, 4 aa, synthetic construct
PGGS

SEQ ID NO: 132 - protein, 4 aa, synthetic construct
PGGG

SEQ ID NO: 133 - protein, 6 aa, synthetic construct
RPPPPP

SEQ ID NO: 134 - protein, 5 aa, synthetic construct
RPPPP

SEQ ID NO: 135 - protein, 4 aa, synthetic construct
RPPG

SEQ ID NO: 136 - protein, 4 aa, synthetic construct
PPPP

SEQ ID NO: 137 - protein, 9 aa, synthetic construct
PPPPPPPPP

SEQ ID NO: 138 - protein, 12 aa, synthetic construct
PPPPPPPPPPPP

SEQ ID NO: 139 - protein, 4 aa, synthetic construct
SGSG

SEQ ID NO: 140 - protein, 6 aa, synthetic construct
SGSGSG

SEQ ID NO: 141 - protein, 8 aa, synthetic construct
SGSGSGSG

SEQ ID NO: 142 - protein, 10 aa, synthetic construct
SGSGSGSGSG

SEQ ID NO: 143 - protein, 16 aa, synthetic construct
SGSGSGSGSGSGSGSG